(19) United States
(12) Patent Application Publication
(10) Pub. No.: US 2024/0156901 A1
(43) Pub. Date: May 16, 2024

(54) GAL3BP POLYPEPTIDE COMPOSITIONS AND METHODS FOR TREATMENT OF CANCER AND DETERMINING TREATMENT RESPONSIVENESS

(71) Applicant: UNIVERSITY OF PITTSBURGH-OF THE COMMONWEALTH SYSTEM OF HIGHER EDUCATION, Pittsburgh, PA (US)

(72) Inventors: Baoli HU, Pittsburgh, PA (US); Apeng CHEN, Pittsburgh, PA (US); Carlos CAMACHO, Pittsburgh, PA (US)

(21) Appl. No.: 18/281,189

(22) PCT Filed: Mar. 10, 2022
(86) PCT No.: PCT/US2022/019736

§ 371 Date: Sep. 8, 2023

Related U.S. Application Data
(60) Provisional application No. 63/159,128, filed on Mar. 10, 2021.

(51) Int. Cl. A61K 38/17; A61K 39/395; A61P 35/00; C07K 14/47; G01N 33/574

(52) U.S. Cl. CPC A61K 38/1709; A61K 39/3955; A61P 35/00; C07K 14/4703; G01N 33/57488; G01N 2333/4724; G01N 2333/924

(57) ABSTRACT

Disclosed herein are compositions and methods for treating cancers. Also disclosed herein are methods for detecting subjects' responsiveness to immunotherapy and for reversing resistance to immunotherapy.

### CROSS-REFERENCE TO RELATED APPLICATIONS

This application claims the benefit of U.S. Provisional Application No. 63/159,128, filed Mar. 10, 2021, which is expressly incorporated herein by reference in its entirety.

### FIELD

The present disclosure relates to the field of cancer treatment.

### BACKGROUND

Glioblastoma (GBM), the most common and lethal primary brain tumor with a median survival rate of only 15 months, remains incurable despite intensive multimodal treatment of surgical resection, radio-chemotherapy and anti-angiogenic therapy with bevacizumab (Desjardins, 2015; Furnari et al., 2007; Wen and Kesari, 2008). While immunotherapies have been highly effective against some types of cancer, the disappointing results of clinical trials for GBM immunotherapy represent continued challenges (Buerki et al., 2018; Lim et al., 2018). Therefore, effective therapies for patients with GBM are urgently needed. The compositions and methods disclosed herein address these and other needs.

### SUMMARY

Many cancers (such as glioblastoma) are resistant to immunotherapies. How cancer cells induce tumor immunosuppression and escape immunosurveillance remains to be explored. It is shown herein that upregulation of cancer-intrinsic Chitinase-3-like-1 (CHI3L1) signaling modulates an immunosuppressive microenvironment by reprogramming immune cells and non-immune cells (e.g, tumor-associated macrophages (TAMs), T cells, and tumor cells). Galectin-3-binding protein (Gal3BP) can negatively regulate this process by competing with Gal3 to bind CHI3L1. Accordingly, in some aspects, disclosed herein is a composition comprising a Galectin-3 (Gal3)-binding protein (Gal3BP) polypeptide and uses thereof for treating a cancer. In some embodiments, the Gal3BP polypeptide sequence is at least 90% identical to SEQ ID NO: 4 or SEQ ID NO: 5. Administration of the Gal3BP polypeptide disclosed herein surprisingly reverses immune suppression and attenuates tumor progression. Further, administration of the Gal3BP polypeptide disclosed herein surprisingly decreases a level of an immune checkpoint molecule on immune cells (e.g., CD45+ cells such as T cells and macrophages) and non-immune cells (e.g., tumor cells). In some embodiments, the subject is determined to have a) a higher level of a CHI3L1 polypeptide and/or a Gal3 polypeptide compared to a reference control, and/or b) a lower level of a Gal3BP polypeptide compared to a reference control. In some embodiments, the methods further comprise administering to the subject a therapeutically effective amount of an immune checkpoint inhibitor (e.g., a PD-1 inhibitor, a PD-L1 inhibitor, or a CLTA-4 inhibitor). In some embodiments, the immune checkpoint inhibitor is a PD-1 inhibitor. In some embodiments, the immune checkpoint inhibitor is selected from nivolumab, pembrolizumab, cemiplimab, atezolizumab, avelumab, durvalumab, and ipilimumab.

Also disclosed herein is a method of identifying a subject's responsiveness to an immune checkpoint inhibitor, said method comprising
a) obtaining a biological sample from the subject,
b) quantifying a level of a biomarker relative to a reference control, wherein the biomarker is selected from a CHI3L1 polypeptide, a Galectin-3 (Gal3) polypeptide, and a Galectin-3(Gal3)-binding protein (Gal3BP) polypeptide; and
c) determining the subject as responsive to the immune checkpoint therapy when the level of one or more of the CHI3L1 polypeptide or the Gal3 polypeptide is lower in the biological sample than its reference control, or the level of the Gal3BP polypeptide is higher in the biological sample than its reference control, or a combination thereof; and
d) determining the subject as non-responsive to the immune checkpoint therapy when the level of one or more of the CHI3L1 polypeptide or the Gal3 polypeptide is higher in the biological sample than its reference control, or the level of the Gal3BP polypeptide is lower in the biological sample than its reference control, or a combination thereof.

In some embodiments, the method disclosed herein further comprises administering to the subject non-responsive to the immune checkpoint inhibitor a therapeutically effective amount of a Gal3PB polypeptide. In some embodiments, the method disclosed herein further comprises subsequently administering to the subject a therapeutically effective amount of an immune checkpoint inhibitor.

### DESCRIPTION OF DRAWINGS

FIGS. 1A-1O show that CHI3L1 upregulation is associated with activation of the PI3K/AKT/mTOR signaling in GBM. (FIG. 1A) Top 10 upregulated genes in hNSC vs hNSC-p53DN-AKT ranked by fold change of gene expression. qRT-PCR for CHI3L1 expression (FIG. 1B) and immunoblot analysis of indicated proteins (FIG. 1C) in hNSC expressing p53DN or/and myr-AKT. (FIG. 1D) Representative H&E and IHC images showing indicated proteins in tumors derived from hNSCs-p53DN-AKT. Scale bar, 50 μm. qRT-PCR (FIG. 1E) and immunoblot analysis (FIG. 1F) of indicated gene and proteins in hNSC-p53DN-AKT with rapamycin (RAPA) treatment (100 nM, 24 hours); CHI3L1 signal was shown in both long and short exposure time. Immunoblot analysis of indicated proteins in U87 treated with NVP-BEZ235 in a dose (FIG. 1G) and time (FIG. 1H) dependent manner. CHI3L1 secretion in the conditioned media (CM) was assessed by ELISA from human GBM neurosphere lines treated with NVP-BEZ235 at indicated concentrations after 12 hours of treatment (FIG. 1I) or at the concentration of 1 μM in indicated times (FIG. 1J). (FIG. 1K) Immunoblot analysis of indicated proteins in human GBM neurosphere line TS543 treated with CM of TS543 overexpressing (OE) CHI3L1 vs control (K) or overexpressing myr-AKT vs control (FIG. 1L). (FIG. 1M) CHI3L1 mRNA expression in TCGA IDHwt GBM tumors compared to non-tumor brain tissues. Gene expression was normalized by RMA and the P-value was calculated by Wilcoxon rank-sum test. (FIG. 1N) Association between CHI3L1 mRNA expression and the PI3K/AKT/mTOR signature score. Gene expression was normalized by RMA and P-value was calculated by Spearman rank correlation. (FIG. 1O) Enrichment of the PI3K/AKT/mTOR signature in IDHwt GBM with high and low levels of CHI3L1 mRNA expression. Data are presented as the mean±SD (n≥3 replicates); P-value was calculated using one-way ANOVA (FIG. 1B) or one-tailed unpaired t test (FIGS. 1E, 1I, and 1J); *P<0.05, **P<0.01, ***P<0.001, and ****P<0.0001.

FIGS. 2A-2J show that tumor progression and the immune microenvironment are implicated in glioma mouse models with alerting CHI3L1 expression. (FIG. 2A) Top 10 biological functional pathways are enriched in CHI3L1 correlated genes in TCGA GBM datasets using the Ingenuity Pathway Analysis (IPA). (FIG. 2B) qRT-PCR and immunoblot analyses of the expression levels of CHI3L1 mRNA and protein in GL216 overexpressing (OE) vector control or human CHI3L1 gene. Data are presented as the mean±SD; P-value was calculated using a one-tailed unpaired t test. (FIG. 2C) Illustration of two orthotopic xenograft models with GL261 CHI3L1 OE or vector control. (FIG. 2D) Representative MRI from mice after intracranial injection of GL261 with CHI3L1 OE or Vector. T2 sequences demonstrate infiltrative tumors in the mouse brain (yellow line). Tumor volume was measured by the T2 MRI scan. (FIG. 2E) Kaplan-Meier tumor-free survival analysis of GL261 models. (FIG. 2F) qRT-PCR and immunoblot analyses of the expression levels of CHI3L1 mRNA and protein in QPP7 cells infected with lentivirus carrying shRNA targeting mouse Chi311 gene (shChi3l1 #1 and #2) vs shRNA scrambled controls (shSC). Data are presented as the mean±SD; P-value was calculated by one-way ANOVA with Dunnett's multiple comparison test. (FIG. 2G) Representative MRI from two orthotopic xenograft glioma mouse models bearing QPP7 with shChi3l1 #2 vs shSC. Tumor volume was measured by the T2 MRI scan. (FIG. 2H) Kaplan-Meier tumor-free survival analysis of QPP7 models. Flow cytometry analyses of the indicated cell populations in GL216 (FIG. 2I) and QPP7(FIG. 2J) syngeneic mouse models with altering CHI3L1 expression. The dots represent mice from the group; data are presented as the mean±SEM; P-value was calculated using a one-tailed unpaired t test (FIG. 2I) or one-way ANOVA (FIG. 2J); *P<0.05, **P<0.01; ns represents no significance.

FIGS. 3A-3I show that CHI3L1 induces M2-like MDM accumulation in vivo. Representative flow cytometry analyses and quantitation showing the percentage of M1- and M2-like MDMs in tumors derived from GL261 (FIG. 3A and FIG. 3B) and QPP7 (FIG. 3C and FIG. 3D) glioma-bearing mice with altering CHI3L1 expression. (FIG. 3E) The ratio of CD206−/CD206+ cells from the CD45+CD68+CD11b+ cell population, and the ratio of iNOS+/Arg1+ cells from the CD45+ cell population in QPP7-derived tumors. Each dot represents 1 mouse; data are presented as the mean±SEM; P-value was calculated using a one-tailed unpaired t test. (FIG. 3F) Representative IF images for F4/80+ and P2Y12+ cells in tumor sections from the syngeneic mice bearing GL261-CHI3L1 vs vector control. (FIG. 3G) Quantitation of the indicated cells in peritumoral and intratumoral regions, respectively. Representative IF images and quantitation for F4/80+ (FIG. 3H) and P2Y12+ (FIG. 3I) cells in tumor sections from QPP7 glioma-bearing mice with shChi3l1 #2 vs shSC. Peritumoral and intratumoral regions were separated using yellow lines. Each dot represents one field of the peritumoral or intratumoral region from indicated tumors (n≥3); Data are presented as the mean SD; P-value was calculated using a one-tailed unpaired t test; ns represents no significance; scale bar, 100 μm.

FIGS. 4A-4D show that CHI3L1 induces cell migration of M2-like MDM in vitro. (FIG. 4A) Representative brightfield images showing cell migration in 0 and 6 hours after treatment with CHI3L1 recombinant protein (rCHI3L1) at the concentration of 0.6 μg/mL in M0, M1, and M2 BMDMs by the scratch-wound healing assay. (FIG. 4B) Cell migration was assessed by quantifying occupied areas by migrated cells. (FIG. 4C) Representative brightfield images for cell migration of M2 BMDMs by the Transwell assay. Migration was assessed by determining the number of migrated cells. Data are presented as the mean±SD from at least three independent experiments; P-value was calculated using a one-tailed unpaired t test; ***P<0.001, ****P<0.0001; ns represents no significance. (FIG. 4D) Association between CHI3L1 mRNA expression and M1/M2-like macrophage scores in IDHwt GBM. Gene expression was normalized by RMA and P-value was calculated by Spearman rank correlation.

FIGS. 5A-5G show that Gal3BP interacts with CHI3L1 for inhibition of BMDM migration in vitro. (FIG. 5A) A binding model of Gal3BP monomer (cyan from PDB 6GFB) and CHI3L1 (green surface with red/blue/white shades corresponding to O/N/H atoms from PDB 1HJV_A). (FIG. 5B) Detailed view from (FIG. 5A) of the binding mode of Ser129-Glu141 of Gal3BP (cyan) and CHI3L1 (green). The 10 hydrogen bonds are indicated with dashed lines and distances. Several hydrophobic contacts are also shown in the protein binding complex. (FIG. 5C) Representative IF images showing co-localization of proteins in TS603 cells; scale bar, 20 μm. (FIG. 5D) Immunoblot (IB) analysis of protein binding complexes after Co-IP with indicated antibodies in TS603 overexpressing V5-tagged CHI3L1. (FIG. 5E) Representative brightfield images from the scratch-wound healing assay showing M2 BMDM cell migration in 0 and 6 hours after treatment with recombinant CHI3L1 protein (rCHI3L1, 2.5 μg/mL) and/or recombinant Gal3BP protein (rGal3BP, 5.0 μg/mL). Cell migration was assessed by quantifying occupied areas by migrated cells. (FIG. 5F) Representative brightfield images from the Transwell assay for M2 BMDM cell migration under treatment with rCHI3L1 (2.5 μg/mL) and/or rGal3BP (5.0 μg/mL). Migration was assessed by determining the number of migrated cells. In (E) and (F), data are presented as the mean±SD from at least three independent experiments. P-value was calculated using one-way ANOVA with Tukey's multiple comparison test; ***P<0.001; ****P<0.0001; ns represents no significance; scale bar, 50 μm. (FIG. 5G) Boxplots showing enrichment of M1/M2-like macrophage signature in two indicated groups of TCGA GBMs. P-value was calculated by Wilcoxon rank-sum test.

FIG. 6A-6E show that Gal3BP competes with Gal3 for binding with CHI3L1. (FIG. 6A) A binding model of N-terminal Gal3 (magenta Asp3-Pro17 from PDB 6FOF) and CHI3L1 (green surface with red/blue/white shades corresponding to O/N/H atoms from PDB 1HJV_A). (FIG. 6B) Detailed view from (FIG. 6A) of the binding mode of Asp3-Asn16 of Gal3 (yellow) and CHI3L1 (green). (FIG. 6C) Representative IF images showing co-localization of proteins in TS603 cells. Scale bar, 20 μm. (FIG. 6D) Immunoblot (IB) analysis of protein binding complexes using Co-IP with Gal3 antibody in TS603-V5-CHI3L1 cells treated with DMSO or TD139 (10 μM for 24 hours). (FIG. 6E) Immunoblot analysis of Gal3 and CHI3L1 protein binding in the mixture of recombinant Gal3 and CHI3L1 (200 ng rGal3+200 ng rCHI3L1) by adding different amounts of recombinant Gal3BP (0, 100, 200, 400, 800 ng/sample) with or without TD139 (10 μM for 1 hour).

FIGS. 7A-7E show that the CHI3L1-Gal3-Gal3BP binding complex regulates BMDM migration. Representative brightfield images and quantitation for cell migration of M0 BMDMs treated with rCHI3L1 (2.5 μg/mL), rGal3 (2.5 μg/mL), rGal3BP (5.0 μg/mL), and combinations in the scratch-wound healing assay (FIG. 7A and FIG. 7B) and the Transwell assay (FIG. 7C and FIG. 7D). Cell migration was assessed by quantifying the occupied area or by counting the number of migrating cells, respectively. Data are presented as the mean±SD from at least three independent experiments. P-value was calculated using one-way ANOVA with Tukey's multiple comparison test; ***P<0.001, ****P<0.0001; ns represents no significance; scale bar, 50 μm. (FIG. 7E) Boxplots showing enrichment of M1/M2-like macrophage signature in two indicated groups of TCGA GBMs. P-value was calculated by Wilcoxon rank-sum test.

FIGS. 8A-8L show that CHI3L1 protein complexes regulate MDM reprogramming in immune suppression and stimulation. (FIG. 8A) Enrichment of top 10 GO biological pathways in TAMs derived from C57BL/6 mice bearing QPP7 with shChi311 compared to shSC. (FIG. 8B) Flow cytometry analysis showing active CD4 and CD8 cells in GL261 tumors with CHI3L1 overexpression compared to vector controls. (FIG. 8C) Flow cytometry analysis showing active CD4 and CD8 cells in QPP7 tumors with Chi311 KD compared to shSC. Depletion antibodies against CD4 and CD8 (10 mg/kg) were injected intraperitoneally every 3 days for a total of 8 times after tumor implantation. Kaplan-Meier tumor-free survival analysis of mice bearing GL261 overexpressing CHI3L1 (FIG. 8D) and mice bearing QPP7 with Chi311 KD (FIG. 8F), respectively. Flow cytometry analysis showing CD4+ and CD8+ cell populations within the tumors from GL261 models (FIG. 8E) and QPP7 models (FIG. 8G) with antibody depletion. Each dot represents 1 mouse; data are presented as the mean±SEM; P-value was calculated using a one-tailed unpaired t test. (FIG. 8H) qRT-PCR for indicated gene expression in M0 BMDMs treated with rCHI3L1 (2.5 μg/mL), rGal3 (2.5 μg/mL), rGal3BP (5.0 μg/mL), and combinations for 24 hours. Data are presented as the mean±SEM from at least two independent experiments. P-value was calculated using one-way ANOVA with Tukey's multiple comparison test. *P<0.05, **P<0.01 ***P<0.001, ****P<0.0001. (FIG. 8I-8K) Immunoblot analysis of indicated protein levels in M0 BMDMs treated with rCHI3L1 (2.5 μg/mL), rGal3 (2.5 μg/mL), rGal3BP (5.0 μg/mL), and combinations for 30 minutes or 4 hours (p-p65 and p65). (FIG. 8L) GSEA plots depicting mTOR1 and TNFA-NFKB signaling pathways in TAMs derived from C57BL/6 mice bearing QPP7 with shChi311 compared to shSC. NES stands for normalized enrichment score.

FIGS. 9A-9H show that a peptide mimicking Gal3BP attenuates BMDM migration and CHI3L1-induced tumor progression. (FIG. 9A) Snapshot from MD of Gal3BP mimetic peptide (GMP) and scrambled control peptide (SCP). (FIG. 9B) Representative brightfield images for cell migration of M2 BMDMs treated with rCHI3L1 (0.6 μg/mL) with/without GMP or SCP at a concentration of 30 μM in the scratch-wound healing assay. (FIG. 9C) Cell migration was assessed by quantifying the occupied area of migrated cells. Data are presented as the mean±SD from at least three independent experiments. P-value was calculated using one-way ANOVA with Tukey's multiple comparison test; ***P<0.001; ****P<0.0001; ns represents no significance. (FIG. 9D) Immunoblot analysis of protein binding complexes using Co-IP with Gal3 antibody in THP-1 cells treated with SCP or GMP (20 μM for 24 hours). (FIG. 9E) Representative MRI from mice after intracranial injection of GL261-CHI3L1 cells after the treatment of SCP and GMP, respectively. Tumor volume was measured by T2 sequences for infiltrative tumors in the mouse brain (yellow line). (FIG. 9F) Kaplan-Meier tumor-free survival analysis of mice bearing GL261-CHI3L1 tumors treating with indicated peptides. Frequency of M1/M2-like MDMs (FIG. 9G) and CD8+ T cells with expression of PD-1 and CTLA-4 (FIG. 9H) in tumors derived from syngeneic mice bearing GL261-CHI3L1 under the treatment with GMP vs SCP. Each dot represents 1 mouse; data are presented as the mean±SEM; P-value was calculated using a one-tailed unpaired t test.

FIGS. 10A-10B show that the levels of CHI3L1, LGALS3, and LGALS3BP mRNA expression predict anti-PD-1 response in GBM patients. (FIG. 10A) Histogram analysis of the distribution of anti-PD-1 treatment responders and non-responders in GBM patients following anti-PD-1 treatment from a previous dataset (Zhao J et al., 2019). The n represents the number of patients characterized with indicted gene expression. (FIG. 10B) Schematic cartoon indicates that glioma cell-intrinsic CHI3L1 binding with Gal3 forms a protein binding complex modulating the TAM-mediated immune microenvironment for tumor progression, which is negatively regulated by Gal3BP or Gal3BP mimetic peptide.

FIGS. 11A-11J show that CHI3L1 is regulated by PI3K/AKT/mTOR signaling and is related to molecular and clinical features of GBM. Immunoblot analysis (IB) of indicated proteins in patient GBM neurosphere line TS603 treated with NVP-BEZ235 in a dose (FIG. 11A) and time (FIG. 11B) dependent manner. CHI3L1 secretion in the conditioned cell culture media was assessed by ELISA from patient GBM neurosphere lines treated with NVP-BKM120 at indicated concentrations after 24 hours of treatment (FIG. 11C) or at the concentration of 1 μM in indicated times (FIG. 11D); error bars represent the mean±SD (n≥3 replicates); P-value was calculated by a one-tailed unpaired t test; *P<0.05, **P<0.01, ***P<0.001, and ****P<0.0001. (FIG. 11E-11G) CHI3L1 expression in various cell types within the TME from GBM patients' specimens based on analyzing a publicly available single-cell RNA seq dataset. The data were generated by a web tool (gbmseq.org/). (FIG. 11H) CHI3L1 mRNA expression in proneural (PN), classical (CL), and mesenchymal (MS) subtypes. P-value was calculated by two-way ANOVA with Tukey's multiple comparison test. (FIG. 11I) Enrichment of CHI3L1 mRNA expression in GBM with PTEN alterations (mutation and deletion) vs PTEN wildtype based on TCGA dataset analysis. P-value was calculated by Wilcoxon rank-sum test. (FIG. 11J) Overall survival analysis of TCGA GBM. Red and blue lines show survival curves of the top 25% tumors with the highest and lowest CHI3L1 mRNA expression, respectively.

FIGS. 12A-12L show that overexpression and knockdown of CHI3L1 affect the frequency of immune cell types in glioma mouse models. (FIG. 12A) Representative in vivo images (IVIS) of luminescence shown in the brain of SCID mice carrying intracranial tumor derived from TS543 overexpressing vector control or CHI3L1 at 39 days after implantation. (FIG. 12B) Representative IHC images showing CHI3L1 expression in tumors derived from TS543 overexpressing CHI3L1 vs vector control. Scale bar, 50 μm. (FIG. 12C) Kaplan-Meier tumor-free survival analysis. P-value was calculated by log-rank test. (FIG. 12D) Representative images showing CHI3L1 levels in tumors derived from QPP7 glioma-bearing mice by IF staining. Scale bar, 50 μm. (FIG. 12E) Representative MRI from a syngeneic glioma mouse model bearing QPP7 with shChi3l1 #1 and shChi3l1 #2 vs shSC. (FIG. 12F) Tumor volume was measured by the T2 MRI scan in mice from (FIG. 12E). P-value was calculated by one-way ANOVA with Dunnett's multiple comparison test. Flow cytometry analyses of the indicated cell populations in GL261 (FIG. 12G, 12H) and QPP7 (FIG. 121) derived syngeneic mouse models, including M1-like TAMs (CD45+CD11b+CD14+Ly6C+), Treg cells (CD45+CD3+CD4+CD25+CD127−), NKT cells (CD45+CD3+NK1.1+), NK cells (CD45+CD3-NK1.1+), monocytic MDSC (mMDSC, CD45+CD11b+Ly6G−Ly6C+), granulocytic MDSC (gMDSC, CD45+CD11b+Ly6G+Ly6C−), and the ratio of mMDSC/gMDSC. Each dot represents one mouse from the groups; error bars represent mean±SEM; P-value was calculated by a one-tailed unpaired t test in (FIG. 12G, 12H) and one-way ANOVA in (FIG. 121); *P<0.05, **P<0.01; NS represents no significance. (FIG. 12J) t-distributed Stochastic Neighbor Embedding (t-SNE) plot of cell types in three pairs of tumors derived QPP7 glioma-bearing mice with shChi3l1 #2 vs shSC. (FIG. 12K) Heatmap showing the degree of expression of genes on indicated clusters to define each immune cell type. (FIG. 12L) Quantitation of the percent of events identified as indicated cell populations from QPP7-derived tumors with shSC and shChi3l1 #2 (n=3). P-value was calculated by a one-tailed unpaired t test; data are presented as the mean±SEM; *P<0.05; NS represents no significance.

FIGS. 13A-13N show that CHI3L1 regulates BMDM and microglial cell migration. Representative flow cytometry analyses (FIG. 13A) and quantitation (FIG. 13B) showing the percentage of M1- and M2-like MDMs in tumors derived from QPP7 glioma-bearing mice with shChi3l1 #1 vs shSC. (FIG. 13C) Quantitation of the percentage of indicated cell populations in tumors derived QPP7 glioma-bearing mice with shChi3l1 #2 vs shSC. Each dot represents 1 mouse; data are presented as the mean±SEM; P-value was calculated by a one-tailed unpaired t test. Representative IF images and quantitation for CD49D and CD206 staining in tumor sections from GL261 (FIGS. 13D and 13E) and QPP7 (FIGS. 13F and 13G) derived glioma models, respectively. Each dot represents one field of indicated tumor regions (n≥3); data are presented as the mean±SD; P-value was calculated by a one-tailed unpaired t test; scale bar, 50 μm. (FIG. 13H) qRT-PCR for the hallmark gene expression of polarized BMDMs. (FIG. 131) Representative brightfield images and quantitation showing microglial cell migration in 0 and 6 hours after treatment with CHI3L1 recombinant protein (rCHI3L1) at the concentration of 0.6 μg/mL in the scratch-wound healing assay. Migration was assessed by quantifying occupied areas by migrated cells. Representative brightfield images and quantitation for cell migration of M0 BMDMs (FIGS. 13J and 13K), M1 BMDMs (FIG. 13L), and microglial cell line (FIG. 13M) in the Transwell assay. Recombinant CCL2 protein (rCCL2, 20 ng/mL) and IL4 protein (rIL4, 20 ng/mL) were used as the positive control. Cell migration was assessed by determining the number of migrated cells. Data are presented as the mean±SD from at least three independent experiments; P-value was calculated by a one-tailed unpaired t test in (FIGS. 13I, 13L, and 13M) or one-way ANOVA with Tukey's multiple comparison test in (FIG. 13K); ****P<0.0001; NS represents no significance; scale bar, 50 μm. (FIG. 13N) Enrichment of MDM and MG signature in TCGA GBM with low and high levels of CHI3L1. P-value was calculated by Wilcoxon rank-sum test.

FIGS. 14A-14D show identification of Gal3BP and association with macrophages. (FIG. 14A) The colorful histogram showing candidate genes of putative binding protein with CHI3L1 and their Pearson correlation coefficient in TCGA IDHwt GBM datasets. (FIG. 14B) Gal3BP dimerization domain (PDB 6GFB). Two monomers are shown in green/cyan. The dimer is mostly stabilized by antiparallel strand between Ser129-Leu135 of one monomer and Arg215 and Thr220 on the other. Confirmation is reminiscent of a domain swapping interaction. (FIG. 14C) Representative IF image showing expression of F4/80 and Gal3BP in tumors derived from syngeneic mice bearing GL261-CHI3L1. Quantitation was based on the percentage of Gal3BP+ cells in the tumor regions with higher and lower levels of F4/80 expression separated by yellow dash lines. Each dot represents one field of indicated regions from indicated tumors (n≥3); data are presented as the mean±SD; P-value was calculated by a one-tailed unpaired t test; scale bar, 50 μm. (FIG. 14D) Correlation between LGALS3BP mRNA expression and M1/M2-like macrophage signature in IDHwt GBM. P-value was calculated by Spearman rank correlation.

FIGS. 15A-15E show sequence alignment and association among CHI3L1, Gal3, and Gal3BP. (FIG. 15A) Sequence alignments of binding domains of CHI3L1, Gal3, and Gal3BP in the human and mouse. The red letters (without star sign) represent different amino acids in the human and mouse but have similar properties. Different amino acids in the human and mouse are indicated with start signs. (FIG. 15B) qRT-PCR for mouse Lgals3 bp gene expression in polarized BMDMs. Data are presented as the mean±SD from three replicates; P-value was calculated by one-way ANOVA with Tukey's multiple comparison test, *P<0.05, ****P<0.0001; NS represents no significance. (FIG. 15C) Immunoblot analysis of Gal3 and Gal3BP levels in polarized BMDMs. (FIG. 15D) Immunoblot analysis of Gal3 and Gal3BP levels in a microglial cell line (SIM-A9) and mouse macrophage cell line (RAW264.7). (FIG. 15E) Boxplots showing enrichment of M1/M2-like macrophage signature in two indicated groups of TCGA GBMs. P-value was calculated by Wilcoxon rank-sum test.

FIG. 16A-16J show that Chi3l1 gene knockdown reprograms MDMs toward a proinflammatory phenotype in tumor regression. Depletion of macrophages in C57BL/6 mice bearing glioma with GL261-CHI3L1 by an intravenous injection of clodronate liposomes vs control liposomes (100 μL per mouse) every 2 days for a total of 8 times. (FIG. 16A) Kaplan-Meier tumor-free survival analysis of the mice with the treatment of clodronate vs control liposomes. (FIG. 16B) Tumor volume was measured by the T2 MRI scan. Each dot represents one mouse in the groups; data are presented as the mean±SD; P-value was calculated by a one-tailed unpaired t test. (FIG. 16C) Representative flow cytometry analysis showing the percentage of CD11b+F4/80+ cells in blood from the mice with the treatment of clodronate vs control liposomes. Representative IF images for CD49D+ (FIG. 16D) and P2Y12+ (FIG. 16E) cells in tumor regions from mice with the treatment of clodronate vs control liposomes. Each dot represents one field of tumor regions from the groups; data are presented as the mean±SD; P-value was calculated by a one-tailed unpaired t test; scale bar 50 μm (FIG. 16D) and 100 μm (FIG. 16E). (FIG. 16F) Top 10 enrich hallmark signaling pathways in TAMs derived from C57BL/6 mice bearing QPP7 with shChi3l1 compared to shSC. (FIG. 16G) GSEA plots depicting indicated signaling pathways in TAMs derived from C57BL/6 mice bearing QPP7 with shChi3l1 compared to shSC. NES stands for normalized enrichment score. (FIG. 16H) RNA-seq data showing indicated gene expression in TAMs isolated from three pairs of C57BL/6 mice bearing QPP7 with shChi3l1 compared to shSC. (FIG. 16I) Representative MRI from syngeneic glioma mouse models bearing QPP7 with shChi3l1 treating with antibodies against CD4 and CD8 (10 mg/kg) by intraperitoneal injection every 2 days for a total of 8 times after tumor implantation. Tumor volume was measured by the T2 MRI scan. Each dot represents 1 mouse; data are presented as the mean±SD; P-value was calculated by a one-tailed unpaired t test. (FIG. 16J) The schematic model depicts that the CHI3L1-Gal3 protein complex regulates the PI3K/AKT/mTOR signaling pathway in MDM, leading to inhibiting NFκB activation, but promoting CEBPβ activation, thereby upregulating protumor factors for immune suppression and tumor growth. Gal3BP can negatively regulate this signaling pathway by competing with Gal3 to bind CHI3L1.

FIG. 17A-17J show that treatment with Gal3BP mimetic peptide leads to inhibiting BMDM migration in vitro and tumor immunity in vivo. (FIG. 17A) Representative brightfield images for cell migration of M0 BMDMs treated with rCHI3L1 (2.5 μg/mL), rGal3 (2.5 μg/mL), GMP (30 μM), SCP (30 μM), and indicated combinations in the scratch-wound healing assay. (FIG. 17B) Cell migration was assessed by quantifying the occupied area of migrated cells. (FIG. 17C) Histogram showing cell migration assessed by quantifying the occupied area of migrated cells in M2 BMDMs under the treatment with rCHI3L1 (0.6 μg/mL) with the different concentrations of GMP or SCP for 6 hours. Data are presented as the mean±SD from at least three independent experiments; P-value was calculated by one-way ANOVA with Tukey's multiple comparison test (FIG. 17B) and one-tailed unpaired t test (FIG. 17C); *P<0.05, **P<0.01; ***P<0.001; ****P<0.0001; NS represents no significance. (FIG. 17D) Representative MRI from mice bearing QPP7 tumors after the treatment with SCP and GMP, respectively. Tumor volume was measured by T2 sequences for infiltrative tumors in the mouse brain (yellow line). (FIG. 17E) Kaplan-Meier tumor-free survival analysis of mice bearing QPP7 tumors treating with indicated peptides. (FIG. 17F-17I) Frequency of indicated immune cell types in tumors derived from mice bearing GL261-CHI3L1 cells under the treatment with GMP vs SCP. Each dot represents 1 mouse; data are presented as the mean±SEM; P-value was calculated by a one-tailed unpaired t test. (FIG. 171) Boxplots showing low and high mRNA expression levels of indicated genes in GBM patients following anti-PD-1 immune checkpoint therapy based on a published RNA-seq dataset (Zhao J et al., 2019).

FIG. 18 shows the correlations between CD274/PD-L1 mRNA expression and CHI3L1 and LGALS3/Gal3 expression in TCGA GBM patient cohorts by the Pearson correlation analysis.

FIG. 19(A-C) shows the correlations between PDCD1LG2/PD-L2 and CHI3L1 or LGALS3/Gal3 expression in TCGA GBM patient cohorts. (FIG. 19A) PDCD1LG2/PD-L2 mRNA expression in GBM vs non-tumor. The p value was calculated by pairwise t test. PDCD1LG2/PD-L2 is positively correlated with CHI3L1 (FIG. 19B) and LGALS3/Gal3 (FIG. 19C). The RNA-seq transcriptomic data were used for the analyses.

### DETAILED DESCRIPTION

Glioblastoma is a highly malignant and incurable brain tumor characterized by intrinsic and adaptive resistance to immunotherapies. However, how glioma cells induce tumor immunosuppression and escape immunosurveillance remains poorly understood. It is shown herein that upregulation of cancer-intrinsic Chitinase-3-like-1 (CHI3L1) signaling modulating an immunosuppressive microenvironment, for example, through reprogramming tumor-associated macrophages (TAMs). Notably, administration of a Galectin-3 (Gal3)-binding protein (Gal3BP) mimetic peptide surprisingly reduces immune suppression and attenuates tumor progression.

Accordingly, disclosed herein are compositions for treating cancers, wherein the composition comprises a Galectin-3 (Gal3)-binding protein (Gal3BP) polypeptide. In some embodiments, the Gal3BP polypeptide comprises a sequence that is at least 90% identical to SEQ ID NO: 4 or SEQ ID NO: 5. In some embodiments, the Gal3BP polypeptide comprises a sequence that is at least 90% identical to SEQ ID NO: 4. Also disclosed herein are methods for treating a cancer in a subject by administering a composition comprising a Gal3BP polypeptide as described herein. Further disclosed are methods for determining whether a subject is responsive or nonresponsive to an immune checkpoint inhibitor, and in some embodiments, administering a treatment appropriate to that determination. The subjects who are non-responsive or less responsive to immune checkpoint inhibitors have a higher level of a CHI3L1 polypeptide and/or a LGALS3 polypeptide compared to its reference control, and/or a lower level of a LGALS3BP polypeptide compared to its reference control. The Gal3BP polypeptides disclosed herein have been shown to be surprisingly effective at improving responsiveness to immune checkpoint inhibitor treatments.

Terms used throughout this application are to be construed with ordinary and typical meaning to those of ordinary skill in the art. However, Applicants desire that the following terms be given the particular definition as provided below.

### Terminology

As used in the specification and claims, the singular form “a,” “an,” and “the” include plural references unless the context clearly dictates otherwise. For example, the term “a cell” includes a plurality of cells, including mixtures thereof.

The term “about” as used herein when referring to a measurable value such as an amount, a percentage, and the like, is meant to encompass variations of 20%, +10%, +5%, or 10% from the measurable value.

“Administration” to a subject includes any route of introducing or delivering to a subject an agent. Administration can be carried out by any suitable route, including oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation, via an implanted reservoir, or via a transdermal patch, and the like. Administration includes self-administration and the administration by another.

The term “biocompatible” generally refers to a material and any metabolites or degradation products thereof that are generally non-toxic to the recipient and do not cause significant adverse effects to the subject.

The term “biological sample” as used herein means a sample of biological tissue or fluid. Such samples include, but are not limited to, tissue isolated from animals. Biological samples can also include sections of tissues such as biopsy and autopsy samples, frozen sections taken for histologic purposes, blood, plasma, serum, sputum, stool, tears, mucus, hair, and skin. Biological samples also include explants and primary and/or transformed cell cultures derived from patient tissues. A biological sample can be provided by removing a sample of cells from an animal, but can also be accomplished by using previously isolated cells (e.g., isolated by another person, at another time, and/or for another purpose), or by performing the methods as disclosed herein in vivo. Archival tissues, such as those having treatment or outcome history can also be used.

As used herein, the term “comprising” is intended to mean that the compositions and methods include the recited elements, but not excluding others. “Consisting essentially of” when used to define compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

The term “cancer” as used herein is defined as a disease characterized by the rapid and uncontrolled growth of aberrant cells. Cancer cells can spread locally or through the bloodstream and lymphatic system to other parts of the body, Examples of various cancers include, but are not limited to, breast cancer, prostate cancer, ovarian cancer, cervical cancer, skin cancer, pancreatic cancer, colorectal cancer, renal cancer, liver cancer, brain cancer, lymphoma, leukemia, and lung cancer.

The term “cancer cells” and “tumor cells” are used interchangeably to refer to cells derived from a cancer or a tumor, or from a tumor cell line or a tumor cell culture.

“Composition” refers to any agent that has a beneficial biological effect. Beneficial biological effects include both therapeutic effects, e.g., treatment of a disorder or other undesirable physiological condition, and prophylactic effects, e.g., prevention of a disorder or other undesirable physiological condition. The terms also encompass pharmaceutically acceptable, pharmacologically active derivatives of beneficial agents specifically mentioned herein, including, but not limited to, a vector, polynucleotide, cells, salts, esters, amides, proagents, active metabolites, isomers, fragments, analogs, and the like. When the term “composition” is used, then, or when a particular composition is specifically identified, it is to be understood that the term includes the composition per se as well as pharmaceutically acceptable, pharmacologically active vector, polynucleotide, salts, esters, amides, proagents, conjugates, active metabolites, isomers, fragments, analogs, etc.

A “control” is an alternative subject or sample used in an experiment for comparison purposes. A control can be “positive” or “negative.”

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom, Thus, a gene encodes a protein if transcription and translation of mRNA.

The “fragments,” whether attached to other sequences or not, can include insertions, deletions, substitutions, or other selected modifications of particular regions or specific amino acids residues, provided the activity of the fragment is not significantly altered or impaired compared to the nonmodified peptide or protein. These modifications can provide for some additional property, such as to remove or add amino acids capable of disulfide bonding, to increase its bio-longevity, to alter its secretory characteristics, etc. In any case, the fragment must possess a bioactive property, such as regulating the transcription of the target gene.

The term “gene” or “gene sequence” refers to the coding sequence or control sequence, or fragments thereof. A gene may include any combination of coding sequence and control sequence, or fragments thereof. Thus, a “gene” as referred to herein may be all or part of a native gene. A polynucleotide sequence as referred to herein may be used interchangeably with the term “gene”, or may include any coding sequence, non-coding sequence or control sequence, fragments thereof, and combinations thereof. The term “gene” or “gene sequence” includes, for example, control sequences upstream of the coding sequence (for example, the ribosome binding site).

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or higher identity over a specified region when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 10 amino acids or 20 nucleotides in length, or more preferably over a region that is 10-50 amino acids or 20-50 nucleotides in length. As used herein, percent (%) nucleotide sequence identity is defined as the percentage of amino acids in a candidate sequence that are identical to the nucleotides in a reference sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

“Immune checkpoints” regulate T cell function in the immune system. T cells play a central role in cell-mediated immunity. Checkpoint proteins interact with specific ligands which send a signal into the T cell and switch off or inhibit T cell function. As used herein, the term “immune checkpoint inhibitor” or “checkpoint inhibitor” refers to a molecule that completely or partially reduces, inhibits, interferes with or modulates one or more checkpoint proteins. Checkpoint proteins include, but are not limited to, PD-1, PD-L1 and CTLA-4.

The term “increased” or “increase” as used herein generally means an increase by a statically significant amount; for the avoidance of any doubt, “increased” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

“Inhibit”, “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

“Inhibitors” of expression or of activity are used to refer to inhibitory molecules, respectively, identified using in vitro and in vivo assays for expression or activity of a described target protein, e.g., ligands, antagonists, and their homologs and mimetics. Inhibitors are agents that, e.g., inhibit expression or bind to, partially or totally block stimulation or activity, decrease, prevent, delay activation, inactivate, desensitize, or down regulate the activity of the described target protein, e.g., antagonists. Control samples (untreated with inhibitors) are assigned a relative activity value of 100%. Inhibition of a described target protein is achieved when the activity value relative to the control is about 80%, optionally 50% or 25, 10%, 5%, or 1% or less.

The term “metastatic tumor” refers to a secondary tumor growing at the site different from the site of the cancer origin.

As used herein, the term “metastasis” is meant to refer to the process in which cancer cells originating in one organ or part of the body, with or without transit by a body fluid, and relocate to another part of the body and continue to replicate.

The term “nucleic acid” as used herein means a polymer composed of nucleotides, e.g. deoxyribonucleotides (DNA) or ribonucleotides (RNA). The terms “ribonucleic acid” and “RNA” as used herein mean a polymer composed of ribonucleotides. The terms “deoxyribonucleic acid” and “DNA” as used herein mean a polymer composed of deoxyribonucleotides.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

“Pharmaceutically acceptable” component can refer to a component that is not biologically or otherwise undesirable, i.e., the component may be incorporated into a pharmaceutical formulation of the invention and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic, and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents.

As used herein, the term “carrier” encompasses any excipient, diluent, filler, salt, buffer, stabilizer, solubilizer, lipid, stabilizer, or other material well known in the art for use in pharmaceutical formulations. The choice of a carrier for use in a composition will depend upon the intended route of administration for the composition. The preparation of pharmaceutically acceptable carriers and formulations containing these materials is described in, e.g., Remington's Pharmaceutical Sciences, 21st Edition, ed. University of the Sciences in Philadelphia, Lippincott, Williams & Wilkins, Philadelphia, P A, 2005. Examples of physiologically acceptable carriers include saline, glycerol, DMSO, buffers such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™ (ICI, Inc.; Bridgewater, New Jersey), polyethylene glycol (PEG), and PLURONICS™ (BASF; Florham Park, NJ). To provide for the administration of such dosages for the desired therapeutic treatment, compositions disclosed herein can advantageously comprise between about 0.10% and 99% by weight of the total of one or more of the subject compounds based on the weight of the total composition including carrier or diluent.

The term “polynucleotide” refers to a single or double stranded polymer composed of nucleotide monomers.

The term “polypeptide” refers to a compound made up of a single chain of D- or L-amino acids or a mixture of D- and L-amino acids joined by peptide bonds.

The terms “peptide,” “protein,” and “polypeptide” are used interchangeably to refer to a natural or synthetic molecule comprising two or more amino acids linked by the carboxyl group of one amino acid to the alpha amino group of another.

The term “primary tumor” refers to a tumor at the site of the cancer origin.

The term “reference control” refers to a level in detected in a subject in general or a study population (e.g., healthy control).

The term “reduced”, “reduce”, “reduction”, “decrease”, or “decreased” as used herein generally means a decrease by a statistically significant amount. However, for avoidance of doubt, “reduced” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level, or at least about a 2-fold, or at least about a 3-fold, or at least about a 4-fold, or at least about a 5-fold or at least about a 10-fold decrease, or any decrease between 2-fold and 10-fold or greater as compared to a reference level.

The terms “specific binding,” “specifically binds,” “selective binding,” and “selectively binds” mean that a polypeptide such as Gal3BP exhibits appreciable affinity for a particular binding partner polypeptide such as Gal3. Appreciable binding affinity includes binding with an affinity of at least 10⁶ M⁻¹, specifically at least 107 M⁻¹, more specifically at least 10⁸ M⁻¹, yet more specifically at least 109 M⁻¹, or even yet more specifically at least 10¹⁰ M⁻¹. A binding affinity can also be indicated as a range of affinities, for example, 10⁶ M⁻¹ to 10¹⁰ M⁻¹, specifically 107 M⁻¹ to 10¹⁰ M⁻¹, more specifically 10⁸ M⁻¹ to 10¹⁰ M⁻¹. Specific binding can be determined according to any art-recognized means for determining such binding. In some embodiments, specific binding is determined according to Scatchard analysis and/or competitive binding assays.

The term “subject” is defined herein to include animals such as mammals, including, but not limited to, primates (e.g., humans), cows, sheep, goats, horses, dogs, cats, rabbits, rats, mice and the like. In some embodiments, the subject is a human.

The terms “treat,” “treating,” “treatment,” and grammatical variations thereof as used herein, include partially or completely alleviating, mitigating or reducing the intensity of one or more attendant symptoms of a disorder or condition and/or alleviating, mitigating or impeding one or more causes of a disorder or condition.

Preventative or prophylactic administrations are given to a subject prior to onset (e.g., before obvious signs of cancer) or during early onset (e.g., upon initial signs and symptoms of cancer). Prophylactic administration can occur for several days to years prior to the manifestation of symptoms.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition. The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition refers to an amount that is effective to achieve a desired therapeutic result. In some embodiments, a desired therapeutic result is the suppression of a cancer or tumor growth. In some embodiments, a desired therapeutic result is the suppression of a glioblastoma, or a symptom thereof. “Suppressing” a cancer or tumor growth means any or all of the following states: slowing, delaying, and stopping cancer or tumor growth, as well as tumor shrinkage, and slowing, delaying, and stopping metastasis of a cancer or tumor. Therapeutically effective amounts will typically vary with respect to factors such as the type and severity of the disorder or disease being treated and the age, gender, and weight of the subject. The term can also refer to an amount of the composition, or a rate of delivery of the composition (e.g., amount over time), effective to facilitate a desired therapeutic effect, such as the suppression of a cancer or tumor growth. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

### Compositions

It is a surprising discovery described herein that Galectin-3-binding protein (Gal3BP) competes with Galectin-3 (Gal3) to specifically bind cancer-intrinsic Chitinase-3-like-1 (CHI3L1). Binding of Gal3 to CHI3LI modulates tumor-associated macrophages (TAMs) toward a protumor phenotype, which supports cancer progression and resistance to treatment. Binding of Gal3BP to CHI3L1 instead reduces or inhibits binding of Gal3 to CHI3LI and thereby inhibits tumor progression.

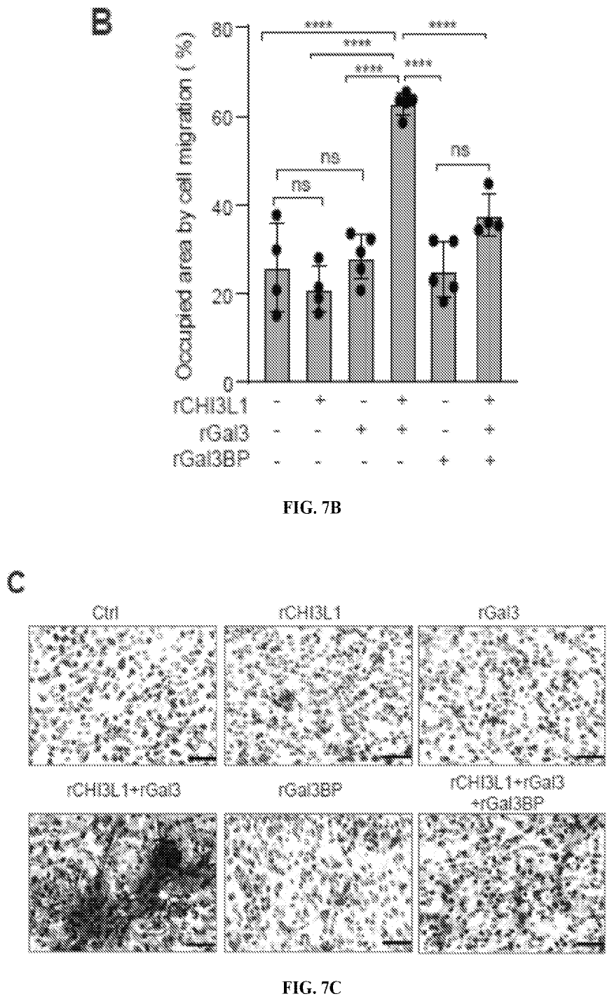

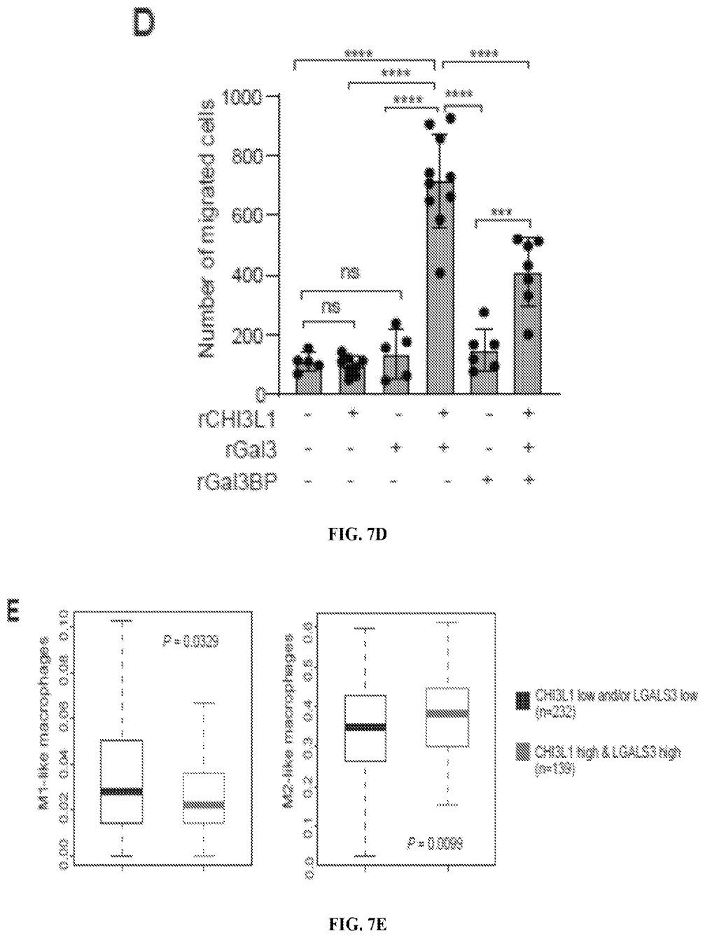

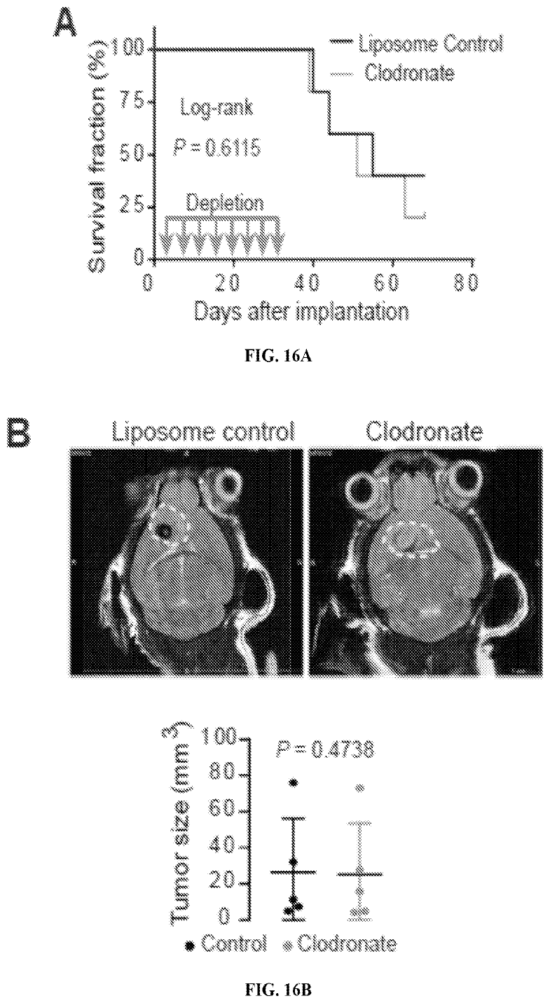

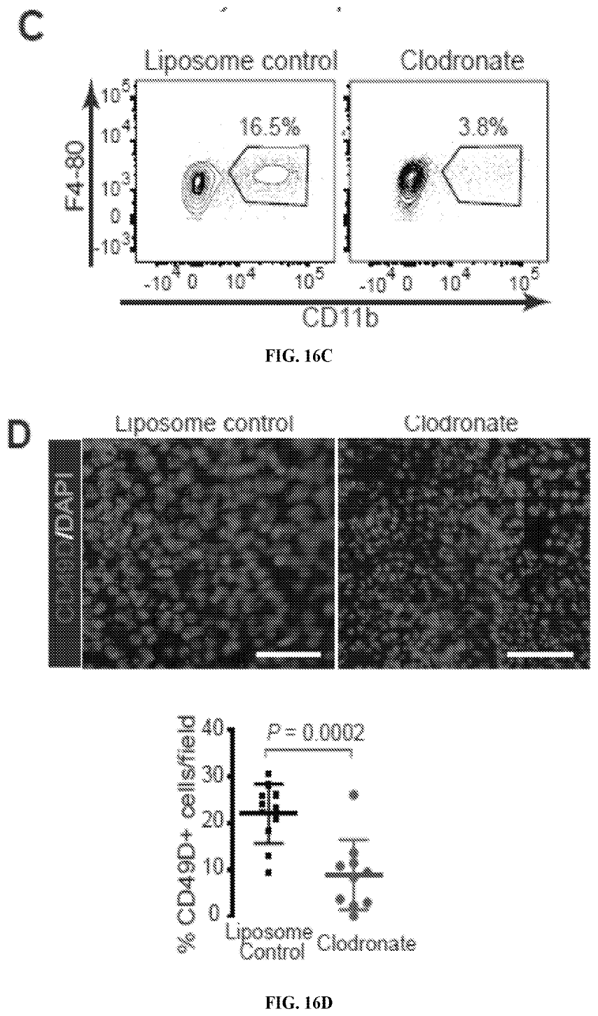

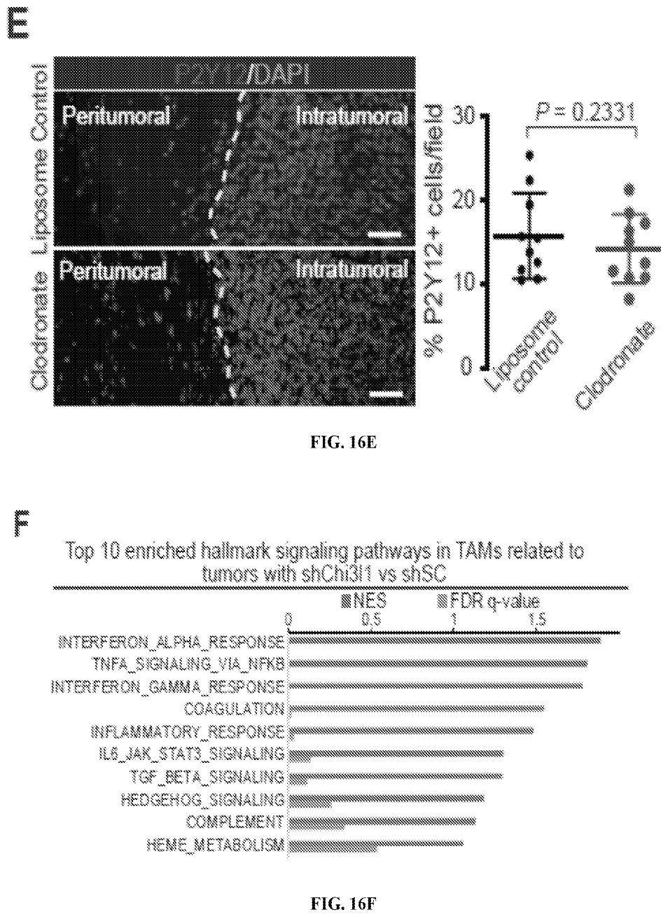

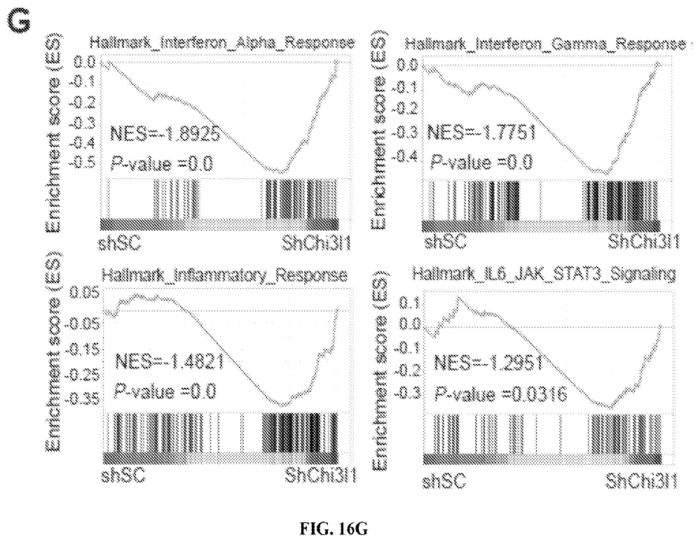

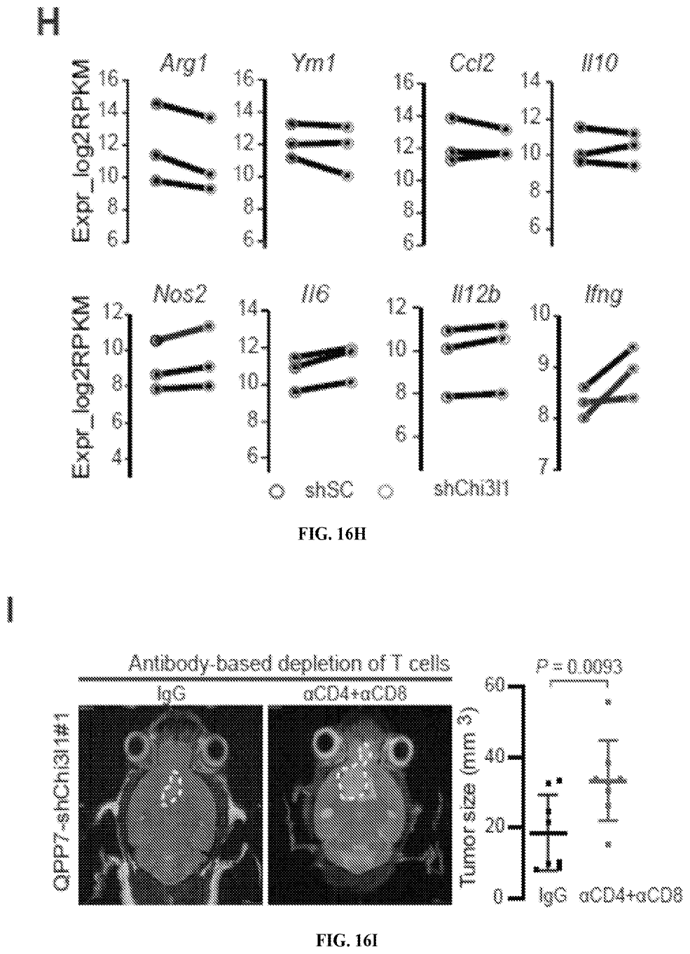

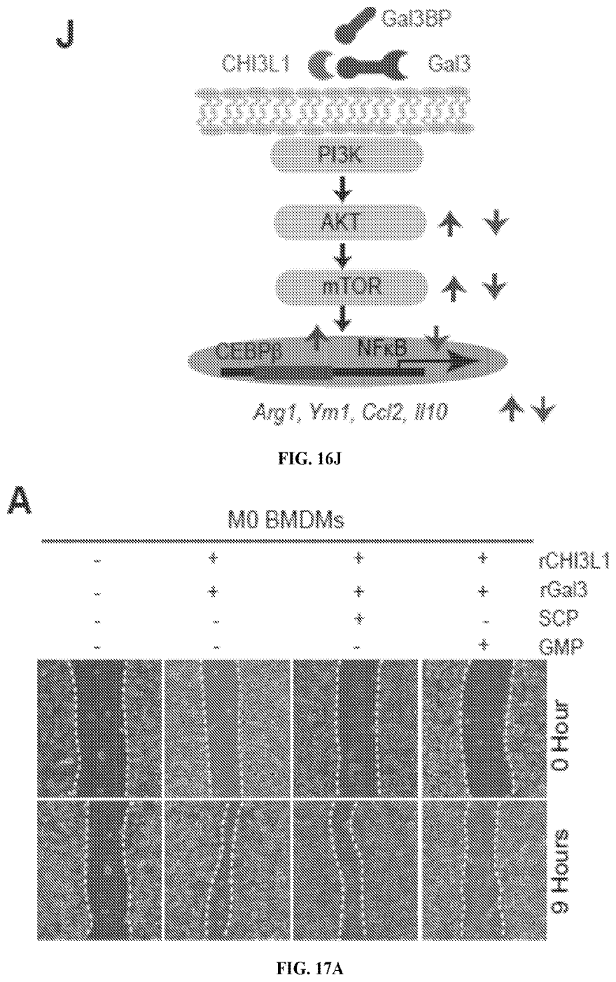

Accordingly, disclosed herein are compositions comprising a Gal3BP polypeptide. In some embodiments, the Galectin-3 (Gal3)-binding protein (Gal3BP) polypeptide comprises a sequence that is at least 80% identical to SEQ ID NO: 4 (TLDLSRELSEALGQI) or SEQ ID NO: 5 (TRSTHTLDLSRELSE). As shown herein in FIGS. 7 and 16, increased inhibition of Gal3/CHI3L1 is obtained with SEQ ID NO: 4 (TLDLSRELSEALGQI) as compared to SEQ ID NO: 5 (TRSTHTLDLSRELSE). Therefore, in some embodiments, the Gal3BP polypeptide is at least 80% identical to SEQ ID NO: 4 (TLDLSRELSEALGQI). Although other studies have proposed the use of compositions that bind to Gal3 such as modified disaccharides (TD139) and large polysaccharides containing galactose (GR-MD-02), those compositions do not mimic the binding of Gal3BP to Gal3 and focus on a different binding domain of Gal3. The presently disclosed Gal3BP polypeptides are therefore more advantageous that the prior known Gal3 binding compositions.

As used herein, “Gal3” or “Galectin-3” refers to a polypeptide that synthesizes and hydrolyzes cyclic adenosine 5′-diphosphate-ribose, and in humans, is encoded by the LGALS3 gene. In some embodiments, the Gal3 polypeptide is that identified in one or more publicly available databases as follows: HGNC: 6563, Entrez Gene: 3958, Ensembl: ENSG00000131981, OMIM: 153619, UniProtKB: P17931. In some embodiments, the Gal3 polypeptide comprises the sequence of SEQ ID NO: 1, or a polypeptide sequence having at or greater than about 80%, about 85%, about 90%, about 95%, or about 98% homology with SEQ ID NO: 1, or a polypeptide comprising a portion of SEQ ID NO: 1. The Gal3 polypeptide of SEQ ID NO: 1 may represent an immature or pre-processed form of mature Gal3, and accordingly, included herein are mature or processed portions of the Gal3 polypeptide in SEQ ID NO: 1.

“CHI3L1” or “cancer-intrinsic chitinase-3-like-1” refers herein to a polypeptide that lacks chitinase activity and is secreted by activated macrophages, and in humans, is encoded by the CHI3L1 gene. In some embodiments, the CHI3L1 polypeptide is that identified in one or more publicly available databases as follows: HGNC: 1932, Entrez Gene: 1116, Ensembl: ENSG00000133048, OMIM: 601525, UniProtKB: P36222. In some embodiments, the CHI3L1 polypeptide comprises the sequence of SEQ ID NO: 3, or a polypeptide sequence having at or greater than about 80%, about 85%, about 90%, about 95%, or about 98% homology with SEQ ID NO: 3, or a polypeptide comprising a portion of SEQ ID NO: 3. The CHI3L1 polypeptide of SEQ ID NO: 3 may represent an immature or pre-processed form of mature CHI3L1, and accordingly, included herein are mature or processed portions of the CHI3L1 polypeptide in SEQ ID NO: 3.

“Gal3BP” or “Galectin-3-binding protein” refers herein to a polypeptide that specifically binds to Galectin-3 and/or CHI3L1, and in humans, is encoded by the LGALS3BP gene. In some embodiments, the Gal3BP polypeptide reduces binding between a Gal3 polypeptide and a CHI3L1 polypeptide. In some embodiments, the Gal3BP polypeptide binds to a CHI3L1 polypeptide and reduces binding between a Gal3 polypeptide and a CHI3L1 polypeptide. In some embodiments, a Gal3BP polypeptide/Gal3 polypeptide/CHI3L1 polypeptide complex is created. In some embodiments, the Gal3BP polypeptide is that identified in one or more publicly available databases as follows: HGNC: 6564, Entrez Gene: 3959, Ensembl: ENSG00000108679, OMIM: 600626, UniProtKB: Q08380. In some embodiments, the Gal3BP polypeptide comprises the sequence of SEQ ID NO: 2, or a polypeptide sequence having at or greater than about 80%, about 85%, about 90%, about 95%, or about 98% homology with SEQ ID NO: 2, or a polypeptide comprising a portion of SEQ ID NO: 2. The Gal3BP polypeptide of SEQ ID NO: 2 may represent an immature or pre-processed form of mature Gal3BP, and accordingly, included herein are mature or processed portions of the Gal3BP polypeptide in SEQ ID NO: 2.

In some embodiments, the Gal3BP polypeptide provided herein comprises SEQ ID NO: 4 (TLDLSRELSEALGQI). In other embodiments, the Gal3BP polypeptide comprises a sequence that is at least 80%, at least 85%, at least 90%, or at least 95% identical to SEQ ID NO:4. In some embodiments, the Gal3BP polypeptide is about 15, about 20, about 25, about 30, about 35, about 40, about 45, or about 50 amino acids in length. In some embodiments, the Gal3BP polypeptide is 10, 11, 12, 13, 14, 15, 16, 17, 18, 19, or 20 amino acids in length. In some embodiments, the Gal3BP polypeptide has the sequence of SEQ ID NO: 4. In some embodiments, the Gal3BP polypeptide consists essentially of, or consists of, SEQ ID NO: 4. In some embodiments, the Gal3BP polypeptide disclosed herein is formulated with a pharmaceutically acceptable carrier. It should be understood that in each of the aforementioned embodiments, the Gal3BP polypeptide specifically binds to Gal3 and/or CHI3L1. Accordingly, in some embodiments, the Gal3BP polypeptide specifically binds to CHI3L1 polypeptide. In some embodiments, the Gal3BP polypeptide specifically binds to both Gal3 polypeptide and CHI3L1 polypeptide. In some embodiments, the Gal3BP polypeptide specifically binds to both Gal3 polypeptide and CHI3L1 polypeptide and modulates or reduces CHI3L1 intracellular signaling.

In some embodiments, the Gal3BP polypeptide provided herein comprises SEQ ID NO: 5 (TRSTHTLDLSRELSE). In other embodiments, the Gal3BP polypeptide comprises a sequence that is at least 80%, at least 85%, at least 90%, or at least 95% identical to SEQ ID NO: 5. In some embodiments, the Gal3BP polypeptide is about 15, about 20, about 25, about 30, about 35, about 40, about 45, or about 50 amino acids in length. In some embodiments, the Gal3BP polypeptide is 10, 11, 12, 13, 14, 15, 16, 17, 18, 19, or 20 amino acids in length. In some embodiments, the Gal3BP polypeptide has the sequence of SEQ ID NO: 5. In some embodiments, the Gal3BP polypeptide consists essentially of, or consists of, SEQ ID NO: 5. In some embodiments, the Gal3BP polypeptide disclosed herein is formulated with a pharmaceutically acceptable carrier. It should be understood that in each of the aforementioned embodiments, the Gal3BP polypeptide specifically binds to Gal3 and/or CHI3L1. Accordingly, in some embodiments, the Gal3BP polypeptide specifically binds to CHI3L1 polypeptide. In some embodiments, the Gal3BP polypeptide specifically binds to both Gal3 polypeptide and CHI3L1 polypeptide. In some embodiments, the Gal3BP polypeptide specifically binds to both Gal3 polypeptide and CHI3L1 polypeptide and modulates or reduces CHI3L1 intracellular signaling.

In some embodiments, the Gal3BP polypeptide disclosed herein has a C-terminal amidation. An example of a peptide with C-terminal amidation is shown below:

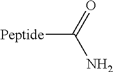

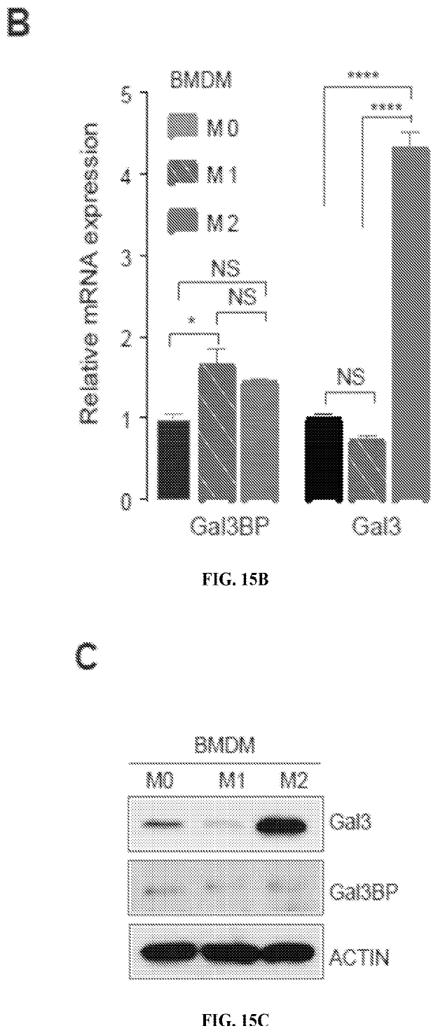

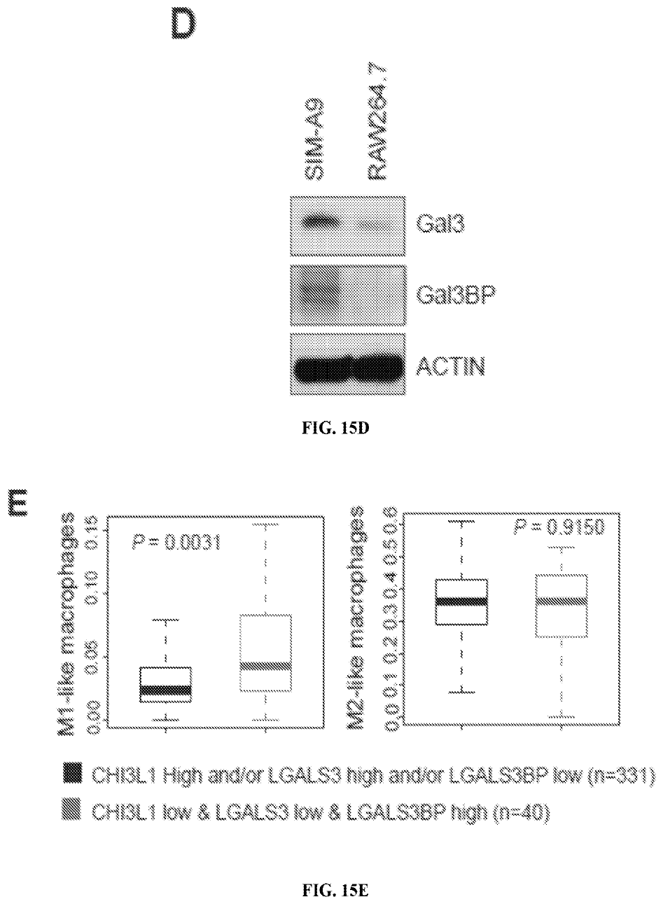

It is postulated herein that Gal3BP binds to Gal3 using its scavenger receptor cysteine-rich (SRCR) domain in a specific carbohydrate-recognition domain-dependent manner but binds to CHI3L1 using its Broad-Complex, Tramtrack and Bric a brac/Pox virus and Zinc finger (BTB/POZ) domain. Inhibition of the carbohydrate recognition domain of Gal3 using TD139 resulted in disrupting the Gal3-Gal3BP interaction but not the Gal3-CHI3L1 interaction. (FIG. 15). Notably, this is the first time identifying Gal3BP as a binding partner of CHI3L1. Therefore, the present disclosure surprisingly shows Gal3BP as binding partner of CHI3L1 in addition to Gal3.

### Methods of Treatment

Also disclosed herein are methods for using the Gal3BP polypeptides disclosed herein for the treatment of a cancer. A representative but non-limiting list of cancers that the disclosed compositions can be used to treat is the following: lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, lung cancers such as small cell lung cancer and non-small cell lung cancer, neuroblastoma/glioblastoma, ovarian cancer, skin cancer, liver cancer, melanoma, squamous cell carcinomas of the mouth, throat, larynx, and lung, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers; testicular cancer; colon cancer, rectal cancer, prostatic cancer, non-small cell lung cancer (NSCLC), pancreatic cancer, acoustic neuroma, astrocytoma, brain metastases, choroid plexus carcinoma, craniopharyngioma, embryonal tumors, ependymoma, glioblastoma, glioma, medulloblastoma, meningioma, oligodendroglioma, pediatric brain tumors, pineoblastoma, or pituitary tumors. In some embodiments, the cancer is a brain cancer. In some embodiments, the cancer is a glioblastoma. In some embodiments, the cancer is a breast cancer. In some embodiments, the cancer is melanoma. In some embodiments, the cancer is gastric cancer or colorectal cancer.

Accordingly, included herein are methods for treating a cancer in a subject comprising administering to the subject a therapeutically effective amount of a Galectin-3 (Gal3)-binding protein (Gal3BP) polypeptide, wherein the Gal3BP polypeptide specifically binds CHI3L1 polypeptide. In some embodiments, the Gal3BP polypeptide binding to CHI3L1 polypeptide modulates CHI3L1 intracellular signaling. In some embodiments, the Gal3BP polypeptide binding to CHI3L1 polypeptide reduces CHI3L1 intracellular signaling. In some embodiments, the Gal3BP polypeptide specifically binds both CHI3L1 polypeptide and Gal3 polypeptide.

In some embodiments, the Gal3BP polypeptide comprises a sequence at least about 80% identical to SEQ ID NO: 4 or SEQ ID NO: 5. In some of the treatment embodiments, the Gal3BP polypeptide comprises a sequence that is at least 80%, at least 85%, at least 90%, or at least 95% identical to SEQ ID NO:4 or SEQ ID NO: 5. In some of the treatment embodiments, the Gal3BP polypeptide is about 15, about 20, about 25, about 30, about 35, about 40, about 45, or about 50 amino acids in length. In some of the treatment embodiments, the Gal3BP polypeptide is 10, 11, 12, 13, 14, 15, 16, 17, 18, 19, or 20 amino acids in length. In some of the treatment embodiments, the Gal3BP polypeptide has the sequence of SEQ ID NO: 4 or SEQ ID NO: 5. In some of the treatment embodiments, the Gal3BP polypeptide consists essentially of, or consists of, SEQ ID NO: 4 or SEQ ID NO: 5. In some of the treatment embodiments, the Gal3BP polypeptide disclosed herein is formulated with a pharmaceutically acceptable carrier.

Also included herein are methods for treating a cancer in a subject comprising administering to the subject a therapeutically effective amount of a Galectin-3 (Gal3)-binding protein (Gal3BP) polypeptide, wherein the Gal3BP polypeptide comprises a sequence at least about 80% identical to SEQ ID NO: 4. In some of the treatment embodiments, the Gal3BP polypeptide comprises a sequence that is at least 80%, at least 85%, at least 90%, or at least 95% identical to SEQ ID NO:4. In some of the treatment embodiments, the Gal3BP polypeptide is about 15, about 20, about 25, about 30, about 35, about 40, about 45, or about 50 amino acids in length. In some of the treatment embodiments, the Gal3BP polypeptide is 10, 11, 12, 13, 14, 15, 16, 17, 18, 19, or 20 amino acids in length. In some of the treatment embodiments, the Gal3BP polypeptide has the sequence of SEQ ID NO: 4. In some of the treatment embodiments, the Gal3BP polypeptide consists essentially of, or consists of, SEQ ID NO: 4.

As discussed further below, it is described herein for the first time that administration of a Gal3BP polypeptide increase the effectiveness of an immune checkpoint inhibitor during the treatment of a cancer. Accordingly, provided herein are methods for treating a cancer that comprise administration of a Gal3BP polypeptide and an immune checkpoint inhibitor. The immune checkpoint inhibitor can be administered concurrently with a Gal3BP polypeptide disclosed herein or after the administration of a Gal3BP polypeptide. Accordingly, in some aspects, the cancer treatment method disclosed herein further comprises administering to the subject a therapeutically effective amount of an immune checkpoint inhibitor (including, for example, a PD-1 inhibitor, a PD-L1 inhibitor, a PD-L2 inhibitor, or a CLTA-4 inhibitor). In some examples, the immune checkpoint inhibitor is a PD-1 inhibitor. In some examples, the immune checkpoint inhibitor is a PD-L 1 inhibitor. In some examples, the immune checkpoint inhibitor is a PD-L2 inhibitor. In some examples, the immune checkpoint inhibitor is a CTLA-4 inhibitor. Checkpoint inhibitors include, but are not limited to, antibodies that reduce binding partner interactions of PD-1 (Nivolumab (BMS-936558 or MDX1106), CT-011, MK-3475), PD-L1 (MDX-1105 (BMS-936559), MPDL3280A, MSB0010718C), PD-L2 (rHIgM12B7), CTLA-4 (Ipilimumab (MDX-010), Tremelimumab (CP-675,206)), IDO, B7-H3 (MGA271), B7-H4, TIM3, or LAG-3 (BMS-986016).

As used herein, the term “PD-1 inhibitor” refers to a composition that binds to PD-1 and reduces or inhibits the interaction between the bound PD-1 and PD-L1. In some embodiments, the PD-1 inhibitor is a monoclonal antibody that is specific for PD-1 and that reduces or inhibits the interaction between the bound PD-1 and PD-L1. Non-limiting examples of PD-1 inhibitors are pembrolizumab, nivolumab, and cemiplimab. In some embodiments, the pembrolizumab is KEYTRUDA or a bioequivalent. In some embodiments, the pembrolizumab is that described in U.S. Pat. Nos. 8,952,136, 8,354,509, or U.S. Pat. No. 8,900,587, all of which are incorporated by reference in their entireties. In some embodiments, the pembrolizumab has the Unique Ingredient Identifier (UNII) of the U.S. Food and Drug Administration of DPT003T46P. In some embodiments, the nivolumab is OPDIVO or a bioequivalent. In some embodiments, the nivolumab has the Unique Ingredient Identifier (UNII) of the U.S. Food and Drug Administration of 31YO63LBSN. In some embodiments, the nivolumab is that described in U.S. Pat. Nos. 7,595,048, 8,738,474, 9,073,994, 9,067,999, 8,008,449, or U.S. Pat. No. 8,779,105, all of which are incorporated by reference in their entireties. In some embodiments, the cemiplimab is LIBTAYO or a bioequivalent. In some embodiments, the cemiplimab has the Unique Ingredient Identifier (UNII) of the U.S. Food and Drug Administration of 6QVL0571NT. In some embodiments, the cemiplimab is that described in U.S. Pat. No. 10,844,137, which is incorporated by reference in its entirety.

The term “PD-L1 inhibitor” refers to refers to a composition that binds to PD-1 and reduces or inhibits the interaction between the bound PD-L1 and PD-1. In some embodiments, the PD-L1 inhibitor is a monoclonal antibody that is specific for PD-L1 and that reduces or inhibits the interaction between the bound PD-L1 and PD-1. Non-limiting examples of PD-L1 inhibitors are atezolizumab, avelumab and durvalumab. In some embodiments, the atezolizumab is TECENTRIQ or a bioequivalent. In some embodiments, the atezolizumab has the Unique Ingredient Identifier (UNII) of the U.S. Food and Drug Administration of 52CMI0WC3Y. In some embodiments, the atezolizumab is that described in U.S. Pat. No. 8,217,149, which is incorporated by reference in its entirety. In some embodiments, the avelumab is BAVENCIO or a bioequivalent. In some embodiments, the avelumab has the Unique Ingredient Identifier (UNII) of the U.S. Food and Drug Administration of KXG2PJ551I. In some embodiments, the avelumab is that described in U.S. Pat. App. Pub. No. 2014321917, which is incorporated by reference in its entirety. In some embodiments, the durvalumab is IMFINZI or a bioequivalent. In some embodiments, the durvalumab has the Unique Ingredient Identifier (UNII) of the U.S. Food and Drug Administration of 28×28×90 KV. In some embodiments, the durvalumab is that described in U.S. Pat. No. 8,779,108, which is incorporated by reference in its entirety.

The term “CTLA-4 inhibitor” refers to a composition that binds to CTLA-4 and reduces or inhibits the interaction between the bound CTLA-4 and B7. In some embodiments, the CTLA-4 inhibitor is a monoclonal antibody that is specific for CTLA-4 and that reduces or inhibits the interaction between the bound CTLA-4 and B7. A non-limiting example of a CTLA-4 inhibitor is ipilimumab. In some embodiments, the ipilimumab is YERVOY or a bioequivalent. In some embodiments, the ipilimumab has the Unique Ingredient Identifier (UNII) of the U.S. Food and Drug Administration of 6T8C155666. In some embodiments, the ipilimumab is that described in U.S. Pat. Nos. 7,605,238, 6,984,720, 5,811,097, 5,855,887, or U.S. Pat. No. 6,051,227, all of which are incorporated by reference in their entireties.

In some embodiments, administration of a Gal3BP polypeptide decreases a level of one ore more immune checkpoint polypeptides on a cell. In some embodiments, the cell is an immune cell (e.g., CD45+ cells that include, for example, a CD4 T cell, a CD8 T cell, and/or a macrophage) or a nonimmune cell (e.g., a tumor cell). The term “immune checkpoint polypeptide” herein refers to a cell surface polypeptide that negatively regulate adaptive immune cell activation. In some embodiments, the one or more immune checkpoint polypeptides is selected from the group consisting of CTLA-4, PD-1, PD-L1, PD-L2, BTLA, HVEM, TIM3, and GAL9. In some embodiments, the immune checkpoint polypeptide is, for example, CTLA-4 (SEQ ID NO: 6) (HGNC: 2505; NCBI Entrez Gene: 1493; Ensembl: ENSG00000163599; OMIM®: 123890; UniProtKB/Swiss-Prot: P16410), PD-1 (SEQ ID NO: 7) (HGNC: 8760; NCBI Entrez Gene: 5133; Ensembl: ENSG00000188389; OMIM®: 600244; UniProtKB/Swiss-Prot: Q15116), PD-L1 (SEQ ID NO: 8) (HGNC: 17635; NCBI Entrez Gene: 29126; Ensembl: ENSG00000120217; OMIM®: 605402; UniProtKB/Swiss-Prot: Q9NZQ7), PD-L2 (SEQ ID NO: 9) (HGNC: 18731; NCBI Entrez Gene: 80380; Ensembl: ENSG00000197646; OMIM®: 605723; UniProtKB/Swiss-Prot: Q9BQ51), BTLA (HGNC: 21087; NCBI Entrez Gene: 151888; Ensembl: ENSG00000186265; OMIM®: 607925; UniProtKB/Swiss-Prot: Q7Z6A9), HVEM (HGNC: 11912; NCBI Entrez Gene: 8764; Ensembl: ENSG00000157873; OMIM®: 602746; UniProtKB/Swiss-Prot: Q92956), TIM3 (HGNC: 18437; NCBI Entrez Gene: 84868; Ensembl: ENSG00000135077; OMIM®: 606652; UniProtKB/Swiss-Prot: Q8TDQ0), or GAL9 (HGNC: 6570; NCBI Entrez Gene: 3965; Ensembl: ENSG00000168961; OMIM®: 601879; UniProtKB/Swiss-Prot: 000182). In some embodiments, administration of a Gal3BP polypeptide decreases a level of a PD-L1 polypeptide on an immune cell. In some embodiments, administration of a Gal3BP polypeptide decreases a level of a PD-L1 polypeptide on a tumor cell. In some embodiments, administration of a Gal3BP polypeptide decreases a level of a PD-L2 polypeptide on an immune cell. In some embodiments, administration of a Gal3BP polypeptide decreases a level of a PD-L2 polypeptide on a tumor cell.

As the timing of a cancer can often not be predicted, it should be understood the disclosed methods of treating, preventing, reducing, and/or inhibiting the disease or disorder described herein can be used prior to or following the onset of the disease or disorder, to treat, prevent, inhibit, and/or reduce the disease or disorder or symptoms thereof. In one aspect, the disclosed methods can be employed 30, 29, 28, 27, 26, 25, 24, 23, 22, 21, 20, 19, 18, 17, 16, 15, 14, 13, 12, 11, 10, 9, 8, 7, 6, 5, 4, 3, 2 years, 12, 11, 10, 9, 8, 7, 6, 5, 4, 3, 2 months, 30, 29, 28, 27, 26, 25, 24, 23, 22, 21, 20, 19, 18, 17, 16, 15, 14, 13, 12, 11, 10, 9, 8, 7, 6, 5, 4, 3 days, 60, 48, 36, 30, 24, 18, 15, 12, 10, 9, 8, 7, 6, 5, 4, 3, 2 hours, 60, 45, 30, 15, 10, 9, 8, 7, 6, 5, 4, 3, 2, or 1 minute prior to onset of the disease or disorder; or 1, 2, 3, 4, 5, 6, 7, 8, 9, 10, 15, 20, 25, 30, 35, 40, 45, 50, 55, 60, 75, 90, 105, 120 minutes, 3, 4, 5, 6, 7, 8, 9, 10, 11, 12, 15, 18, 24, 30, 36, 48, 60 hours, 3, 4, 5, 6, 7, 8, 9, 10, 11, 12, 13, 14, 15, 16, 17, 18, 19, 20, 21, 22, 23, 24, 25, 26, 27, 28, 29, 30 days, 2, 3, 4, 5, 6, 7, 8, 9, 10, 11, 12 months, 2, 3, 4, 5, 6, 7, 8, 9, 10, 15, 20, 25, 30, 35, 40, 45, 50, 55, 60 or more years after onset of the disease or disorder.

Dosing frequency for the composition of any preceding aspects, includes, but is not limited to, at least once every year, once every two years, once every three years, once every four years, once every five years, once every six years, once every seven years, once every eight years, once every nine years, once every ten year, at least once every two months, once every three months, once every four months, once every five months, once every six months, once every seven months, once every eight months, once every nine months, once every ten months, once every eleven months, at least once every month, once every three weeks, once every two weeks, once a week, twice a week, three times a week, four times a week, five times a week, six times a week, daily, two times per day, three times per day, four times per day, five times per day, six times per day, eight times per day, nine times per day, ten times per day, eleven times per day, twelve times per day, once every 12 hours, once every 10 hours, once every 8 hours, once every 6 hours, once every 5 hours, once every 4 hours, once every 3 hours, once every 2 hours, once every hour, once every 40 minutes, once every 30 minutes, once every 20 min, or once every 10 minutes. Administration can also be continuous and adjusted to maintaining a level of the compound within any desired and specified range.

### Methods of Identifying a Subject's Responsiveness to an Immune Checkpoint Inhibitor

The present disclosure shows a correlation between the level of CHI3L1, Gal3, and/or Gal3BP with the subject's responsiveness to an immune checkpoint inhibitor. More specifically, disclosed herein is the surprising finding that an increased expression of LGALS3BP increases a subject's responsiveness to an immune checkpoint inhibitor. It is a further surprising finding that a decreased expression of CHI3L1 and/or LGALS3 decrease a subject's responsiveness to an immune checkpoint inhibitor.

Accordingly, included herein is a method for identifying a subject's responsiveness to an immune checkpoint inhibitor, said method comprising 1) obtaining a biological sample from the subject, 2) quantifying a level of a biomarker relative to a reference control, wherein the biomarker is selected from a CHI3L1 polypeptide, a Gal3 polypeptide, and a Gal3BP polypeptide; and 3) determining the subject as responsive to the immune checkpoint inhibitor when the level of one or more of the CHI3L1 polypeptide and/or the Gal3 polypeptide is lower in the biological sample than a CHI3L1 reference control and/or a Gal3 reference control, or the level of the Gal3BP polypeptide is higher in the biological sample than a Gal3BP reference control, or a combination thereof, or 4) determining the subject as non-responsive to the immune checkpoint inhibitor when the level of one or more of the CHI3L1 polypeptide or the Gal3 polypeptide is higher in the biological sample than its reference control, or the level of the Gal3BP polypeptide is lower in the biological sample than its reference control, or a combination thereof. The term “reference control” refers to a level detected in general or a study population as representative of a particular attribute, such as, for example, a level representative of being responsive to an immune checkpoint inhibitor. In some embodiments, the biological sample is selected from serum, plasma, whole blood, cerebrospinal fluid (CSF), and tumor tissue.

The disclosure also includes the above-described method for determining a subject's responsiveness to an immune checkpoint inhibitor, further comprising administering a therapeutically effective amount of an immune checkpoint inhibitor to the subject deemed responsive to the immune checkpoint inhibitor. Still further included herein is above described method for determining a subject's responsiveness to an immune checkpoint inhibitor, further comprising administering a therapeutically effective amount of a Gal3PB polypeptide to the subject deemed non-responsive to the immune checkpoint inhibitor, and in some embodiments, further administering to the subject a therapeutically effective amount of an immune checkpoint inhibitor. The immune checkpoint inhibitor can be administered after the Gal3PB polypeptide is administered or concurrently with the Gal3PB polypeptide.

The Gal3PB polypeptide and the immune checkpoint inhibitor used in these methods can be any described herein. These methods can be used to treat a cancer, including, but not limited to a brain cancer, such as, for example, acoustic neuroma, astrocytoma, brain metastases, choroid plexus carcinoma, craniopharyngioma, embryonal tumors, ependymoma, glioblastoma, glioma, medulloblastoma, meningioma, oligodendroglioma, pediatric brain tumors, pineoblastoma, or pituitary tumors. In some embodiments, the brain cancer is a glioblastoma.

As the timing of a cancer can often not be predicted, it should be understood the disclosed methods of treating, preventing, reducing, and/or inhibiting the disease or disorder described herein can be used prior to or following the onset of the disease or disorder, to treat, prevent, inhibit, and/or reduce the disease or disorder or symptoms thereof. In one aspect, the disclosed methods can be employed 30, 29, 28, 27, 26, 25, 24, 23, 22, 21, 20, 19, 18, 17, 16, 15, 14, 13, 12, 11, 10, 9, 8, 7, 6, 5, 4, 3, 2 years, 12, 11, 10, 9, 8, 7, 6, 5, 4, 3, 2 months, 30, 29, 28, 27, 26, 25, 24, 23, 22, 21, 20, 19, 18, 17, 16, 15, 14, 13, 12, 11, 10, 9, 8, 7, 6, 5, 4, 3 days, 60, 48, 36, 30, 24, 18, 15, 12, 10, 9, 8, 7, 6, 5, 4, 3, 2 hours, 60, 45, 30, 15, 10, 9, 8, 7, 6, 5, 4, 3, 2, or 1 minute prior to onset of the disease or disorder; or 1, 2, 3, 4, 5, 6, 7, 8, 9, 10, 15, 20, 25, 30, 35, 40, 45, 50, 55, 60, 75, 90, 105, 120 minutes, 3, 4, 5, 6, 7, 8, 9, 10, 11, 12, 15, 18, 24, 30, 36, 48, 60 hours, 3, 4, 5, 6, 7, 8, 9, 10, 11, 12, 13, 14, 15, 16, 17, 18, 19, 20, 21, 22, 23, 24, 25, 26, 27, 28, 29, 30 days, 2, 3, 4, 5, 6, 7, 8, 9, 10, 11, 12 months, 2, 3, 4, 5, 6, 7, 8, 9, 10, 15, 20, 25, 30, 35, 40, 45, 50, 55, 60 or more years after onset of the disease or disorder.

Dosing frequency for the composition of any preceding aspects, includes, but is not limited to, at least once every year, once every two years, once every three years, once every four years, once every five years, once every six years, once every seven years, once every eight years, once every nine years, once every ten year, at least once every two months, once every three months, once every four months, once every five months, once every six months, once every seven months, once every eight months, once every nine months, once every ten months, once every eleven months, at least once every month, once every three weeks, once every two weeks, once a week, twice a week, three times a week, four times a week, five times a week, six times a week, daily, two times per day, three times per day, four times per day, five times per day, six times per day, eight times per day, nine times per day, ten times per day, eleven times per day, twelve times per day, once every 12 hours, once every 10 hours, once every 8 hours, once every 6 hours, once every 5 hours, once every 4 hours, once every 3 hours, once every 2 hours, once every hour, once every 40 minutes, once every 30 min, once every 20 minutes, or once every 10 minutes. Administration can also be continuous and adjusted to maintaining a level of the compound within any desired and specified range.

Included herein are methods of using a Gal3BP polypeptide described herein as a medicament. Also included is a Gal3BP polypeptide described herein for use in diagnostics. In some embodiments, a Gal3BP polypeptide described herein is for use in a method of treating cancer in a subject, the method comprising administering the Gal3BP polypeptide to the subject. In some embodiments, a Gal3BP polypeptide described herein is for use in a method of treating glioblastoma in a subject, the method comprising administering the Gal3BP polypeptide to the subject.

### EXAMPLES

The following examples are set forth below to illustrate the compositions, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1. Cancer-Cell-Intrinsic CHI3L1 is Regulated by the PI3K/AKT/mTOR Pathway in a Positive Feedback Loop

GBM is highly immunosuppressive and resistant to immunotherapy because glioma cells escape effective antitumor immunity by programing the tumor microenvironment (TME) (Lim et al., 2018; Sampson et al., 2020). Tumor associated macrophages/microglia (TAMs), the major component of the GBM TME, account for up to 30%-50% of total tumor composition (Hambardzumyan et al., 2016). GBM TAMs originate from bone marrow-derived blood monocytes (monocyte-derived macrophages, MDMs) and brain resident microglia (MG) (Ginhoux et al., 2010; Hambardzumyan et al., 2016). Previous studies reported that MG account for approximately 15% of TAMs and mainly localize in peritumoral areas, whereas MDMs preferentially localize in intratumoral regions and constitute approximately 85% of the total TAM population. MDMs significantly contribute to the immunosuppressive microenvironment of high-grade glioma (Chen et al., 2017; Pinton et al., 2019), showing different functions of MG and MDMs within the GBM TME. Increasing evidence indicates that protumor M2-like TAMs are frequently accumulated and associated with higher grade tumors (Komohara et al., 2008; Quail and Joyce, 2017; Wang et al., 2017). In contrast, repolarization of TAMs toward an antitumor M1-like phenotype results in tumor regression by producing pro-inflammatory cytokines and key molecules to stimulate T cell antitumor response. This indicates a potential therapeutic strategy of converting M2-like to M1-like TAMs for the treatment of GBM (Hambardzumyan et al., 2016; Quail and Joyce, 2017). Therefore, the classification of M1/M2-like TAM phenotypes and the functional plasticity of TAMs regulated through glioma cell-intrinsic mechanisms remain an area of active investigation. The instant study shows that CHI3L1, also known as human homolog YKL-40, predominately modulates the GBM TME using unbiased approaches. CHI3L1 signaling selectively regulates tumor infiltration and cell migration of MDMs and MG by forming distinct protein binding complexes. CHI3L1 protein complexes further reprogram TAMs to regulate T cell-mediated immune response in GBM progression. Importantly, a peptide was developed to disrupt CHI3L1 protein complexes, which can promote tumor regression in a syngeneic mouse GBM model, providing a therapeutic strategy to eradicate this devastating brain tumor.

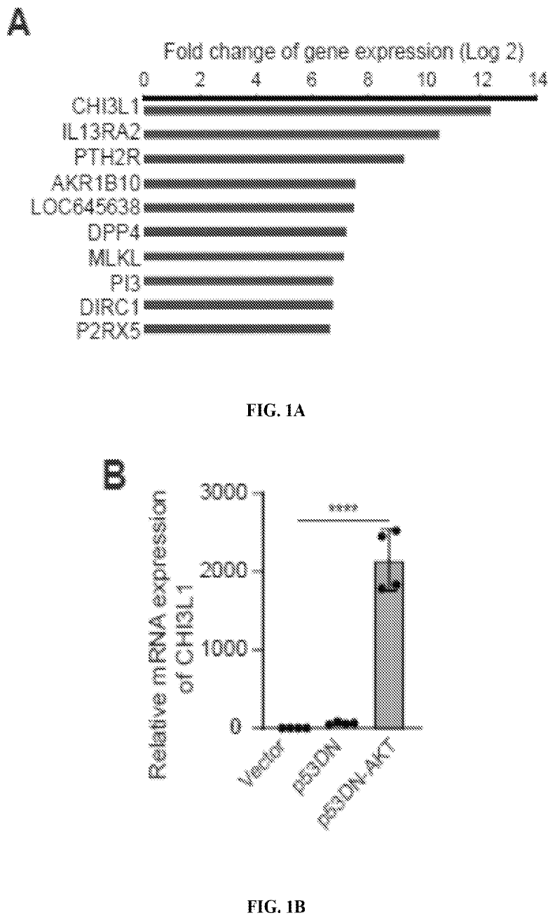

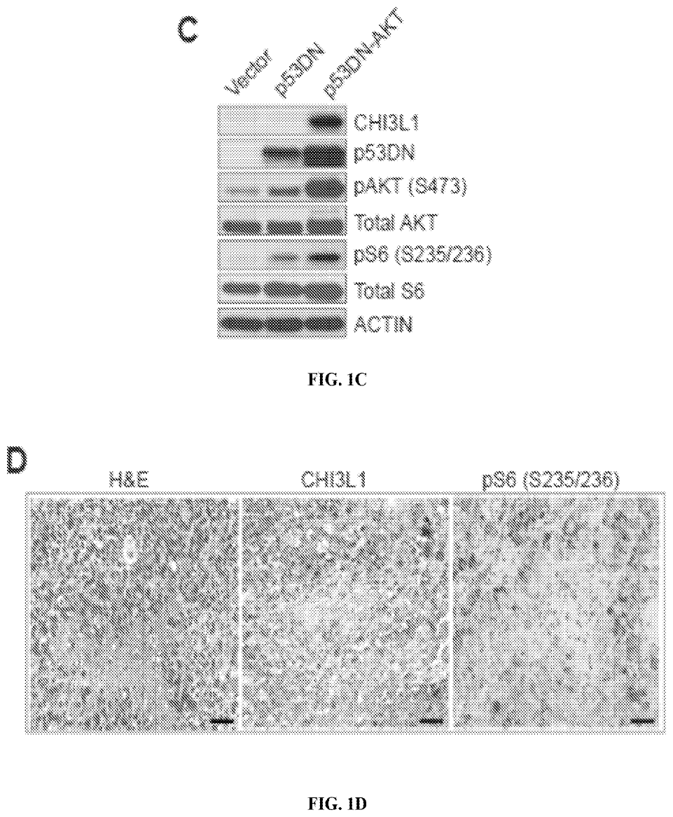

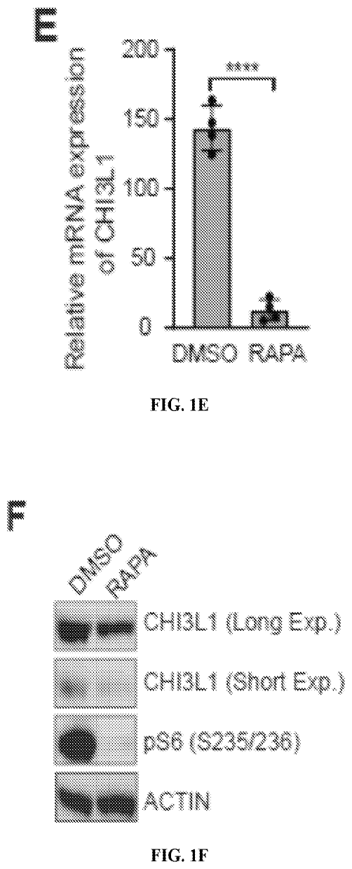

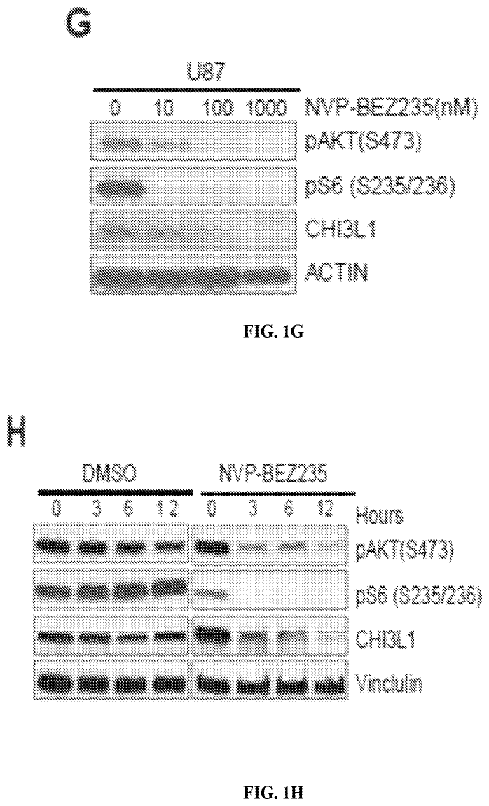

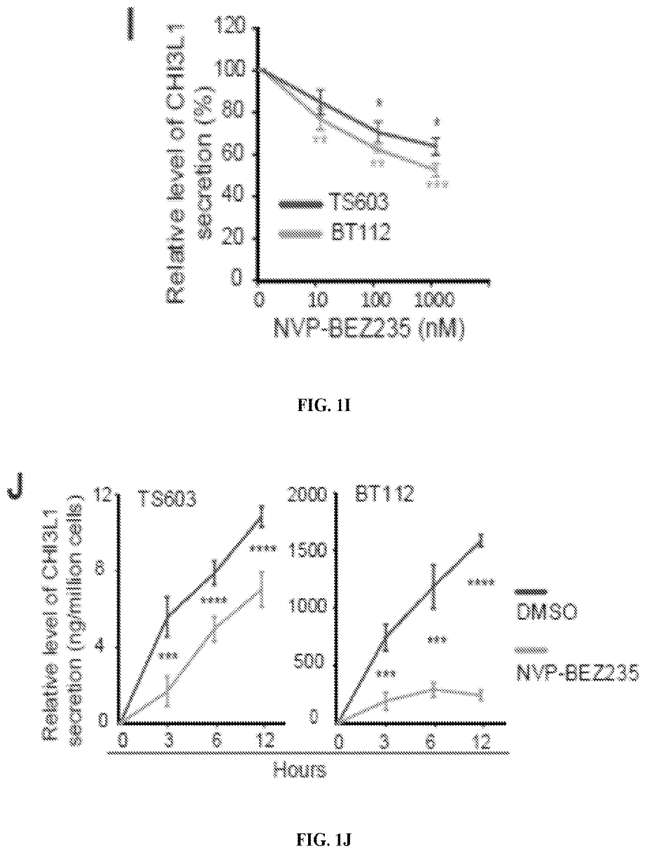

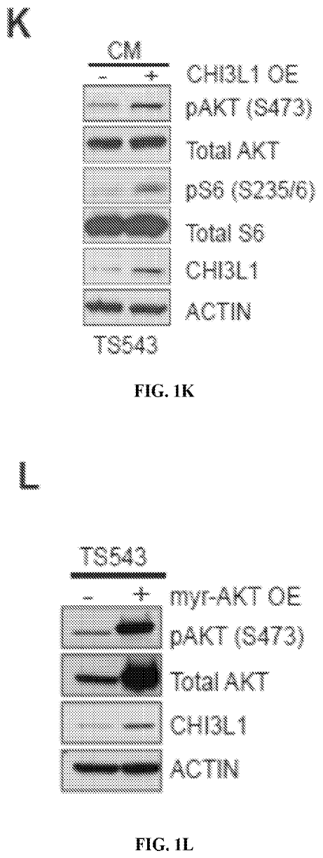

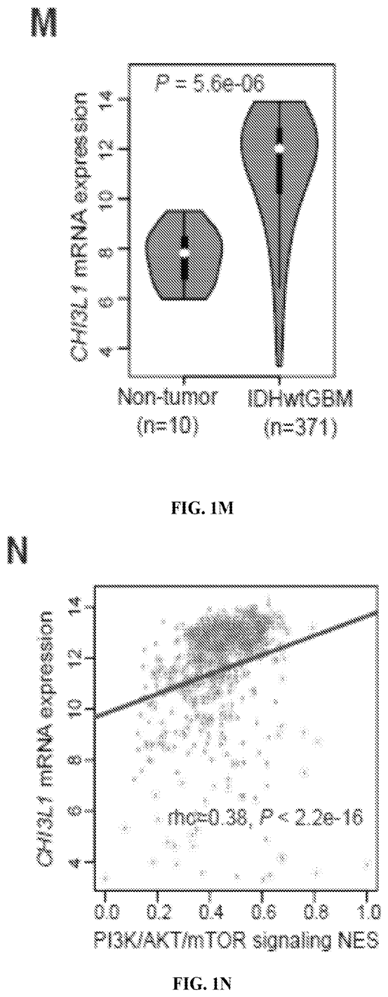

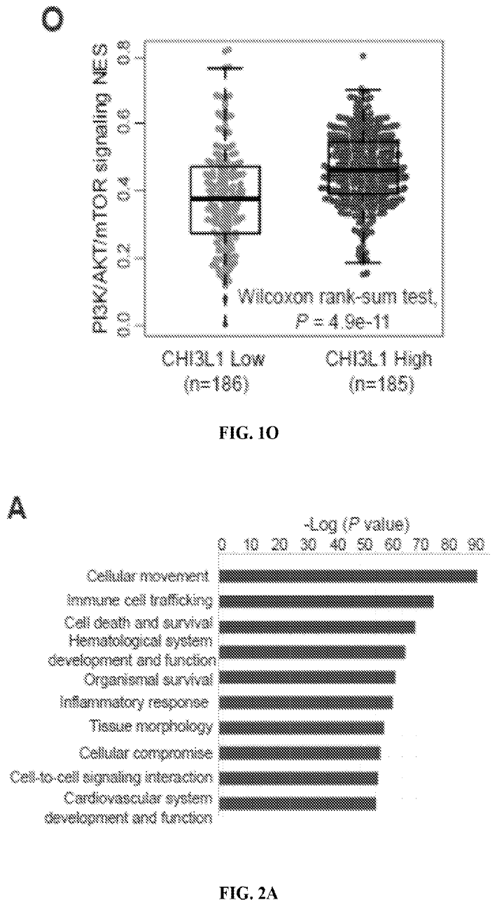

A previous study developed a de novo GBM model using a myristoylated form of AKT (myr-AKT) and dominant-negative p53 (p53DN)-engineered human neural stem cells (hNSCs), thereby enabling a performance of precise system-level comparisons between hNSCs and their derived glioblastoma stem cells (GSCs) (Hu et al., 2016). To identify cancer-cell-intrinsic factors for malignant transformation, global analyses of differentially expressed genes were performed between hNSCs and GSCs. CHI3L1 is the most significantly upregulated gene in GSCs derived from hNSCs overexpressing myr-AKT and p53DN (FIG. 1A). In vitro and in vivo validations revealed that CHI3L1 is highly expressed in GSCs and their derived tumors with activated AKT signaling (FIGS. 1B-1D). By contrast, inhibiting AKT/mTOR signaling by rapamycin decreased CHI3L1 mRNA and protein expression in hNSC-p53DN-AKT (FIGS. 1E-1F).

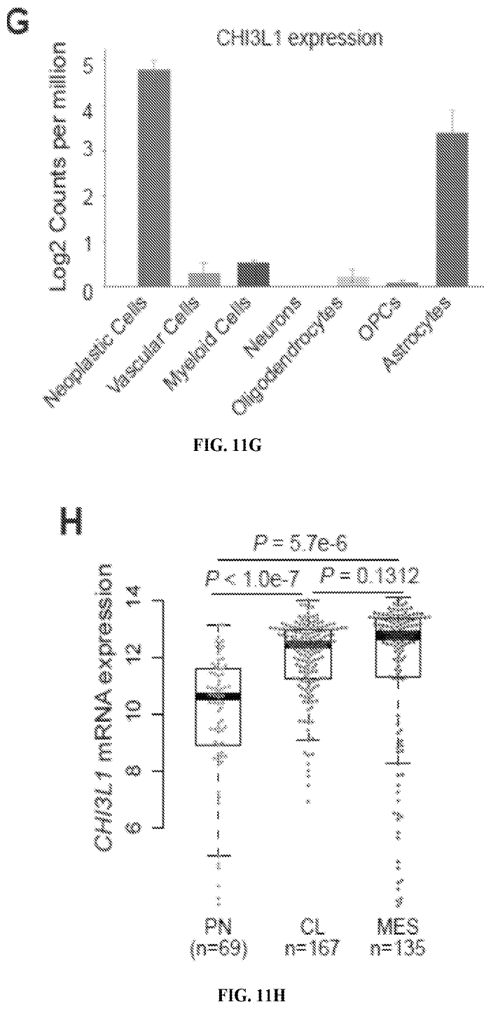

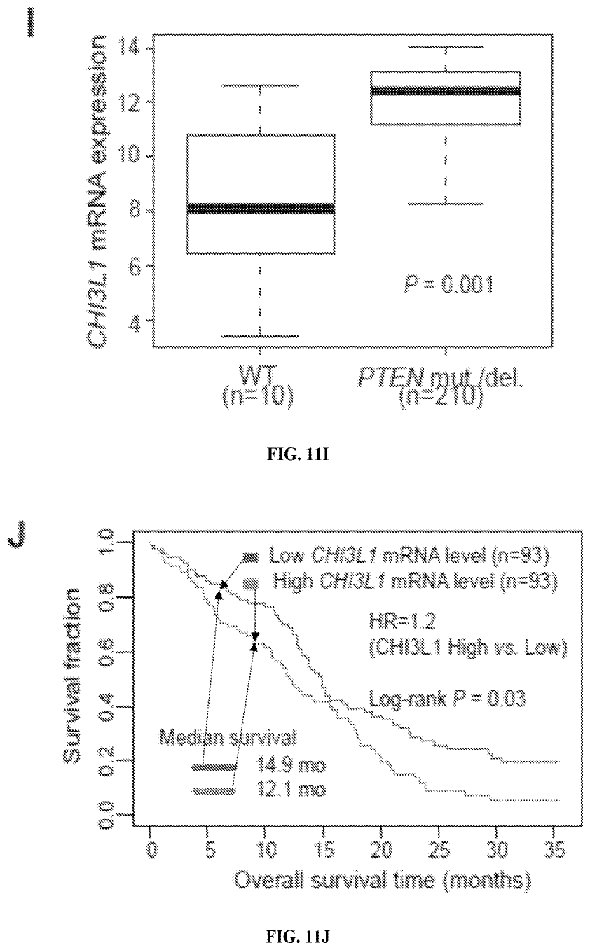

CHI3L1 is a secreted glycoprotein with chitin binding capacity but lacking chitinase activity (Fusetti et al., 2003), which plays a role in tissue remodeling, inflammation and cancer (Kamba et al., 2013; Lee et al., 2011). Although CHI3L1 is highly expressed and associated with a poor clinical outcome in GBM patients (Iwamoto et al., 2011; Pelloski et al., 2005), CHI3L1 regulation and its molecular mechanism(s) of action remain undefined. To test whether CHI3L1 is predominantly upregulated by the PI3K/AKT/mTOR signaling pathway, the GBM neurosphere line TS603 and U87 cells were treated with NVP-BEZ235 (a dual PI3K and mTOR inhibitor). Immunoblotting analysis revealed that CHI3L1 expression was regulated by PI3K/AKT/mTOR signaling in a time- and dose-dependent manner (FIGS. 1 G, 1H; FIGS. 11A and 11B). Furthermore, CHI3L1 levels were measured in the conditioned medium (CM) of two GBM neurosphere lines treated with NVP-BEZ235 or NVP-BKM120 (a pan-PI3K kinase inhibitor). Inhibition of PI3K/AKT/mTOR activation decreased CHI3L1 secretion in a dose- and time-dependent manner (FIGS. 11, 1J; FIGS. 11C, 11D). Importantly, the CM from GBM neurosphere line TS543 overexpressing CHI3L1 enhanced pAKT, pS6 and CHI3L1 levels over control in TS543 cells (FIG. 1K). Conversely, overexpression of myr-AKT dramatically increased CHI3L1 levels in TS543 (FIG. 1 L). These results demonstrate a positive feedback loop between CHI3L1 and the PI3K/AKT/mTOR signaling.

A previously published single-cell GBM transcriptomic patient dataset was analyzed (Darmanis et al., 2017), finding that glioma cells express high levels of CHI3L1 and represent a major source of CHI3L1 in the GBM TME (FIGS. 11E-11G). In the Cancer Genome Atlas (TCGA) GBM datasets, CHI3L1 is highly expressed in tumors versus non-tumor tissues and in mesenchymal versus proneural and classical subtypes (FIG. 1M; FIG. 11H). Moreover, higher levels of CHI3L1 mRNA expression are significantly associated with PTEN deletions/mutations, PI3K/AKT/mTOR signaling activation, and poor outcome in patients with IDH wildtype (IDHwt) GBM (FIGS. 1N, 1O; FIGS. 11I, 11J). Together, these results reinforce that a positive regulatory feedback loop of the PI3K/AKT/mTOR-CHI3L1 signaling plays a pivotal role in regulating GBM progression and treatment response.

### Example 2. CHI3L1 Plays a Predominant Role in Shaping the Landscape of the GBM Immune TME

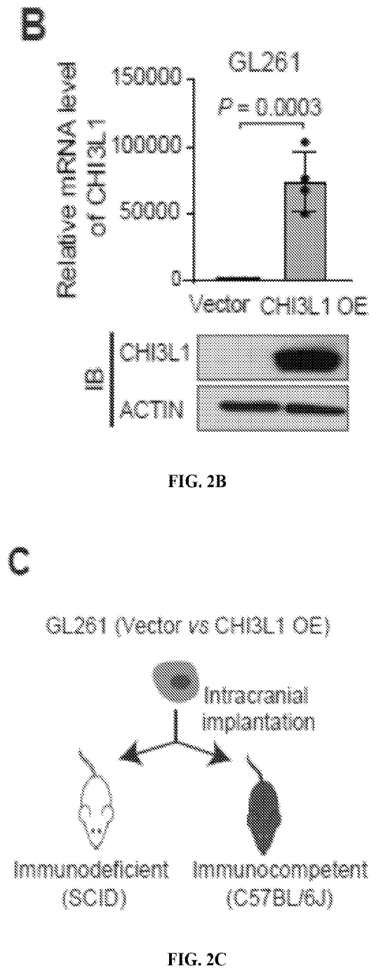

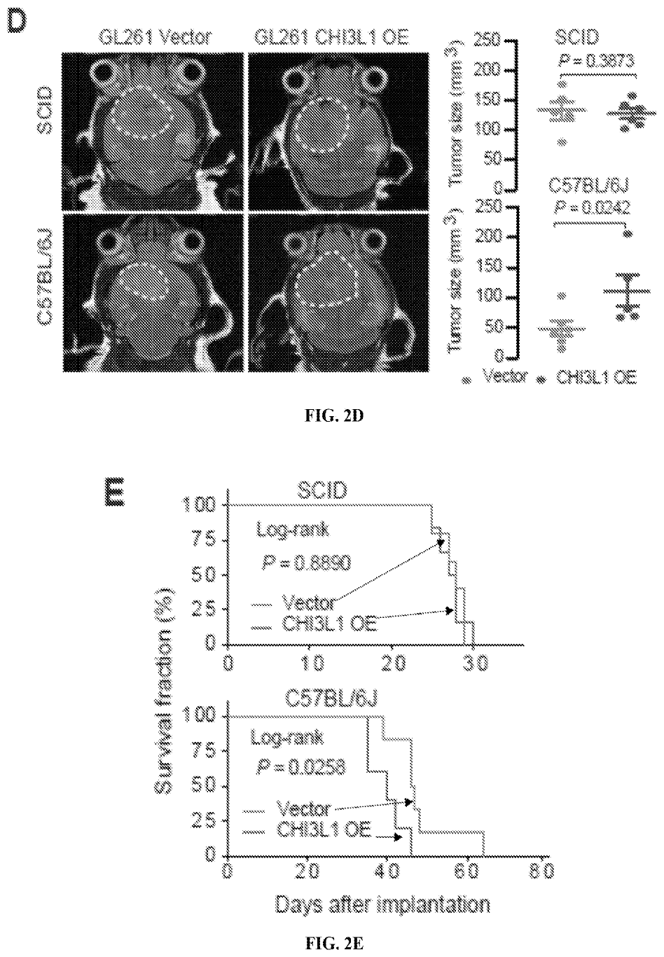

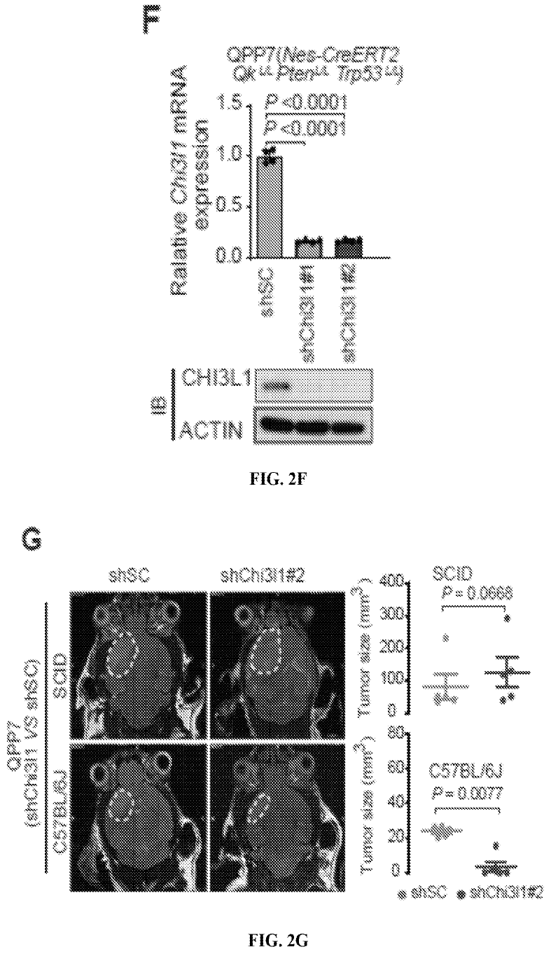

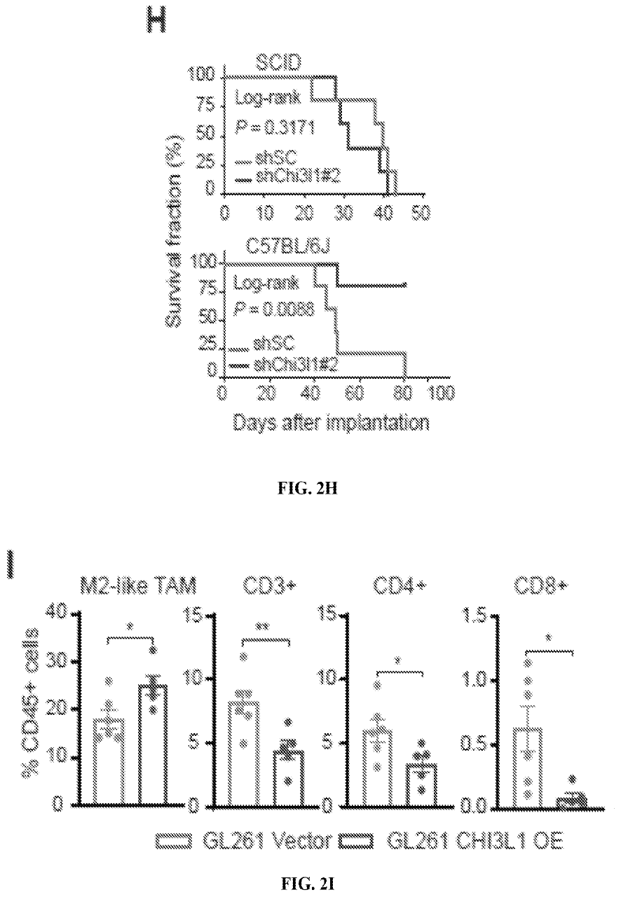

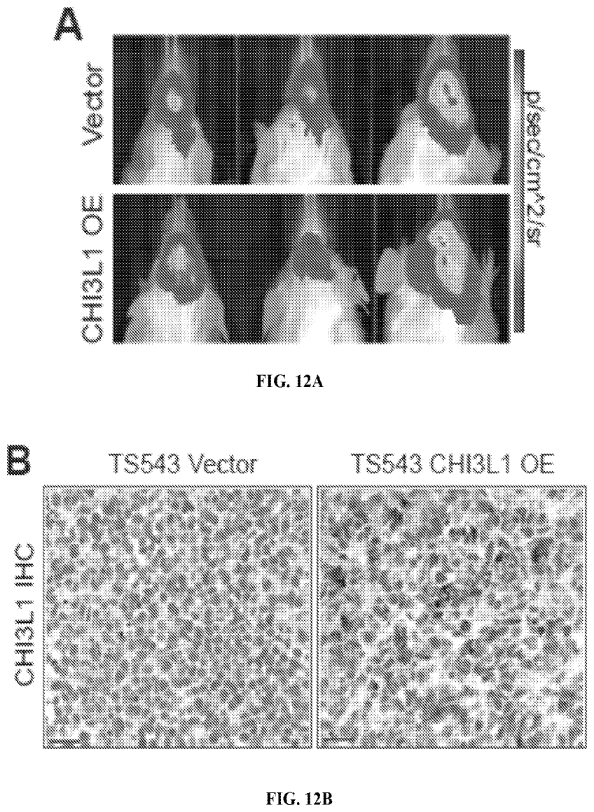

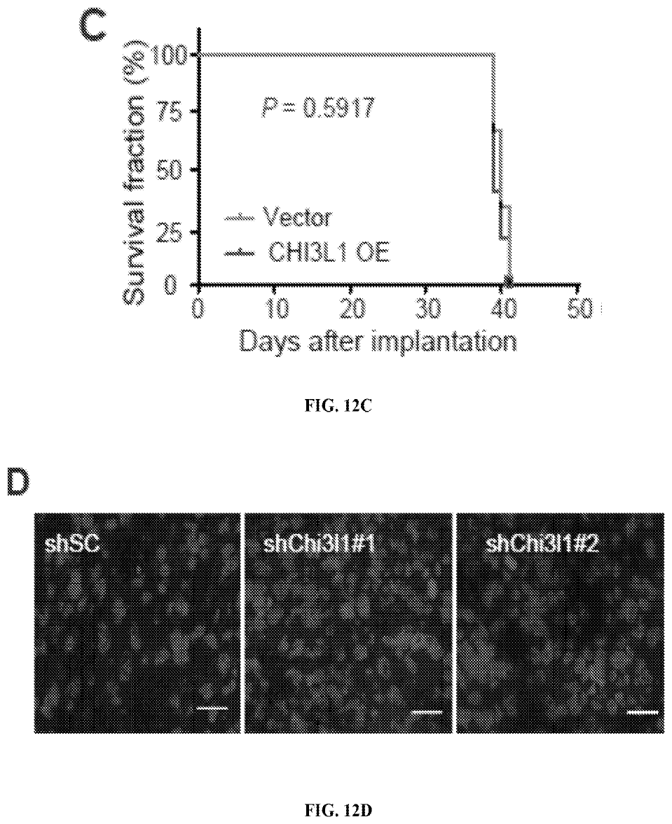

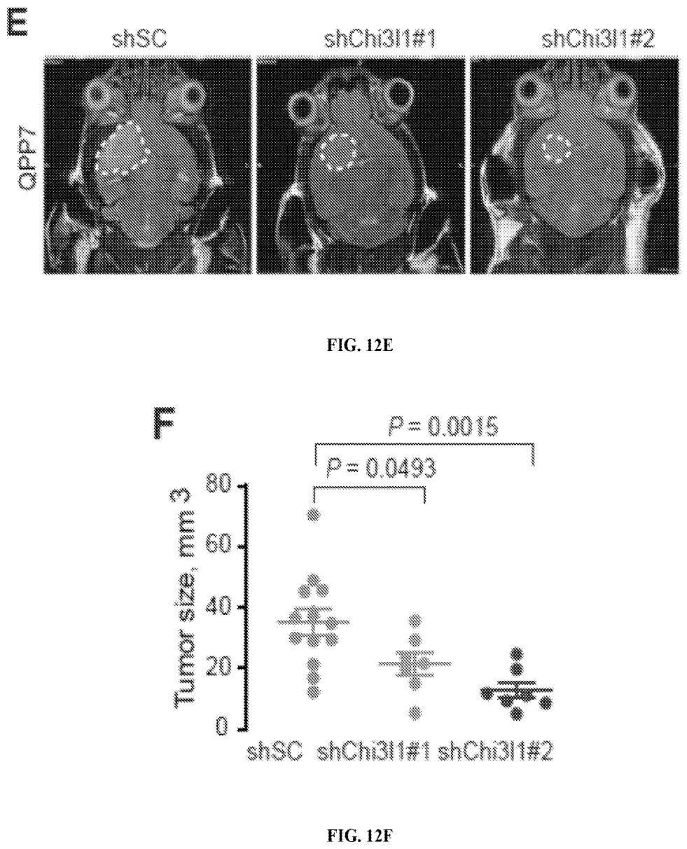

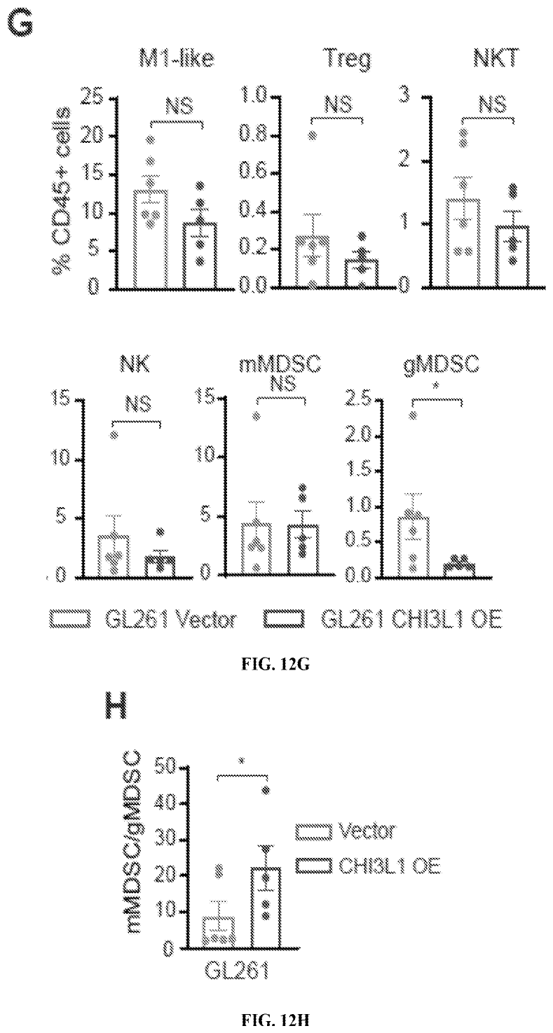

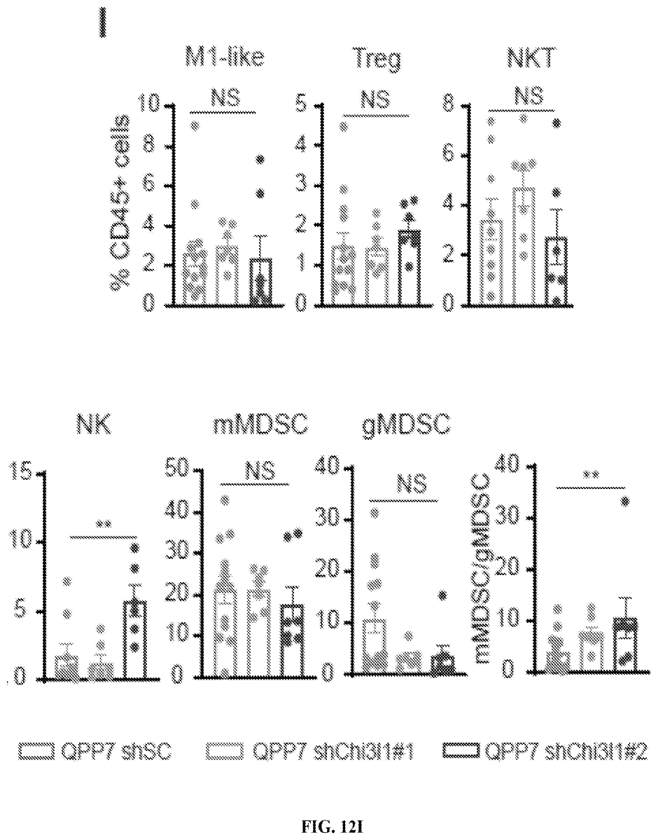

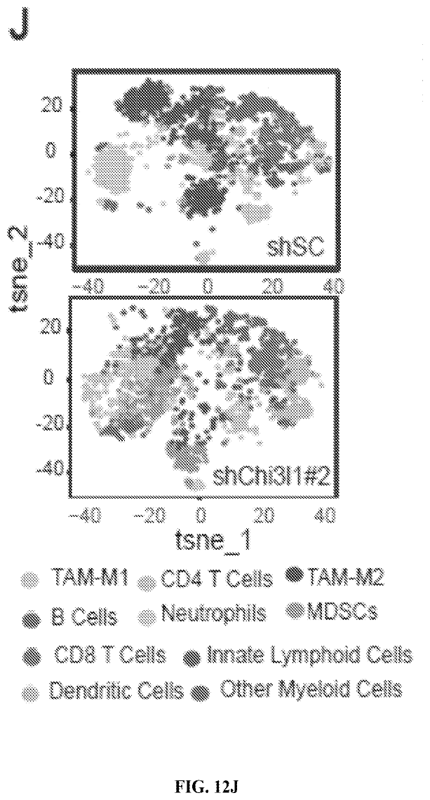

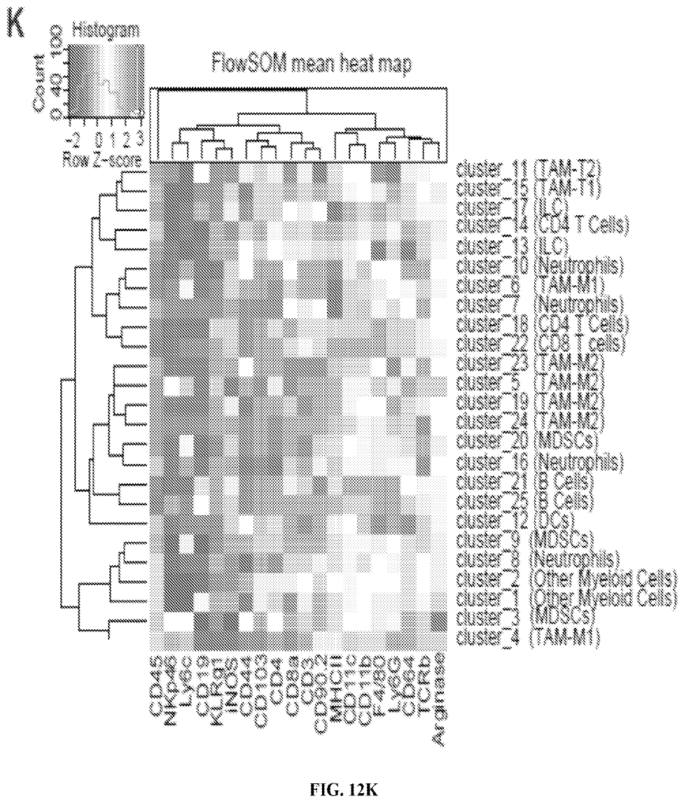

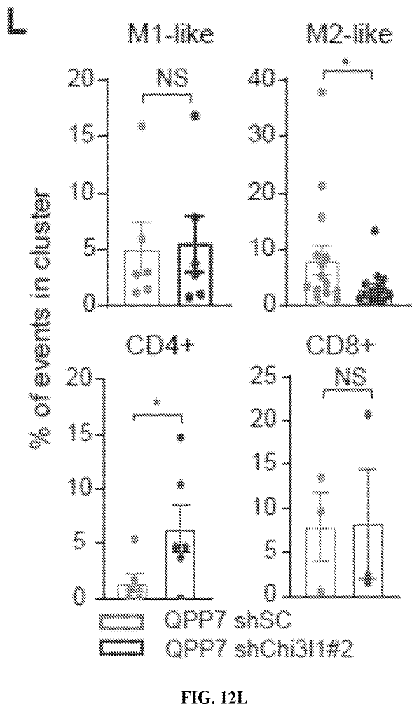

To determine the main function of CHI3L1 in GBM, in vivo CHI3L1 gain-of-function studies were performed in an orthotopic xenograft model of TS543 intracranially implanted in severe combined immunodeficiency (SCID) mice. Enforced CHI3L1 expression did not significantly promote tumor progression compared with vector controls (FIGS. 12A-12C). From the GBM TCGA dataset, CHI3L1 correlated genes (1,960 genes) are mainly associated with cellular movement, immune cell trafficking, and cell-to-cell signaling by Ingenuity Pathway Analysis (IPA) (FIG. 2A and Table 2). These data indicate that CHI3L1 plays a pivotal role in regulating the GBM immune TME. To this end, this study compared tumor progression between SCID and immunocompetent (C57BL/6) mice intracranially implanted with murine glioma GL261 cells that have low levels of endogenous CHI3L1 but forced expression of human CHI3L1 gene (GL261-CHI3L1) (FIGS. 2B and 2C). Notably, CHI3L1 overexpression increased tumor size and decreased survival in C57BL/6 mice, but not in SCID mice (FIGS. 2D and 2E). Conversely, an orthotopic syngeneic mouse model of GBM was generated using QPP7 cells that were derived from a spontaneous murine glioma model Nes-CreER^{T2}Qk^{L/L}Pten^{L/L}Trp53^{L/L} with high levels of endogenous CHI3L1 (FIG. 2F and FIG. 12D). In vivo loss-of-function studies revealed that knockdown (KD) of the mouse Chi311 gene (shChi3l1 #1 and #2) in QPP7 cells significantly repressed tumor growth in the syngeneic mice by magnetic resonance imaging (MRI) 4 weeks after implantation (FIG. 2F, FIGS. 2E and 2F). Importantly, comparative analyses of tumor progression in SCID and C57BL/6 mice bearing QPP7 tumors demonstrated that Chi311 KD decreased tumor size and extended the survival of immunocompetent mice, but not immunodeficient mice (FIGS. 2G and 2H). Together, these results show that CHI3L1 predominantly regulates the tumor immune TME, rather than tumor cells per se.

To determine the effect of CHI3L1 on immune cell distribution in the TME of GBM, we analyzed the major cell populations, including TAMs, T cells, Natural Killer (NK) cells, and myeloid-derived suppressor cells (MDSCs). Flow cytometry of tumors revealed that enforced CHI3L1 expression in GL261 mouse models significantly increased the M2-like TAMs (CD45⁺CD11b⁺CD14⁺MHCII⁺Ly6C⁻) but decreased CD3+, CD4+, and CD8⁺ T cell populations (FIG. 2I and FIGS. 12G and 12H). By contrast, Chi3l1 KD in QPP7 syngeneic models significantly decreased the M2-like TAMs but increased CD3⁺ and CD4⁺ T cell populations (FIG. 2J and FIG. 121). These findings were further validated by performing single-cell mass cytometry (CyTOF) analysis in the QPP7 tumors with Chi3l1 KD vs controls using the additional markers to identify cell populations (e.g., Arg1, iNOS, F4/80, CD90.2, etc.) (FIGS. 12J-12L). Notably, QPP7 tumors, compared to GL216 tumors, did not show significant changes of CD8⁺ T cells, which can support the low mutational load in QPP7 but not in GL261 cells determining the modest CD8⁺ T cell infiltration in these syngeneic GBM mouse models. Collectively, the gain/loss-of-function studies reveal a predominant role of CHI3L1 in regulating GBM progression by reprogramming the tumor immune microenvironment.

### Example 3. CHI3L1 Selectively Promotes Infiltration of M2-Like Versus M1-Like MDMs and MG

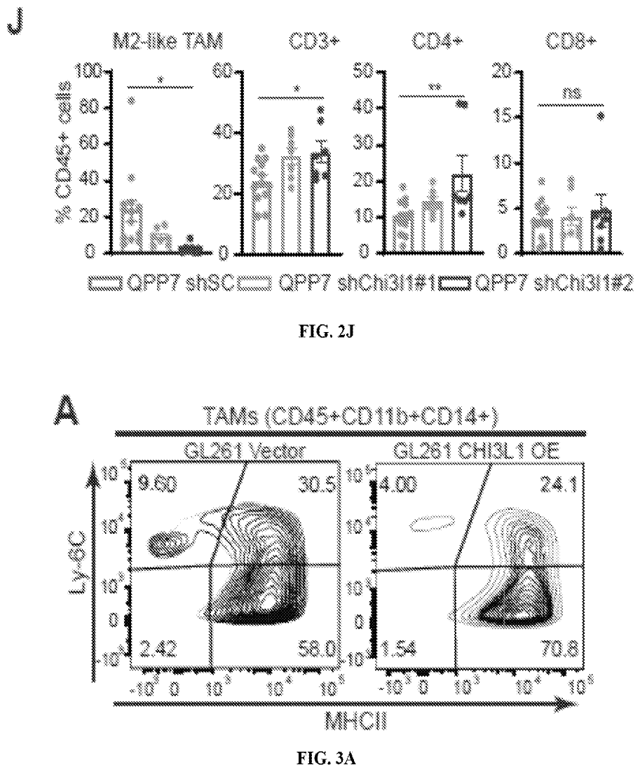

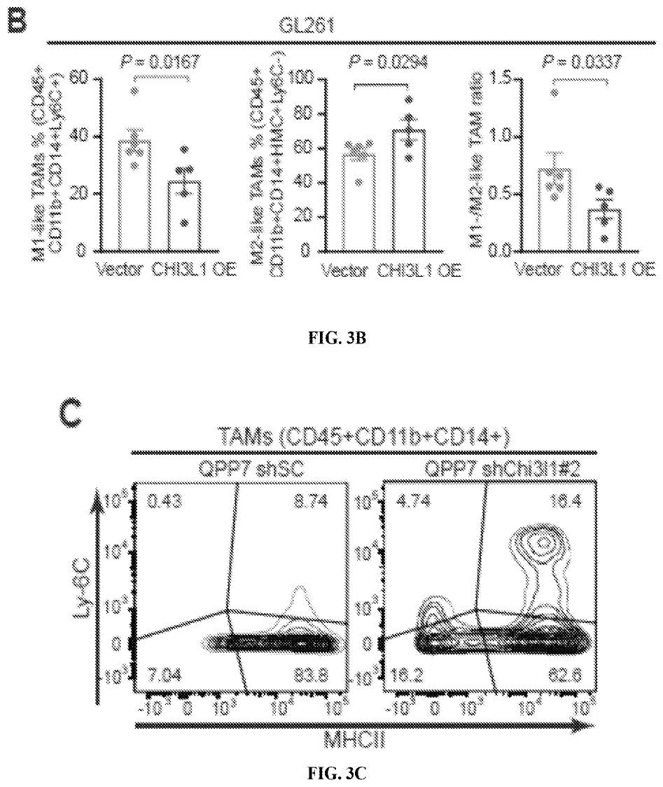

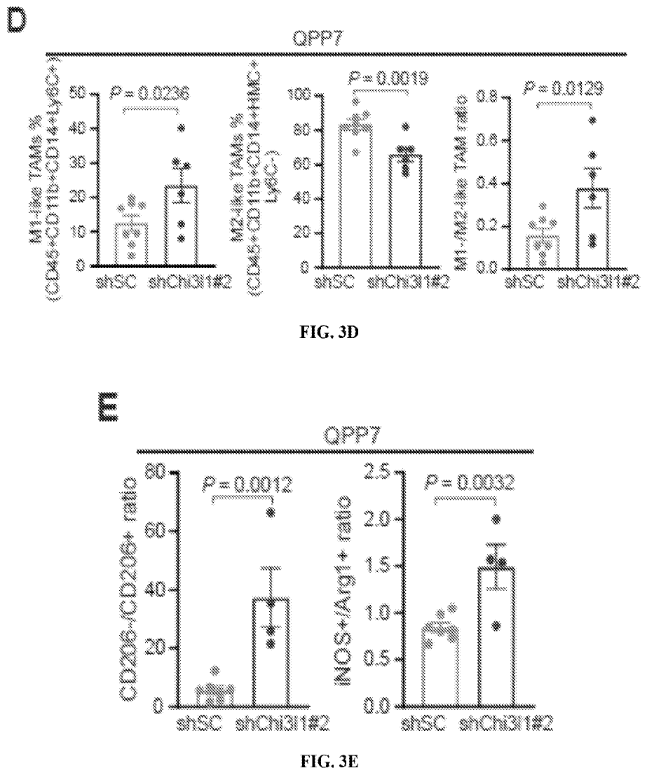

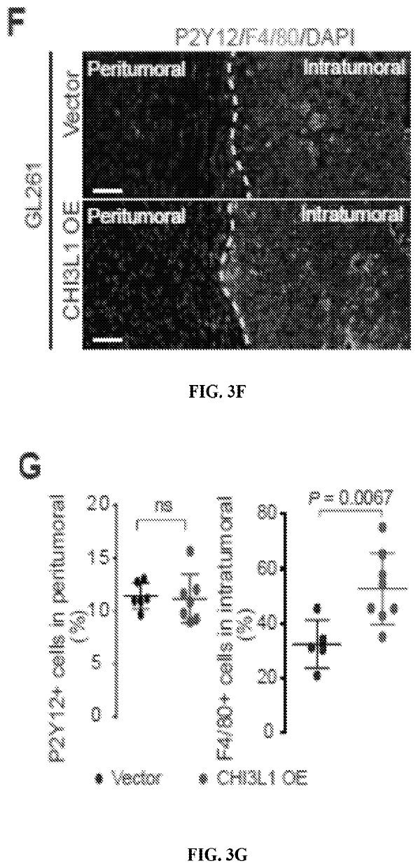

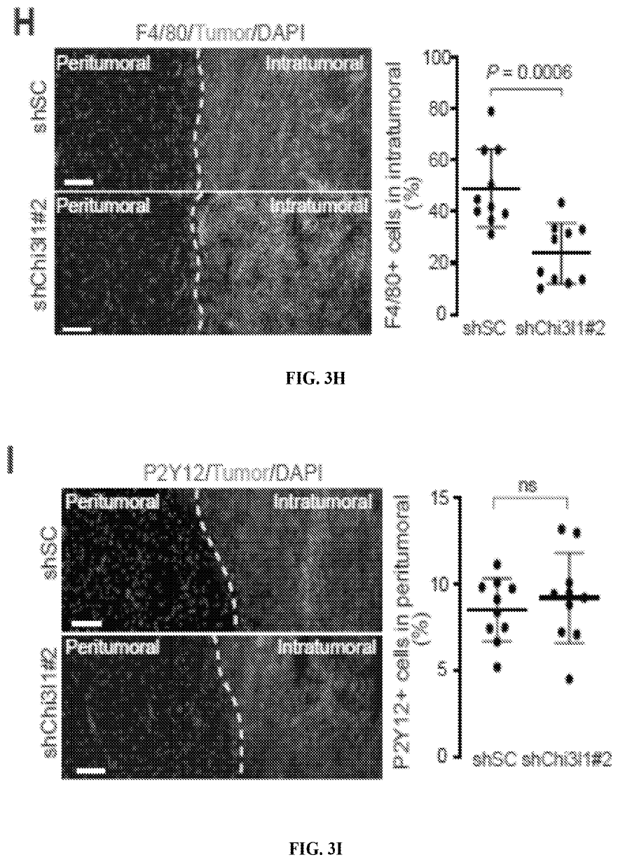

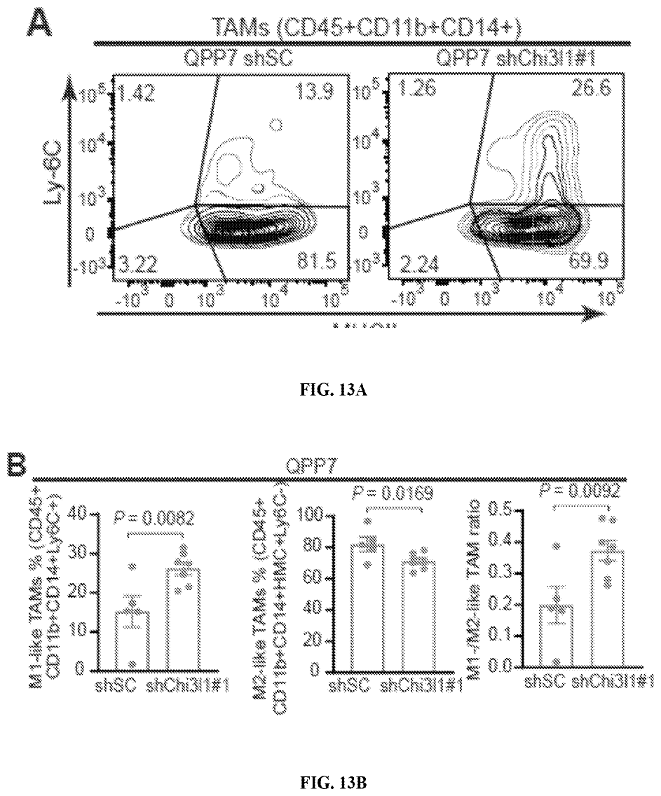

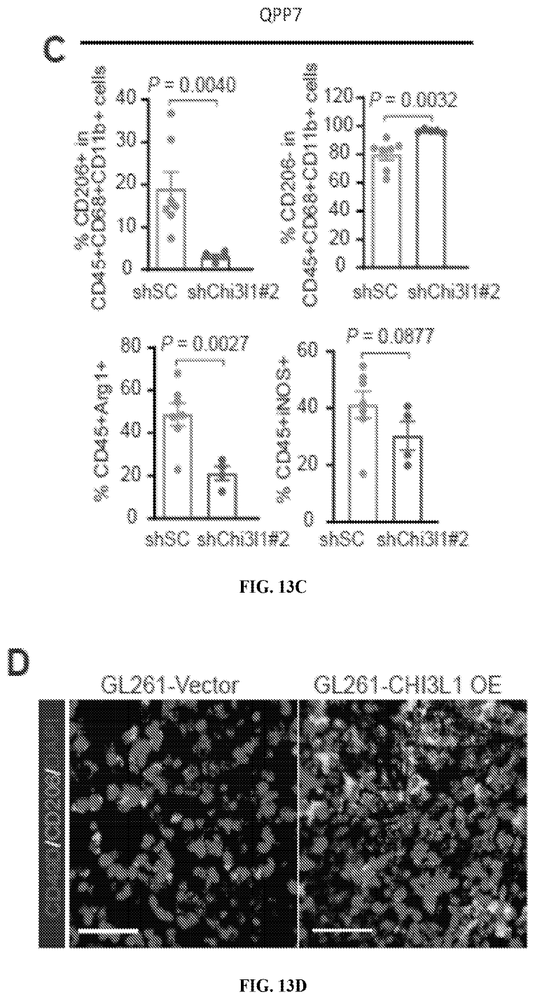

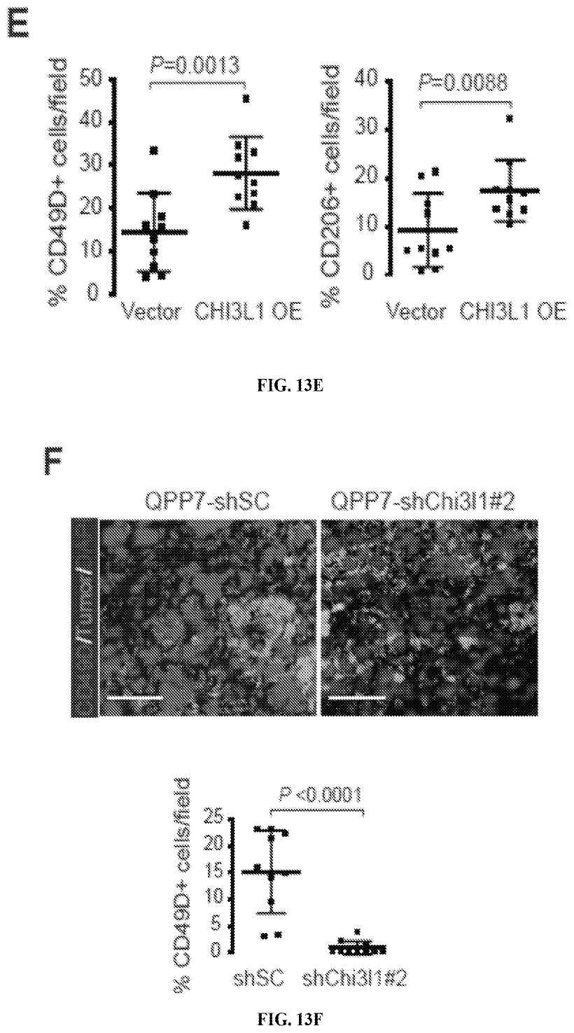

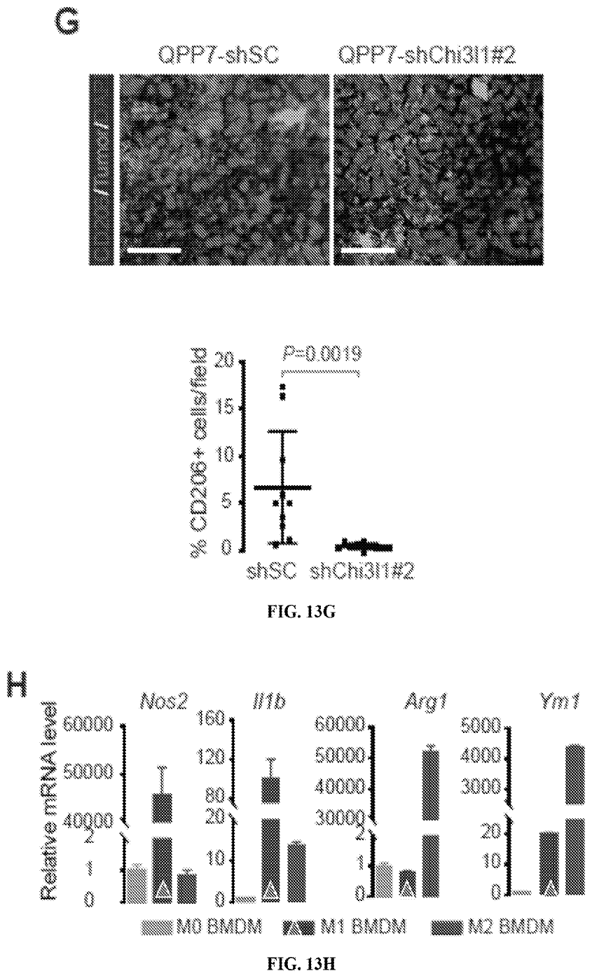

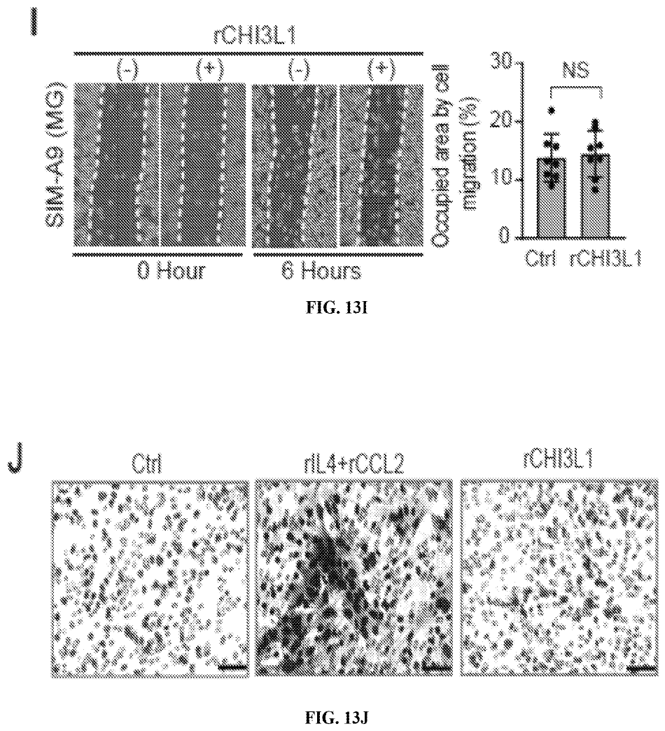

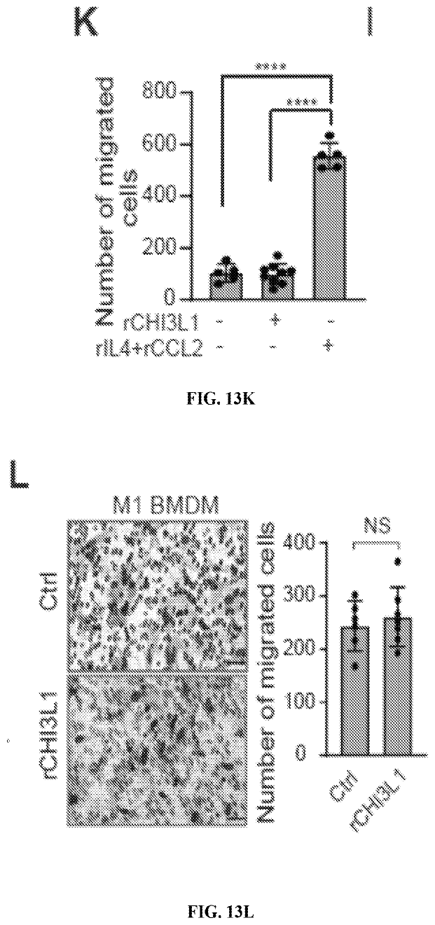

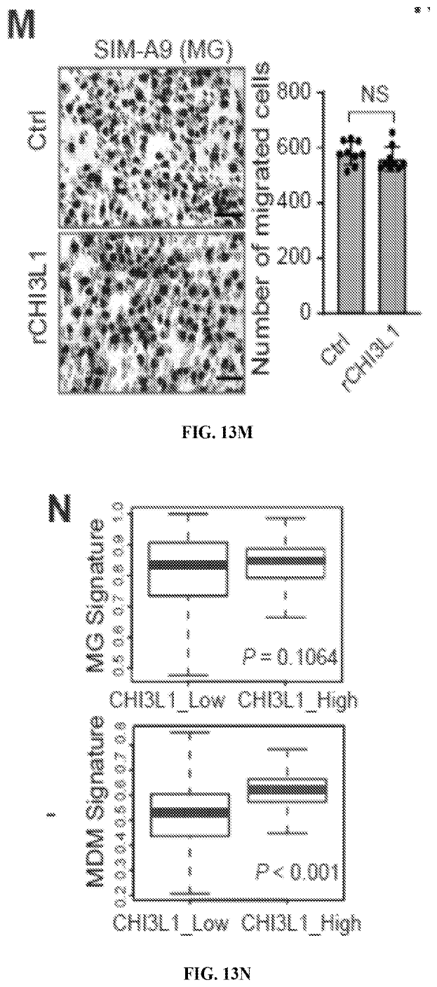

Previous studies reported the involvement of CHI3L1 in macrophage differentiation and recruitment associated with other pathological conditions although the mechanism of its action remains elusive. Furthermore, the M2-like TAMs consistently and significantly changed in response to CHI3L1 expression in our syngeneic mouse models. Therefore, this study focused on how CHI3L1 reprograms TAMs in the GBM TME. Further flow cytometry analyses showed that overexpression of CHI3L1 increased the percentage of M2-like TAMs but decreased M1-like TAMs (CD45⁺CD11b⁺CD14⁺Ly6C⁺) and the M1-/M2-like TAM ratio in GL261 tumor models (FIGS. 3A and 3B). Conversely, Chi3l1 KD decreased the percentage of M2-like TAMs but increased M1-like TAMs and the M1-/M2-like TAM ratio in QPP7-derived GBM models by flow cytometry (FIGS. 3C and 3D, and FIGS. 13A and 13B). Moreover, additional markers used for TAM polarization analyses revealed a significant decrease of the CD45⁺CD68⁺CD11b⁺CD206⁺ and CD45⁺Arg1⁺ populations but an increase in the ratios of CD206⁻/CD206⁺ and iNOS⁺/Arg1⁺ cells in QPP7 tumors (FIG. 3E and FIG. 13C). These results show that CHI3L1 regulates TAM polarization toward M2-like phenotype in the GBM TME.

To specifically investigate CHI3L1-regulated MDMs and MG tumoral infiltration, the study performed co-immunofluorescence (IF) staining to detect F4/80, a mature phagocytic cell marker, and P2Y12, a classic marker for microglia (Butovsky et al., 2014; Haynes et al., 2006). Notably, overexpression of CHI3L1 in GL261-derived glioma models greatly increased F4/80+ cell accumulation in intratumoral regions but did not significantly change infiltration of P2Y12+ MG, which predominantly reside in peritumoral regions (FIGS. 3F and 3G). In contrast, there was a decreased F4/80+ cell infiltration in QPP7-derived gliomas without a significant change of peritumoral P2Y12+MG in Chi3l1 KD versus controls (FIGS. 3H and 3I). Furthermore, intratumoral MDMs (M2-like) were significantly accumulated in GL216-CHI3L1 tumors but reduced in Chi311 KD QPP7 tumors, respectively, based on IF staining using additional markers (CD206 and CD49D) (FIGS. 13D-13G). These data reveal that cancer-cell-intrinsic CHI3L1 promotes accumulation of MDMs over MG within the tumor, which provides a mechanistic explanation for the observation of abundant MDM infiltration in tumor lesions while the preferential occupation of MG in the periphery (Chen et al., 2017; Darmanis et al., 2017; Pinton et al., 2019).

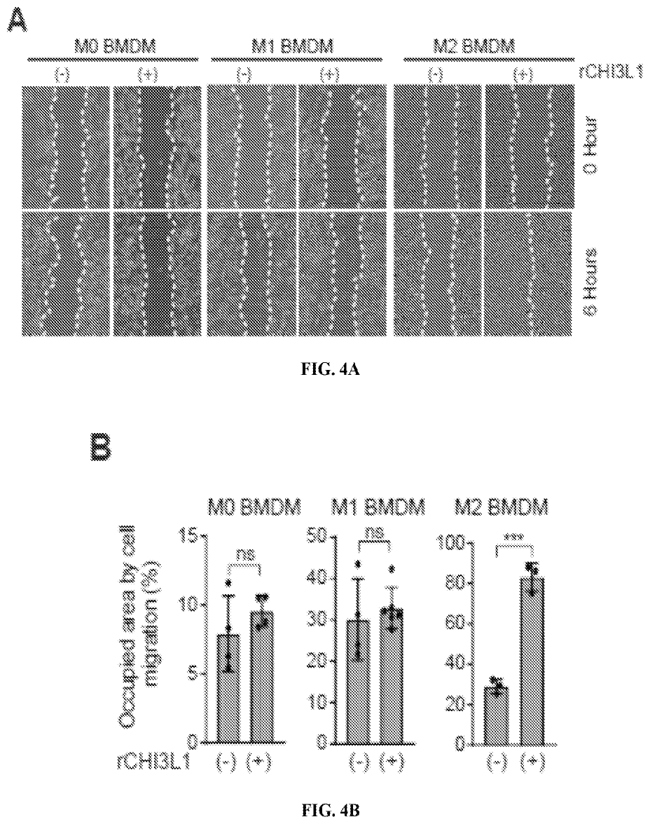

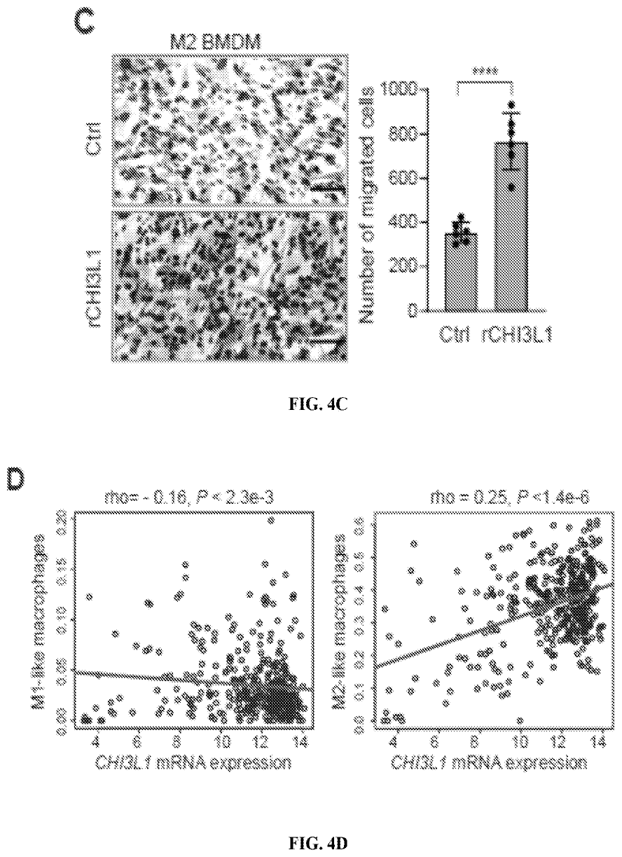

To further verify the effect of CHI3L1 on TAM infiltration, in vitro scratch-wound healing and transwell assays were performed to examine cell migration of bone-marrow derived macrophages (BMDMs) and microglial cells treated with recombinant CHI3L1 protein (rCHI3L1). the polarization states of M0, M1 and M2 macrophages were generated and confirmed using isolated BMDMs from C57BL/6 mice according to a previous standard protocol (Ying et al., 2013) (FIG. 13H). Strikingly, rCHI3L1 treatment promoted M2 BMDM migration but not M0 and M1 BMDMs (FIGS. 4A-4B). However, rCHI3L1 treatment did not increase cell migration in a mouse microglial cell line (SIM-A9) (FIG. 3I), supporting the observation that MG tumor infiltration is unaffected by CHI3L1. Moreover, a transwell assay confirmed that rCHI3L1 promoted cell migration in M2 BMDMs, but not in M0 BMDMs, M1 BMDMs, or microglial cells (FIG. 4C; FIGS. 13J-13M).

Based on CIBERSORT for gene signatures and correlation analysis in the GBM TCGA datasets (Chen et al., 2018; Wang et al., 2017), CHI3L1 mRNA expression is positively correlated with tumor-promoting M2-like macrophages but negatively correlated with tumor-killing M1-like macrophages (FIG. 4D). Furthermore, analysis of gene set signatures (Muller et al., 2017) revealed that MDMs, rather than MG, are significantly enriched in tumors with higher levels of CHI3L1 expression in GBM patients (FIG. 3N). These results demonstrate that CHI3L1 regulates TAM polarization and selectively promotes the migration and accumulation of M2-like MDMs in GBM.

### Example 4. Gal3BP Binding to CHI3L1 Negatively Regulates M2-Like Macrophage Migration

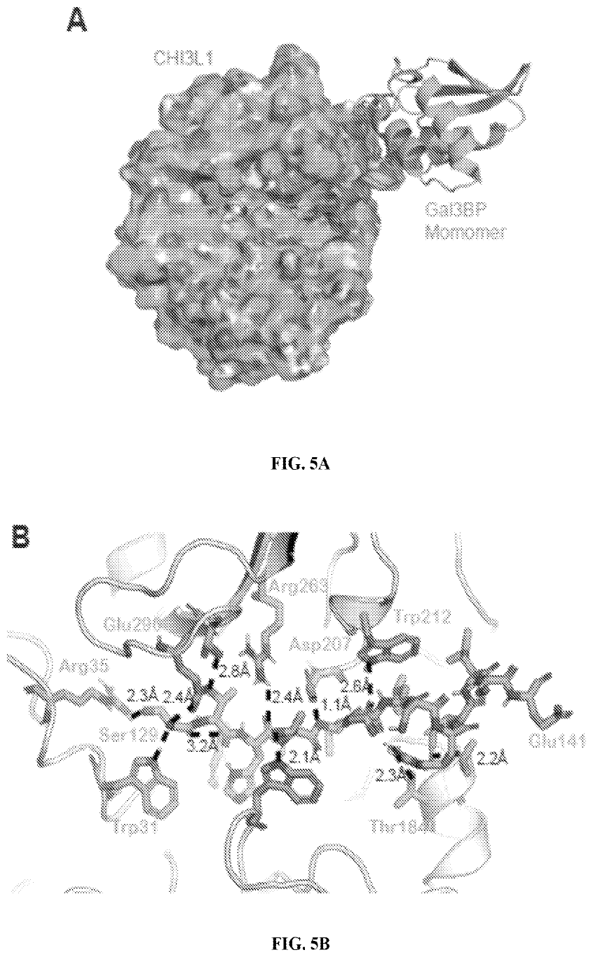

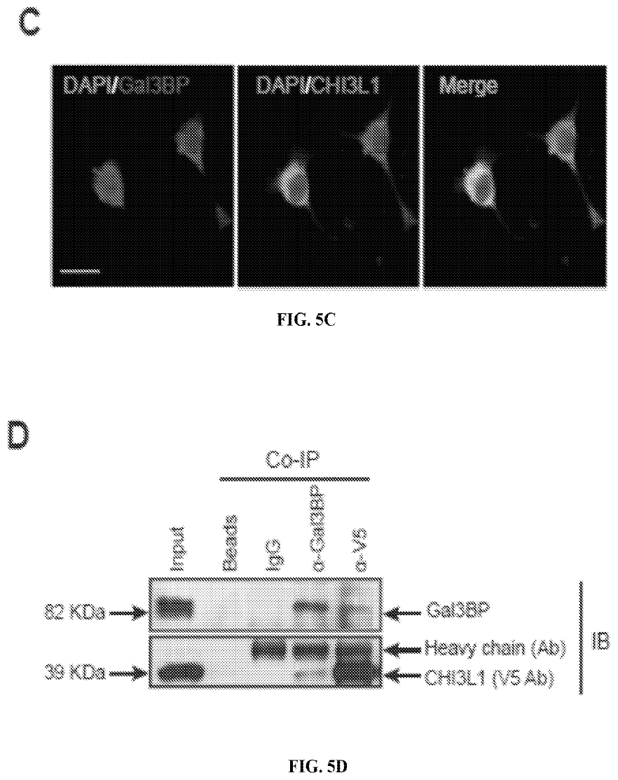

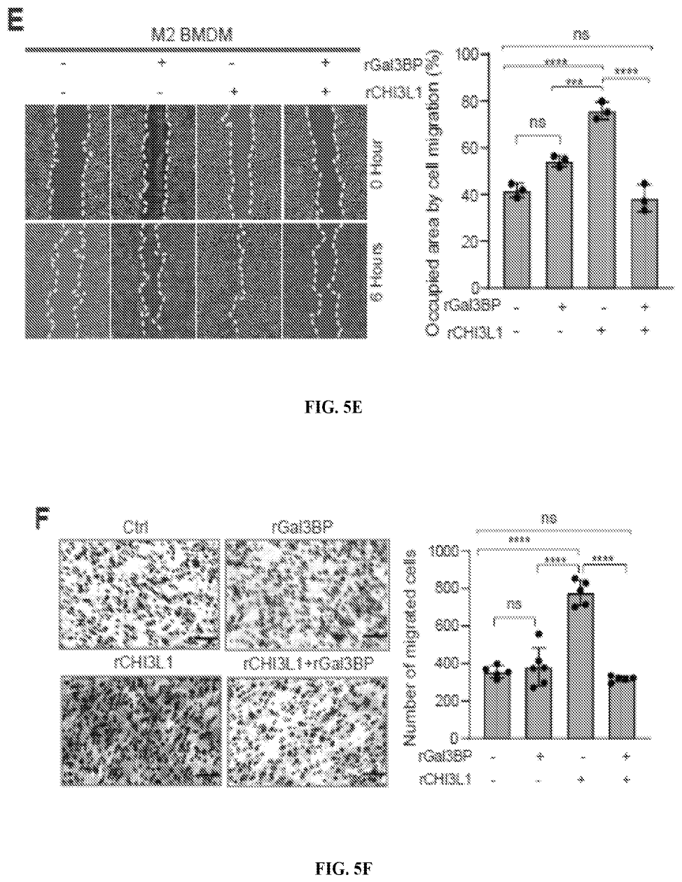

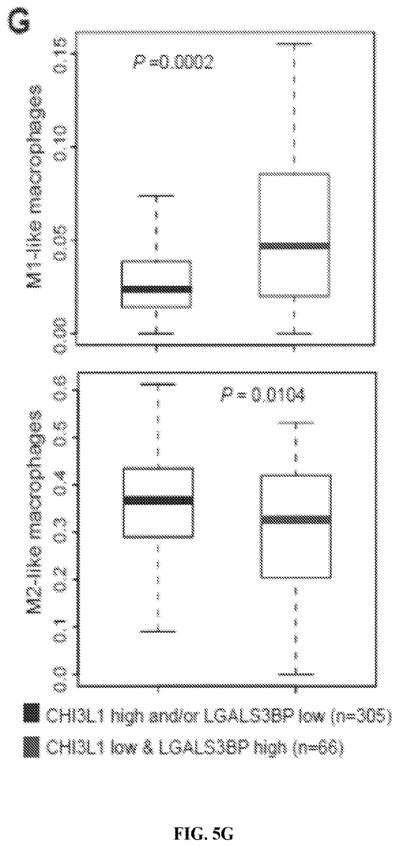

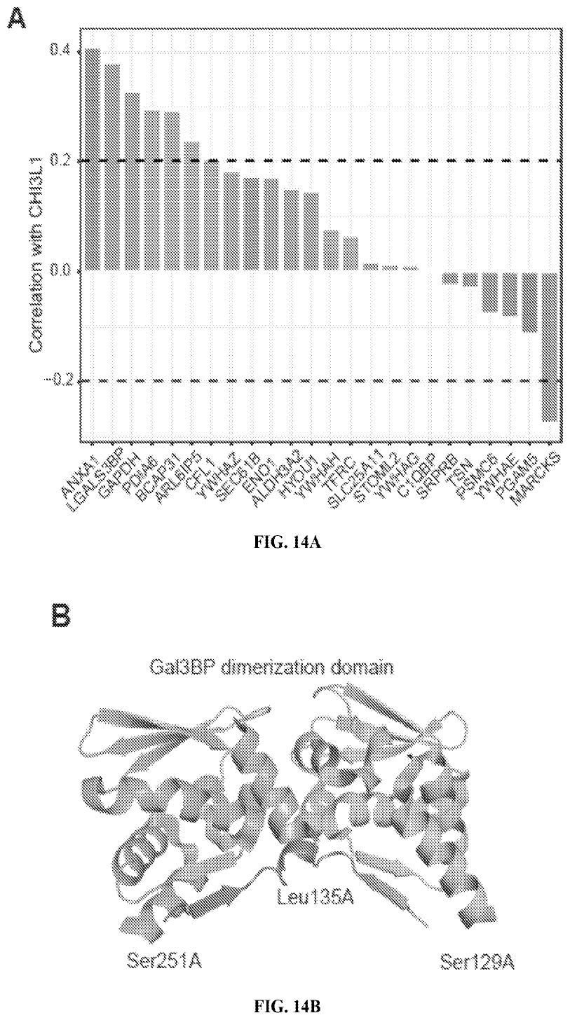

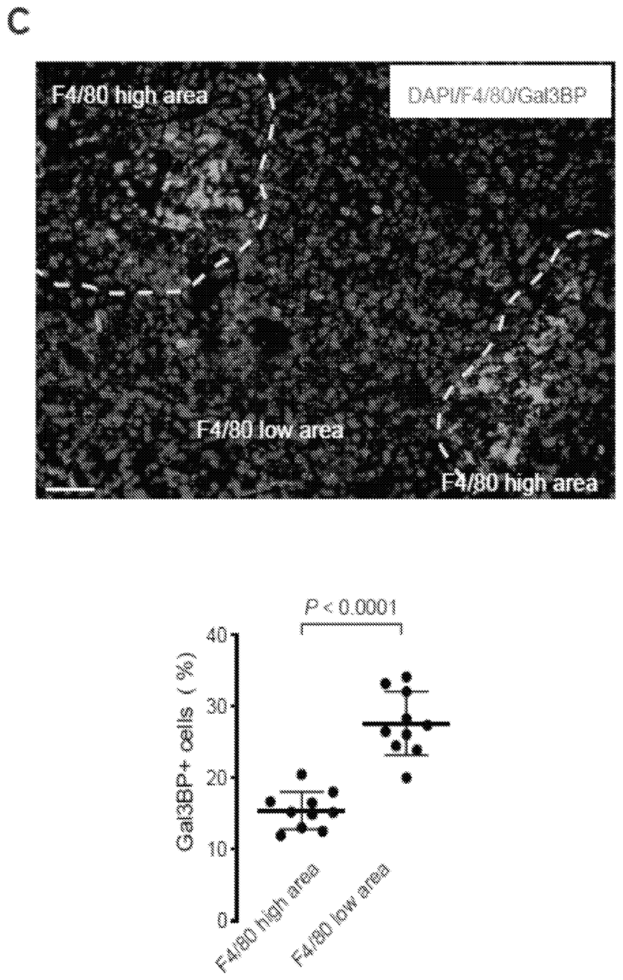

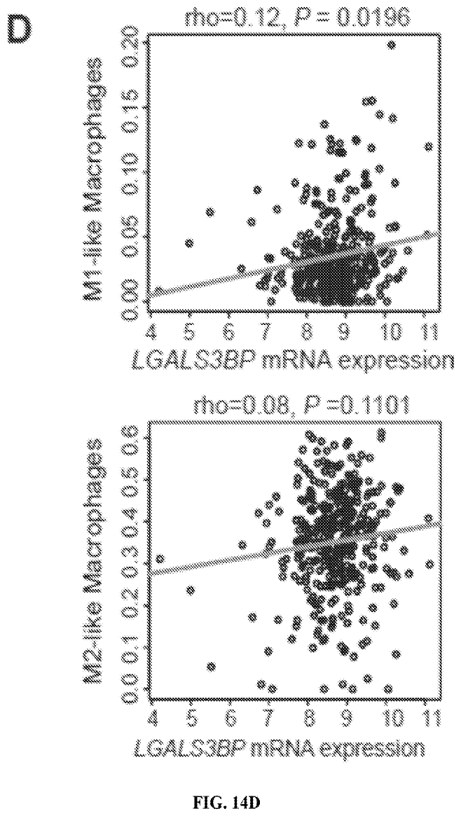

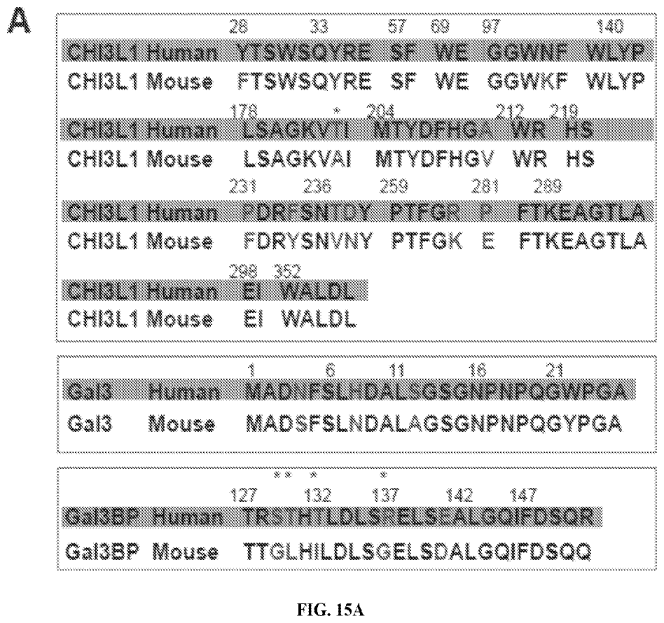

To elucidate how CHI3L1 promotes M2-like MDM migration, CHI3L1 binding proteins were explored using immunoprecipitation coupled to liquid chromatography-mass spectrometry (LC-MS). LC-MS analysis of extracellular or membrane-associated proteins revealed 7 putative binding proteins encoded by the ANXA1, LGALS3BP, GAPDH, PDIA6, BCAP31, ARL6IP5, and MARCKS gene, which are highly associated with CHI3L1 in GBM (FIG. 14A; Table 3). None of these genes have been previously identified as binding partners of CHI3L1. An orthogonal structure-based screening identified Gal3BP, encoded by the LGALS3BP gene, as a binding partner of CHI3L1 (FIGS. 5A-5B). Gal3BP, also known as 90K or Mac-2-binding protein, is a secreted glycoprotein upregulated and involved in innate immunity against viral and bacterial infections (Loimaranta et al., 2018). The domain of Gal3BP predicted to interact with CHI3L1 corresponds to Gal3BP main dimerization domain (FIG. 14B), indicating that CHI3L1 can bind monomeric Gal3BP to disrupt its dimerization. Co-IF staining demonstrated strong colocalization of Gal3BP and CHI3L1, which was further supported in live cells by co-immunoprecipitation (Co-IP) (FIGS. 5C, 5D).

To test whether Gal3BP binding to CHI3L1 promotes MDM migration, M2 BMDMs were treated with rCHI3L1 and recombinant Gal3BP protein (rGal3BP). rGal3BP significantly attenuated rCHI3L1-induced M2 BMDM migration, as found by scratch-wound healing assay (FIG. 4E) and transwell migration assay (FIG. 4F). Analyzing IF staining of tumors derived from GL261-CHI3L1 bearing syngeneic mice shows the mutually exclusive expression patterns of Gal3BP and F4/80 (FIG. 11C), indicating a negative correlation between Gal3BP expression levels and MDM distribution in glioma. Assessment of correlation between Gal3BP expression and gene signatures of M1/M2-like macrophages revealed a significant positive correlation between LGALS3BP mRNA expression and M1-like macrophages compared with M2-like macrophages in the GBM TCGA database (FIG. 11D). Furthermore, M1-like macrophage signature was found to be highly enriched in GBMs with low levels of CHI3L1 expression but high levels of LGALS3BP expression, whereas a decrease of M2-like macrophage signature was shown in these tumors (FIG. 4G). These results revealed that Gal3BP binding to CHI3L1 negatively regulates CHI3L1-induced M2-like MDM infiltration in the GBM TME.

### Example 5. CHI3L1 Binds to Gal3 Resulting in Selective Migration of MDMs, which is Negatively Regulated by Gal3BP

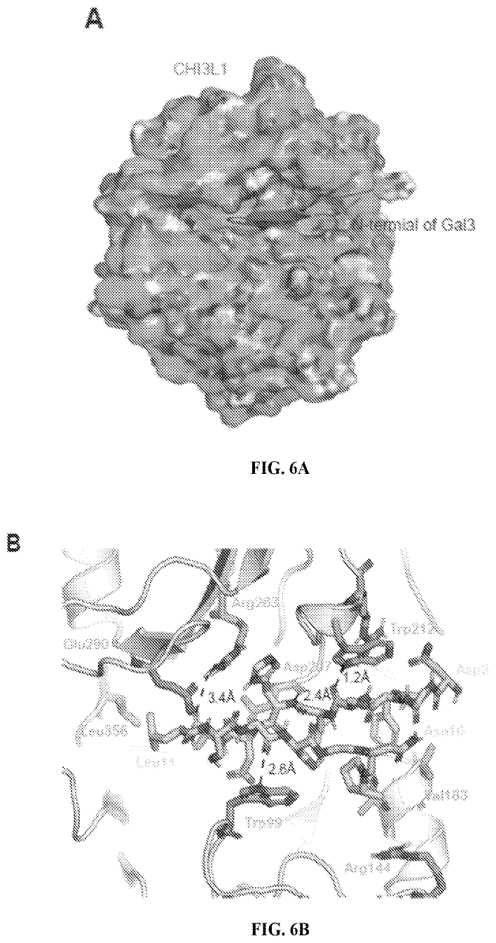

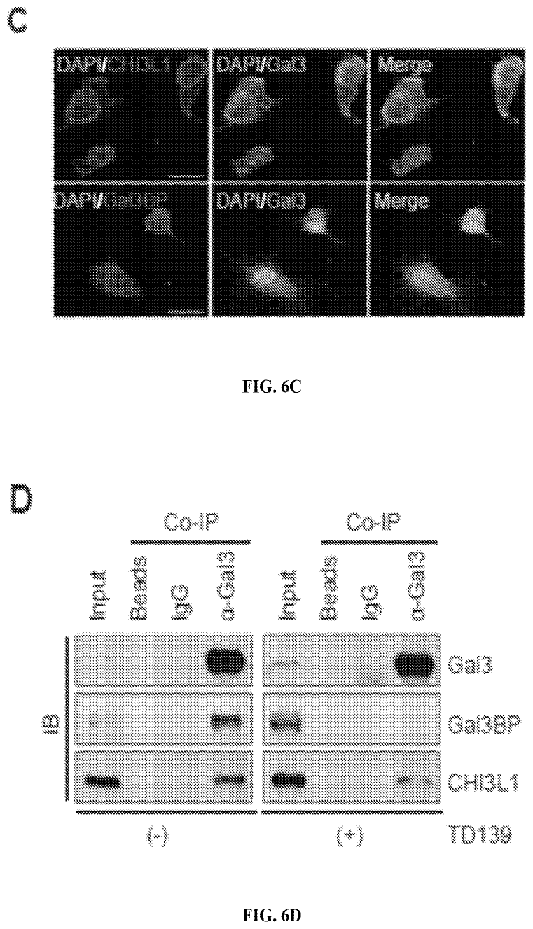

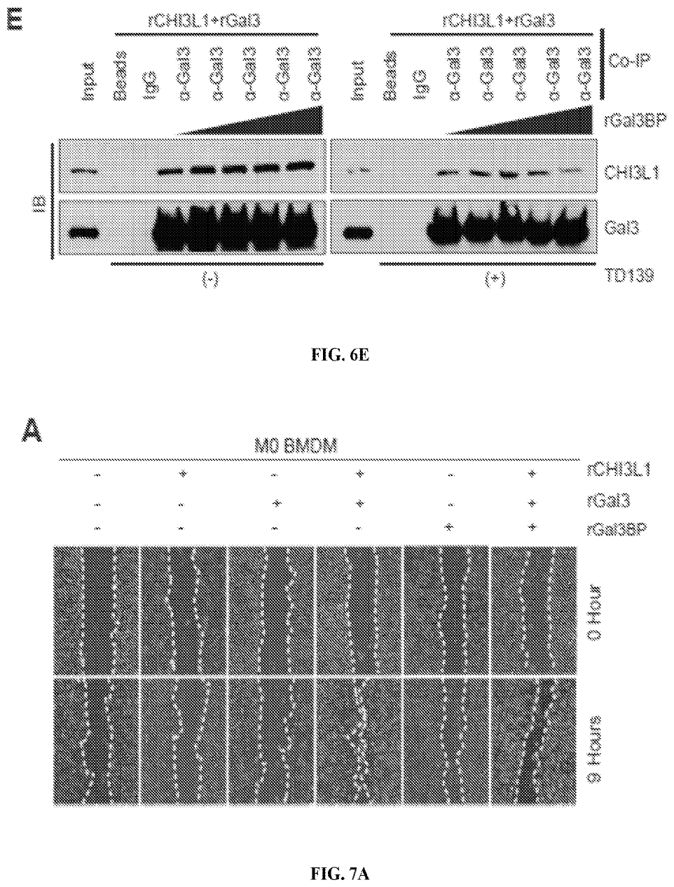

Gal3, encoded by the LGALS3 gene as a binding partner of Gal3BP, plays a critical role in macrophage migration and activation (Inohara et al., 1996; MacKinnon et al., 2008; Sano et al., 2000). Therefore, this study assessed whether Gal3 can be also involved in CHI3L1-mediated MDM migration. In silico docking of the N-terminal domain of Gal3 and CHI3L1 shows that Gal3 interacts with CHI3L1 in the same binding pocket as Gal3BP (FIGS. 6A, 6B). Consistent with this observation, Gal3-Gal3BP binding as well as Gal3-CHI3L1 binding were verified by Co-IP and Co-IF assays (FIGS. 6C, 6D). Gal3BP was shown to bind with a conserved carbohydrate recognition domain (CRD) at the C-terminal domain of Gal3. The Co-IP assay demonstrated that TD139, a high-affinity and potent inhibitor of Gal3, completely disrupted Gal3-Gal3BP but not Gal3-CHI3L1 interactions, indicating a novel binding mechanism of Gal3 and CHI3L1(FIG. 6D). Importantly, Gal3 and Gal3BP can compete for the same binding site in CHI3L1, which was validated by a Co-IP assay by adding an increasing amount of rGal3BP into the mixture of rCHI3L1 plus recombinant Gal3 protein (rGal3) with or without TD139 treatment in vitro (FIG. 6E). Gal3 and Gal3BP compete for the same binding site in CHI3L1. Computational estimates of the interaction free energy between these proteins using the FastContact server (Champ and Camacho, 2007) shows that Gal3BP binds to CHI3L1 more strongly than Gal3 (ΔG_{binding}=−9.9 kcal/mol and −4.3 kcal/mol, respectively). Of note, sequence alignments of the binding domains in CHI3L1, Gal3 and Gal3BP showed high conservation between human and mouse, indicating the evolutionarily conserved functions of these genes (FIG. 15A).

To examine whether Gal3 and Gal3BP are associated with selective migration of M1/M2-like MDMs, expression levels of Gal3 and Gal3BP were detected in polarized BMDMs. qRT-PCR and immunoblotting analyses revealed highly expressed Gal3 in M2 BMDMs compared to M0 and M1 BMDMs (FIGS. 15E and 15B). It was observed that a microglia cell line (SIM-A9) expresses higher levels of Gal3BP compared with polarized BMDMs and the murine macrophage cell line RAW264.7 (FIGS. 15C and 15D), indicating a mechanism by which CHI3L1 effectively induces cell migration in M2-like MDMs but not MG. Therefore, CHI3L1 can cooperates with Gal3 to selectively promote M2 BMDM migration. To this end, scratch-wound healing and transwell assays revealed that treatment of M0 BMDMs (lower endogenous levels of Gal3 expression) with rCHI3L1+rGal3 significantly increased cell migration (FIGS. 7A-7D). To test whether Gal3BP inhibits CHI3L1-Gal3 induced MDM migration, M0 BMDMs were treated with rCHI3L1+rGal3+rGal3BP, resulting in a significant decrease in cell migration compared with those treated with rCHI3L1+rGal3 (FIGS. 7A-7D). These data show that Gal3BP competes with Gal3 to bind CHI3L1, leading to inhibition of MDM migration by disrupting the CHI3L1-Gal3 protein complex. Enrichment of M1/M2-like macrophage signatures associated with expression of these genes in TCGA GBM samples was analyzed and increased M2- and decreased M1-like macrophage signatures were observed in tumors with high mRNA expression levels of CHI3L1 and LGALS3 (FIG. 7E). Furthermore, a significant increase of M1-like macrophage signatures was shown in the GBMs with low levels of CHI3L1 and LGALS3 expression but high levels of LGALS3BP expression (FIG. 15D), indicating a predominant association between Gal3BP and a proinflammatory M1-like phenotype. Collectively, these results show that CHI3L1 binds with Gal3 forming a protein complex, promoting infiltration of M2-like MDMs, which is negatively regulated by Gal3BP via a competitive interaction.

### Example 6. CHI3L1-Gal3 Protein Complex Induces Tumor Immunosuppression by Reprogramming MDMs

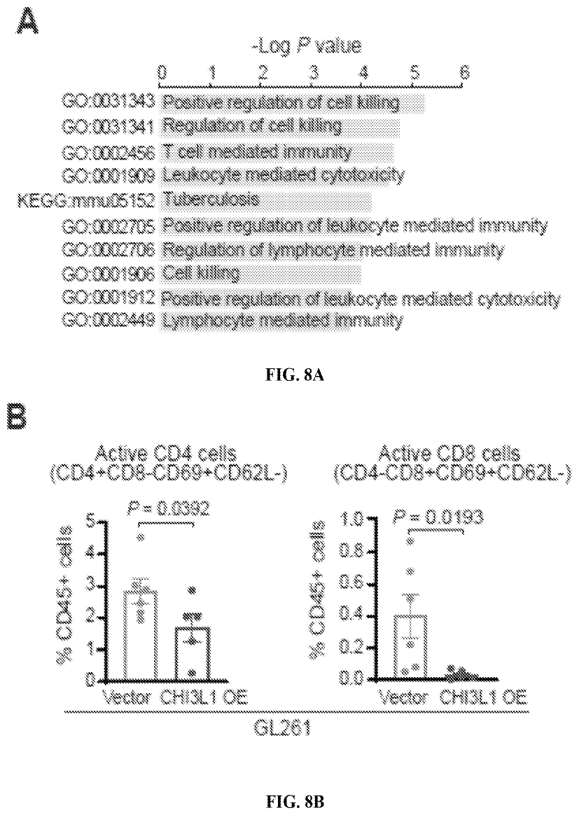

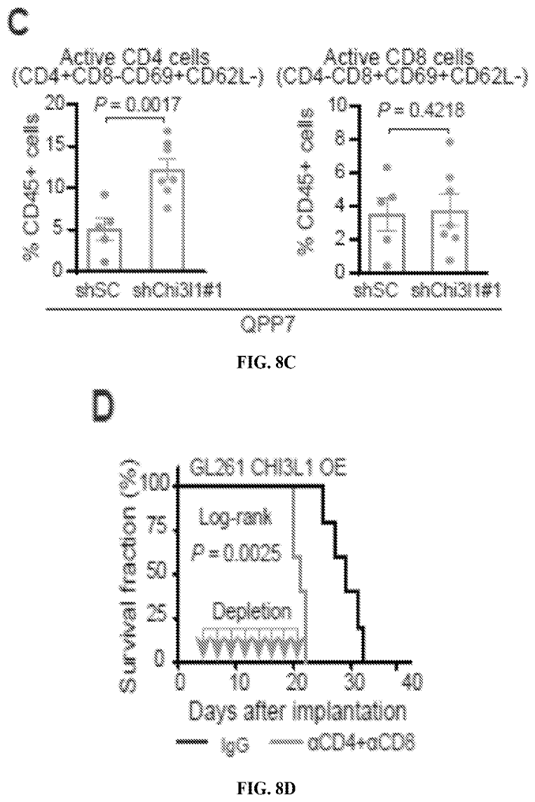

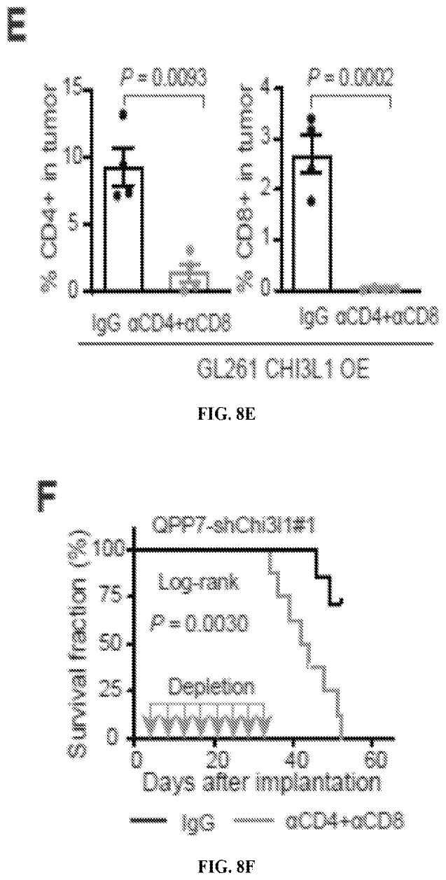

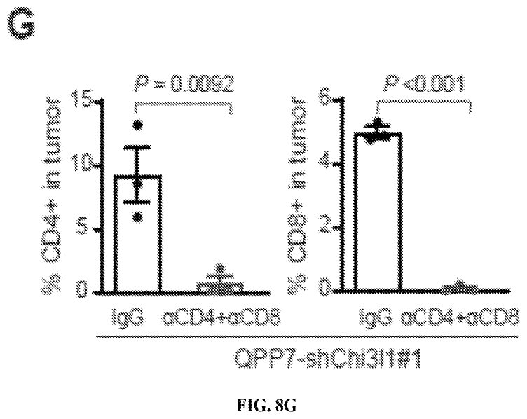

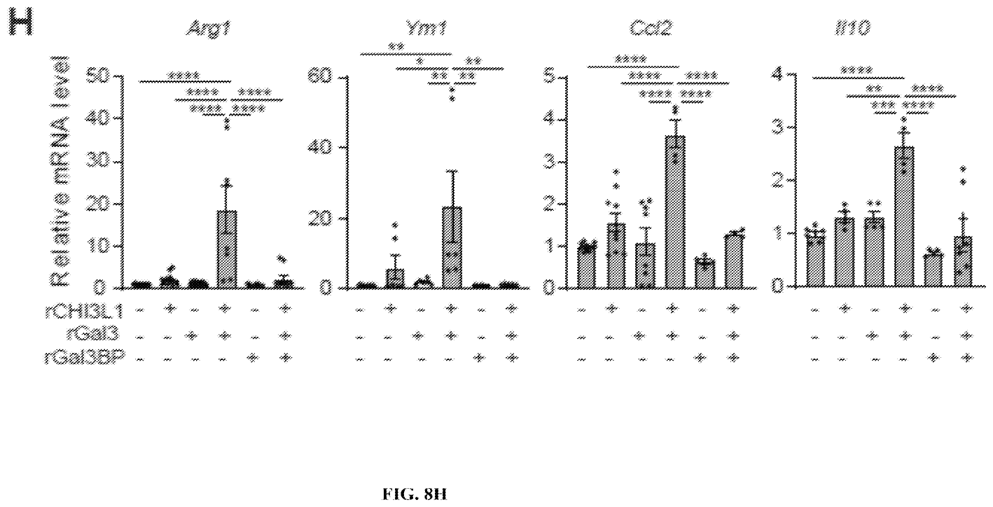

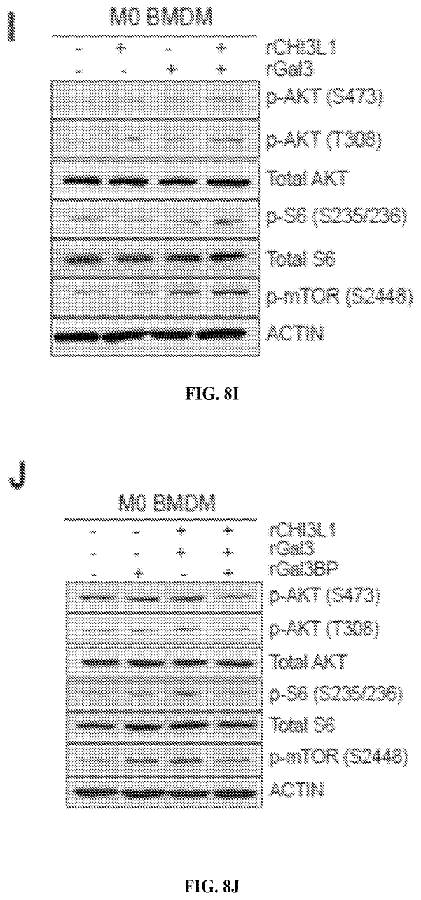

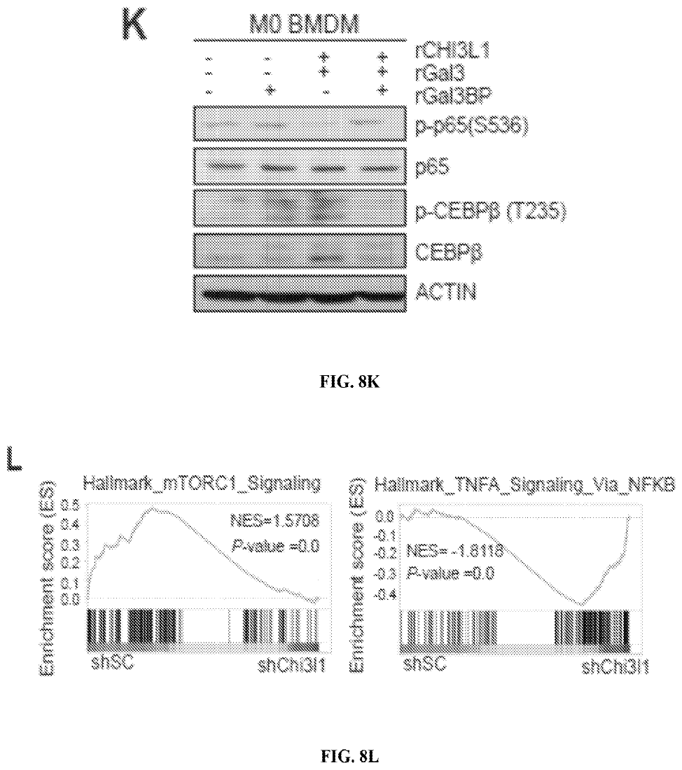

To delineate molecular mechanisms of CHI3L1 protein complexes-induced tumor progression, RNA-seq analysis was performed on TAMs isolated from orthotopic xenograft glioma models in C57BL/6 mice bearing the isogenic line QPP7 with shChi3l1, relative to QPP7 with shSC. Gene-Ontology (GO) analysis showed that signaling pathways regulating cell killing, leukocyte-mediated cytotoxicity, and lymphocyte-mediated immunity were enriched in TAMs derived from Chi3l1 KD tumors (FIG. 8A). This study assessed whether CHI3L1 signaling reprograms TAMs toward a protumor phenotype, leading to dysregulation of the tumor-infiltrating lymphocyte (TIL)-mediated antitumor immune responses. To this end, it was observed that CHI3L1 overexpression significantly decreased the active CD4⁺ (CD69⁺CD62L⁻) and CD8⁺ (CD69⁺CD62L⁻) TILs in GL261 tumors (FIG. 8B). Conversely, the active CD4⁺ T cells were significantly increased in QPP7 Chi3l1 KD tumors while enrichment of active CD8⁺ T cells did not reach significance (FIG. 8C). These results show that dysregulation of T cell-mediated antitumor immune response contributes to CHI3L1-induced tumor progression.

Previous studies revealed that TAM depolarization, rather than depletion, profoundly affects cancer progression by changing gene expression and switching between phenotypes of immune suppression and immune stimulation (Pyonteck S M et al., 2013; Kaneda M M et al., 2016). The study further demonstrated that depleting peripheral and intratumoral MDMs but not MG by systemic delivery of clodronate liposomes (van Rooijen N et al., 1988; Fulci G et al., 2007) did not repress tumor progression in the syngeneic orthotopic glioma model of C57BL/6 mice bearing GL261-CHI3L1 (FIG. 16A-16E). Gene set enrichment analysis (GSEA) showed that hallmark pathways in immune stimulation, including IFNα, IFNγ, IL6-JAK-STAT3 signaling, and inflammatory response, were enriched in TAMs derived from tumors with Chi3l1 KD compared to controls (FIGS. 16F and 16G). Moreover, genes associated with immune suppression (Arg1, Ym1, Ccl2, Il10) were downregulated in TAMs from shChi311 tumors; however, genes associated with immune stimulation (Nos2, Il6, Il12b, Ifng) were upregulated in these TAMs (FIG. 16H). To further examine the involvement of T cells in CHI3L1-mediated tumor progression, we performed antibody depletion studies of CD4⁺ and CD8⁺ cells in GL261-CHI3L1 and QPP7-shChi3l1 #1 derived syngeneic tumor models, respectively. Treatment with anti-CD4 and anti-CD8 antibodies significantly enhanced CHI3L1 overexpression-induced tumor progression in GL261 models (FIGS. 8D and 8E) but attenuated Chi3l1 KD-mediated tumor regression in QPP7 models (FIGS. 8F and 8G; FIG. 16I). Together, these results reveal that silencing CHI3L1 reprograms a TAM switch from immune suppression to stimulation, which is required for T cell-mediated antitumor response.

To elucidate the downstream signaling pathways of CHI3L1 protein complex-regulated TAM reprogramming, M0 BMDMs were treated with rCHI3L1+rGal3. Genes related to anti-inflammation (Arg1, Ym1, Ccl2, Il10) were increased compared to any single agent treatment by qRT-PCR assessment. Notably, upregulation of these genes by rCHI3L1+rGal3 treatment was significantly inhibited in M0 BMDMs treated with rGal3BP (FIG. 8H). These data indicate that the CHI3L1-Gal3 protein complex reprograms TAMs toward a M2-like phenotype by regulating gene expression associated with immune suppression, which is negatively regulated by Gal3BP. The positive feedback loop of the CHI3L1-PI3K/AKT/mTOR singling (FIG. 1) indicates that CHI3L1-Gal3 protein complex activates the PI3K/AKT/mTOR pathway, which significantly controls a macrophage switch between immune stimulation and suppression by regulating NFκB and CEBPβ activation (Kaneda et al., 2016). Immunoblot analysis showed that rCHI3L1+rGal3 treatment increased the levels of p-AKT (T473 and S308), p-S6, and p-mTOR compared to either agent alone (FIG. 8I), which was inhibited by the addition of rGal3BP in M0 BMDMs (FIG. 8J). To evaluate the activation of PI3K/AKT/mTOR downstream transcription factors, it was found that rCHI3L1 plus rGal3 stimulated C/EBPβ expression and simultaneously inhibited p65-RelA phosphorylation in M0 BMDMs (FIG. 8K). GSEA showed enrichment of mTOR1 signaling in TAMs derived from tumors with shSC, and enrichment of TNFA signaling via NFκB pathway in TAMs derived from tumors with shChi3l1, further supporting the involvement of these transcription factors in MDM reprogramming by CHI3L1 protein complexes (FIG. 8L). Altogether, these results indicate that CHI3L1-Gal3 protein complex reprograms MDMs toward an immunosuppression or immunostimulation phenotype by controlling a transcriptional regulatory program of PI3K/AKT/mTOR-NFκB/CEBPβ (FIG. 16J).

### Example 7. Gal3BP Mimetic Peptide Inhibits CHI3L1-Gal3 Complex-Induced Tumor Progression

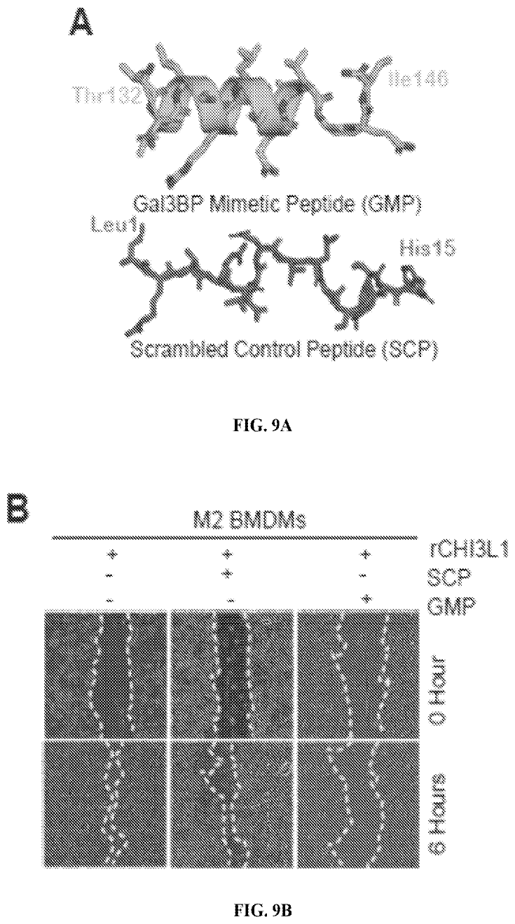

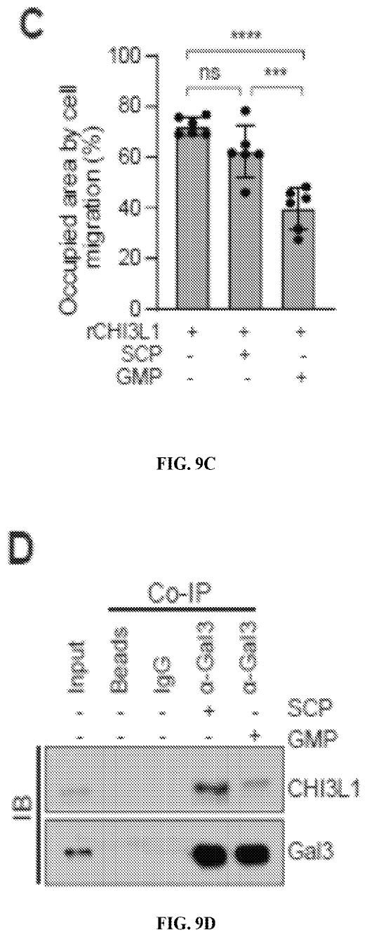

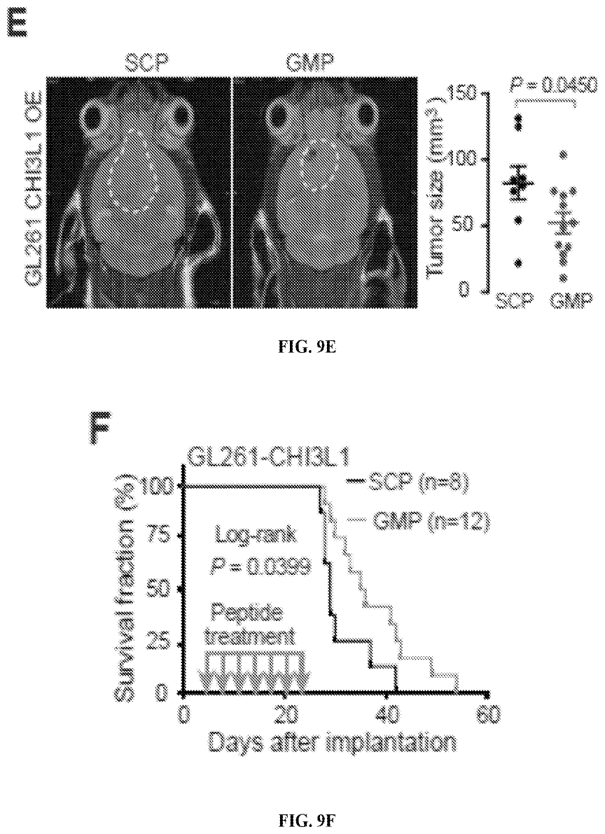

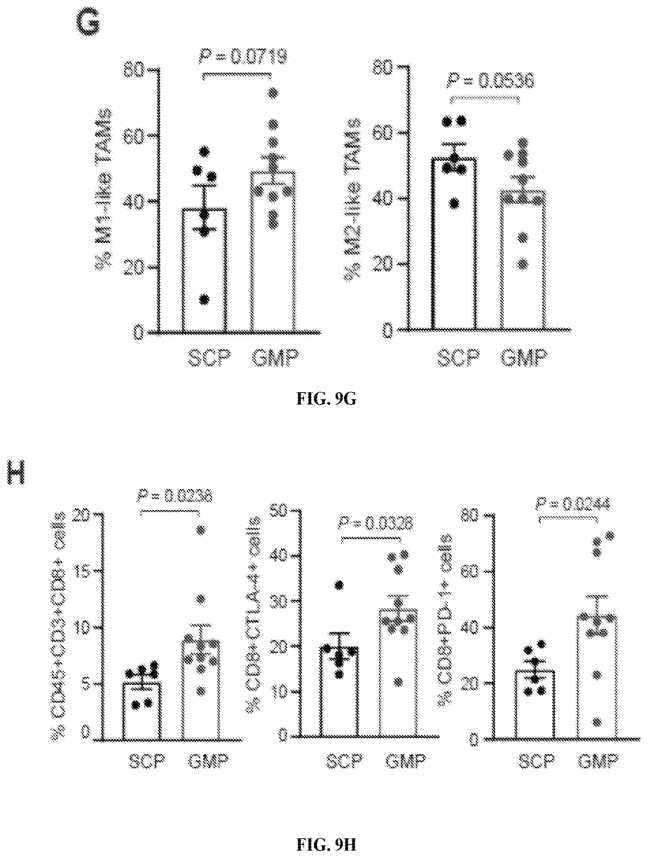

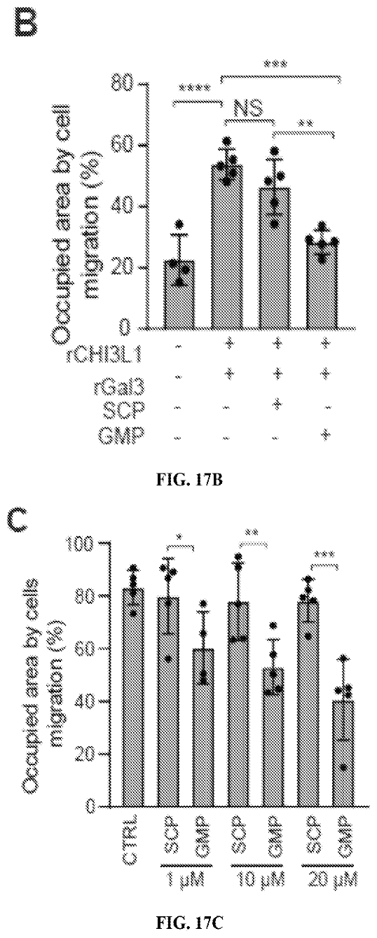

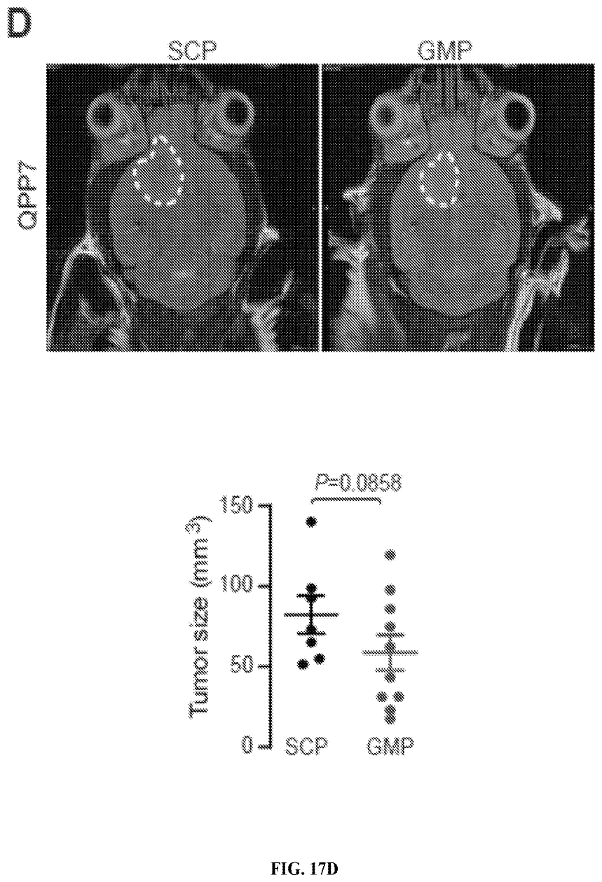

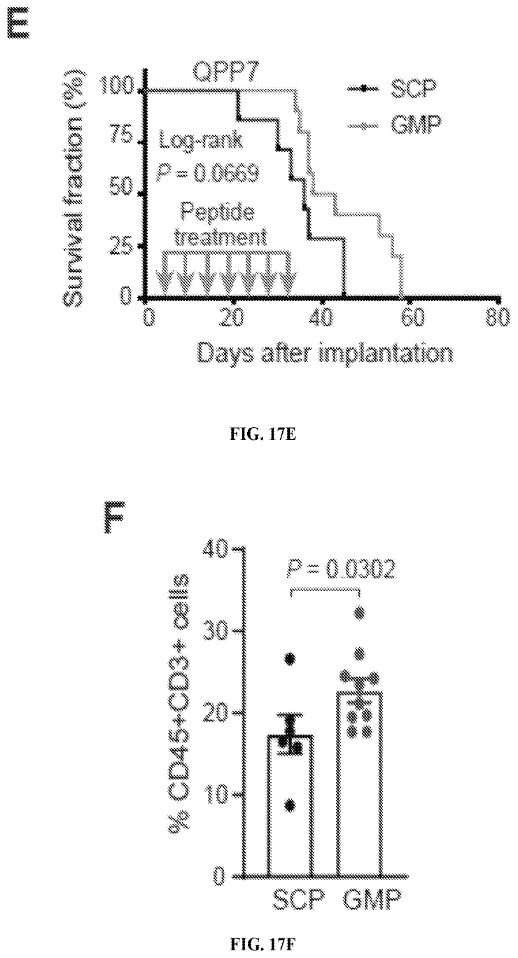

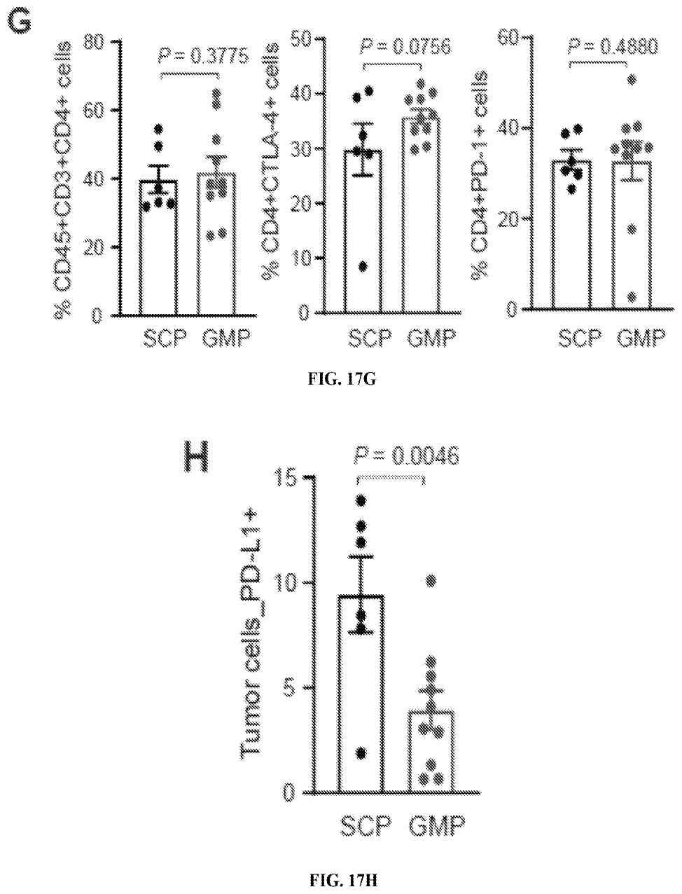

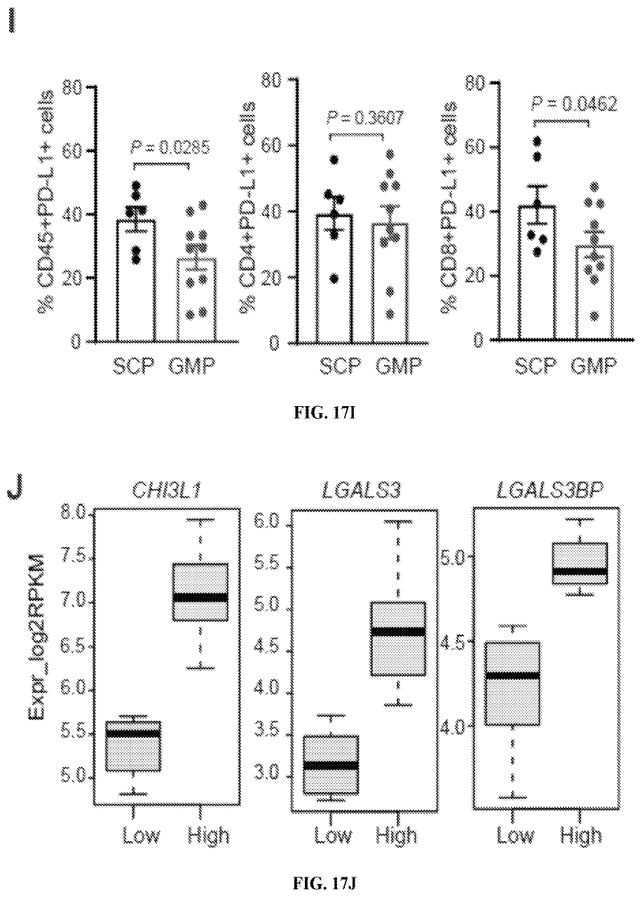

To investigate whether disruption of CHI31L-Gal3 protein binding complex can reverse MDM-mediated immune suppression and thereby attenuate glioma progression, a Gal3BP Mimetic Peptide (GMP) was designed to disrupt the interaction between Gal3 and CHI3L1. Molecular dynamics showed that GMP (T₁₃₂LDLSRELSEALGQI₁₄₆) (SEQ ID NO: 4), rather than scrambled control peptide (SCP, L₁RTRLEETLSSDTSH₁₅) (SEQ ID NO: 20), behaves as a linear peptide capable of recapitulating interaction with CHI3L1 (FIG. 9A). To test GMP inhibiting CHI3L1-Gal3-induced macrophage migration in vitro, M2 BMDMs were treated with rCHI3L1 in combination with GMP and SCP, respectively. Of note, scratch-wound healing assay analysis revealed that rCHI3L1-promoted M2 BMDM migration was significantly inhibited by GMP compared with SCP treatment (FIGS. 9B and 9C). In contrast to SCP, GMP also attenuated rCHI3L1+rGal3-induced M0 BMDM migration (FIGS. 17A-17B). To verify that GMP recapitulates Gal3BP to compete with Gal3 binding with CHI3L1, this study found that GMP treatment resulted in decreasing binding of CHI3L1 and Gal3 in THP-1 cells by the Co-IP assay (FIG. 9D). Moreover, GMP inhibits rCHI3L1-induced BMDM migration in a dose-dependent manner (FIG. 17C). These data demonstrate that this new-developed peptide can mimic Gal3BP to disrupt CHI31L-Gal3 protein interaction and BMDM migration.

To assess the antitumor effect of GMP in vivo, GMP and SCP were administered directly into brain tumors by an implantable guide-screw system (Lal et al., 2000) in C57BL/6 mice bearing GL261-CHI3L1 orthotopic tumors. Notably, GMP treatment reduced tumor growth and extended animal survival (median survival of 36 days) compared to SCP (median survival of 29 days) (FIGS. 9E and 9F). To validate the antitumor effect of GMP on tumors with the endogenous level of CHI3L1, the orthotopic syngeneic mice bearing QPP7 glioma were treated by local delivery of GMP and SCP into the brain, respectively. Consistently, we found that the treatment of GMP decreased tumor size and increased mouse survival in the QPP7 model (FIGS. 7D and 7E).

To evaluate the changes of immune cell populations following peptide treatment, the study performed flow cytometry of cells harvested from syngeneic C57BL/6 mice bearing GL261-CHI3L1 glioma. Following GMP treatment, an increase of M1-like TAMs (49.5±4.0% vs 38.2±6.7%, P=0.0719) and decrease of M2-like TAMs (42.8±3.8% vs 52.7±3.9%, P=0.0536) were observed compared to SCP treatment (FIG. 9G). In contrast to SCP, GMP treatment significantly increased T cells (CD3+), particularly CD8+ cells (FIG. 9H; FIG. 17F). The CD4+ cell population increased under GMP versus SCP treatment (41.9±4.5% vs 39.8±4.0%) indicating that Tregs, a subset of CD4+ cells, can influence the total CD4 cell composition, proliferation and recruitment (FIG. 17G). These results show that GMP can reprogram TAMs from protumor to antitumor phenotype, which indirectly promotes CD8+ T cell-mediated antitumor immune response.

Despite increasing the tumor-infiltrating T cells after GMP treatment, T cell exhaustion is a hallmark of GBM local immune dysfunction due to the upregulation of multiple immune checkpoints such as PD-1 and CTLA-4 (Medikonda et al., 2020; Woroniecka et al., 2018). Therefore, expression levels of these immune checkpoints were assessed in CD4+ and CD8+ T cells; and found that both PD-1 and CTLA-4 were significantly upregulated in CD8+ T cells from GMP-treated tumors compared to those in the SCP-treated tumors in the GL216-CHI3L1 model (FIG. 9H). The CD4+ T cells from tumor-bearing mice displayed a trend of elevated levels of PD-1 and CTLA-4 following the treatment with GMP vs SCP, respectively (FIG. 17G). Expression of PD-L1 was evaluated. It is a ligand of PD-1, which is upregulated in activated leukocytes and cancer cells (Topalian et al., 2015). GMP treatment significantly decreased PD-L1 expression in glioma cells, while only a decreasing trend of PD-L1 expression in CD45+, CD8+ and CD4+ cells was observed and glioma cells, which suggests that disrupting CHI3L1-Gal3 interaction can lead to the reduction of T cell exhaustion (FIGS. 17H and 17I).

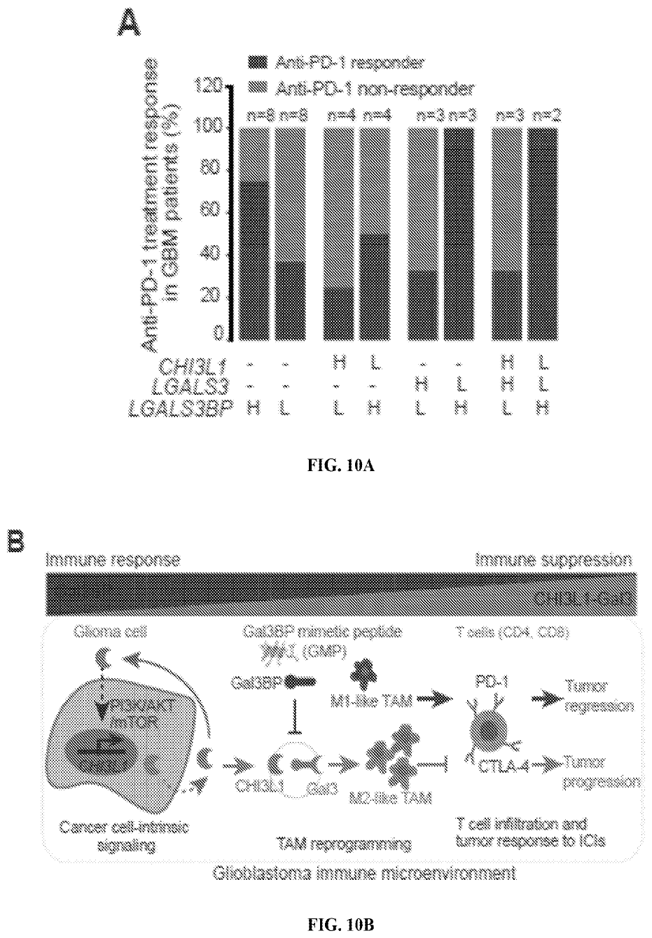

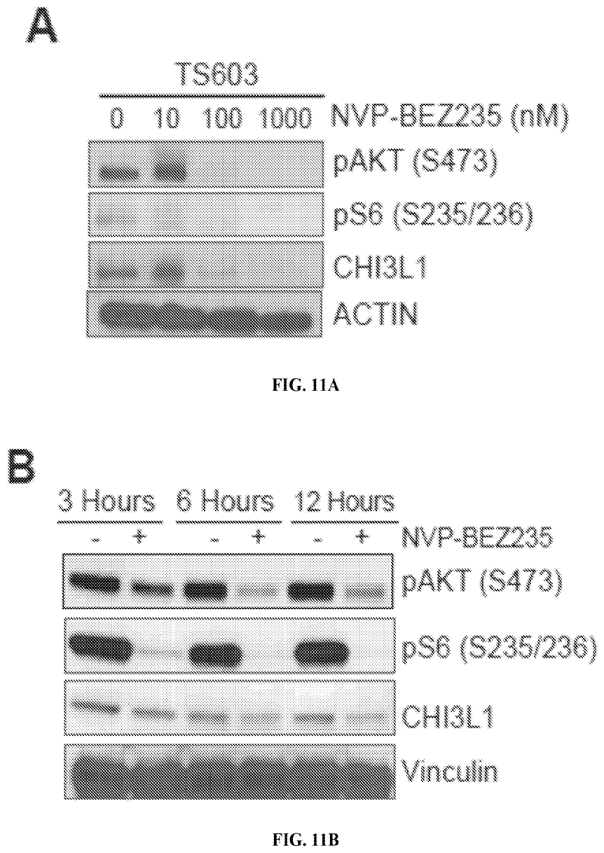

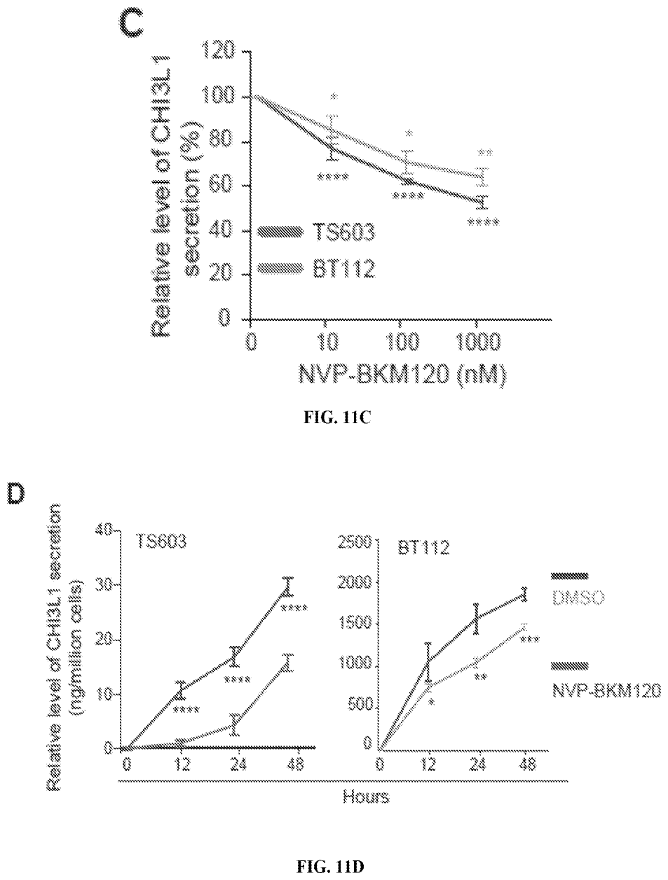

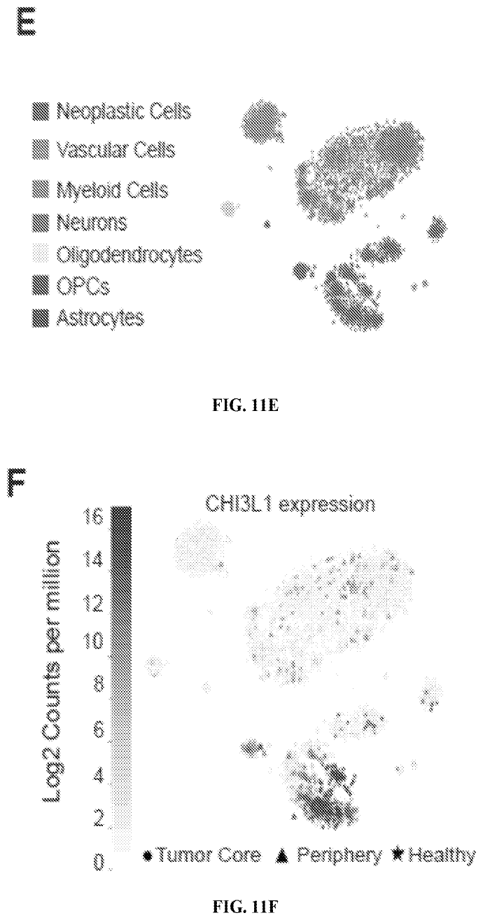

Together, these results indicate that CHI3L1 protein binding complexes with Gal3 or Gal3BP modulate TAM-mediated immune suppression and stimulation, leading to resistance or response to immune checkpoint therapy. To evaluate whether gene expression of CHI3L1, LGALS3 and LGALS3BP is associated with patient response to immunotherapy with immune checkpoint inhibitors (ICIs), bulk RNA-sequencing profiles of GBM were analyzed from 16 GBM patients with treatment of PD-1 inhibitors (nivolumab or pembrolizumab) (Zhao et al., 2019). Higher levels of LGALS3BP expression are associated with anti-PD-1 responders, whereas lower levels of LGALS3BP expression are associated with anti-PD-1 non-responders (FIG. 10A; FIG. 17J). Moreover, higher levels of LGALS3BP combined with lower levels of LGALS3 and/or lower levels of CHI3L1 are associated with anti-PD-1 responders and vice versa (FIG. 10A). Collectively, these data show that CHI3L1 protein binding complex modulates TAM and T cell-mediated immunity, which underlies these proteins as the key determinants of the response to immune checkpoint therapy. Therapeutically, disrupting the CHI3L1-Gal3 protein complex using GMP can synergize with ICIs to effectively promote tumor regression for GBM patients (FIG. 10B).

Although the GBM TME plays a crucial role in regulating tumor progression and is increasingly recognized as a therapeutic target, understanding the underlying cellular and molecular mechanisms governing glioma cells and their surrounding components remains challenging. This study discovered that cancer-cell-intrinsic CHI3L1 plays a predominant role in modulating the GBM TME by forming a protein complex with Gal3 or Gal3BP to promote a macrophage-mediated immune suppression. The efforts to understand the mechanisms governing GBM immune suppression resulted in a newly developed peptide as an immunostimulatory drug candidate and pharmacological modifications of CHI3L1-Gal3/Gal3BP protein complexes as potential therapeutics for patients with GBM.

Increasing evidence shows that tumor-intrinsic mechanisms dictate various non-cancerous cells within the tumor microenvironment, which exert multifaceted functions, ranging from antitumor to protumor activities (Quail and Joyce, 2017; Wang et al., 2017; Wellenstein and de Visser, 2018). The findings in this study demonstrate that cancer-cell intrinsic CHI3L1 is upregulated by the PI3K/AKT/mTOR signaling axis in a positive feedback loop, which plays a predominant role in modulating the GBM immune microenvironment by inducing M2-like MDM infiltration and repolarization in a paracrine mechanism. Genetically, CHI3L1 gene expression is significantly associated with loss of chromosome 10q encompassing PTEN in GBM (Pelloski et al., 2005). The work herein reinforces the positive correlation between CHI3L1 gene expression and PTEN deletions/mutations or other mechanisms leading to PI3K/AKT/mTOR activation (e.g. NF1 mutations). These findings deepen the understanding of tumor-intrinsic signaling pathways driven by genetic alterations in regulation of the GBM immune microenvironment for tumor progression and treatment response.

In exploring the role of CHI3L1 for regulating the GBM immune microenvironment, it was discovered that CHI3L1 binding with Gal3 promotes MDM infiltration and reprograms MDMs toward a tumor-promoting M2-like phenotype, which are negatively regulated by Gal3BP. Increased expression and secretion of Gal3 were observed in both human and mouse M2-polarized macrophages compared to monocytes and M1-polarized macrophages (MacKinnon et al., 2008; Novak et al., 2012). However, increased levels of Gal3BP and a proinflammatory phenotype were observed in human monocyte-derived M1 macrophages in vitro and in plasma from patients with cardiovascular disease or hepatitis C infection (Gleissner et al., 2017; Shaked et al., 2014). In this study, the finding of CHI3L1-Gal3 protein complex-induced selective migration of M2-polarized BMDMs provides a mechanistic explanation for a long-standing observation, namely highly infiltrating M2-like MDMs associated with both human and mouse GBM (Chen et al., 2017; Darmanis et al., 2017; Pinton et al., 2019; Wang et al., 2017). In addition to promoting M2-like MDM accumulation, the present study also provides the mechanisms for immunosuppression that enables GBM to escape immune surveillance, by which CHI3L1-Gal3 protein complex activates AKT/mTOR-mediated transcriptional regulatory network (NFκB and CEBPβ), leading to a macrophage switch toward immune suppression from immune stimulation (Kaneda et al., 2016).

Reducing immunosuppression and overcoming immunotherapy resistance are becoming therapeutic areas of great interest for the treatment of GBM as well as other solid tumors (Jackson et al., 2019; Tomaszewski et al., 2019). These findings provide a rationale for disrupting CHI3L1-Gal3 protein complex by the addition of Gal3BP to reduce the degree of tumor immunosuppression and improve antitumor immune response in the GBM TME. Local and systemic increase in Gal3BP levels inhibited tumor growth by stimulation of the residual cell-mediated immune defense of the nude mouse (Jallal et al., 1995). Although the function of Gal3BP is controversial in physiologic and pathologic conditions, elevated levels of Gal3BP in bacterial and viral infections and in the neoplastic context show its crucial role in immune response as an immunostimulatory molecule (Kalayci et al., 2004; Loimaranta et al., 2018; Ullrich et al., 1994). In the instant study, GMP being locally delivered into brain tumor led to tumor regression in the treated animals combined with reduced M2-like MDMs and increased M1-like MDMs and CD8+ T cells in the TME, indicating that this peptide can modify CHI3L1 protein complexes and thereby reprogram the immune microenvironment. Although CD8+ T cells were significantly increased by GMP treatment, elevated levels of CTLA-4 and PD-1 expression were observed in these T cells, a hallmark feature of T-cell exhaustion, showing that GMP can synergize with immune checkpoint inhibitors to form a more effective immunotherapy for GBM treatment. Based on analyzing a publicly available clinical dataset (Zhao et al., 2019), the higher and lower levels of LGALS3BP combined with LGALS3 or CHI3L1 gene expression are associated with response to anti-PD-1 immunotherapy, reinforcing the mechanism of CHI3L1-Gal3/Gal3BP protein complexes in regulating protumor or antitumor immunity in GBM. In summary, the findings in this study shed light on a crucial molecular mechanism of macrophage-mediated immunosuppression in GBM, indicating the development of a more effective treatment for patients with this devasting brain cancer.

### Example 8. CHI3L1-Gal3 Protein Complex Regulates CD274/PD-L1 Expression in Tumor Cells

Programmed death-1 (CD279/PD-1) binds to its ligand, programmed death-ligand 1 (CD274/PD-L1), leading to activation of their downstream signaling pathways and subsequent inhibition of T cell function. The inhibitors of PD-1 and PD-L1 can block the activity of PD-1 and PD-L1 immune checkpoint protein present on the surface of cells, which are emerging as a front-line treatment for many types of cancer.

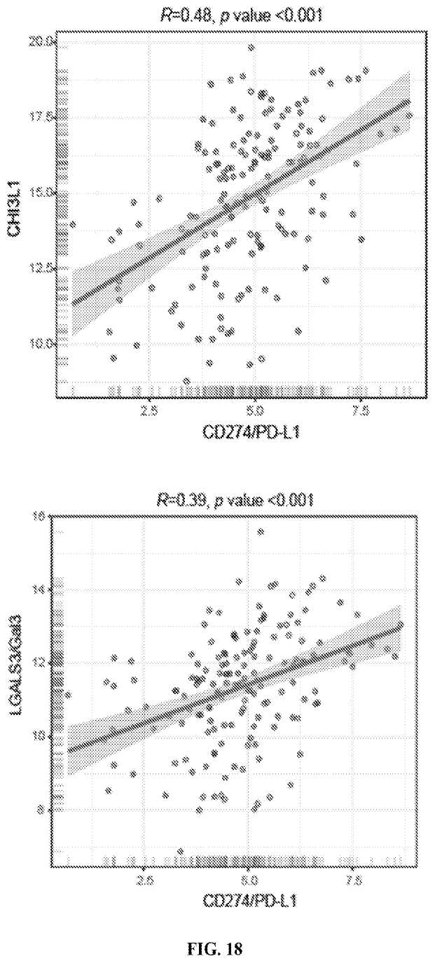

This study discovered that the treatment with our newly-developed peptide (GMP) in the preclinical mouse models can significantly decrease PD-L1 expression in CD45+, CD8+ immune cells, and glioma cells (FIGS. 17I and 17H). These results indicate that the CHI3L-Gal3 protein complex may upregulate PD-L1 expression in immune cells (such as macrophages, dendritic cells, MDSCs), and cancer cells. We analyzed the transcriptomic datasets of The Cancer Genome Atlas (TCGA) glioblastoma patient cohorts and found the significant correlations between CD274/PD-L1 mRNA expression and CHI3L1 and Gal3 (encoded by the LGALS3 gene) expression (FIG. 18), respectively, which further indicates the regulatory association between the CHI3L1-Gal3 protein complex and CD274/PD-L1 in tumor cells.

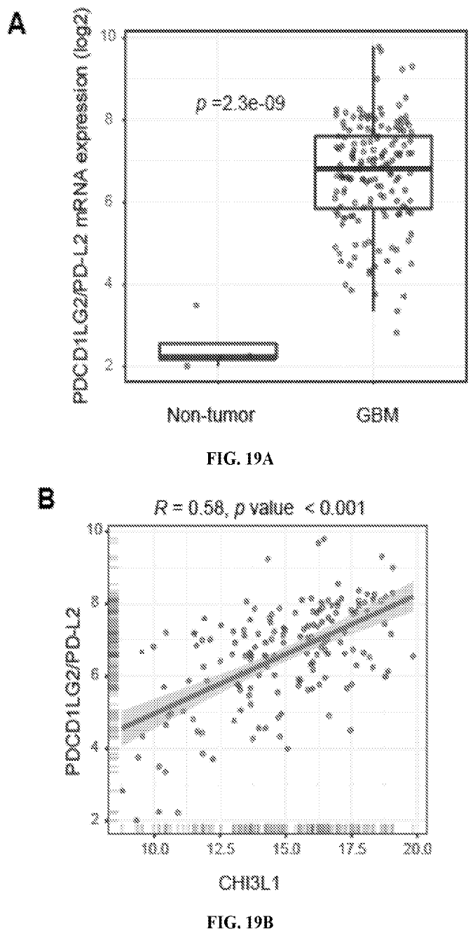

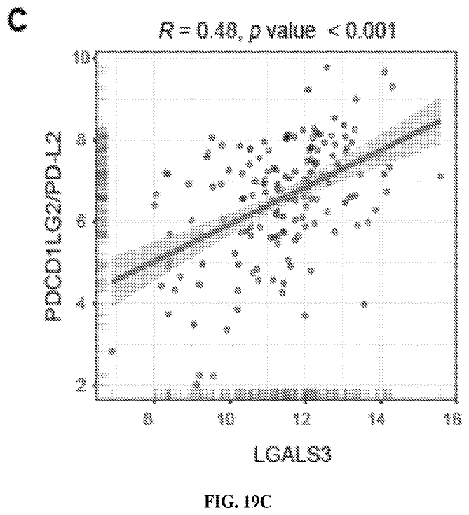

We also found that Programmed cell death 1 ligand 2 (PD-L2, also called CD273, encoded by the PDCD1LG2 gene), the other ligand of CD279/PD-1, is highly expressed in GBM compared to the normal brain tissues (FIG. 19A). Additionally, the significance of positive correlations between PDCD1LG2/PD-L2 mRNA expression and CHI3L1 and Gal3 (encoded by the LGALS3 gene) expression based on the transcriptomic datasets of TCGA glioblastoma patient cohorts (FIGS. 19B and 19C).

In summary, our data suggest that the CHI3L1-Gal3 protein complex upregulates both CD274/PD-L1 and PDCD1LG2/PD-L2 transcriptional expression in GBM, which inhibits T cell-mediating anti-tumor immunity.

### Example 9. Experimental Model and Methods

Cell Lines.

GBM patient-derived neurosphere lines (TS543, TS603, BT112) and human neural stem cell lines (hNSCs) were used and cultured as described previously (Hu B et al., 2016). Mouse glioma cell line QPP7 provided by Dr. Jian Hu (MD Anderson Cancer Center, Houston, TX) was cultured in the serum-free NSC medium. U87, GL261, RAW246.7, and 293T from ATCC were cultured in DMEM supplemented with 10% FBS (Sigma-Aldrich,) and penicillin/streptomycin (P/S) (Gibco). SIM-A9 from ATCC was cultured in DMEM/F12 supplemented with 10% FBS, 5% horse serum, and P/S. THP-1 cell line was purchased from ATCC and cultured in RPMI-1640 supplemented with 10% FBS, 0.05 mM 2-mercaptoethanol, and P/S. All cell lines were verified to be mycoplasma-free using MyCoAlert PLUS Mycoplasma Detection Kit (Lonza, Cat #LT07-710), and cultured at 37° C. with 5% CO₂.

BMDM Culture and Polarization.

BMDMs were isolated from male and female C57BL/6 mice as previously described (Ying W et al., 2013). Briefly, femur bones were isolated from mice, and IMDM (ATCC) supplemented with 10% FBS and P/S was used to flush the bone marrow into a petri dish. After 4-6-hour incubation, the floating cells were collected and resuspended in the medium with 20 ng/mL M-CSF (PrproTech). On day 6, the fully differentiated cells were designated as an M0 state. To induce BMDM polarization toward an M1 state, 100 ng/mL LPS (Invitrogen) and 50 ng/mL IFNγ (PrproTech) were added to the M0 cells for 24 hours. To induce BMDM polarization toward an M2 state, 20 ng/mL IL-4 (PrproTech) was added to the M0 cells for 72 hours.

Intracranial Xenograft Tumor Models, Macrophage Depletion, T Cell Depletion, and Peptide Treatment.

Male and female ICR SCID and C57BL/6 mice (4-6 weeks of age) were purchased from Taconic Biosciences and The Jackson Laboratory, respectively. The intracranial xenograft tumor models were established as previously described (Hu B et al., 2016). Cells in 5 μL DPBS were injected at the following numbers: TS543 vector control or CHI3L1 overexpression (OE), 1×10⁴ cells; QPP7 scrambled control or CHI3L1 knockdown (KD), 1×10⁵ cells; and GL261 vector control or CHI3L1 OE, 1×10⁵ cells. For tumor models with macrophage depletion, Chodrosome or control liposome (Clodrosome, Cat #CLD-8901) was injected into animals through the tail vein. For tumor models with T cell depletion, IgG (BioXCell, Cat #BE0090) or anti-CD4 (BioXCell, Cat #BE0003-1) and anti-CD8 (BioXCell, Cat #BE0061) antibodies were injected into animals through intraperitoneal injection. For mice treated with peptides, 5 uL of 20 uM SCP or GMP was delivered into the mouse brain every 4 days with a total of seven times.

Enzyme-Linked Immunosorbent Assay (ELISA).

Secreted CHI3L1 protein in the cell culture supernatant was measured by using the Quantikine Human CHI3L1 Immunoassay (R&D Systems, Cat #DC3L10). CHI3L1 content in the conditioned media was quantified per million cells and no CHI3L1 was detected in DMEM or NSC medium supplemented with EGF and bFGF.

Co-Immunoprecipitation (Co-IP) and Mass Spectrometry (MS).

TS603 cells overexpressing CHI3L1 with V5 tag (TS603 CHI3L1_V5_OE) or THP-1 cells treated with the peptides were collected and protein-protein interaction was crosslinked with 2 mM Dithiobis (succinimidyl propionate, DSP). Membrane proteins were extracted with the Membrane Protein Extraction Kit (ThermoFisher, Cat #89842) and ˜500 μg of protein was used for Co-IP assay by the Co-Immunoprecipitation Kit (ThermoFisher, Cat #26149). For mass spectrometry, 10 μg of each of the TS603 CHI3L1_V5_OE Co-IP samples were separated in a 12% SDS-PAGE gel and analyzed by MS at the University of Pittsburgh Biomedical MS Center.

Immunoblotting (IB), Immunohistochemistry (IHC), and Immunofluorescence (IF).

Cells were lysed on ice using RIPA buffer (Millipore) supplemented with protease and phosphatase inhibitors (Roche). The protein concentration was determined by the BCA method and 15˜30 ug of total proteins were loaded and analyzed by Western blotting with indicated antibodies. For IHC staining, brain tissues were fixed in 10% formalin overnight and embedded in paraffin. For IF staining, fresh brain tissues were immediately frozen in OCT on dry ice. IHC and IF staining were performed as we described previously (Hu B et al., 2016). Additional information about antibodies is provided in the Supplementary Material and Methods section

Scratch-Wound Healing Assay.

BMDM were polarized to the indicated status (M0, M1, or M2) and seeded in 12-well plates at 80-90% confluency. The cells were switched to the medium without FBS for 6 hours of starvation. Scratches were made using pipettor tips and fresh IMDM with indicated recombinant proteins and/or peptides were added. Images of the scratches were captured at indicated times. For the SIM-A9 scratch-wound healing assay, 12-well plates were coated with 10 μg/mL fibronectin (Sigma-Aldrich) at 37° C. overnight before seeding cells.

Transwell Migration Assay.

Polarized BMDMs were starved by removing FBS for 6 hours. Cells were collected and 2×10⁵ cells in 200 μL of IMDM were added into each transwell insert (Millipore, Cat #MCMP24H48). 700 μL of IMDM containing 2% FBS and the indicated recombinant proteins was added to the bottom of the plates. After 14-hour incubation, transwell inserts were stained with HEMA 3 Stain Set (Fisher Scientific). Insert membranes were separated and mounted on glass slides with CYTOSEAL XYL (ThermoFisher) and images were taken by an inverted microscope (Leica DM 2500). For the SIM-A9 cells, the inserts were coated with 10 μg/mL fibronectin overnight in advance of seeding cells.

RNA Isolation, qRT-PCR, and RNA-Seq.

RNA was extracted and cDNA was synthesized as described previously (Hu B et al., 2016). qRT-PCR was performed using PowerSYBR Green PCR Master Mix (Applied Biosystems) and detected with a StepOnePlus Real-Time PCR System (Applied Biosystems). Primers are listed in Table 4. Each reaction was performed in duplicate or triplicate. The relative expression of genes was normalized to human RPL39 or mouse 18S ribosomal RNA. For RNA-seq experiments, cells from intracranial xenograft tumors were isolated and incubated with antibodies for immune cell types. Macrophages were isolated by FACS and RNA was then isolated and sent to Health Sciences Sequencing Core at UPMC Children's Hospital of Pittsburgh for RNA-seq. RNA-seq data are available in the NCBI's GEO (accession number GSE174177)

MRI and Bioluminescent Imaging.

MRI and bioluminescent imaging of mice were performed at Rangos Research Center Animal Imaging Core. The tumor size of mice detected by MRI was analyzed with ITK-SNAP. For bioluminescent imaging, mice were intraperitoneally injected with D-Luciferin (150 mg/Kg; GoldBio), and images were captured by the IVIS Lumina S5 system (PerkinElmer).

Brain Tumor Cell Isolation.

Mice with neurological deficits or moribund appearance were sacrificed. Tumors were separated and homogenized for 15 min at 37° C. in Collagenase IV Cocktail (3.2 mg/mL collagenase type IV, 2 mg/mL soybean trypsin inhibitor, and 1.0 mg/mL deoxyribonuclease I; Worthington Biochemical). Red blood cells were lysed using ACK lysing buffer (Gibco). Cell suspensions were filtered through 70-μm strainers, centrifuged, and resuspended in cold FACS buffer (DPBS with 1% BSA) for further analysis.

Flow Cytometry and CyTOF.

About 3×10⁶ cells were used for each staining panel. Cells were incubated with 1.0 μg of TruStain fcX (BioLegend) for 10 min on ice to block Fc receptors, followed by staining with the combination of indicated antibodies. After staining, cells were washed with FACS buffer three times and incubated with Fixation Buffer (BioLegend) at RT for 20 min. Cells were washed with FACS buffer, resuspended in Cyto-Last Buffer (BioLegend), and analyzed by either a BD LSRFortessa or BD FACSAria II SORP. For CyTOF, samples were prepared as described above for flow cytometry. Three pairs of samples (scrambled shRNA vs Chi3l1 KD) with similar tumor sizes were chosen and the staining procedure was followed as previously described (Mitsialis V, et al., 2020). The samples were analyzed on a Helios2 CyTOF system (Fluidigm) at the Longwood Medical Area CyTOF Core. Additional information about flow cytometry antibodies is provided in the Supplementary Material and Methods section.

Structure Analysis of Protein-Protein Interaction.

Prediction of protein-protein interaction was based on available human protein structures for binding of CHI3L1 and other putative protein candidates. Protein structures of CHI3L1 (PDB 1HJV), Gal3 (PDB 6FOF), and Gal3BP monomer (PDB 6GFB) were used for protein-protein interaction analyses. Docked poses of CHI3L1 with Gal3BP (monomer of dimerization domain) and CHI3L1 with Gal3 were predicted using ClusPro (Comeau S R et al., 2004; Kozakov et al., 2017) and further analyzed with FastContact for energetic complementarity (Champ P C et al., 2007).

Peptide Design.

GMP was designed using molecular dynamics (MD) simulations in AMBER18 on the GPU-accelerated code with AMBER ff14SB force field (Salomon-Ferrer R et al., 2013; Maier J A et al., 2015). The tLeap binary was used to solve structures in an octahedral TIP3P water box with a 15 Å distance from the peptide surface to the box edges and a closeness parameter of 0.75 Å. The system was neutralized and solvated in 150 mM NaCl. The non-bonded interaction cutoff was set to 8 Å. Hydrogen bonds were constrained using the SHAKE algorithm and an integration time step of 2 fs. Simulations were carried out by equilibrating the system for 5 ns at NPT, using a Berendsen thermostat to maintain a constant pressure of 1 atm followed by 300 ns NVT production at 300 K.

Data analysis. Data are calculated with GraphPad Prism and presented as the mean±SD or ±SEM. P<0.05 was considered as the statistical significance and it was determined by the indicated tests in figure legends. Scratch-wound healing areas of cell migration, Transwell migration assay cell number, and IF staining positive cells were analyzed by using Fiji software (ImageJ). Flow cytometry data were gated, analyzed, and visualized using FlowJo software (BD). CyTOF data were analyzed with Cytobank (Cytobank Inc.). TCGA GBM datasets were used for clinical GBM analysis, and RNA-seq data (Topalian S L et al., 2019) were used for correlation analysis between gene expression (CHI3L1, LGALS3, and LGALS3BP) and GBM patient response to anti-PD-1 treatment.

Lentivirus Production.

The expression vectors pLenti6.3-GFP, -p53DN, -myr-AKT, -CHI3L1, and -CHI3L1_V5 were generated by cloning the respective open reading frame (ORF) into the vector using the Gateway Cloning System. The pLKO.1 target gene set for the mouse Chi3l1 gene was purchased from Sigma-Aldrich. Lentiviruses were generated in 293T cells with a packaging system including pCMVR8.74, pMD2.0G, and pRSV-Rev according to the manufacturer's protocol (Invitrogen). Lentiviruses from the medium of infected 293T cells were concentrated to a final concentration of 13% of polyethylene glycol (PEG)-8000 and 0.5 M NaCl. Target cells were infected with lentivirus with 1:1000 dilution of polybrene (Sigma-Aldrich, Cat #TR-1003). Gene expression was confirmed by qRT-PCR and immunoblotting.

Macrophage and T Cell Depletion.

Three days after intracranial implantation of tumor cells, 100 μL/20 g per mouse of Chodrosome or control liposome was injected into animals through the tail vein. Clodronate liposomes or control liposomes were injected every three days for a total of eight times. Blood was collected after four injections, and monocytes were treated with RBC Lysis Buffer (Invitrogen, Cat #00-4300-54), stained with F4/80-APC (Invitrogen, Cat #47-4801-80) and CD11b-PE (BD Biosciences, Cat #553311), and analyzed by flow cytometry.

For T cell depletion, one day after intracranial implantation of tumor cells, 200 μg/20 g per mouse of IgG or anti-mouse CD4 and anti-mouse CD8 antibodies were injected into animals through intraperitoneal injection. IgG or anti-mouse CD4 and anti-mouse CD8 antibodies were injected every three days for a total of eight times. Brain tumors and spleens were collected for cell isolation and subsequent staining with CD45-PerCP-Cy5.5 (Cat #103132, 30-F11), CD4-APC-Cy7 (Cat #100414, GK1.5), CD8a-BV711 (Cat #100747, 53-6.7), and Live/Dead Fixable Blue Dead Cell Stain Kit for UV excitation (Invitrogen, Cat #L34961), followed by flow cytometry analysis.

Peptide Delivery to Mouse Brain In Situ.

For intracranial xenograft tumor models treated with peptides, mice were implanted with a guide screw prior to intracranial injection. To install the screw, mice were anesthetized by intraperitoneal (IP) injection with ketamine/xylazine solution (1.75 mL of 100 mg/mL ketamine and 0.25 mL of 100 mg/mL xylazine in 8 mL sterile water) at a dosage of 100 μL/20 g body weight. The screw was covered by sealing the skin, and mice were allowed to recover for one week before intracranial implantation. GL261 cells overexpressing CHI3L1 (1×10⁵) were injected in 5 μL of DPBS containing 20 μM of either scrambled control peptide (SCP) or Gal3BP mimetic peptide (GMP). SCP (20 μM) or GMP (20 μM) was subsequently given once every 3 days with a total of seven times by intracranial injection via the guide screw.

Co-Immunoprecipitation (Co-IP) and Mass Spectrometry (MS).

To confirm Co-IP experiments in TS603 cells by immunoblotting, the magnetic beads of protein G (Bio-Rad, Cat #161-4023) were used following the manufacturer's protocol. The following antibodies were used for Co-IP confirmation experiments: Rabbit anti-V5 (Abcam, Cat #ab9116), Mouse anti-Galectin-3 (Santa Cruz Biotechnology, Cat #SC-32790), Rabbit anti-Galectin-3BP (Proteintech, Cat #10281-1-AP), Mouse IgG (Santa Cruz Biotechnology, Cat #SC-2025), and Rabbit IgG (R&D Systems, Cat #AF008). For in vivo THP-1 cell or in vitro recombinant protein Co-IP experiments, additional following antibodies were used: Mouse anti-CHI3L1 (Santa Cruz Biotechnology, Cat #SC-393590), Rat anti-Galectin-3 (Santa Cruz Biotechnology, Cat #SC-23938), anti-Galectin-3BP (Santa Cruz Biotechnology, Cat #SC-374541), Rabbit anti-CHI3L1 (Cell Signaling Technology, Cat #47066), and Rat IgG (BioXCell, Cat #BE0090).

For protein identification by MS, the short gel fractionation method was used for sample preparation. The SDS-PAGE gel was run at 150 V for about 30 min until all samples fully migrated into the resolving gel. The gel was washed with ultrapure water twice, stained with Coomassie Blue for 4 hours at RT, and then de-stained until the bands appeared. The bands were excised and placed in 1.5 mL Eppendorf microcentrifuge tubes containing 200 μL of ultrapure water. The samples were digested with Sequencing Grade TPCK-Treated Trypsin (Promega, Cat #V511A) followed by liquid chromatography (LC) (Dionex, nanoLC; NEW Objective, nano-ESI) separation and MS (ThermoFisher, LTQ XL linear ion trap) analysis. MS results were searched using MASCOT (Matrix Sciences) and returned using Scaffold (Proteome Software, Inc.). Only extracellular and cell membrane-associated proteins (Table 3) from the LC-MS list were used for correlation analysis with CHI3L1.

Immunoblotting (IB), Immunohistochemistry (IHC), and Immunofluorescence (IF). The antibodies used for IB were purchased from Cell Signaling Technology unless otherwise noted: Phospho-Akt (Thr308; Cat #4056; RRID: AB_331163), Phospho-Akt (Ser 473; Cat #9271; RRID: AB_329825), AKT (Cat #9272; RRID: AB_329827), Phospho-S6 Ribosomal Protein (Ser235/236; Cat #4858S; RRID: AB_916156), Phospho-NF-κB p65 (Ser536; Cat #3033; RRID: AB_331284), NF-κB p65 (Cat #8242S; RRID: AB_10859369), Phospho-C/EBPβ (Thr235; Cat #3084; RRID: AB_2260359), Phospho-mTOR (Ser2448; Cat #5536; RRID: AB_10691552), C/EBP β (Santa Cruz, Cat #sc-150; RRID: AB_2260363), p53 (Santa Cruz, Cat #sc-6243; RRID: AB_653753), β-Actin (Sigma-Aldrich, Cat #A2228; RRID: AB_476697), and Vinculin (Santa Cruz, Cat #sc-25336; RRID: AB_628438). The following antibodies were used for IB, IHC, and/or IF: Galectin-3 (Santa Cruz, Cat #sc-32790; RRID: AB_627657), Lgals3 bp (Galectin-3BP; Proteintech; Cat #10281-1-AP; RRID: AB_2137066), CHI3L1 (Santa Cruz, Cat #sc-30465; RRID: AB_2081268), and V5 (Abcam, Cat #ab9116; RRID: AB_307024). The following antibodies were used for IF staining: TMEM119 (Proteintech; Cat #27585-1-AP), P2Y12 (ANASPEC; Cat #AS-55043 Å), F4/80 (Invitrogen; Cat #MF48000), CD49D (Invitrogen; Cat #PA5-20599), CD206 (Invitrogen; Cat #MA5-16871), Alexa Fluor 594 donkey anti-rabbit IgG (Invitrogen; Cat #A21207; RRID: AB_141637), Alexa Fluor 594 donkey anti-mouse IgG (Invitrogen; Cat #A21203; RRID: AB_141633), Cy3 AffiniPure Donkey anti-Goat IgG (Jackson Immuno Research; Cat #705-165-147; RRID: AB_2307351), Alexa Fluor 488 AffiniPure Donkey anti-Mouse IgG (Jackson Immuno Research; Cat #715-545-151; RRID: AB_2341099), Alexa Fluor 488 AffiniPure Donkey anti-Goat IgG (Jackson Immuno Research; Cat #705-545-147; RRID: AB_2336933), and Alexa Fluor 488 donkey anti-rabbit IgG (Invitrogen; Cat #A21206; RRID: AB_2535792).

Magnetic Resonance Imaging (MRI). Anesthesia for In Vivo MRI:

All mice received general inhalation anesthesia with Isoflurane for in vivo brain imaging. Mice were placed in a clear plexiglass anesthesia induction box that allowed unimpeded visual monitoring of the animals. Induction was achieved by administration of 3% Isoflurane mixed with oxygen for a few minutes. Depth of anesthesia was monitored by toe reflex (extension of limbs, spine positioning) and respiration rate. Once the plane of anesthesia was established, it was maintained with 1-2% Isoflurane in oxygen via a nose cone, and the mouse was transferred to the animal bed for imaging. Respiration was monitored using a pneumatic sensor placed between the animal bed and the mouse's abdomen, rectal temperature was measured with a fiberoptic sensor, and core temperature was maintained at 36.8±0.2° C. with a feedback-controlled warm air source (SA Instruments). In vivo MRI Acquisition: In vivo MRI brain image was carried out using a Bruker BioSpec 70/30 USR spectrometer (Bruker BioSpin MRI) operating at 7-Tesla field strength, equipped with an actively shielded B-GA12S2 gradient system with 440 mT/m gradient strength and slew rate of 3440 T/m/s, as well as a quadrature radiofrequency volume coil with an inner diameter of 35 mm. Multi-planar T2-weighted anatomical imaging of 11 to 21 slices (depending on tumor size and to cover the whole brain volume) was acquired with Rapid Imaging with Refocused Echoes (RARE) pulse sequence with the following parameters: field of view (FOV)=2.0 cm, matrix=256×256, slice thickness=0.6 mm, in-plane resolution=78 μm×78 μm, RARE factor=8, echo time (TE)=12 msec, effective echo time (TE)=48 msec, repetition time (TR)=1600 msec, and flip angle (FA)=180°. Volumetric Analysis of T₂-weighted MR: The multi-planar T₂-weighted RARE images were exported to DICOM format and analyzed by blinded independent observers using the open-source ITK-SNAP (www.itksnap.org) brain segmentation software. Tumor volume was defined as areas of hyperintensity; hemorrhage volume was defined as areas of hypointensity

Mice and Animal Housing.

Female ICR SCID mice at 4 weeks of age were purchased from Taconic Biosciences. Male or female C57BL/6 mice at 4-6 weeks of age were purchased from The Jackson Laboratory. Female and male mice were separated by sex into groups of 5 and 4 animals, respectively, and housed in large plastic cages under pathogen-free conditions. All animal experiments were performed with the approval of the University of Pittsburgh's Institutional Animal Care and Use Committee (IACUC).

Flow Cytometry and CyTOF.

The antibodies used for flow cytometry were purchased from BioLegend unless otherwise noted: CD45-PerCP-Cy5.5 (Cat #103132, 30-F11), CD3-APC (Cat #100236, 17 Å2), CD4-APC-Cy7 (Cat #100414, GK1.5), CD8a-BV711 (Cat #100747, 53-6.7), CD25-BV650 (Cat #102037, PC61), NK1.1-PE (Cat #108708, PK136), CD127-PE-Dazzle 594 (Cat #135032, A7R34), PD-1-PE (Cat #135206, 29F.1 Å12), PD-L1-BV421 (Cat #124315, 1° F.9G2), CTLA-4-PE-Dazzle 594 (Cat #106318, UC10-4B9), CD14-PE (Cat #123310, Sa14-2), Ly-6G-BV421 (Cat #127627, 1 Å8), Ly-6C-BV711 (Cat #128037, HK1.4), MHC II-APC-Cy7 (Cat #107628, M5/114.15.2), CD11b-PE-Cy7 (Cat #101216, M1/70), F4/80-APC (Cat #123116, BM8), CD68-BV421(Cat #137017, FA-11), CD206-PE (Cat #141706, C068C2), iNOS-APC-eFluor 780 (Thermo Fisher Scientific, Cat #47-5920-82, CXNFT), Arginase 1-APC (Thermo Fisher Scientific, Cat #17-3697-82, AlexF5), and Live/Dead Fixable Blue Dead Cell Stain Kit for UV excitation (Invitrogen, Cat #L34961).

For CyTOF, 500 mM of Rh103 intercalator (Fluidigm, Cat #201103 Å) was used to stain dead cells (10 min at RT), followed by incubation with 5 μL of FcX-Block (BioLegend, Cat #422302) for an additional 10 min at RT. Then a cocktail of surface marker antibodies was added, and samples were incubated for 30 min at RT. All CyTOF antibodies were commercially available and purchased directly from the CyTOF Core-Lederer Lab (Brigham Women's Hospital, Harvard Medical School) with conjugated metals (http://ledererlab.bwh.harvard.edu/cytof-core/). Samples were washed twice with 500 μL of CyTOF staining buffer (CSB; 500 mL low-barium PBS containing 2.5 g BSA [Sigma-Aldrich, Cat #A3059] and 100 mg sodium azide [Sigma-Aldrich, Cat #71289]). Cells were then resuspended in 500 μL of FoxP3 fix/perm (ThermoFisher, Cat #00-5523) and incubated for 45 min at RT. After cells were washed twice with 1 mL of FoxP3 permeabilization buffer (ThermoFisher, Cat #00-5523), a cocktail of intracellular marker antibodies was added to each sample and incubated for 45 min at RT. Cells were washed twice with 500 μL of CSB followed by incubation in 500 μL of 1.6% paraformaldehyde for 10 min at RT. Cells were pelleted and resuspended in 500 μL of CSB. Prior to the acquisition, samples were pelleted, resuspended in 500 μL of CSB containing 125 μM iridium intercalator (Fluidigm, Cat #201192A), and incubated for 20 min at RT. Cells were washed twice with 500 μL of CSB, twice with 500 μL of water (Fluidigm, Cat #201069), and resuspended in water at [a final concentration of] ˜5×10⁵ cells/mL. Then EQ beads (Fluidigm, Cat #201078) were added to samples (1:1000 dilution) for normalization, followed by acquisition on a Helios2 CyTOF system (Fluidigm).

Clinical GBM Datasets Analysis.

TCGA GBM datasets include gene mutations, copy number, gene expression, proteomics (RPPA), tumor subtypes, and patient survival information (tcga-data.nci.nih.gov). Data from 10 normal and 371 IDH wild-type GBM samples were analyzed. Wilcoxon rank-sum tests were used to examine the significance of the differences between groups (Figures IM and 10; FIG. 5G; FIG. 7E; FIGS. 11H and 11H; FIG. 13N; FIG. 15E). Spearman's tests were used to assess the significance of the correlation between groups (FIG. 1N; FIG. 4D; FIG. 14D). A log-rank test was used to examine the significance of patient overall survival (FIG. 11J).

Regarding correlation analysis between gene expression (CHI3L1, LGALS3, and LGALS3BP) and GBM patient response to anti-PD-1 treatment, a published clinical dataset contains RNA-seq-based gene expression filing from 16 patients with one or more biospecimens before treatment with anti-PD-1 inhibitors (nivolumab or pembrolizumab). The gene expression mean was used for patients with more than one biospecimen for RNA-seq. Gene expression was ranked and used to separate patients into two groups with equal size, designated as high and low expression groups, for each gene (FIG. 17J). The number of patients in the group with a combination of genes with high and low expression is counted by taking the intersection of patients in the corresponding high/low gene expression groups. The anti-PD-1 treatment response rate in each patient group is calculated as the number of positive responders over the total number of patients in the group (FIG. 10A).

TABLE 1
Key resource table

| | | |
|---|---|---|
| REAGENT or RESOURCE | SOURCE | IDENTIFIER |

Antibodies

| | | |
|---|---|---|
| IB: Rabbit anti-Phospho-Akt (Thr308) | Cell Signaling Technology | Cat# 4056; RRID: AB_331163 |
| IB: Rabbit anti-Phospho-Akt (Ser 473) | Cell Signaling Technology | Cat# 9271; RRID: AB_329825 |
| (244F9) | | |
| IB: Rabbit anti-AKT | Cell Signaling Technology | Cat# 9272; RRID: AB_329827 |
| IB and IHC: Rabbit monoclonal anti-Phospho- | Cell Signaling Technology | Cat# 4858S; RRID: |
| S6 Ribosomal Protein (Ser235/236) | | AB_916156 |
| (D57.2.2E) XP | | |
| IB: Rabbit anti-S6 Ribosomal Protein (5G10) | Cell Signaling Technology | Cat# 2217S; RRID: |
| | | AB_331355 |
| IB: Rabbit anti-Phospho-NF-κB p65 (Ser536) | Cell Signaling Technology | Cat# 3033; RRID: AB_331284 |
| (93H1) | | |
| IB: Rabbit anti-NF-κB p65 (D14E12) XP | Cell Signaling Technology | Cat# 8242S; RRID: |
| | | AB_10859369 |
| IB: Rabbit anti-Phospho-C/EBPβ (Thr235) | Cell Signaling Technology | Cat# 3084; RRID: |
| | | AB_2260359 |
| IB: Rabbit anti-Phospho-mTOR (Ser2448) | Cell Signaling | Cat# 5536; RRID: |
| (D9C2) | Technology | AB_10691552 |
| IB: Rabbit anti-C/EBP β | Santa Cruz Biotechnology | Cat# sc-150; RRID: |
| | | AB_2260363 |
| IB: Rabbit anti-p53 (FL393) | Santa Cruz Biotechnology | Cat# sc-6243; RRID: |
| | | AB_653753 |
| IB: Mouse anti-β-Actin (AC-74) | Sigma-Aldrich | Cat# A2228; RRID: |
| | | AB_476697 |
| IB: Mouse anti-Vinculin (H-10) | Santa Cruz Biotechnology | Cat# sc-25336; RRID: |
| | | AB_628438 |
| IB: Goat anti-Rabbit IgG (H + L) Secondary | Invitrogen | Cat# 31460; RRID: |
| Antibody, HRP | | AB_228341 |
| IB: Goat anti-Mouse IgG (H + L) Secondary | Invitrogen | Cat# 31430; RRID: |
| Antibody, HRP | | AB_228307 |
| IB: Rabbit anti-Goat IgG (H + L) Secondary | Invitrogen | Cat# 31402; RRID: |
| Antibody, HRP | | AB_228395 |
| IB: Goat anti-Rat IgG (H + L) Secondary | Invitrogen | Cat# 31470; RRID: |
| Antibody, HRP | | AB_228356 |
| IB, IP, and IF: Mouse anti-Galectin-3 | Santa Cruz Biotechnology | Cat# sc-32790; RRID: |
| | | AB_627657 |
| IB, IP, and IF: Rabbit anti-Lgals3bp (Galectin- | Proteintech | Cat# 10281-1-AP; RRID: |
| 3BP) | | AB_2137066 |
| IB, IP, IHC, and IF: Goat anti-CHI3L1 (S-18) | Santa Cruz Biotechnology | Cat# sc-30465; RRID: |
| | | AB_2081268 |
| IB and IP: Rabbit anti-V5 | Abcam | Cat# ab9116; RRID: |
| | | AB_307024 |
| IP: Mouse IgG | Santa Cruz Biotechnology | Cat# sc-2025; RRID: |
| | | AB_737182 |
| IP: Rabbit IgG | R&D Systems | Cat# AF008; RRID: |
| | | AB_354521 |
| IP: Rabbit IgG | NeoMarkers | Cat# NC-100-P1; |
| | | RRID: AB_60827 |
| IF: Rabbit anti-TMEM119 | Proteintech | Cat# 27585-1-AP |
| IF: Rabbit anti-mouse P2Y12 | ANASPEC | Cat# AS-55043A |
| IF: Rabbit anti-Iba1 | Abcam | Cat# ab178846; RRID: |
| | | AB_2636859 |
| IF: Mouse F4/80 (BM8) | Invitrogen | Cat# MF48000 |
| IF: Alexa Fluor 594 donkey anti-rabbit IgG | Invitrogen | Cat# A21207; RRID: |
| (H + L) | | AB_141637 |
| IF: Alexa Fluor 594 donkey anti-mouse IgG | Invitrogen | Cat# A21203; RRID: |
| (H + L) | | AB_141633 |
| IF: Cy3 AffiniPure Donkey anti-Goat IgG | Jackson Immuno Research | Cat# 705-165-147; RRID: |
| (H + L) | | AB_2307351 |
| IF: Alexa Fluor 488 AffiniPure Donkey anti- | Jackson Immuno Research | Cat# 715-545-151; RRID: |
| Mouse IgG (H + L) | | AB_2341099 |
| IF: Alexa Fluor 488 AffiniPure Donkey | Jackson Immuno Research | Cat# 705-545-147; RRID: |
| anti-Goat IgG (H + L) | | AB_2336933 |
| IF: Alexa Fluor 488 donkey anti-rabbit IgG | Invitrogen | Cat# A21206; RRID: |
| (H + L) | | AB_2535792 |
| Flow: Rat anti-mouse CD45-PerCP-Cy5.5 | BioLegend | Cat# 103132; RRID: |
| | | AB_893340 |
| Flow: Rat anti-mouse CD3-APC | BioLegend | Cat# 100236; RRID: |
| | | AB_2561456 |
| Flow: Rat anti-mouse CD4-APC-Cy7 | BioLegend | Cat# 100414; RRID: |
| | | AB_312699 |
| Flow: Rat anti-mouse CD8a-BV711 | BioLegend | Cat# 100747; RRID: |
| | | AB_11219594 |
| Flow: Rat anti-mouse CD25-BV650 | BioLegend | Cat# 102037; RRID: |
| | | AB_11125760 |
| Flow: Rat anti-mouse NK1.1-PE | BioLegend | Cat# 108708; RRID: |
| | | AB_313395 |
| Flow: Rat anti-mouse CD127-PE-Dazzle 594 | BioLegend | Cat# 135032; RRID: |
| | | AB_2564217 |
| Flow: Rat anti-mouse PD-1-PE | BioLegend | Cat# 135206; RRID: |
| | | AB_1877231 |
| Flow: Rat anti-mouse PD-L1-BV421 | BioLegend | Cat# 124315; RRID: |
| | | AB_10897097 |
| Flow: Rat anti-mouse CTLA-4-PE-Dazzle | BioLegend | Cat# 106318; RRID: |
| 594 | | AB_2564496 |
| Flow: Rat anti-mouse CD14-PE | BioLegend | Cat# 123310; RRID: |
| | | AB_940584 |
| Flow: Rat anti-mouse Ly-6G-BV421 | BioLegend | Cat# 127627; RRID: |
| | | AB_10897944 |
| Flow: Rat anti-mouse Ly-6C-BV711 | BioLegend | Cat# 128037; RRID: |
| | | AB_2562630 |
| Flow: Rat anti-mouse MHC II-APC-Cy7 | BioLegend | Cat# 107628; RRID: |
| | | AB_2069377 |
| Flow: Rat anti-mouse CD11b-PE-Cy7 | BioLegend | Cat# 101216; RRID: |
| | | AB_312799 |
| Flow: Rat anti-mouse F4/80-APC | BioLegend | Cat# 123116; RRID: |
| | | AB_893481 |
| Flow: Rat anti-mouse F4/80-APC | Invitrogen | Cat# 47-4801-82; |
| | | AB_2735036 |
| Flow: Rat anti-mouse CD11b-PE | BD Biosciences | Cat# 553311; |
| | | RRID: AB_394775 |

Chemicals, Peptides, and Recombinant Proteins

| | | |
|---|---|---|
| Human CHI3L1 Recombinant Protein | R&D Systems | Cat# 2599-CH-050 |
| Human Galectin-3 Recombinant Protein | R&D Systems | Cat# 8259-GA-050 |
| pLenti6.3-myr-AKT | Hu et al. Cell, 2016 | N/A |
| pLenti6.3_CHI3L1_V5 | This paper | N/A |
| pLenti6.3_CHI3L1 | This paper | N/A |
| pLKO.1 target gene set (CHI3L1) | Sigma-Aldrich | SHCLNG- NM_007695 |

Software and Algorithms

| | | |
|---|---|---|
| FlowJo_v10.7.1 | FlowJo | www.flowjo.com; RRID: |
| | | SCR_008520 |
| CytoBank | CytoBank Inc. | www.cytoban k.org; RRID: |
| | | RRID:SCR_014043 |
| Fiji | ImageJ | imagej.net/Fiji; |
| | | RRID: SCR_002285 |
| R environment, v3.6.3 | R core team | rstudio.com |
| MASCOT | Matrix Science Inc | www.matrixsci ence.com; |
| | | RRID: SCR_014322 |
| Scaffold | Proteome Software Inc | www.proteome |
| | | software.com/products/scaffold/; |
| | | RRID: SCR_014345 |
| GraphPad Prism | GraphPad Software | www.graphpa d.com; RRID: |
| | | SCR_002798 |
| Fiji MRI wound heal tool | ImageJ-macros | dev.mri.cnrs.fr; RRID: N/A |
| ITK-SNAP (v.3.8.0) | Yushkevich et al., 2016 | www.itksnap.or g; RRID: |
| | | SCR_002010 |

Other

| | | |
|---|---|---|
| Smart-Seq v4 ultra Low input RNA Kit | Takara | Cat# 634891 |
| Nestera XT | Illumina | Cat# FC-131-1096 |
| CyTOF Core-Lederer Lab (Conjugated | Brigham Women's Hospital, | ledererlab.bwh. |
| Antibodies) | Harvard Medical School | harvard.edu/cytof-core/ |

TABLE 2
Genes are significantly correlated to CHI3L1 in TCGA GBM dataset

| Gene | case.mean | control.mean | s2n | p | FDR |
|---|---|---|---|---|---|
| CHI3L1 | 12.6953396 | 8.963619543 | 1.33038382 | 4.76E−73 | 5.74E−69 |
| PLA2G5 | 7.17859191 | 4.940500435 | 0.83306618 | 7.86E−42 | 9.46E−38 |
| PBEF1 | 10.9067009 | 9.282892573 | 0.80299784 | 9.24E−42 | 1.11E−37 |
| RNASE2 | 7.18101322 | 5.645627102 | 0.7563372 | 5.21E−37 | 6.27E−33 |
| HRH1 | 7.53076212 | 6.217493106 | 0.74758178 | 3.27E−37 | 3.93E−33 |
| PTX3 | 8.8135652 | 6.455513002 | 0.74552202 | 2.69E−37 | 3.23E−33 |
| CHI3L2 | 9.28194518 | 6.802699691 | 0.73843268 | 1.60E−36 | 1.93E−32 |
| MAN1C1 | 9.36784354 | 7.498898898 | 0.72934947 | 1.66E−36 | 1.99E−32 |
| TIMP1 | 11.8400831 | 10.51626828 | 0.71441624 | 3.22E−38 | 3.87E−34 |
| TNFRSF1A | 8.52074945 | 7.611531774 | 0.70252371 | 6.70E−35 | 8.06E−31 |
| PDPN | 8.9515335 | 7.110154077 | 0.68780607 | 2.65E−33 | 3.19E−29 |
| FCGBP | 10.3520235 | 8.104821549 | 0.68047071 | 2.56E−31 | 3.08E−27 |
| CEBPD | 8.46340582 | 7.604622564 | 0.67134104 | 2.52E−32 | 3.03E−28 |
| SERPINA3 | 12.4101076 | 10.35777802 | 0.65916496 | 1.51E−31 | 1.81E−27 |
| STEAP3 | 7.69133605 | 6.414198684 | 0.65342489 | 1.64E−30 | 1.97E−26 |
| CA12 | 7.96352599 | 6.329209587 | 0.65278869 | 2.58E−31 | 3.10E−27 |
| CXCL14 | 9.24083534 | 6.698386634 | 0.6513333 | 1.88E−30 | 2.27E−26 |
| SOD2 | 8.13638176 | 6.769062406 | 0.64941343 | 4.94E−31 | 5.94E−27 |
| CLEC5A | 6.50774426 | 5.105047372 | 0.64864938 | 1.96E−30 | 2.35E−26 |
| LTF | 9.55214681 | 6.379816619 | 0.64169903 | 5.82E−30 | 7.00E−26 |
| DPYD | 8.8856625 | 7.328917712 | 0.64104848 | 2.68E−32 | 3.23E−28 |
| CHST2 | 8.89886404 | 7.687568659 | 0.64086132 | 3.51E−32 | 4.22E−28 |
| EMP3 | 9.4544323 | 7.992512062 | 0.63808785 | 6.68E−30 | 8.02E−26 |
| EFEMP2 | 8.1677509 | 7.050469169 | 0.63042925 | 1.47E−29 | 1.76E−25 |
| C21orf62 | 7.58331744 | 5.814259535 | 0.62826617 | 1.80E−26 | 2.16E−22 |
| PLTP | 9.65916208 | 8.278175279 | 0.62206116 | 3.67E−27 | 4.41E−23 |
| CCL2 | 9.48877403 | 7.465487826 | 0.62175537 | 1.60E−28 | 1.92E−24 |
| LGALS3 | 11.8177087 | 10.49960509 | 0.62070081 | 5.28E−30 | 6.34E−26 |
| PMP22 | 11.2380702 | 10.13386308 | 0.60459724 | 7.59E−30 | 9.13E−26 |
| CD44 | 8.92317691 | 7.688712909 | 0.60242432 | 2.21E−27 | 2.66E−23 |
| PLA2G2A | 7.59760808 | 5.42437722 | 0.60146788 | 3.72E−29 | 4.47E−25 |
| EFEMP1 | 10.2560276 | 8.541825843 | 0.59390981 | 1.93E−26 | 2.32E−22 |
| RHOG | 8.69520281 | 7.967732469 | 0.59318556 | 9.53E−28 | 1.14E−23 |
| FPR1 | 6.32091846 | 5.688231389 | 0.59133725 | 1.46E−26 | 1.76E−22 |
| CFI | 8.08891828 | 6.641087851 | 0.59059157 | 3.51E−27 | 4.22E−23 |
| CHPT1 | 9.44734605 | 8.621008248 | 0.58925641 | 5.24E−29 | 6.30E−25 |
| ABCC3 | 6.77436499 | 5.422688862 | 0.58746074 | 3.06E−27 | 3.68E−23 |
| VAMP5 | 6.80622516 | 5.975032855 | 0.58632505 | 9.54E−27 | 1.15E−22 |
| C5AR1 | 6.84227451 | 5.724859176 | 0.58415392 | 2.79E−27 | 3.35E−23 |
| SERPING1 | 9.06862183 | 7.602303704 | 0.58217825 | 1.20E−25 | 1.44E−21 |
| C1RL | 6.95094076 | 5.836031604 | 0.57971471 | 1.61E−25 | 1.93E−21 |
| HAMP | 7.67090827 | 6.044289351 | 0.57809147 | 6.95E−26 | 8.34E−22 |
| C1S | 9.82394733 | 8.320822144 | 0.57563779 | 3.76E−25 | 4.51E−21 |
| MRC2 | 7.98005186 | 7.092448145 | 0.57390182 | 5.67E−26 | 6.80E−22 |
| CCDC109B | 8.32849431 | 7.358296331 | 0.57357386 | 5.38E−27 | 6.45E−23 |
| CTSB | 10.6616567 | 9.860565076 | 0.57185034 | 3.08E−28 | 3.70E−24 |
| CD63 | 12.6276631 | 12.16043846 | 0.56616911 | 1.50E−25 | 1.80E−21 |
| TPP1 | 8.80677417 | 7.959364046 | 0.56415846 | 6.57E−26 | 7.88E−22 |
| DIRAS3 | 7.32309718 | 5.826391284 | 0.56237297 | 1.85E−25 | 2.22E−21 |
| STAB1 | 7.92761907 | 6.83647896 | 0.55951021 | 7.61E−25 | 9.11E−21 |
| NR2E1 | 7.82842743 | 6.075190181 | 0.5590407 | 9.88E−20 | 1.17E−15 |
| FCGR2A | 7.77477694 | 6.650291106 | 0.55862813 | 8.58E−26 | 1.03E−21 |
| PTRF | 7.41259487 | 6.46158509 | 0.55862561 | 8.55E−25 | 1.02E−20 |
| DPY19L1 | 9.20491951 | 8.265651876 | 0.55837638 | 3.05E−24 | 3.65E−20 |
| TNC | 10.7283844 | 9.272990918 | 0.55741657 | 2.89E−24 | 3.46E−20 |
| PYGL | 7.93206766 | 6.907096313 | 0.55651198 | 4.05E−24 | 4.85E−20 |
| C1orf54 | 9.17494623 | 8.222044342 | 0.55646838 | 2.05E−26 | 2.45E−22 |
| NRCAM | 9.13011281 | 7.988490855 | 0.55623885 | 4.46E−22 | 5.32E−18 |
| SRPX2 | 7.00416422 | 5.655734381 | 0.55444154 | 7.20E−25 | 8.63E−21 |
| SCG2 | 9.67272414 | 7.916335277 | 0.55292685 | 1.64E−23 | 1.96E−19 |
| GLIPR1 | 7.88139956 | 6.746399958 | 0.5507341 | 5.53E−24 | 6.62E−20 |
| CPVL | 8.31844681 | 7.125762793 | 0.55000935 | 4.12E−25 | 4.93E−21 |
| GBP2 | 7.96732764 | 6.915312559 | 0.55000308 | 3.30E−25 | 3.95E−21 |
| LHFPL2 | 8.23634045 | 7.452322742 | 0.54873016 | 3.69E−24 | 4.41E−20 |
| C3 | 11.5281995 | 10.09685559 | 0.54545827 | 6.17E−26 | 7.40E−22 |
| SERPINE1 | 7.86257991 | 6.436710435 | 0.54524872 | 2.39E−24 | 2.86E−20 |
| TIPARP | 9.7102123 | 8.897557541 | 0.54499216 | 9.30E−24 | 1.11E−19 |
| BHLHB3 | 9.03638001 | 7.817110279 | 0.54415569 | 1.45E−21 | 1.72E−17 |
| DKFZP586H2123 | 7.58323923 | 6.293481601 | 0.54227448 | 2.92E−23 | 3.48E−19 |
| ANGPTL4 | 6.8101082 | 5.606362971 | 0.54156645 | 8.93E−24 | 1.07E−19 |
| CD163 | 9.87899835 | 8.149671167 | 0.541449 | 1.34E−25 | 1.61E−21 |
| CDKN1A | 8.51529808 | 7.563167362 | 0.54134268 | 3.90E−23 | 4.66E−19 |
| SLC11A1 | 5.60629168 | 5.064832597 | 0.54025908 | 1.19E−24 | 1.43E−20 |
| MAOB | 10.1190345 | 8.423654883 | 0.54024142 | 2.36E−23 | 2.82E−19 |
| ARSJ | 5.83690697 | 4.756101225 | 0.53962062 | 4.50E−25 | 5.40E−21 |
| CD14 | 10.0691678 | 8.70774506 | 0.53724438 | 1.81E−26 | 2.18E−22 |
| LOC26010 | 7.67202788 | 7.087453364 | 0.53669905 | 1.17E−24 | 1.40E−20 |
| SLA | 7.80414969 | 6.72359688 | 0.53589449 | 8.18E−23 | 9.77E−19 |
| GADD45A | 10.2166133 | 9.280926924 | 0.53589098 | 3.56E−23 | 4.25E−19 |
| FZD7 | 7.67751299 | 6.456056908 | 0.53466047 | 5.88E−24 | 7.03E−20 |
| F3 | 8.72377976 | 7.660787781 | 0.53396104 | 1.20E−23 | 1.43E−19 |
| CSF1R | 8.52256851 | 7.409005637 | 0.53352755 | 2.44E−23 | 2.91E−19 |
| CCR1 | 6.93950938 | 6.014655122 | 0.53303906 | 1.08E−23 | 1.29E−19 |
| ITGA5 | 7.46444174 | 6.642937958 | 0.53301022 | 4.47E−25 | 5.36E−21 |
| VSIG4 | 10.1932438 | 8.638554407 | 0.53277426 | 4.87E−24 | 5.82E−20 |
| ALOX5AP | 10.1009853 | 8.546222942 | 0.53250924 | 2.93E−25 | 3.51E−21 |
| M6PRBP1 | 8.86545787 | 8.158925021 | 0.53046233 | 1.13E−23 | 1.35E−19 |
| SLC39A14 | 9.45501488 | 8.522999725 | 0.52844964 | 1.34E−24 | 1.60E−20 |
| CLU | 11.962617 | 10.70301618 | 0.52817908 | 2.44E−23 | 2.91E−19 |
| EMP1 | 9.74829451 | 8.59798662 | 0.52744423 | 1.00E−23 | 1.20E−19 |
| SLC2A5 | 6.88306435 | 5.86550867 | 0.52724433 | 8.23E−23 | 9.83E−19 |
| TRIP6 | 8.28099389 | 7.370639665 | 0.52719244 | 8.94E−21 | 1.06E−16 |
| CSRP2 | 10.84429 | 9.723842973 | 0.52590801 | 1.10E−22 | 1.31E−18 |
| ITGAM | 5.61227751 | 5.152434015 | 0.52269749 | 1.27E−23 | 1.52E−19 |
| FLJ21963 | 6.87721514 | 5.713068377 | 0.52218229 | 4.10E−21 | 4.88E−17 |
| TNFSF12 | 5.96206672 | 5.47456119 | 0.52202693 | 2.66E−21 | 3.17E−17 |
| SH3BGRL3 | 9.3749445 | 8.759764899 | 0.52177873 | 3.79E−24 | 4.53E−20 |
| LGALS1 | 11.727482 | 10.90338964 | 0.51989859 | 6.63E−25 | 7.94E−21 |
| ILK | 8.75766162 | 8.229700147 | 0.51574413 | 5.26E−23 | 6.28E−19 |
| ADORA1 | 5.6184696 | 5.116587744 | 0.51480473 | 1.11E−21 | 1.33E−17 |
| PIPOX | 8.22060358 | 6.739152482 | 0.5141336 | 5.81E−20 | 6.89E−16 |
| IQGAP1 | 8.79985539 | 8.034942682 | 0.51327291 | 6.20E−22 | 7.39E−18 |
| NNMT | 9.50904244 | 7.647626721 | 0.51315793 | 3.87E−21 | 4.61E−17 |
| SEC14L2 | 6.13392204 | 5.318039618 | 0.51300018 | 3.99E−21 | 4.75E−17 |
| HMOX1 | 8.92249574 | 7.673176563 | 0.51298289 | 1.69E−23 | 2.02E−19 |
| GAL3ST4 | 7.37727288 | 6.735786116 | 0.51146658 | 2.76E−22 | 3.29E−18 |
| GLUL | 10.5867344 | 9.678923293 | 0.51133116 | 8.13E−23 | 9.71E−19 |
| LRP10 | 8.95914807 | 8.322827314 | 0.5109314 | 9.85E−22 | 1.17E−17 |
| RAMP1 | 9.24018044 | 7.941057593 | 0.51085358 | 1.02E−18 | 1.21E−14 |
| SIPA1L1 | 7.4924026 | 6.757137465 | 0.51020174 | 2.03E−21 | 2.41E−17 |
| MYO1F | 6.5818967 | 5.876559829 | 0.5099277 | 3.94E−22 | 4.70E−18 |
| GADD45B | 7.15289831 | 6.368353511 | 0.50990851 | 4.97E−24 | 5.94E−20 |
| KCNMB1 | 5.97765079 | 5.047060942 | 0.50984484 | 1.48E−23 | 1.77E−19 |
| GAP43 | 9.64461393 | 8.055460062 | 0.5092569 | 8.25E−18 | 9.73E−14 |
| TAGLN | 9.0582132 | 7.622517101 | 0.5080596 | 2.15E−21 | 2.56E−17 |
| FLOT1 | 7.79114542 | 7.459227625 | 0.50758919 | 3.33E−22 | 3.98E−18 |
| ZYX | 8.54240789 | 7.58099265 | 0.50649451 | 1.53E−21 | 1.82E−17 |
| F13A1 | 8.33044636 | 6.55641997 | 0.50645464 | 1.96E−20 | 2.33E−16 |
| ITGA7 | 7.28376067 | 6.239474178 | 0.5059957 | 2.47E−20 | 2.93E−16 |
| ITGB2 | 8.98350736 | 7.908498056 | 0.50426814 | 2.03E−22 | 2.43E−18 |
| CD4 | 6.87032079 | 6.095237992 | 0.5041994 | 1.13E−21 | 1.34E−17 |
| ADORA3 | 7.28663573 | 6.194521611 | 0.50357895 | 5.24E−21 | 6.23E−17 |
| ACTN1 | 9.40875166 | 8.439833006 | 0.50283319 | 1.12E−20 | 1.33E−16 |
| TLR2 | 7.07824042 | 6.108257441 | 0.50270638 | 3.10E−21 | 3.69E−17 |
| SPOCK2 | 7.15754813 | 6.384570897 | 0.50195899 | 2.22E−21 | 2.64E−17 |
| ANXA1 | 10.6551161 | 9.440293034 | 0.50174955 | 4.57E−18 | 5.39E−14 |
| OSBPL3 | 6.76508577 | 5.959825337 | 0.50121634 | 2.53E−20 | 3.00E−16 |
| IFITM3 | 10.8850343 | 10.10807652 | 0.499051 | 2.60E−22 | 3.11E−18 |
| CA2 | 10.1161566 | 8.83910827 | 0.49884346 | 3.63E−22 | 4.32E−18 |
| NDP | 8.55245786 | 7.112912887 | 0.49746347 | 1.20E−17 | 1.41E−13 |
| CD151 | 7.31025887 | 6.647151705 | 0.49720084 | 9.51E−21 | 1.13E−16 |
| MT1G | 9.42636892 | 8.452809443 | 0.49717436 | 1.38E−20 | 1.63E−16 |
| TMBIM1 | 7.67034066 | 7.006971867 | 0.49710234 | 5.83E−21 | 6.94E−17 |
| BBOX1 | 8.78546809 | 7.184873352 | 0.49684908 | 9.98E−18 | 1.18E−13 |
| CEBPB | 9.68065166 | 8.90411578 | 0.49555806 | 1.14E−22 | 1.36E−18 |
| CARD9 | 5.2610044 | 4.915996275 | 0.49552992 | 3.85E−20 | 4.57E−16 |
| RCAN1 | 8.81067453 | 7.585003067 | 0.49511711 | 3.92E−21 | 4.67E−17 |
| FCER1G | 9.57679315 | 8.378691713 | 0.4945374 | 9.45E−23 | 1.13E−18 |
| FCGRT | 8.14458029 | 7.405014105 | 0.49407987 | 2.60E−21 | 3.09E−17 |
| LAMP2 | 9.50669522 | 8.879204326 | 0.49364681 | 3.97E−22 | 4.73E−18 |
| PCSK5 | 6.26149323 | 5.375504939 | 0.49186258 | 5.83E−21 | 6.94E−17 |
| LAIR1 | 6.17796314 | 5.470953035 | 0.49153956 | 5.59E−22 | 6.67E−18 |
| LCP2 | 6.69837933 | 5.892682213 | 0.49134528 | 7.00E−21 | 8.32E−17 |
| SERPINB6 | 9.33527077 | 8.764287083 | 0.4909522 | 3.56E−23 | 4.25E−19 |
| CHRNA9 | 6.17127076 | 5.024969731 | 0.49010797 | 1.24E−24 | 1.48E−20 |
| FEM1C | 7.56147934 | 6.779328603 | 0.48919549 | 9.13E−20 | 1.08E−15 |
| STBD1 | 6.05750337 | 5.589655224 | 0.48821836 | 1.70E−21 | 2.02E−17 |
| ITPKB | 8.82142723 | 7.681757176 | 0.48755213 | 8.44E−18 | 9.95E−14 |
| UPP1 | 7.18729197 | 6.252261619 | 0.48753084 | 1.28E−19 | 1.51E−15 |
| PKM2 | 10.4292957 | 9.726919413 | 0.48720131 | 8.19E−21 | 9.73E−17 |
| RAB13 | 10.6901312 | 10.15288374 | 0.48698053 | 1.37E−20 | 1.62E−16 |
| FAM129A | 7.95575768 | 6.861202117 | 0.48668767 | 2.62E−20 | 3.12E−16 |
| TREM1 | 6.88236324 | 5.634679501 | 0.48625405 | 2.62E−22 | 3.13E−18 |
| ITPKC | 5.89919109 | 5.409109032 | 0.48536578 | 4.71E−21 | 5.60E−17 |
| ANXA5 | 11.160568 | 10.58919413 | 0.48533764 | 2.05E−19 | 2.43E−15 |
| PRUNE2 | 8.02176189 | 7.026121834 | 0.48520878 | 1.23E−17 | 1.44E−13 |
| LOX | 5.713575 | 4.734456346 | 0.48429658 | 1.99E−22 | 2.38E−18 |
| AKAP12 | 8.96338234 | 7.679154243 | 0.48253794 | 1.61E−19 | 1.90E−15 |
| HCLS1 | 8.64868111 | 7.561738951 | 0.48251411 | 2.95E−20 | 3.50E−16 |
| C3AR1 | 8.01411181 | 7.064260858 | 0.4822867 | 4.56E−21 | 5.43E−17 |
| TNFRSF1B | 6.68282854 | 5.874532921 | 0.48158744 | 7.02E−20 | 8.33E−16 |
| CST3 | 12.3105008 | 11.29025162 | 0.48125304 | 2.46E−16 | 2.88E−12 |
| CTSZ | 6.3759595 | 5.856777785 | 0.47992833 | 1.25E−21 | 1.49E−17 |
| ACOX2 | 6.05280545 | 5.190435073 | 0.47959046 | 3.56E−22 | 4.24E−18 |
| COPZ2 | 7.53618425 | 6.572096868 | 0.4792159 | 7.37E−20 | 8.74E−16 |
| ENDOD1 | 7.6271072 | 6.836158557 | 0.47921303 | 2.97E−19 | 3.52E−15 |
| IBSP | 5.71080885 | 4.816836306 | 0.47843861 | 1.12E−19 | 1.32E−15 |
| LMO2 | 9.0895242 | 7.946887738 | 0.47827828 | 8.89E−18 | 1.05E−13 |
| LIF | 5.77189786 | 4.845103182 | 0.47751635 | 4.46E−23 | 5.32E−19 |
| HTRA1 | 11.5839268 | 10.54963532 | 0.47542771 | 1.17E−17 | 1.38E−13 |
| SRPX | 10.3027825 | 8.804436371 | 0.47408717 | 6.13E−20 | 7.28E−16 |
| CENTA2 | 7.5843213 | 6.895333193 | 0.47393312 | 2.64E−19 | 3.12E−15 |
| SLC2A10 | 7.89739233 | 6.900155543 | 0.47386862 | 2.51E−16 | 2.94E−12 |
| CNIH3 | 6.69938137 | 5.680812251 | 0.47382387 | 9.13E−20 | 1.08E−15 |
| APH1B | 6.36266377 | 5.931199598 | 0.47373688 | 3.16E−19 | 3.74E−15 |
| SIGLEC9 | 4.55822017 | 4.309747295 | 0.47259714 | 6.29E−21 | 7.48E−17 |
| GDPD2 | 6.47741252 | 5.725201795 | 0.47257051 | 5.78E−19 | 6.85E−15 |
| TBXAS1 | 6.06445369 | 5.353317437 | 0.47143946 | 8.80E−20 | 1.04E−15 |
| ABCA1 | 7.97958926 | 7.149234483 | 0.47066314 | 5.32E−19 | 6.30E−15 |
| PGCP | 7.05801062 | 6.381512637 | 0.47049921 | 1.47E−18 | 1.73E−14 |
| RNASET2 | 9.72319594 | 8.971247296 | 0.4703803 | 1.21E−20 | 1.44E−16 |
| PLOD3 | 8.47355174 | 7.904769694 | 0.4698071 | 5.46E−20 | 6.48E−16 |
| TPST1 | 9.25362364 | 8.392314277 | 0.46967061 | 2.73E−17 | 3.21E−13 |
| LAPTM5 | 10.1078115 | 9.090302222 | 0.46956801 | 2.29E−21 | 2.72E−17 |
| KCTD12 | 10.213615 | 9.23529993 | 0.46850174 | 1.79E−18 | 2.12E−14 |
| KCNE4 | 6.4073697 | 5.551832101 | 0.46843066 | 3.52E−18 | 4.15E−14 |
| CLIC4 | 8.96954066 | 8.205708923 | 0.46825321 | 3.18E−18 | 3.76E−14 |
| SPI1 | 5.66666229 | 5.097795783 | 0.46727231 | 2.56E−21 | 3.05E−17 |
| CXorf9 | 6.23038996 | 5.371085824 | 0.46709877 | 4.19E−19 | 4.96E−15 |
| RAB31 | 11.2456012 | 10.44768551 | 0.46521225 | 5.44E−16 | 6.36E−12 |
| VIM | 13.1220354 | 12.32535627 | 0.46489026 | 3.97E−22 | 4.73E−18 |
| CIQA | 9.79170528 | 8.598100592 | 0.46439062 | 5.66E−20 | 6.72E−16 |
| SIGLEC7 | 5.21745781 | 4.898475475 | 0.46423612 | 1.14E−17 | 1.34E−13 |
| AIF1 | 7.82461362 | 7.017661488 | 0.46383467 | 5.99E−19 | 7.09E−15 |
| EHD2 | 6.43204523 | 5.839078986 | 0.46376016 | 6.75E−19 | 7.99E−15 |
| ZFP36 | 9.12907814 | 8.232289416 | 0.46322338 | 1.18E−20 | 1.40E−16 |
| HSPA6 | 6.12840032 | 5.352359648 | 0.46314229 | 5.94E−21 | 7.07E−17 |
| RRAS | 7.18636493 | 6.447428944 | 0.46254493 | 1.47E−22 | 1.75E−18 |
| CSRP1 | 10.2608961 | 9.4181888 | 0.46152869 | 9.53E−19 | 1.13E−14 |
| SPP1 | 12.9829211 | 11.92087856 | 0.46131281 | 3.94E−22 | 4.70E−18 |
| TMEM158 | 9.77086425 | 8.492465124 | 0.46065222 | 9.05E−18 | 1.07E−13 |
| ADAM9 | 9.75770581 | 9.074199611 | 0.4606217 | 1.89E−20 | 2.24E−16 |
| SLC4A3 | 6.23571532 | 5.480995686 | 0.46011052 | 3.39E−17 | 3.98E−13 |
| MAFB | 9.83589498 | 8.704419269 | 0.45916505 | 2.06E−19 | 2.44E−15 |
| MT1X | 11.1553045 | 10.21627097 | 0.45883382 | 4.57E−17 | 5.36E−13 |
| SAT1 | 10.5371839 | 9.979125162 | 0.45875434 | 7.12E−19 | 8.43E−15 |
| MLC1 | 8.66039921 | 7.393040421 | 0.45858706 | 1.06E−13 | 1.22E−09 |
| S100A8 | 7.38139742 | 6.069002213 | 0.45829431 | 1.55E−18 | 1.83E−14 |
| SYNPO | 7.00859865 | 6.064164626 | 0.45801943 | 1.72E−19 | 2.04E−15 |
| DNAJB1 | 9.08865565 | 8.416213497 | 0.45790284 | 2.53E−20 | 3.00E−16 |
| TRIM22 | 9.18822894 | 8.143802038 | 0.45666053 | 4.66E−19 | 5.52E−15 |
| RNASE6 | 8.53278761 | 7.518828268 | 0.45658845 | 6.67E−19 | 7.90E−15 |
| ANXA2 | 11.9338461 | 11.09158075 | 0.45600692 | 5.04E−18 | 5.95E−14 |
| FGF1 | 6.34872272 | 5.54256371 | 0.45597901 | 7.79E−20 | 9.24E−16 |
| C10orf10 | 7.25837099 | 6.436457525 | 0.45571294 | 5.10E−20 | 6.06E−16 |
| GLRX | 8.11264134 | 7.488125719 | 0.4549694 | 2.23E−21 | 2.66E−17 |
| ATP1B2 | 9.75915777 | 8.264661211 | 0.45475431 | 4.27E−17 | 5.01E−13 |
| S100A10 | 11.6502071 | 10.6302368 | 0.45465663 | 9.41E−20 | 1.12E−15 |
| PLK3 | 5.88519317 | 5.470015992 | 0.4538368 | 9.76E−19 | 1.15E−14 |
| SLC1A3 | 11.7637333 | 10.41099425 | 0.45373991 | 2.19E−11 | 2.46E−07 |
| NPC2 | 11.1075962 | 10.40115537 | 0.45368286 | 7.65E−19 | 9.05E−15 |
| FCGR1A | 7.1165608 | 6.201127756 | 0.45359969 | 3.36E−18 | 3.96E−14 |
| CNTNAP1 | 7.2575697 | 6.546525223 | 0.45359422 | 7.48E−18 | 8.82E−14 |
| ADM | 9.35875533 | 8.020859351 | 0.45357741 | 2.19E−17 | 2.57E−13 |
| TNFRSF12A | 7.77434544 | 6.999538148 | 0.45291778 | 6.32E−18 | 7.46E−14 |
| PLEKHQ1 | 7.03945683 | 6.495325168 | 0.45290562 | 5.75E−17 | 6.75E−13 |
| C1QB | 10.7125617 | 9.486083795 | 0.45231586 | 3.57E−20 | 4.24E−16 |
| LAMA2 | 6.38424576 | 5.481124186 | 0.45151823 | 5.69E−21 | 6.77E−17 |
| CLCF1 | 5.33851944 | 4.944834283 | 0.44972257 | 2.16E−20 | 2.57E−16 |
| GPR65 | 6.23797691 | 5.405489763 | 0.44970707 | 1.28E−17 | 1.51E−13 |
| HRASLS3 | 8.74807837 | 8.043196722 | 0.4496784 | 5.71E−19 | 6.76E−15 |
| CD33 | 5.41460294 | 4.919673415 | 0.4479033 | 9.93E−19 | 1.17E−14 |
| CYBRD1 | 7.28282759 | 6.415108581 | 0.44788623 | 7.14E−18 | 8.42E−14 |
| ADAM12 | 5.41626461 | 4.959450794 | 0.44732217 | 9.88E−20 | 1.17E−15 |
| TGFB1I1 | 8.1437137 | 7.329951187 | 0.44653551 | 1.57E−18 | 1.86E−14 |
| IFITM2 | 9.67333645 | 8.882558491 | 0.44531614 | 3.54E−19 | 4.19E−15 |
| MICALL2 | 6.87317493 | 6.241996387 | 0.44519323 | 1.14E−18 | 1.35E−14 |
| CYFIP1 | 8.6702992 | 8.277910859 | 0.44277856 | 7.27E−18 | 8.57E−14 |
| A2M | 11.3550309 | 10.34201711 | 0.44254907 | 2.27E−22 | 2.71E−18 |
| JUNB | 8.08838516 | 7.391626599 | 0.44240811 | 8.66E−19 | 1.03E−14 |
| MT1E | 9.80660032 | 8.926628859 | 0.44225802 | 5.47E−16 | 6.39E−12 |
| FLJ11286 | 8.25552095 | 7.535641487 | 0.4419871 | 1.10E−15 | 1.28E−11 |
| OXTR | 6.85021781 | 5.616906859 | 0.44192059 | 9.98E−18 | 1.18E−13 |
| FJX1 | 9.07300163 | 8.039149861 | 0.44138011 | 2.43E−16 | 2.85E−12 |
| CHL1 | 9.02901478 | 7.543611083 | 0.44045877 | 2.51E−14 | 2.90E−10 |
| TFE3 | 7.27013936 | 6.843910077 | 0.43931907 | 1.30E−16 | 1.53E−12 |
| CD93 | 7.70122726 | 6.766878962 | 0.43909387 | 7.88E−18 | 9.29E−14 |
| CAPG | 8.05260297 | 7.221996298 | 0.43891845 | 1.99E−16 | 2.34E−12 |
| ZFP36L2 | 5.9841764 | 5.374410917 | 0.43820478 | 8.71E−17 | 1.02E−12 |
| GFPT2 | 7.95677431 | 6.846773007 | 0.4381413 | 3.50E−18 | 4.13E−14 |
| CH25H | 6.7353148 | 5.516988948 | 0.43778408 | 2.45E−19 | 2.91E−15 |
| RARRES3 | 8.23384711 | 7.243795476 | 0.43734634 | 4.55E−16 | 5.31E−12 |
| FCGR2B | 6.87601527 | 5.712759328 | 0.43685383 | 4.02E−18 | 4.75E−14 |
| GBP1 | 8.96000738 | 7.842322019 | 0.43637693 | 5.20E−17 | 6.11E−13 |
| FAS | 6.54061804 | 5.708564938 | 0.43575865 | 6.40E−20 | 7.60E−16 |
| PAM | 10.6623467 | 10.03294031 | 0.43537302 | 1.37E−18 | 1.63E−14 |
| CASP1 | 7.35953388 | 6.520352191 | 0.43528731 | 8.39E−18 | 9.90E−14 |
| BHLHB2 | 7.49883289 | 6.690208346 | 0.43499957 | 4.41E−18 | 5.21E−14 |
| HP | 5.91311938 | 4.769516251 | 0.43391711 | 7.62E−17 | 8.94E−13 |
| RHBDF2 | 6.53999386 | 6.058798772 | 0.43352544 | 2.61E−17 | 3.07E−13 |
| JAM2 | 9.55621441 | 8.613131747 | 0.43296015 | 5.12E−13 | 5.86E−09 |
| IGFBP7 | 12.4338942 | 11.68389488 | 0.432533 | 2.00E−24 | 2.39E−20 |
| PTPN9 | 6.85578892 | 6.32915847 | 0.43201614 | 1.97E−16 | 2.31E−12 |
| S100A11 | 9.37071308 | 8.514201885 | 0.43176677 | 7.65E−18 | 9.03E−14 |
| LIMS1 | 8.49263724 | 7.992892828 | 0.43174056 | 1.36E−18 | 1.61E−14 |
| TREM2 | 8.31195351 | 7.300374736 | 0.43156576 | 4.21E−16 | 4.92E−12 |
| FAH | 6.09942033 | 5.500728623 | 0.43155983 | 6.40E−20 | 7.60E−16 |
| RYR3 | 6.05342122 | 4.842417994 | 0.43153885 | 4.34E−17 | 5.10E−13 |
| LAP3 | 10.1001634 | 9.514618334 | 0.43146167 | 1.81E−18 | 2.14E−14 |
| CAMK2N1 | 10.713114 | 9.67670117 | 0.43104827 | 1.90E−16 | 2.22E−12 |
| UGCG | 9.10000274 | 8.479880059 | 0.4308697 | 5.66E−17 | 6.64E−13 |
| RFTN1 | 9.18561064 | 8.298329662 | 0.43067597 | 6.51E−18 | 7.68E−14 |
| SWAP70 | 7.3301357 | 6.673265945 | 0.4306314 | 1.02E−16 | 1.20E−12 |
| HCK | 6.75282362 | 5.854149293 | 0.43060074 | 1.17E−17 | 1.37E−13 |
| CROT | 7.7007939 | 7.075384316 | 0.43015021 | 1.40E−16 | 1.64E−12 |
| GBE1 | 9.57730086 | 8.926174378 | 0.43012286 | 2.01E−16 | 2.35E−12 |
| MSN | 9.99306136 | 9.278068023 | 0.42986451 | 1.02E−15 | 1.19E−11 |
| GALNT2 | 7.06109165 | 6.527653633 | 0.42984373 | 2.93E−18 | 3.47E−14 |
| LAMB2 | 7.57430241 | 6.876674917 | 0.42967404 | 6.73E−16 | 7.85E−12 |
| LILRB1 | 5.82045283 | 5.098340079 | 0.42916691 | 3.90E−17 | 4.58E−13 |
| RNASE3 | 4.947474 | 4.576081302 | 0.42881766 | 2.09E−17 | 2.46E−13 |
| BCL3 | 5.33650348 | 4.995522381 | 0.42849222 | 1.11E−18 | 1.31E−14 |
| FABP7 | 10.9807899 | 9.158891479 | 0.42840516 | 3.00E−11 | 3.37E−07 |
| PLEKHA4 | 5.52808151 | 5.077500617 | 0.42787236 | 3.09E−17 | 3.63E−13 |
| PDLIM4 | 5.94988137 | 5.267313978 | 0.42785297 | 3.22E−16 | 3.77E−12 |
| SPATA20 | 8.04568753 | 7.303191845 | 0.42772227 | 2.87E−15 | 3.34E−11 |
| FYB | 6.23524943 | 5.585263385 | 0.42749934 | 1.75E−17 | 2.06E−13 |
| IL6ST | 7.00507418 | 6.421878296 | 0.42686853 | 7.41E−15 | 8.60E−11 |
| SERPINA1 | 8.28431181 | 7.094868198 | 0.42644598 | 1.61E−18 | 1.91E−14 |
| FHL3 | 5.85971157 | 5.476520201 | 0.42524566 | 3.22E−16 | 3.77E−12 |
| SLC7A7 | 7.39384954 | 6.698352713 | 0.42499971 | 3.86E−17 | 4.53E−13 |
| RIN3 | 5.1495143 | 4.824574274 | 0.42429068 | 2.94E−17 | 3.45E−13 |
| APOC2 | 8.95627944 | 7.821608547 | 0.4235504 | 6.47E−15 | 7.51E−11 |
| DYNLT3 | 9.84573968 | 9.130887539 | 0.42317811 | 1.77E−17 | 2.08E−13 |
| REEP5 | 9.9488547 | 9.538117743 | 0.42307608 | 9.91E−17 | 1.16E−12 |
| SAMSN1 | 8.02149442 | 7.069139034 | 0.42237486 | 2.85E−16 | 3.34E−12 |
| CLIC1 | 10.5983035 | 9.969088265 | 0.42218256 | 9.18E−15 | 1.06E−10 |
| SDCBP | 11.6879523 | 11.26436226 | 0.42199111 | 3.61E−16 | 4.22E−12 |
| NFKBIA | 10.313756 | 9.724551931 | 0.42165881 | 1.97E−17 | 2.32E−13 |
| GIMAP4 | 7.84923784 | 7.018313483 | 0.42144537 | 1.58E−16 | 1.85E−12 |
| CORO2B | 8.61496086 | 7.488859542 | 0.4199121 | 1.34E−12 | 1.53E−08 |
| FAM26B | 5.93135191 | 5.40541402 | 0.41982496 | 9.92E−18 | 1.17E−13 |
| CD86 | 6.15704561 | 5.493270937 | 0.41971807 | 6.02E−17 | 7.06E−13 |
| PLSCR1 | 8.59556867 | 7.868723942 | 0.41963787 | 6.99E−16 | 8.16E−12 |
| STAT3 | 9.57863235 | 9.037087367 | 0.41962689 | 3.94E−16 | 4.61E−12 |
| SH2B3 | 7.35238573 | 6.68938098 | 0.41916892 | 4.68E−18 | 5.52E−14 |
| GPR37 | 8.88551164 | 7.373769977 | 0.41898954 | 5.27E−12 | 5.97E−08 |
| GYS1 | 7.24734573 | 6.60852406 | 0.41802205 | 2.43E−15 | 2.83E−11 |
| ODZ4 | 8.04191467 | 7.123485815 | 0.4177829 | 1.86E−14 | 2.15E−10 |
| SLCO2B1 | 6.42233972 | 5.864381706 | 0.4177484 | 3.48E−16 | 4.07E−12 |
| CHPF | 6.93254505 | 6.470408599 | 0.41702356 | 1.30E−15 | 1.52E−11 |
| MAP1B | 10.6530945 | 9.626924048 | 0.41687103 | 3.03E−13 | 3.48E−09 |
| MS4A6A | 8.88152308 | 7.897120654 | 0.41645437 | 5.84E−16 | 6.82E−12 |
| MAF | 6.57997938 | 6.086748798 | 0.41586112 | 2.39E−14 | 2.77E−10 |
| FEZ2 | 7.96336696 | 7.649405493 | 0.41543772 | 9.58E−18 | 1.13E−13 |
| MVP | 7.10042461 | 6.412563976 | 0.41503617 | 1.15E−17 | 1.36E−13 |
| LZTS1 | 4.74256215 | 4.56236509 | 0.41499889 | 1.34E−15 | 1.57E−11 |
| PLOD1 | 8.38278994 | 7.773452378 | 0.41482299 | 4.55E−16 | 5.31E−12 |
| SH3TC1 | 5.49158105 | 4.956641228 | 0.41469264 | 3.33E−16 | 3.89E−12 |
| TLR1 | 6.11051661 | 5.469936487 | 0.41446344 | 6.34E−16 | 7.40E−12 |
| PLP2 | 9.24567887 | 8.313110067 | 0.41420059 | 9.77E−16 | 1.14E−11 |
| TGFB1 | 6.14660065 | 5.585191675 | 0.41409075 | 2.93E−16 | 3.43E−12 |
| TMEPAI | 6.96251116 | 6.29506164 | 0.41387915 | 6.13E−16 | 7.16E−12 |
| MS4A4A | 8.55208353 | 7.364441852 | 0.41369217 | 9.74E−17 | 1.14E−12 |
| ELOVL2 | 6.92925622 | 5.780613841 | 0.4136572 | 4.67E−17 | 5.49E−13 |
| TYROBP | 9.94422179 | 8.928320746 | 0.41337065 | 2.14E−16 | 2.51E−12 |
| CSGlcA-T | 7.02872525 | 6.520653558 | 0.41302999 | 1.55E−16 | 1.82E−12 |
| MTMR11 | 6.53044627 | 5.914535296 | 0.41289699 | 1.50E−15 | 1.75E−11 |
| CD53 | 9.49086965 | 8.536832334 | 0.41277027 | 6.19E−17 | 7.27E−13 |
| SPRY2 | 9.37989042 | 8.409908433 | 0.41265204 | 1.88E−13 | 2.16E−09 |
| AEBP1 | 9.23912202 | 8.13014334 | 0.41258738 | 1.71E−14 | 1.98E−10 |
| DENND3 | 5.56201566 | 5.130810189 | 0.41224446 | 2.74E−17 | 3.23E−13 |
| IL13RA1 | 7.24172693 | 6.588712274 | 0.41128125 | 1.80E−16 | 2.10E−12 |
| GSTK1 | 9.0463021 | 8.560193875 | 0.41111307 | 5.27E−16 | 6.15E−12 |
| tcag7.1314 | 7.57671736 | 6.790827382 | 0.41051211 | 1.27E−15 | 1.48E−11 |
| MOXD1 | 8.13045264 | 6.724120096 | 0.41008019 | 6.34E−16 | 7.40E−12 |
| GNG5 | 10.6585647 | 10.267414 | 0.4100099 | 2.81E−16 | 3.28E−12 |
| PROS1 | 8.55022304 | 7.63621725 | 0.409839 | 1.70E−15 | 1.98E−11 |
| SYNC1 | 6.64969392 | 5.723758074 | 0.40953423 | 4.14E−16 | 4.84E−12 |
| ARHGDIB | 9.76857732 | 9.066271209 | 0.40934529 | 6.55E−18 | 7.72E−14 |
| CHSY1 | 8.73497655 | 8.152010356 | 0.40906546 | 7.96E−16 | 9.29E−12 |
| PLAUR | 6.64827481 | 5.918471593 | 0.40882927 | 3.90E−18 | 4.61E−14 |
| FNDC4 | 7.38622989 | 6.7875727 | 0.40866159 | 1.78E−15 | 2.07E−11 |
| TGFB3 | 6.48242981 | 5.931316083 | 0.40840095 | 8.87E−16 | 1.03E−11 |
| ANGPT2 | 7.07656112 | 6.097546868 | 0.408376 | 3.66E−14 | 4.22E−10 |
| ITGAV | 10.5343057 | 9.971342202 | 0.40815004 | 8.53E−15 | 9.90E−11 |
| AK3L1 | 8.80651178 | 8.036579843 | 0.40772872 | 2.27E−14 | 2.63E−10 |
| AP1B1 | 6.90502241 | 6.556384722 | 0.40772313 | 9.72E−16 | 1.13E−11 |
| SMAD1 | 9.00758778 | 8.29807218 | 0.4077066 | 3.26E−15 | 3.79E−11 |
| DTNA | 7.50644686 | 6.619051069 | 0.40744396 | 3.57E−12 | 4.05E−08 |
| DMN | 6.47470757 | 6.05826239 | 0.40714119 | 9.38E−15 | 1.09E−10 |
| LY86 | 8.36889371 | 7.399360623 | 0.40691007 | 8.40E−15 | 9.75E−11 |
| PSRC1 | 9.4285263 | 8.50715888 | 0.40641961 | 1.52E−13 | 1.75E−09 |
| WAS | 6.2313978 | 5.989000482 | 0.40605112 | 2.12E−17 | 2.50E−13 |
| ARHGAP29 | 8.09210894 | 7.326379479 | 0.40515561 | 5.47E−18 | 6.46E−14 |
| NRP1 | 5.77588765 | 5.299750821 | 0.40463667 | 6.65E−16 | 7.77E−12 |
| NFIC | 7.36582409 | 6.701450809 | 0.40455038 | 3.78E−15 | 4.39E−11 |
| CIQTNF1 | 6.29338811 | 5.624481354 | 0.40446801 | 9.64E−18 | 1.14E−13 |
| LILRA2 | 5.3403609 | 5.043361732 | 0.40446581 | 6.40E−17 | 7.51E−13 |
| ANKRD25 | 7.30852375 | 6.737534198 | 0.40345182 | 8.58E−16 | 1.00E−11 |
| CSF1 | 5.03232708 | 4.862559089 | 0.403403 | 9.70E−19 | 1.15E−14 |
| PFKFB3 | 9.46508453 | 8.742576495 | 0.40299089 | 1.02E−14 | 1.18E−10 |
| C1orf38 | 6.23239976 | 5.525158357 | 0.40283708 | 5.71E−16 | 6.68E−12 |
| PHLDA3 | 6.27362075 | 5.831640834 | 0.4028366 | 3.44E−16 | 4.02E−12 |
| AGTRL1 | 8.16765926 | 6.933180636 | 0.40281777 | 4.72E−15 | 5.48E−11 |
| TNFAIP6 | 8.00538726 | 6.925403729 | 0.402647 | 7.15E−14 | 8.24E−10 |
| ANG | 6.28309054 | 5.616424426 | 0.40260339 | 6.94E−18 | 8.18E−14 |
| MAN2B1 | 7.51193921 | 7.011256859 | 0.40255846 | 1.26E−15 | 1.47E−11 |
| SQSTM1 | 9.27934351 | 8.762424378 | 0.40244845 | 9.72E−16 | 1.13E−11 |
| SLC16A4 | 6.70928663 | 5.911689403 | 0.40237784 | 1.13E−15 | 1.32E−11 |
| CCR5 | 6.19429877 | 5.686651343 | 0.40224654 | 1.08E−17 | 1.27E−13 |
| DOCK2 | 6.03824985 | 5.349095042 | 0.40109283 | 9.53E−17 | 1.12E−12 |
| KCNJ8 | 6.50504158 | 5.77904125 | 0.40037519 | 8.71E−15 | 1.01E−10 |
| RENBP | 5.30214144 | 4.867041909 | 0.40004075 | 1.21E−14 | 1.40E−10 |
| PCDH9 | 8.66205642 | 7.525495114 | 0.39938765 | 4.62E−12 | 5.24E−08 |
| CLEC7A | 6.81174125 | 6.117232789 | 0.39932496 | 4.92E−15 | 5.72E−11 |
| EBI2 | 6.24813261 | 5.37081017 | 0.39929076 | 2.64E−16 | 3.09E−12 |
| JUN | 8.4144482 | 7.831061915 | 0.39834636 | 7.11E−15 | 8.25E−11 |
| RAB20 | 7.57447235 | 6.931256871 | 0.3980976 | 5.05E−15 | 5.87E−11 |
| RNASE4 | 6.79089178 | 6.006636449 | 0.39790911 | 1.29E−16 | 1.52E−12 |
| CYBB | 7.03581849 | 6.35556105 | 0.39785215 | 3.77E−16 | 4.41E−12 |
| PLOD2 | 9.03706627 | 8.308621132 | 0.39782432 | 4.55E−16 | 5.31E−12 |
| DFNA5 | 9.71701359 | 8.993094689 | 0.3969682 | 4.78E−11 | 5.35E−07 |
| MYBPC1 | 7.52841861 | 6.222388704 | 0.39674979 | 1.15E−13 | 1.33E−09 |
| NPL | 7.07689178 | 6.281392524 | 0.39661914 | 1.26E−14 | 1.46E−10 |
| IL10RA | 6.75245488 | 5.919202591 | 0.39648659 | 1.55E−14 | 1.80E−10 |
| NRN1 | 8.96177235 | 7.939407229 | 0.39616337 | 1.74E−11 | 1.96E−07 |
| CD300A | 5.72890475 | 5.305494719 | 0.39614315 | 2.54E−16 | 2.98E−12 |
| P2RY5 | 7.65280577 | 7.029126051 | 0.39606059 | 9.23E−15 | 1.07E−10 |
| LY96 | 7.87259222 | 6.887829707 | 0.39601019 | 3.45E−15 | 4.02E−11 |
| RGS19 | 7.3648146 | 6.997800039 | 0.39585307 | 2.17E−14 | 2.51E−10 |
| GIMAP6 | 7.01899275 | 6.327485052 | 0.39545332 | 6.23E−15 | 7.24E−11 |
| CAMK1 | 6.32434841 | 5.726877946 | 0.3953938 | 3.09E−13 | 3.55E−09 |
| ACP2 | 7.84348185 | 7.358283046 | 0.39499017 | 8.06E−17 | 9.45E−13 |
| ARHGEF6 | 9.98456798 | 9.127328263 | 0.39460067 | 4.80E−08 | 0.00050627 |
| TXNDC15 | 8.53760384 | 8.096748512 | 0.39417411 | 4.05E−16 | 4.74E−12 |
| GATM | 10.0937373 | 8.927634111 | 0.39395685 | 7.71E−12 | 8.73E−08 |
| SSBP2 | 8.28120041 | 7.662958581 | 0.39347129 | 1.06E−13 | 1.22E−09 |
| ITPR2 | 6.25561574 | 5.720407346 | 0.39275571 | 2.24E−14 | 2.59E−10 |
| CAST | 7.34338108 | 6.877406046 | 0.39275441 | 8.94E−18 | 1.05E−13 |
| TLR7 | 5.78887199 | 5.037838406 | 0.39183745 | 1.93E−15 | 2.25E−11 |
| HLA-E | 11.0251542 | 10.49405401 | 0.39151818 | 2.00E−14 | 2.31E−10 |
| MKL2 | 9.09461185 | 8.545373819 | 0.3906745 | 2.65E−17 | 3.12E−13 |
| MAP3K6 | 5.28803735 | 4.865071413 | 0.39052815 | 3.90E−17 | 4.58E−13 |
| CD74 | 11.1658724 | 10.23900879 | 0.39021461 | 4.43E−15 | 5.15E−11 |
| KIAA0247 | 8.35373459 | 7.773845261 | 0.38996898 | 7.26E−15 | 8.42E−11 |
| METTL7A | 9.37944791 | 8.590188604 | 0.38995055 | 2.10E−13 | 2.42E−09 |
| STOM | 7.98493413 | 7.440086422 | 0.38929715 | 6.13E−16 | 7.16E−12 |
| GNAI2 | 9.51301795 | 8.944187637 | 0.38880606 | 3.86E−13 | 4.42E−09 |
| TNFRSF11B | 5.76373245 | 5.047773459 | 0.38872661 | 5.16E−18 | 6.09E−14 |
| FCGR3B | 6.05911479 | 5.471927364 | 0.3885932 | 5.88E−15 | 6.83E−11 |
| CD302 | 8.51428991 | 7.751437553 | 0.38843666 | 2.73E−14 | 3.16E−10 |
| HLA-A | 12.4356471 | 12.03257336 | 0.38838658 | 1.52E−13 | 1.75E−09 |
| TWSG1 | 6.32813408 | 5.693972705 | 0.38836611 | 3.87E−14 | 4.47E−10 |
| CD37 | 5.92753407 | 5.208000061 | 0.38828547 | 6.81E−17 | 7.99E−13 |
| SMPD1 | 5.54116753 | 5.264192232 | 0.38795787 | 3.16E−14 | 3.64E−10 |
| CHST7 | 7.08819215 | 6.304250318 | 0.38788013 | 7.75E−14 | 8.93E−10 |
| GALC | 6.81342868 | 6.368858707 | 0.38756762 | 1.13E−13 | 1.30E−09 |
| ST5 | 7.87533565 | 7.268402443 | 0.38732195 | 1.51E−12 | 1.72E−08 |
| BCKDK | 7.10289848 | 6.697685486 | 0.38561751 | 1.55E−13 | 1.79E−09 |
| SLC2A3 | 7.18465841 | 6.375745483 | 0.38557755 | 2.80E−14 | 3.24E−10 |
| SP100 | 5.98268861 | 5.590164971 | 0.3854431 | 7.00E−15 | 8.12E−11 |
| PTGS1 | 6.37343608 | 5.651610078 | 0.38519637 | 1.32E−20 | 1.56E−16 |
| ARNTL | 7.42504945 | 6.765667847 | 0.38502863 | 8.48E−14 | 9.77E−10 |
| IRS2 | 10.1346787 | 9.411358586 | 0.38429508 | 1.03E−12 | 1.18E−08 |
| DDR2 | 6.77116108 | 5.986810351 | 0.38422814 | 1.75E−14 | 2.02E−10 |
| SNFT | 6.75354278 | 6.044943246 | 0.38416923 | 1.77E−14 | 2.05E−10 |
| GALNT10 | 6.93166213 | 6.252511838 | 0.38407924 | 9.10E−14 | 1.05E−09 |
| MFSD1 | 9.99310566 | 9.558414166 | 0.38376294 | 3.78E−15 | 4.39E−11 |
| CENTD1 | 8.32660905 | 7.46424341 | 0.38347618 | 6.93E−12 | 7.85E−08 |
| SLC22A4 | 6.13468821 | 5.522577882 | 0.38329247 | 2.11E−15 | 2.45E−11 |
| TCIRG1 | 6.6023916 | 6.032038547 | 0.38299787 | 5.14E−17 | 6.04E−13 |
| GNA12 | 7.47022745 | 6.888500219 | 0.38270334 | 5.57E−12 | 6.31E−08 |
| AMPD3 | 5.79368642 | 5.496641141 | 0.38261415 | 4.22E−14 | 4.87E−10 |
| SRGN | 10.4077127 | 9.554467306 | 0.38253625 | 8.49E−15 | 9.85E−11 |
| PODXL | 8.44427415 | 7.714093273 | 0.38234648 | 1.13E−12 | 1.29E−08 |
| MREG | 8.22276466 | 7.434655925 | 0.38222111 | 2.19E−14 | 2.54E−10 |
| SDC2 | 7.78705298 | 7.004292603 | 0.38201344 | 6.89E−15 | 8.00E−11 |
| NUCB1 | 7.56019365 | 7.010231986 | 0.38178081 | 2.19E−13 | 2.51E−09 |
| CREBL2 | 7.8891287 | 7.362813577 | 0.38161276 | 2.76E−14 | 3.19E−10 |
| NDRG1 | 9.0518357 | 8.202615974 | 0.38128687 | 6.63E−14 | 7.64E−10 |
| ERBB2IP | 10.0142983 | 9.488737368 | 0.38108766 | 1.12E−11 | 1.26E−07 |
| CALR | 8.18462625 | 7.892739342 | 0.38064118 | 4.01E−14 | 4.63E−10 |
| IGFBP2 | 9.58857184 | 8.474780578 | 0.38052856 | 2.03E−11 | 2.28E−07 |
| C13orf18 | 6.85230036 | 6.03249409 | 0.38050928 | 4.64E−13 | 5.32E−09 |
| SCG5 | 10.3431604 | 9.244451447 | 0.38016323 | 3.26E−10 | 3.60E−06 |
| TMEM140 | 6.76948532 | 6.049469481 | 0.38013173 | 1.01E−14 | 1.18E−10 |
| PLS3 | 10.2426551 | 9.508679947 | 0.38011982 | 9.70E−13 | 1.11E−08 |
| ARL4C | 7.79332583 | 7.143525225 | 0.38001993 | 1.29E−12 | 1.48E−08 |
| ACTB | 12.6373061 | 12.27150785 | 0.37922155 | 1.35E−12 | 1.54E−08 |
| CTSL1 | 8.96236782 | 8.412949823 | 0.37890885 | 6.26E−17 | 7.35E−13 |
| LTBP3 | 7.99011376 | 7.3465577 | 0.37815105 | 5.92E−13 | 6.77E−09 |
| PDGFA | 7.88663195 | 7.008546329 | 0.37779135 | 5.56E−13 | 6.36E−09 |
| PLXND1 | 6.90694828 | 6.387966414 | 0.37753465 | 4.03E−13 | 4.62E−09 |
| MNDA | 6.84885764 | 5.974204547 | 0.37748923 | 6.36E−14 | 7.34E−10 |
| HSPB1 | 11.0476053 | 10.47562273 | 0.37741604 | 1.26E−13 | 1.45E−09 |
| GPC1 | 7.08741589 | 6.463745477 | 0.37721593 | 1.68E−12 | 1.91E−08 |
| DUSP3 | 6.40492433 | 6.058586432 | 0.3769742 | 9.24E−13 | 1.06E−08 |
| DOK5 | 9.90092345 | 8.877514557 | 0.37651007 | 5.21E−08 | 0.00054894 |
| PPAP2B | 9.98412404 | 9.212397226 | 0.37599841 | 5.25E−11 | 5.87E−07 |
| GJA1 | 11.8947293 | 10.97517599 | 0.37571293 | 2.98E−11 | 3.34E−07 |
| TAPBP | 7.04763397 | 6.718404562 | 0.37542813 | 5.79E−15 | 6.73E−11 |
| REXO2 | 9.97547759 | 9.551539168 | 0.37542258 | 5.32E−15 | 6.19E−11 |
| IDS | 6.47720901 | 6.148944774 | 0.37403631 | 1.60E−14 | 1.85E−10 |
| SOCS3 | 4.54571521 | 4.401934391 | 0.37395564 | 1.70E−13 | 1.95E−09 |
| LYPLA3 | 6.04974309 | 5.74171011 | 0.37386514 | 1.10E−12 | 1.25E−08 |
| GRB10 | 7.47994132 | 6.776549716 | 0.37375914 | 1.26E−13 | 1.45E−09 |
| OLFML3 | 9.09713589 | 8.274995611 | 0.37373146 | 7.88E−13 | 9.01E−09 |
| PTN | 11.6985235 | 10.53833785 | 0.37363714 | 1.91E−07 | 0.00196823 |
| BIN2 | 5.75684696 | 5.295321474 | 0.37361149 | 1.11E−16 | 1.31E−12 |
| FSTL1 | 10.0504331 | 9.320506501 | 0.37339686 | 1.49E−13 | 1.72E−09 |
| HEBP1 | 9.32905841 | 8.730032163 | 0.37319599 | 1.36E−12 | 1.55E−08 |
| PTPRC | 7.16911415 | 6.404855105 | 0.37315455 | 9.47E−15 | 1.10E−10 |
| LGI1 | 6.68724678 | 5.516008954 | 0.37301561 | 3.33E−13 | 3.82E−09 |
| PCSK1 | 6.7316198 | 5.691666525 | 0.37296267 | 2.54E−14 | 2.94E−10 |
| EPAS1 | 7.72537926 | 7.277805834 | 0.37284853 | 1.36E−15 | 1.59E−11 |
| LILRB4 | 5.3246876 | 4.797028735 | 0.3727822 | 2.97E−14 | 3.43E−10 |
| SMOX | 6.13235201 | 5.796518592 | 0.37268075 | 8.19E−13 | 9.36E−09 |
| VWA1 | 6.26164567 | 5.744716172 | 0.37263639 | 1.24E−12 | 1.41E−08 |
| PGDS | 6.50322231 | 5.655605457 | 0.37241465 | 5.84E−13 | 6.68E−09 |
| PJA2 | 10.1962049 | 9.637841413 | 0.3720823 | 2.71E−12 | 3.07E−08 |
| GGTLA1 | 5.60464977 | 5.086373673 | 0.37195079 | 1.99E−14 | 2.30E−10 |
| PLEKHF1 | 5.78966281 | 5.345698053 | 0.37191441 | 1.23E−16 | 1.44E−12 |
| GPX1 | 10.8534699 | 10.45285098 | 0.37133631 | 1.74E−14 | 2.01E−10 |
| EMR2 | 5.18788912 | 4.646139354 | 0.37126011 | 1.85E−14 | 2.14E−10 |
| GPSM3 | 5.74357131 | 5.423058234 | 0.37106023 | 9.77E−15 | 1.13E−10 |
| NCF4 | 5.25933089 | 4.742554207 | 0.37075957 | 6.96E−15 | 8.08E−11 |
| SPARC | 11.5485208 | 10.82560807 | 0.37070996 | 2.10E−11 | 2.36E−07 |
| PHC2 | 8.84414977 | 8.439213593 | 0.37064556 | 3.69E−12 | 4.19E−08 |
| FLJ13236 | 4.89613008 | 4.437341599 | 0.37058599 | 2.46E−18 | 2.91E−14 |
| SSPN | 8.01236201 | 7.245346794 | 0.37030562 | 1.78E−11 | 2.00E−07 |
| AHNAK2 | 6.2391607 | 5.247285645 | 0.37021608 | 1.19E−17 | 1.40E−13 |
| DLC1 | 5.74853747 | 5.448790324 | 0.37017339 | 1.68E−15 | 1.96E−11 |
| G6PC3 | 8.31781045 | 7.862892437 | 0.37017237 | 1.64E−11 | 1.85E−07 |
| EVI2B | 7.52320192 | 6.702216133 | 0.36979883 | 6.11E−14 | 7.05E−10 |
| HLA-DMA | 9.04334178 | 8.168199301 | 0.36958029 | 6.93E−14 | 7.99E−10 |
| TCTN1 | 7.85231789 | 7.392898482 | 0.36947671 | 1.38E−12 | 1.57E−08 |
| PPP1CB | 9.20684266 | 8.811511839 | 0.36895454 | 2.48E−13 | 2.84E−09 |
| FBLN5 | 7.11147429 | 6.159255482 | 0.36878965 | 8.19E−13 | 9.36E−09 |
| OGFRL1 | 6.59220352 | 6.03264677 | 0.3684915 | 9.57E−15 | 1.11E−10 |
| EMID1 | 6.59493345 | 6.069775684 | 0.36810437 | 1.71E−13 | 1.96E−09 |
| LOXL1 | 6.67479769 | 5.787986392 | 0.36804992 | 3.04E−14 | 3.52E−10 |
| GMFG | 7.89751841 | 7.194171121 | 0.36787048 | 9.15E−14 | 1.05E−09 |
| ANK2 | 8.15546663 | 7.25265 | 0.36744826 | 2.73E−08 | 0.00028948 |
| MR1 | 5.3410301 | 5.017342512 | 0.36703113 | 1.54E−15 | 1.80E−11 |
| TMEM22 | 7.9524863 | 7.179435755 | 0.36699665 | 1.78E−10 | 1.97E−06 |
| SH3BP2 | 5.63171475 | 5.341458116 | 0.36660399 | 1.89E−13 | 2.17E−09 |
| DHRS3 | 8.48525906 | 7.863284609 | 0.36649679 | 3.66E−11 | 4.10E−07 |
| CTBS | 6.80969833 | 6.267475664 | 0.36648915 | 6.85E−13 | 7.83E−09 |
| FKBP15 | 6.30134433 | 6.083096161 | 0.36637834 | 2.46E−13 | 2.83E−09 |
| BCAP29 | 6.00489479 | 5.738379087 | 0.36618742 | 4.72E−14 | 5.44E−10 |
| LRP1 | 7.21253327 | 6.586037657 | 0.36618585 | 1.24E−14 | 1.44E−10 |
| CX3CR1 | 9.24982076 | 7.980338236 | 0.36580151 | 1.89E−10 | 2.10E−06 |
| CD180 | 4.81501117 | 4.468484828 | 0.36560246 | 9.96E−14 | 1.15E−09 |
| PTP4A2 | 10.3887801 | 10.04756855 | 0.36524269 | 8.85E−15 | 1.03E−10 |
| ERMAP | 6.22869828 | 5.859096858 | 0.36486396 | 3.95E−13 | 4.53E−09 |
| LYN | 7.76717108 | 7.130019074 | 0.36452903 | 9.56E−13 | 1.09E−08 |
| ICAM1 | 5.84044739 | 5.219819529 | 0.36450734 | 1.22E−18 | 1.44E−14 |
| AXL | 6.61940323 | 6.138616188 | 0.36445262 | 1.17E−14 | 1.35E−10 |
| C21orf7 | 6.19839776 | 5.453256913 | 0.36400455 | 1.09E−16 | 1.28E−12 |
| PTTG1IP | 11.8571237 | 11.46674286 | 0.36393106 | 8.00E−12 | 9.05E−08 |
| MXRA8 | 7.28890156 | 6.641692859 | 0.3638356 | 1.71E−15 | 1.99E−11 |
| RAB36 | 5.46909765 | 5.025047303 | 0.3632426 | 3.15E−13 | 3.62E−09 |
| HOPX | 11.3739347 | 10.20412075 | 0.36314659 | 8.39E−13 | 9.59E−09 |
| CNN3 | 10.6343825 | 9.919167782 | 0.3629668 | 1.38E−11 | 1.56E−07 |
| MPP1 | 7.28219119 | 6.778285996 | 0.36236001 | 1.48E−12 | 1.69E−08 |
| TGFB2 | 5.05219739 | 4.590175435 | 0.3622814 | 5.24E−16 | 6.12E−12 |
| TGFBI | 10.3417569 | 9.423923235 | 0.36186991 | 2.88E−14 | 3.32E−10 |
| SIPA1 | 5.75909124 | 5.423455589 | 0.36184926 | 8.26E−12 | 9.35E−08 |
| EML3 | 6.01758256 | 5.709418639 | 0.36170419 | 1.08E−11 | 1.22E−07 |
| FAM134B | 5.96612317 | 5.661124129 | 0.36117897 | 1.97E−10 | 2.19E−06 |
| UBE2L6 | 9.75390171 | 9.280027964 | 0.36091113 | 1.10E−12 | 1.25E−08 |
| RGS2 | 10.0036081 | 9.234237133 | 0.36080118 | 1.29E−13 | 1.48E−09 |
| CCDC102B | 5.88772253 | 5.229976581 | 0.36050664 | 8.19E−13 | 9.36E−09 |
| CAP1 | 10.9043699 | 10.65346339 | 0.36035634 | 2.38E−13 | 2.73E−09 |
| MRAS | 6.9959361 | 6.385463273 | 0.36024949 | 9.91E−09 | 0.00010644 |
| EMILIN1 | 5.12576044 | 4.641248533 | 0.3599215 | 1.36E−14 | 1.58E−10 |
| CECR1 | 8.09583618 | 7.267719073 | 0.3599205 | 2.20E−12 | 2.50E−08 |
| SYDE1 | 5.48052704 | 5.274916143 | 0.35988134 | 2.00E−15 | 2.32E−11 |
| STK17A | 7.54255475 | 6.944065508 | 0.3597163 | 3.20E−13 | 3.67E−09 |
| SH3GLB1 | 9.41571193 | 8.987567294 | 0.35964121 | 9.10E−14 | 1.05E−09 |
| SIRPA | 6.45133461 | 6.027779496 | 0.35953604 | 7.40E−13 | 8.46E−09 |
| RRAGC | 9.20991818 | 8.855592263 | 0.3586494 | 6.11E−14 | 7.05E−10 |
| DKK3 | 7.52615679 | 6.856149744 | 0.35845996 | 2.67E−10 | 2.96E−06 |
| TCN2 | 6.28253359 | 5.858128407 | 0.35841122 | 8.19E−12 | 9.27E−08 |
| SCARA3 | 6.86911424 | 6.279922206 | 0.35820162 | 2.28E−12 | 2.59E−08 |
| MYL9 | 7.05527076 | 6.231430548 | 0.35806404 | 2.49E−13 | 2.86E−09 |
| MGAT4A | 6.16897013 | 5.64297371 | 0.35795161 | 9.52E−13 | 1.09E−08 |
| LOH11CR2A | 7.40364397 | 6.853316271 | 0.3579379 | 6.43E−12 | 7.29E−08 |
| RGS1 | 8.79218905 | 7.831513129 | 0.35745732 | 1.57E−11 | 1.77E−07 |
| C8orf4 | 7.37462598 | 6.514313019 | 0.35745468 | 3.70E−14 | 4.27E−10 |
| ST8SIA4 | 4.94960313 | 4.606116733 | 0.35736636 | 2.47E−11 | 2.78E−07 |
| FTL | 10.7514683 | 10.50293696 | 0.35668387 | 8.08E−13 | 9.23E−09 |
| COL4A2 | 9.23056714 | 8.373063406 | 0.35654831 | 4.83E−11 | 5.40E−07 |
| ANXA2P2 | 7.38488356 | 6.852510105 | 0.35634837 | 7.91E−14 | 9.11E−10 |
| PRSS23 | 8.34888626 | 7.570356545 | 0.35634792 | 2.67E−13 | 3.06E−09 |
| ECM2 | 7.97517187 | 7.055578989 | 0.356335 | 4.99E−13 | 5.72E−09 |
| TMEM132A | 7.01171092 | 6.464108897 | 0.35616975 | 5.78E−13 | 6.61E−09 |
| AQP9 | 6.02682178 | 5.322730602 | 0.35580208 | 4.47E−15 | 5.20E−11 |
| COL4A1 | 9.66360482 | 8.738400337 | 0.35553485 | 2.62E−11 | 2.95E−07 |
| HEXB | 10.0744333 | 9.634986593 | 0.35544463 | 2.57E−16 | 3.01E−12 |
| ENG | 5.88413825 | 5.538243608 | 0.35536576 | 3.71E−13 | 4.25E−09 |
| CP | 7.36604557 | 6.307606082 | 0.35524204 | 6.10E−16 | 7.12E−12 |
| FLJ20273 | 7.20519255 | 6.450930641 | 0.35503327 | 9.31E−16 | 1.09E−11 |
| SECTM1 | 5.99746052 | 5.574908406 | 0.35430033 | 3.26E−15 | 3.79E−11 |
| CALD1 | 8.19507499 | 7.672406721 | 0.35418563 | 1.47E−11 | 1.66E−07 |
| PIK3CG | 4.36630038 | 4.186201737 | 0.35405565 | 2.28E−15 | 2.66E−11 |
| HSPA5 | 11.2769603 | 10.88465802 | 0.35389332 | 5.87E−13 | 6.71E−09 |
| NDFIP1 | 10.2882542 | 9.819700918 | 0.35385491 | 1.24E−09 | 1.35E−05 |
| PLEKHC1 | 9.0708215 | 8.480907431 | 0.35382533 | 2.77E−12 | 3.15E−08 |
| SRI | 11.0091253 | 10.43858908 | 0.35353401 | 1.01E−07 | 0.00105698 |
| CYB5R1 | 8.77016045 | 8.278895241 | 0.35345573 | 4.09E−14 | 4.72E−10 |
| FOSL1 | 5.37158739 | 4.800722983 | 0.35337158 | 8.26E−19 | 9.78E−15 |
| NOTCH3 | 5.93887022 | 5.730058137 | 0.35300753 | 6.62E−12 | 7.49E−08 |
| MAFF | 8.10659055 | 7.378505283 | 0.35294362 | 5.17E−12 | 5.86E−08 |
| LAT2 | 5.58291457 | 5.306407856 | 0.35270789 | 1.48E−15 | 1.72E−11 |
| BACE1 | 8.51400311 | 7.97848593 | 0.35243144 | 1.91E−09 | 2.08E−05 |
| IFNGR2 | 8.75841095 | 8.382462661 | 0.35238857 | 4.39E−12 | 4.98E−08 |
| WTAP | 7.61031049 | 7.167335191 | 0.35207497 | 3.00E−13 | 3.44E−09 |
| AGT | 11.2023656 | 9.920918392 | 0.35202906 | 8.80E−07 | 0.00889515 |
| LDHA | 12.5286448 | 12.07612715 | 0.35191809 | 4.45E−11 | 4.99E−07 |
| LGALS9 | 7.09000067 | 6.605973606 | 0.35189233 | 1.90E−12 | 2.16E−08 |
| CIDEB | 5.4466936 | 5.152888635 | 0.35180691 | 7.52E−14 | 8.66E−10 |
| MSR1 | 4.87620885 | 4.561224998 | 0.35171869 | 1.26E−17 | 1.48E−13 |
| LRRC2 | 4.98563188 | 4.398733588 | 0.35164535 | 1.26E−13 | 1.45E−09 |
| APIS2 | 9.47001981 | 8.810958961 | 0.35162915 | 2.24E−09 | 2.44E−05 |
| ALDH9A1 | 10.2322827 | 9.907403272 | 0.35144272 | 2.81E−11 | 3.15E−07 |
| CSF3R | 5.04500616 | 4.777779288 | 0.35140368 | 6.30E−16 | 7.36E−12 |
| COL8A2 | 5.3975467 | 4.887280686 | 0.35115867 | 1.22E−14 | 1.42E−10 |
| MAP4 | 8.40103951 | 7.90621794 | 0.35089281 | 9.38E−09 | 0.00010086 |
| SEP7 | 12.1245144 | 11.78111935 | 0.3508733 | 1.87E−08 | 0.00019944 |
| RNF130 | 9.58399796 | 9.104439115 | 0.35065652 | 3.64E−12 | 4.13E−08 |
| GUSB | 9.69868514 | 9.249831063 | 0.35013928 | 2.58E−12 | 2.93E−08 |
| LILRB2 | 5.02064192 | 4.58693799 | 0.35001875 | 7.10E−16 | 8.29E−12 |
| HOM-TES-103 | 7.34205695 | 6.783212276 | 0.34974392 | 1.13E−09 | 1.24E−05 |
| HHLA3 | 6.81810576 | 6.246825572 | 0.34937436 | 3.53E−10 | 3.90E−06 |
| PEPD | 8.14298056 | 7.730642303 | 0.34936913 | 4.62E−13 | 5.29E−09 |
| RUFY1 | 8.40330598 | 7.999330599 | 0.34935693 | 5.04E−14 | 5.82E−10 |
| SPEG | 6.19497385 | 5.830213385 | 0.34893377 | 1.93E−11 | 2.17E−07 |
| SCIN | 8.03782038 | 7.395508609 | 0.34888525 | 4.25E−13 | 4.87E−09 |
| ITGA3 | 5.83989921 | 5.240586589 | 0.34884589 | 8.15E−18 | 9.62E−14 |
| CD81 | 11.4886843 | 11.08514111 | 0.34872246 | 1.22E−10 | 1.36E−06 |
| IL4R | 6.09287222 | 5.621877699 | 0.34871448 | 7.08E−14 | 8.16E−10 |
| PALLD | 9.75529628 | 9.231247315 | 0.34848236 | 5.07E−11 | 5.67E−07 |
| ADFP | 8.96141147 | 8.102377631 | 0.34821284 | 6.38E−11 | 7.13E−07 |
| FLNA | 7.18317965 | 6.589646955 | 0.34814725 | 2.89E−12 | 3.28E−08 |
| SLC4A4 | 7.42522768 | 6.565037187 | 0.34811912 | 3.72E−11 | 4.17E−07 |
| HLA-B | 11.3738437 | 10.83249773 | 0.3477611 | 3.33E−11 | 3.73E−07 |
| CD84 | 4.83650563 | 4.688604634 | 0.34773395 | 1.30E−12 | 1.48E−08 |
| NAGA | 6.58429302 | 6.206482897 | 0.34729959 | 1.77E−11 | 1.99E−07 |
| PTPRE | 7.65509779 | 7.036728719 | 0.34697378 | 1.51E−11 | 1.70E−07 |
| EGR1 | 9.04630694 | 8.28091156 | 0.3469021 | 3.72E−11 | 4.17E−07 |
| C1orf78 | 6.21643653 | 5.717483202 | 0.34677723 | 8.35E−13 | 9.54E−09 |
| PRDX6 | 10.5163711 | 10.17051179 | 0.34677091 | 2.96E−12 | 3.36E−08 |
| CTNNA1 | 9.9814542 | 9.643810734 | 0.3467297 | 2.11E−12 | 2.41E−08 |
| IL8 | 7.30052268 | 6.14107478 | 0.34659102 | 2.82E−13 | 3.23E−09 |
| TMEM176A | 8.39741111 | 7.487779977 | 0.34651982 | 1.01E−11 | 1.14E−07 |
| CYR61 | 8.19529333 | 7.331156407 | 0.34636002 | 1.70E−12 | 1.93E−08 |
| MGAT1 | 7.25166626 | 6.852981936 | 0.34605401 | 7.52E−11 | 8.39E−07 |
| FOSL2 | 5.14999781 | 4.853519546 | 0.34603326 | 1.55E−13 | 1.78E−09 |
| PADI2 | 8.06323841 | 7.181837491 | 0.3457568 | 1.38E−08 | 0.00014822 |
| DENND2A | 7.34062242 | 6.597495233 | 0.34522822 | 1.06E−09 | 1.16E−05 |
| C21orf25 | 7.3091983 | 6.802870732 | 0.34505236 | 1.03E−11 | 1.16E−07 |
| FER1L3 | 7.5426847 | 6.816572816 | 0.34505136 | 6.78E−15 | 7.87E−11 |
| LPXN | 7.22595365 | 6.675457645 | 0.34423993 | 6.30E−14 | 7.26E−10 |
| RALBP1 | 8.67087511 | 8.217270234 | 0.34402848 | 3.44E−13 | 3.95E−09 |
| PLAU | 6.28366545 | 5.476067105 | 0.34399241 | 7.14E−18 | 8.42E−14 |
| SGSH | 6.68082349 | 6.345998565 | 0.3439815 | 2.60E−12 | 2.96E−08 |
| IFI30 | 9.6785526 | 8.9372204 | 0.34388613 | 2.67E−12 | 3.03E−08 |
| ROM1 | 5.62720451 | 5.299734941 | 0.34371342 | 3.77E−11 | 4.23E−07 |
| CDH2 | 8.23310566 | 7.540201793 | 0.34364326 | 1.04E−08 | 0.00011151 |
| JAM3 | 9.1520685 | 8.441176926 | 0.34350332 | 8.57E−09 | 9.23E−05 |
| UBC | 12.7621681 | 12.48637994 | 0.34346441 | 8.11E−14 | 9.34E−10 |
| FAM46A | 8.72073192 | 8.064184623 | 0.34332133 | 8.85E−12 | 1.00E−07 |
| ALDH1L1 | 7.46552957 | 6.479906985 | 0.34298934 | 1.77E−11 | 1.99E−07 |
| RIN1 | 5.02548958 | 4.792635422 | 0.34266935 | 2.81E−11 | 3.15E−07 |
| CHMP2A | 10.3059769 | 9.983706788 | 0.34195034 | 3.52E−12 | 4.00E−08 |
| IGFBP6 | 6.31800557 | 5.473993563 | 0.34125422 | 1.22E−14 | 1.42E−10 |
| TACC1 | 6.99102705 | 6.631640868 | 0.34119294 | 3.38E−13 | 3.87E−09 |
| MXRA7 | 11.0035552 | 10.48102331 | 0.34095037 | 2.63E−13 | 3.02E−09 |
| PARVA | 7.48935439 | 6.983958837 | 0.34093961 | 3.61E−11 | 4.05E−07 |
| PHF11 | 8.7546513 | 8.187849636 | 0.34089006 | 8.03E−10 | 8.82E−06 |
| VCAM1 | 8.71245832 | 7.609100756 | 0.34074672 | 8.53E−12 | 9.66E−08 |
| RIC8A | 8.96045372 | 8.628549191 | 0.3407172 | 2.39E−11 | 2.68E−07 |
| KLF9 | 6.69475552 | 6.151998717 | 0.34061874 | 3.41E−09 | 3.70E−05 |
| CARS | 9.04877391 | 8.704355154 | 0.34059216 | 7.65E−11 | 8.53E−07 |
| SLC1A2 | 7.29713549 | 6.398178211 | 0.34010203 | 4.96E−11 | 5.54E−07 |
| CAPNS1 | 10.3044511 | 9.93998739 | 0.34007773 | 1.05E−12 | 1.20E−08 |
| EXT2 | 8.15631201 | 7.815310819 | 0.34006925 | 5.66E−11 | 6.33E−07 |
| MSX1 | 7.66332595 | 6.987034784 | 0.33972868 | 2.90E−08 | 0.00030741 |
| RGL1 | 9.26954488 | 8.747348233 | 0.33943476 | 1.34E−10 | 1.49E−06 |
| TAF10 | 10.1089202 | 9.801842163 | 0.33893334 | 5.44E−11 | 6.08E−07 |
| SCAMP4 | 6.64909992 | 6.085396948 | 0.33886895 | 3.12E−10 | 3.45E−06 |
| EFNB2 | 8.24913675 | 7.511465686 | 0.33862793 | 1.77E−11 | 1.99E−07 |
| LTBP1 | 7.69985186 | 6.988386867 | 0.33851722 | 6.22E−11 | 6.94E−07 |
| C2 | 6.43180916 | 5.784557579 | 0.3385047 | 4.51E−13 | 5.16E−09 |
| DSE | 7.68347607 | 6.968158285 | 0.33838582 | 1.12E−11 | 1.26E−07 |
| IL6R | 4.88011753 | 4.673106964 | 0.33805417 | 1.39E−15 | 1.63E−11 |
| CITED1 | 7.47006021 | 6.595485155 | 0.33730452 | 1.74E−10 | 1.93E−06 |
| GEM | 8.75826603 | 8.008256736 | 0.33727344 | 4.98E−11 | 5.57E−07 |
| UGP2 | 10.3049456 | 9.878687915 | 0.33718445 | 5.27E−13 | 6.03E−09 |
| MAST4 | 6.75375235 | 6.241163978 | 0.33714229 | 1.53E−12 | 1.74E−08 |
| FES | 4.52069014 | 4.257783555 | 0.33664011 | 1.45E−14 | 1.68E−10 |
| ARID5A | 5.87152305 | 5.563568802 | 0.33628501 | 2.46E−12 | 2.80E−08 |
| TICAM1 | 5.47328853 | 5.173733094 | 0.33594361 | 1.50E−11 | 1.69E−07 |
| SDC4 | 8.31697976 | 7.492386514 | 0.33583093 | 2.13E−11 | 2.40E−07 |
| SHC1 | 7.34028166 | 6.820905225 | 0.33576536 | 4.67E−14 | 5.39E−10 |
| S100A13 | 9.94938886 | 9.2204559 | 0.33571478 | 1.29E−10 | 1.44E−06 |
| C20orf29 | 6.76752547 | 6.445491993 | 0.33564139 | 6.13E−11 | 6.85E−07 |
| GNA14 | 4.94919784 | 4.593361966 | 0.3355758 | 7.13E−12 | 8.07E−08 |
| LTC4S | 5.50044183 | 5.109017063 | 0.33540358 | 2.01E−11 | 2.26E−07 |
| IL1RAP | 6.28516929 | 5.685532251 | 0.33534656 | 2.62E−12 | 2.97E−08 |
| VEGFA | 8.48914358 | 7.60588683 | 0.33524886 | 2.73E−11 | 3.07E−07 |
| BMP2K | 5.94377841 | 5.501407518 | 0.33522082 | 1.59E−11 | 1.80E−07 |
| IGFBP5 | 7.68491337 | 7.026854194 | 0.3351146 | 4.44E−10 | 4.90E−06 |
| KLHL4 | 6.26472095 | 5.327168052 | 0.33501733 | 3.66E−12 | 4.15E−08 |
| NEDD9 | 7.37350252 | 6.83204471 | 0.33483499 | 3.10E−12 | 3.52E−08 |
| HEXA | 8.7351401 | 8.301817889 | 0.33468483 | 2.17E−12 | 2.46E−08 |
| SLAMF8 | 5.64101463 | 5.104601325 | 0.33442969 | 3.80E−12 | 4.31E−08 |
| CADM1 | 9.31246203 | 8.707990231 | 0.33409026 | 5.84E−08 | 0.00061476 |
| AQP4 | 8.68609014 | 7.573831214 | 0.33356207 | 1.39E−07 | 0.00143734 |
| CAPN2 | 10.8744451 | 10.46794336 | 0.33350375 | 2.34E−14 | 2.71E−10 |
| HECTD3 | 7.29739128 | 6.886974871 | 0.33328687 | 1.85E−10 | 2.05E−06 |
| CXCL2 | 7.11351604 | 6.095261305 | 0.33317966 | 1.94E−15 | 2.26E−11 |
| TM9SF1 | 7.90330222 | 7.49490089 | 0.33309703 | 1.77E−12 | 2.02E−08 |
| RAC1 | 12.2155052 | 11.95898867 | 0.33302686 | 3.65E−10 | 4.03E−06 |
| FN1 | 9.51551426 | 8.946964344 | 0.33280632 | 1.19E−11 | 1.34E−07 |
| FMNL1 | 4.93193175 | 4.65046613 | 0.3323824 | 1.24E−12 | 1.41E−08 |
| DYRK3 | 5.962499 | 5.639710279 | 0.33197615 | 1.07E−11 | 1.21E−07 |
| TRIM5 | 6.13502925 | 5.702257799 | 0.33182659 | 3.67E−11 | 4.12E−07 |
| DOCK4 | 9.24910981 | 8.640728069 | 0.33136761 | 1.02E−08 | 0.00010937 |
| SMPDL3A | 7.15791718 | 6.63164695 | 0.33087491 | 2.47E−11 | 2.78E−07 |
| MMP19 | 5.5239183 | 5.231468202 | 0.33081037 | 5.27E−12 | 5.97E−08 |
| S100A6 | 11.1976077 | 10.68346254 | 0.33067907 | 4.62E−11 | 5.17E−07 |
| RHOC | 9.29410298 | 8.928156745 | 0.33065501 | 4.31E−10 | 4.75E−06 |
| RPS27L | 10.1303372 | 9.770173019 | 0.33045338 | 1.59E−11 | 1.79E−07 |
| TAPBPL | 6.06208696 | 5.603811243 | 0.33042041 | 1.35E−12 | 1.54E−08 |
| POLR2L | 9.36524531 | 9.001009793 | 0.33038092 | 3.67E−10 | 4.05E−06 |
| FOS | 9.1760502 | 8.33030067 | 0.32973782 | 2.61E−11 | 2.93E−07 |
| GNA15 | 5.69517337 | 5.251614754 | 0.32969466 | 3.11E−17 | 3.65E−13 |
| APLP2 | 8.53888156 | 8.11892419 | 0.32968922 | 1.08E−10 | 1.20E−06 |
| S100A9 | 7.51901996 | 6.567306149 | 0.32951916 | 4.55E−15 | 5.28E−11 |
| EEA1 | 5.57576663 | 5.252046099 | 0.32938274 | 1.74E−11 | 1.96E−07 |
| HMGCL | 8.02214275 | 7.684045157 | 0.32936444 | 4.20E−11 | 4.71E−07 |
| LYVE1 | 5.19171234 | 4.574867146 | 0.32926273 | 3.13E−12 | 3.56E−08 |
| RASSF2 | 10.0348743 | 9.129654704 | 0.32891931 | 5.91E−07 | 0.00600189 |
| TLR5 | 5.7003308 | 5.164106119 | 0.32883596 | 4.98E−12 | 5.65E−08 |
| FLVCR2 | 5.50805294 | 5.158465599 | 0.32883179 | 5.07E−11 | 5.67E−07 |
| TMEM176B | 8.70910257 | 7.764698783 | 0.32881765 | 3.21E−10 | 3.55E−06 |
| LEPROT | 9.58787996 | 9.30829381 | 0.32881663 | 1.94E−10 | 2.15E−06 |
| FADS3 | 6.45700916 | 6.068103391 | 0.32839942 | 1.28E−09 | 1.40E−05 |
| FGR | 5.87833371 | 5.375037934 | 0.32823215 | 3.60E−13 | 4.13E−09 |
| ARRB2 | 6.72972458 | 6.343220718 | 0.32818453 | 5.72E−10 | 6.29E−06 |
| HLA-C | 11.7433371 | 11.28703045 | 0.32802755 | 2.56E−10 | 2.84E−06 |
| TANK | 7.42311337 | 7.051654069 | 0.32778741 | 2.79E−11 | 3.14E−07 |
| HIF1A | 11.6929518 | 11.32872817 | 0.32772311 | 1.23E−10 | 1.37E−06 |
| PRNP | 9.3724744 | 8.895790146 | 0.3276596 | 4.36E−09 | 4.72E−05 |
| TNFRSF10C | 4.19869466 | 4.032601439 | 0.32754157 | 3.96E−10 | 4.37E−06 |
| PILRA | 5.3055751 | 4.926547705 | 0.32746187 | 4.03E−13 | 4.62E−09 |
| 2-Mar | 7.53454975 | 7.021774049 | 0.32733217 | 4.20E−09 | 4.55E−05 |
| PFKL | 6.80314363 | 6.462239633 | 0.32714682 | 3.11E−10 | 3.44E−06 |
| MAN2B2 | 7.51847139 | 7.046917104 | 0.32713236 | 2.66E−11 | 2.99E−07 |
| GFAP | 8.70867994 | 8.341365021 | 0.32701313 | 9.74E−07 | 0.00981994 |
| APOBEC3G | 7.14041047 | 6.474440549 | 0.32677471 | 5.09E−11 | 5.69E−07 |
| ABCA8 | 7.80118308 | 6.7606358 | 0.32674599 | 4.50E−10 | 4.96E−06 |
| GAPDH | 13.0860555 | 12.77198963 | 0.32668978 | 2.01E−11 | 2.26E−07 |
| C17orf60 | 4.75270285 | 4.394758435 | 0.32652021 | 2.67E−12 | 3.03E−08 |
| FNDC3B | 8.59328073 | 8.019762666 | 0.32624173 | 1.83E−11 | 2.07E−07 |
| HIG2 | 9.6711927 | 8.831480265 | 0.32604686 | 1.64E−11 | 1.85E−07 |
| GLT25D2 | 8.31585499 | 7.415617079 | 0.3259596 | 2.44E−08 | 0.00025978 |
| CALCOCO2 | 8.91605256 | 8.566742473 | 0.32562483 | 4.33E−12 | 4.91E−08 |
| WIPI1 | 7.19254066 | 6.554643956 | 0.3255511 | 7.26E−13 | 8.30E−09 |
| MCFD2 | 8.33540259 | 8.00806633 | 0.32514187 | 2.36E−13 | 2.71E−09 |
| CTSA | 9.27908781 | 8.801353904 | 0.32463743 | 2.61E−13 | 3.00E−09 |
| TBC1D1 | 5.9626484 | 5.732969824 | 0.32444284 | 1.73E−10 | 1.92E−06 |
| IL17RA | 5.9660212 | 5.621202713 | 0.32377332 | 4.67E−10 | 5.15E−06 |
| WWTR1 | 5.8218902 | 5.302229958 | 0.32364594 | 7.40E−14 | 8.53E−10 |
| MYO1E | 5.99193604 | 5.604270905 | 0.32314848 | 1.20E−14 | 1.39E−10 |
| TK2 | 6.23487855 | 5.947172036 | 0.32308745 | 5.83E−09 | 6.29E−05 |
| BLVRB | 8.0177058 | 7.516017897 | 0.32284538 | 8.04E−12 | 9.10E−08 |
| GNB2 | 8.52914776 | 8.213704156 | 0.32277759 | 1.02E−09 | 1.12E−05 |
| SNX3 | 12.1999835 | 11.87328001 | 0.32266777 | 4.54E−10 | 5.00E−06 |
| LILRB3 | 5.8751749 | 5.623025764 | 0.32262972 | 3.52E−12 | 4.00E−08 |
| ETV5 | 7.28004144 | 6.717595952 | 0.32250179 | 1.09E−09 | 1.20E−05 |
| GMPPA | 6.27015162 | 5.959160179 | 0.32249763 | 2.39E−11 | 2.68E−07 |
| MYD88 | 8.67775352 | 8.262710938 | 0.32234527 | 1.21E−10 | 1.35E−06 |
| SORT1 | 6.27516964 | 5.952631534 | 0.32187456 | 4.24E−11 | 4.75E−07 |
| SQRDL | 8.40175122 | 7.748934367 | 0.32181925 | 1.92E−11 | 2.16E−07 |
| CTDSP1 | 7.32136753 | 6.978681599 | 0.32181764 | 1.86E−10 | 2.06E−06 |
| ASL | 6.99762798 | 6.559774685 | 0.32181049 | 3.93E−11 | 4.40E−07 |
| ARID5B | 9.21321982 | 8.671927274 | 0.32163555 | 1.51E−11 | 1.70E−07 |
| CORO1A | 6.99091167 | 6.376963299 | 0.32153474 | 2.54E−11 | 2.86E−07 |
| GAA | 6.89995717 | 6.479324117 | 0.3208595 | 2.94E−09 | 3.19E−05 |
| CD59 | 9.66037059 | 9.232095793 | 0.32072889 | 5.87E−11 | 6.55E−07 |
| GRN | 8.76903707 | 8.331212913 | 0.32051463 | 5.22E−12 | 5.91E−08 |
| SREBF1 | 6.67085181 | 6.231425616 | 0.32038811 | 1.20E−10 | 1.34E−06 |
| LPP | 6.82524962 | 6.452754717 | 0.32021611 | 1.26E−10 | 1.40E−06 |
| DUSP1 | 7.47870552 | 6.919339316 | 0.32016441 | 2.29E−11 | 2.58E−07 |
| FGL2 | 6.66320572 | 6.096351432 | 0.31984091 | 8.85E−11 | 9.86E−07 |
| TNFAIP3 | 7.00017997 | 6.419159175 | 0.31974856 | 6.59E−13 | 7.54E−09 |
| ACTN4 | 7.51386443 | 7.119388164 | 0.31968111 | 6.91E−11 | 7.72E−07 |
| PSCD4 | 4.96178928 | 4.784035877 | 0.31950282 | 1.53E−10 | 1.70E−06 |
| SERPINH1 | 8.08223484 | 7.469211746 | 0.31946123 | 4.87E−10 | 5.37E−06 |
| DERL2 | 8.33350867 | 8.023878394 | 0.319068 | 2.89E−10 | 3.20E−06 |
| CTSD | 7.07106353 | 6.605071643 | 0.31895879 | 6.97E−10 | 7.66E−06 |
| PYCARD | 7.20397719 | 6.571483426 | 0.31894768 | 5.20E−11 | 5.82E−07 |
| SAMD4A | 5.64581236 | 5.416356858 | 0.31881754 | 1.44E−10 | 1.60E−06 |
| DCTD | 7.90955912 | 7.494249239 | 0.31866268 | 6.33E−09 | 6.83E−05 |
| PI3 | 5.75178123 | 4.655180226 | 0.31829595 | 4.44E−17 | 5.22E−13 |
| HS2ST1 | 7.56090271 | 7.149363505 | 0.31827474 | 3.02E−10 | 3.34E−06 |
| APOE | 8.15343936 | 7.663104975 | 0.31808554 | 4.82E−07 | 0.00491106 |
| STIM1 | 5.61493541 | 5.343335075 | 0.31789597 | 3.07E−09 | 3.34E−05 |
| NPC1 | 8.57220533 | 8.17495677 | 0.31787444 | 2.88E−12 | 3.27E−08 |
| COL5A3 | 6.34350217 | 5.796368242 | 0.31714321 | 3.42E−11 | 3.84E−07 |
| HK3 | 4.65248974 | 4.330065447 | 0.3166021 | 2.62E−14 | 3.03E−10 |
| LPL | 9.75702795 | 8.716157683 | 0.31646592 | 5.40E−08 | 0.00056941 |
| RAB7L1 | 6.93868134 | 6.400330801 | 0.31627181 | 4.75E−10 | 5.24E−06 |
| TRIM21 | 6.1215792 | 5.736288996 | 0.31610686 | 9.53E−11 | 1.06E−06 |
| KIAA1199 | 6.10028641 | 5.374909391 | 0.31595118 | 6.95E−13 | 7.95E−09 |
| P2RY13 | 5.95987524 | 5.280278792 | 0.31591147 | 6.03E−11 | 6.73E−07 |
| TIMP2 | 7.95321351 | 7.543867166 | 0.31590902 | 9.49E−11 | 1.06E−06 |
| SLN | 7.87209081 | 6.586953535 | 0.31583558 | 2.52E−11 | 2.83E−07 |
| KLHL26 | 6.5551109 | 6.03762464 | 0.31578973 | 6.68E−10 | 7.34E−06 |
| ATP6V0E1 | 8.5261087 | 8.272192838 | 0.31561879 | 2.13E−10 | 2.36E−06 |
| PPP1R3C | 8.28815388 | 7.627214039 | 0.31560166 | 1.21E−07 | 0.00125956 |
| STOML1 | 6.73983673 | 6.388937656 | 0.31556485 | 1.80E−09 | 1.97E−05 |
| TNFAIP2 | 5.57346254 | 5.150766497 | 0.31551811 | 2.56E−13 | 2.94E−09 |
| GNS | 8.35461548 | 7.919626493 | 0.31539212 | 1.40E−12 | 1.59E−08 |
| KCNJ2 | 7.23555594 | 6.654209351 | 0.31538405 | 3.20E−10 | 3.54E−06 |
| PDZD2 | 7.81414149 | 6.878182432 | 0.3151728 | 2.99E−10 | 3.31E−06 |
| HLA-DRB1 | 10.8908544 | 10.08280322 | 0.31502802 | 1.54E−10 | 1.71E−06 |
| TDO2 | 6.04645287 | 5.098346406 | 0.31495497 | 4.66E−11 | 5.21E−07 |
| CYP27A1 | 7.0074745 | 6.641979363 | 0.31446547 | 3.47E−10 | 3.83E−06 |
| SUCLG2 | 7.08154516 | 6.752699473 | 0.31433105 | 1.20E−09 | 1.31E−05 |
| LST1 | 6.60840319 | 6.037406641 | 0.31419993 | 7.93E−10 | 8.71E−06 |
| ARHGAP26 | 5.36767021 | 5.090525117 | 0.31417072 | 1.99E−10 | 2.21E−06 |
| CPD | 7.32680576 | 6.765535108 | 0.31411831 | 4.11E−12 | 4.66E−08 |
| ACP6 | 7.16098065 | 6.627347055 | 0.31404115 | 3.00E−11 | 3.37E−07 |
| MGC14376 | 6.93184078 | 6.417364386 | 0.31403676 | 2.06E−11 | 2.32E−07 |
| HEPH | 6.79778391 | 5.944319845 | 0.31368169 | 2.48E−10 | 2.74E−06 |
| FTHP1 | 9.29140237 | 9.00927953 | 0.31340711 | 3.43E−10 | 3.79E−06 |
| LHFP | 9.09221839 | 8.389385855 | 0.31333674 | 2.07E−07 | 0.00213329 |
| SCAMP2 | 8.02028737 | 7.673711988 | 0.31333671 | 4.20E−10 | 4.64E−06 |
| CTNS | 6.57215221 | 6.213135582 | 0.31330089 | 1.01E−09 | 1.10E−05 |
| BCL2A1 | 6.04433131 | 5.305072214 | 0.31322261 | 9.49E−12 | 1.07E−07 |
| B4GALT5 | 8.88083587 | 8.435506475 | 0.31273607 | 2.93E−10 | 3.24E−06 |
| SNAPC2 | 5.2050388 | 5.024530822 | 0.31252001 | 2.95E−10 | 3.27E−06 |
| NCF1 | 5.16750951 | 4.789045261 | 0.31239876 | 9.74E−17 | 1.14E−12 |
| RBMS1 | 8.1440716 | 7.613914512 | 0.31235575 | 8.65E−11 | 9.65E−07 |
| ALOX5 | 5.14283575 | 4.844125479 | 0.31232671 | 6.21E−20 | 7.37E−16 |
| AGPAT5 | 9.98303536 | 9.511293365 | 0.31225248 | 3.73E−08 | 0.00039482 |
| TUBB6 | 9.57081175 | 8.829659915 | 0.31217314 | 8.51E−10 | 9.34E−06 |
| IGFBP3 | 9.46002217 | 8.479987765 | 0.31204123 | 1.19E−09 | 1.30E−05 |
| TMEM112B | 6.98545546 | 6.731159735 | 0.3119822 | 5.77E−10 | 6.35E−06 |
| FLJ20699 | 6.18628911 | 5.905367338 | 0.31196206 | 3.11E−10 | 3.44E−06 |
| IL10 | 4.30874196 | 4.143767981 | 0.31164297 | 4.33E−12 | 4.91E−08 |
| HSPB6 | 5.41565229 | 4.89881825 | 0.31133815 | 1.25E−14 | 1.45E−10 |
| PPARD | 6.30662598 | 6.103799228 | 0.31105023 | 2.93E−10 | 3.24E−06 |
| SLC15A3 | 5.71178835 | 5.281854767 | 0.31090602 | 1.15E−11 | 1.30E−07 |
| ARPC1B | 8.61638605 | 8.103632782 | 0.31069877 | 5.98E−13 | 6.84E−09 |
| TBC1D9B | 7.00968439 | 6.744984332 | 0.31045068 | 1.64E−10 | 1.82E−06 |
| PTPN6 | 6.70324304 | 6.241683221 | 0.31019065 | 1.11E−12 | 1.27E−08 |
| ACTA2 | 9.65447604 | 8.917377614 | 0.31002424 | 7.58E−11 | 8.46E−07 |
| NCF2 | 6.00005475 | 5.405312925 | 0.31001746 | 1.50E−12 | 1.71E−08 |
| IFI35 | 7.29730932 | 6.725185009 | 0.30988953 | 1.08E−09 | 1.19E−05 |
| BICD1 | 6.73126838 | 6.380264722 | 0.30987031 | 2.72E−09 | 2.96E−05 |
| GPR3 | 4.66526196 | 4.433731374 | 0.30974792 | 1.00E−09 | 1.10E−05 |
| DRAM | 7.10180861 | 6.53234208 | 0.30972476 | 2.29E−11 | 2.58E−07 |
| HLA-DPA1 | 10.8981773 | 10.11687131 | 0.30892818 | 6.85E−11 | 7.65E−07 |
| ATP1A1 | 9.63054854 | 9.233944474 | 0.30867117 | 5.94E−10 | 6.54E−06 |
| COTL1 | 8.38635581 | 7.896667999 | 0.30862196 | 1.83E−09 | 1.99E−05 |
| PTPN12 | 6.54901292 | 6.313858103 | 0.30849329 | 5.13E−09 | 5.55E−05 |
| WIPF1 | 6.56362924 | 6.192182182 | 0.30820641 | 7.62E−11 | 8.50E−07 |
| LILRA1 | 4.58119047 | 4.471827328 | 0.30774385 | 1.65E−09 | 1.80E−05 |
| TNFRSF11A | 4.6133152 | 4.404549602 | 0.30705992 | 2.25E−09 | 2.45E−05 |
| ATF3 | 8.34696578 | 7.637910062 | 0.30677613 | 4.77E−10 | 5.26E−06 |
| CXCL3 | 5.4325594 | 4.837702059 | 0.30669954 | 1.25E−15 | 1.45E−11 |
| ARHGEF3 | 8.33295038 | 7.890693338 | 0.30649068 | 1.69E−08 | 0.00018002 |
| POLD4 | 6.32994672 | 5.980080175 | 0.30644528 | 5.54E−11 | 6.19E−07 |
| OSMR | 5.88607999 | 5.299868619 | 0.30633753 | 5.95E−12 | 6.74E−08 |
| ARL6IP5 | 10.3388581 | 10.06617619 | 0.3062139 | 6.21E−09 | 6.70E−05 |
| CD97 | 6.37925743 | 5.8777708 | 0.30607009 | 9.10E−12 | 1.03E−07 |
| SLC9A1 | 6.37039248 | 6.120852867 | 0.30586608 | 1.46E−10 | 1.63E−06 |
| THBD | 5.82943484 | 5.271919942 | 0.30565403 | 3.37E−14 | 3.89E−10 |
| FKBP5 | 5.99382521 | 5.327253729 | 0.30551333 | 3.99E−10 | 4.41E−06 |
| COL6A1 | 5.32047894 | 5.115201165 | 0.30527883 | 6.40E−13 | 7.32E−09 |
| ETHE1 | 7.85223056 | 7.428107491 | 0.30504356 | 2.86E−10 | 3.17E−06 |
| SLC2A4RG | 6.94418277 | 6.522487206 | 0.3045769 | 8.95E−10 | 9.82E−06 |
| PSCDBP | 5.62822425 | 5.053120919 | 0.30446153 | 1.04E−14 | 1.21E−10 |
| CPE | 10.5400855 | 9.767808228 | 0.30405787 | 3.70E−07 | 0.00378461 |
| KLHDC8A | 6.92674808 | 6.204893066 | 0.30346369 | 3.42E−08 | 0.00036249 |
| GAS7 | 7.08022571 | 6.500953285 | 0.30341377 | 1.16E−09 | 1.27E−05 |
| SP110 | 6.86050751 | 6.454749153 | 0.30324144 | 6.24E−11 | 6.97E−07 |
| SORL1 | 9.20102794 | 8.602380125 | 0.30323577 | 7.90E−09 | 8.51E−05 |
| ENPEP | 5.61307916 | 5.182175339 | 0.30310945 | 8.48E−10 | 9.30E−06 |
| MERTK | 6.02089839 | 5.558513806 | 0.30293765 | 1.60E−09 | 1.74E−05 |
| CXCR4 | 8.1386871 | 7.477450703 | 0.30287782 | 4.22E−09 | 4.57E−05 |
| HMHA1 | 6.12428393 | 5.737355652 | 0.30276359 | 3.13E−11 | 3.51E−07 |
| ANGPT1 | 6.65194888 | 5.907049923 | 0.3026942 | 5.82E−11 | 6.50E−07 |
| XKR8 | 7.50692821 | 7.216928175 | 0.30263285 | 2.73E−09 | 2.97E−05 |
| SPSB1 | 5.91592267 | 5.527634348 | 0.30253482 | 4.17E−09 | 4.52E−05 |
| TPM4 | 8.17143891 | 7.70975633 | 0.30234078 | 2.15E−08 | 0.00022936 |
| KIF13B | 7.19990566 | 6.69338754 | 0.30229258 | 5.64E−09 | 6.10E−05 |
| ELL2 | 4.93135104 | 4.735904136 | 0.30219488 | 1.28E−12 | 1.45E−08 |
| TOMM7 | 11.9781138 | 11.74806785 | 0.30199277 | 2.66E−09 | 2.89E−05 |
| LYL1 | 5.67630071 | 5.408509208 | 0.3016843 | 1.29E−09 | 1.41E−05 |
| MANBA | 6.91883259 | 6.626532085 | 0.30167408 | 4.91E−10 | 5.42E−06 |
| NTAN1 | 7.35913987 | 6.95065857 | 0.30161642 | 2.48E−09 | 2.70E−05 |
| RHOB | 10.4585355 | 9.821951336 | 0.3015113 | 4.04E−07 | 0.00412461 |
| MAN2A1 | 8.01046139 | 7.493760051 | 0.30115539 | 8.44E−10 | 9.27E−06 |
| TCTA | 7.820773 | 7.37876513 | 0.30106818 | 2.05E−08 | 0.0002184 |
| ZFP106 | 8.54536741 | 8.08424735 | 0.30101228 | 8.41E−10 | 9.23E−06 |
| FBXO17 | 6.3679196 | 5.896468372 | 0.30073573 | 1.22E−07 | 0.00126401 |
| PLEK | 6.53860096 | 6.069265716 | 0.30065706 | 2.46E−11 | 2.77E−07 |
| RAB4B | 7.1684553 | 6.82621259 | 0.30043116 | 1.19E−08 | 0.00012719 |
| HYPE | 6.505636 | 6.08171389 | 0.30034138 | 1.11E−08 | 0.00011959 |
| VAV1 | 5.20122665 | 4.899350461 | 0.29991618 | 3.87E−12 | 4.39E−08 |
| SERPINF1 | 8.3491913 | 7.566490386 | 0.29983886 | 1.78E−10 | 1.98E−06 |
| NCKAP1L | 5.8010182 | 5.350092538 | 0.29972218 | 2.53E−12 | 2.88E−08 |
| TAGLN2 | 8.69680236 | 8.188757958 | 0.29965098 | 3.27E−08 | 0.00034668 |
| SLC22A18 | 6.48699719 | 5.95856278 | 0.29956556 | 2.27E−11 | 2.55E−07 |
| CTSO | 8.61943116 | 8.062189269 | 0.29937763 | 2.00E−07 | 0.002067 |
| GLB1L | 5.49751867 | 5.222412925 | 0.2992959 | 1.67E−09 | 1.82E−05 |
| ORAI2 | 5.4643389 | 5.224872877 | 0.29926278 | 1.57E−08 | 0.00016744 |
| ARHGAP25 | 6.07681062 | 5.720320488 | 0.29924886 | 8.30E−10 | 9.12E−06 |
| HLA-DMB | 8.6798074 | 8.039122716 | 0.29877846 | 1.60E−09 | 1.75E−05 |
| LSP1 | 5.80776772 | 5.406025116 | 0.298737 | 2.67E−11 | 3.00E−07 |
| LAPTM4A | 11.6214745 | 11.40108434 | 0.29868688 | 3.49E−10 | 3.85E−06 |
| BNIP3L | 10.5517271 | 10.21588616 | 0.29841442 | 5.53E−09 | 5.98E−05 |
| NPAS2 | 5.03231157 | 4.757366063 | 0.2982716 | 1.97E−11 | 2.22E−07 |
| KCNF1 | 6.06839609 | 5.425636086 | 0.2981688 | 8.95E−10 | 9.82E−06 |
| GAS2L1 | 6.91154669 | 6.530232145 | 0.29807995 | 7.43E−08 | 0.00077893 |
| IER5 | 9.14351099 | 8.672635827 | 0.29806674 | 1.70E−09 | 1.86E−05 |
| CHN1 | 9.84351095 | 9.183854266 | 0.2980031 | 6.75E−08 | 0.0007089 |
| CYP19A1 | 4.592221 | 4.315317052 | 0.29789533 | 3.14E−07 | 0.00321942 |
| SPRY1 | 8.03670896 | 7.327732347 | 0.29772781 | 1.54E−08 | 0.00016491 |
| TGFBR2 | 6.23569252 | 5.840475273 | 0.29742628 | 3.97E−13 | 4.55E−09 |
| BST2 | 7.84458647 | 7.113905009 | 0.29706996 | 3.67E−09 | 3.98E−05 |
| SLC2A1 | 8.75453288 | 8.194078447 | 0.29700647 | 5.44E−10 | 5.99E−06 |
| BGN | 6.64140749 | 6.224094558 | 0.29677452 | 2.45E−08 | 0.00026075 |
| MOSPD2 | 6.9788354 | 6.654190128 | 0.29675836 | 2.15E−08 | 0.00022936 |
| SPRY4 | 7.89429669 | 7.07634606 | 0.29674636 | 4.73E−08 | 0.00049891 |
| MEF2A | 6.89210976 | 6.575720815 | 0.2967272 | 1.74E−09 | 1.90E−05 |
| NOD1 | 5.65532971 | 5.355590413 | 0.29661702 | 2.14E−10 | 2.37E−06 |
| APBB1IP | 5.09144735 | 4.823280203 | 0.29657943 | 1.74E−11 | 1.96E−07 |
| PIGT | 8.44640007 | 8.140945861 | 0.29650412 | 2.37E−09 | 2.58E−05 |
| SCRN1 | 9.60734789 | 8.994078989 | 0.29646988 | 1.45E−07 | 0.00150542 |
| CFL1 | 12.6017777 | 12.38693948 | 0.29638823 | 1.56E−09 | 1.70E−05 |
| LGALS3BP | 8.82751275 | 8.350880326 | 0.29614152 | 6.41E−10 | 7.04E−06 |
| SEC14L1 | 8.46729876 | 7.946661267 | 0.29611528 | 1.13E−09 | 1.24E−05 |
| ZFP36L1 | 7.95487829 | 7.503500177 | 0.29598371 | 2.00E−08 | 0.00021273 |
| ZMAT3 | 8.52673718 | 7.868093365 | 0.29596948 | 1.52E−08 | 0.00016243 |
| ADAM28 | 5.43340157 | 5.100830047 | 0.29594511 | 5.07E−13 | 5.80E−09 |
| MAPKAPK2 | 5.98759485 | 5.758374015 | 0.29545136 | 5.34E−08 | 0.00056326 |
| VAMP3 | 9.08449317 | 8.631930053 | 0.29525425 | 1.63E−09 | 1.78E−05 |
| CD248 | 6.68548765 | 6.278952385 | 0.29502519 | 5.83E−09 | 6.29E−05 |
| YIPF1 | 7.46862022 | 7.183156391 | 0.29470162 | 5.76E−08 | 0.00060583 |
| HSD17B12 | 10.5567797 | 10.22983562 | 0.29467387 | 8.13E−10 | 8.93E−06 |
| COL5A2 | 8.57904781 | 7.775340137 | 0.29461106 | 2.71E−09 | 2.95E−05 |
| FGF2 | 6.18405232 | 5.775998677 | 0.29428636 | 1.53E−09 | 1.67E−05 |
| ITGB8 | 5.50527259 | 5.060933631 | 0.29401292 | 2.44E−11 | 2.74E−07 |
| RAP1A | 9.33664791 | 9.081930528 | 0.29389598 | 1.01E−09 | 1.11E−05 |
| RGN | 6.64282591 | 6.013251761 | 0.29383361 | 1.24E−08 | 0.00013315 |
| NUAK2 | 5.96550218 | 5.468444152 | 0.29383078 | 1.44E−12 | 1.65E−08 |
| ELTD1 | 7.25649685 | 6.637610239 | 0.29371686 | 3.61E−08 | 0.0003819 |
| CCPG1 | 6.75537799 | 6.349859402 | 0.29364435 | 3.46E−10 | 3.82E−06 |
| CNGA3 | 6.30001006 | 5.493697054 | 0.2935876 | 2.28E−10 | 2.53E−06 |
| DDEFL1 | 6.27296124 | 5.938273591 | 0.2932473 | 5.09E−09 | 5.50E−05 |
| COMMD9 | 8.22945885 | 7.939697641 | 0.29316444 | 5.57E−08 | 0.0005862 |
| SIGLEC5 | 4.86227004 | 4.674281676 | 0.292956 | 2.55E−08 | 0.0002706 |
| ATP8B4 | 4.46108686 | 4.159235783 | 0.2928848 | 8.77E−12 | 9.92E−08 |
| GLB1 | 8.43778488 | 8.075768833 | 0.29288073 | 5.19E−10 | 5.72E−06 |
| SNX5 | 9.06024042 | 8.692627482 | 0.29262978 | 5.29E−08 | 0.00055712 |
| UBTD1 | 4.91109214 | 4.706435897 | 0.2925918 | 2.48E−10 | 2.74E−06 |
| CNDP2 | 9.8266651 | 9.518507279 | 0.29237613 | 1.51E−08 | 0.00016122 |
| PKD2 | 7.53897919 | 7.123849555 | 0.292302 | 6.04E−09 | 6.52E−05 |
| CLEC2B | 7.39786709 | 6.686895522 | 0.2922744 | 2.25E−10 | 2.50E−06 |
| RASSF4 | 6.70408898 | 6.273972179 | 0.291941 | 5.29E−08 | 0.00055712 |
| CAV1 | 9.46937148 | 8.635727354 | 0.29129925 | 1.95E−09 | 2.13E−05 |
| TMEM51 | 6.73554864 | 6.403778723 | 0.29115614 | 4.13E−10 | 4.56E−06 |
| EGR2 | 6.66678981 | 6.099636072 | 0.29092137 | 8.12E−09 | 8.74E−05 |
| SERPINB8 | 5.19365145 | 4.837807698 | 0.29075637 | 4.16E−11 | 4.66E−07 |
| C20orf116 | 8.16194478 | 7.868968309 | 0.29068493 | 6.20E−10 | 6.81E−06 |
| CSDA | 7.45912121 | 7.114482232 | 0.29059911 | 2.01E−10 | 2.23E−06 |
| OLR1 | 6.30698905 | 5.604511393 | 0.29019952 | 4.87E−10 | 5.37E−06 |
| HLA-DRA | 10.8393139 | 9.971618893 | 0.29013071 | 1.42E−09 | 1.55E−05 |
| TMED10 | 10.3839491 | 10.08078037 | 0.28997867 | 2.18E−10 | 2.41E−06 |
| ADAMTS1 | 6.63030277 | 5.973966038 | 0.28996805 | 3.11E−10 | 3.44E−06 |
| TM6SF1 | 6.23121555 | 5.802063818 | 0.28981316 | 6.13E−09 | 6.62E−05 |
| DAB2 | 7.59855923 | 7.010518919 | 0.28946529 | 2.07E−09 | 2.26E−05 |
| ZDHHC24 | 5.82853667 | 5.549334698 | 0.28934388 | 7.11E−10 | 7.82E−06 |
| BCL6 | 7.81358932 | 7.345227782 | 0.28876917 | 1.08E−08 | 0.00011639 |
| SLC4A7 | 5.46689346 | 5.310646108 | 0.28876793 | 8.92E−09 | 9.59E−05 |
| GABARAP | 11.1723464 | 10.92661913 | 0.2885372 | 2.52E−07 | 0.00259685 |
| GYPC | 7.83732442 | 7.234972231 | 0.28851785 | 4.66E−09 | 5.04E−05 |
| MFAP3L | 5.52066308 | 5.282958823 | 0.28782366 | 8.99E−09 | 9.67E−05 |
| BBS1 | 8.28857278 | 7.795396755 | 0.28770036 | 5.09E−07 | 0.00518703 |
| MEOX2 | 7.0391589 | 5.872283149 | 0.28761766 | 1.25E−08 | 0.00013417 |
| NME5 | 7.41430375 | 6.784940622 | 0.28760961 | 1.85E−07 | 0.00190763 |
| CPEB1 | 6.39458097 | 5.914339092 | 0.28757788 | 2.12E−08 | 0.00022599 |
| YAP1 | 6.0424881 | 5.453274878 | 0.2875662 | 5.17E−08 | 0.00054497 |
| TRIM6-TRIM34 | 5.94351534 | 5.52121761 | 0.28725757 | 2.07E−09 | 2.26E−05 |
| HLA-DPB1 | 9.94568858 | 9.19901709 | 0.28718563 | 1.78E−09 | 1.94E−05 |
| SIL1 | 6.72719756 | 6.415517714 | 0.287026 | 7.71E−09 | 8.31E−05 |
| TLN1 | 6.98019407 | 6.508633458 | 0.28701891 | 4.35E−08 | 0.00045997 |
| GPC4 | 6.2154262 | 5.744642784 | 0.28692689 | 5.82E−10 | 6.40E−06 |
| NUPR1 | 7.86850368 | 7.289861267 | 0.28689007 | 7.96E−09 | 8.57E−05 |
| CTSS | 7.33003705 | 6.705973204 | 0.2868824 | 5.37E−10 | 5.91E−06 |
| PCGF1 | 7.24873123 | 6.899215835 | 0.28672876 | 8.02E−09 | 8.64E−05 |
| MTM1 | 6.00252931 | 5.63998072 | 0.28664185 | 1.76E−08 | 0.00018771 |
| PGLS | 8.86749376 | 8.570155406 | 0.28651369 | 3.83E−08 | 0.00040523 |
| RPS6KA2 | 7.35845365 | 6.853418592 | 0.28649509 | 2.61E−07 | 0.00268869 |
| ARHGAP1 | 6.46648905 | 6.284148691 | 0.28645878 | 1.44E−08 | 0.00015457 |
| OBSL1 | 6.00767485 | 5.713908598 | 0.28632481 | 7.94E−08 | 0.00083118 |
| RGS6 | 5.05382288 | 4.77273261 | 0.28612616 | 4.30E−09 | 4.66E−05 |
| TFEC | 5.47778175 | 4.996804697 | 0.28611745 | 1.54E−10 | 1.71E−06 |
| MAP3K14 | 5.58949169 | 5.297016282 | 0.28596977 | 2.86E−08 | 0.00030284 |
| TPI1 | 10.4365507 | 10.15513167 | 0.28583422 | 6.43E−08 | 0.00067611 |
| CDH11 | 8.95797282 | 8.350047659 | 0.28580937 | 8.79E−08 | 0.00091885 |
| BDH2 | 8.88324857 | 8.470033183 | 0.28542454 | 4.24E−09 | 4.59E−05 |
| MAP1LC3B | 9.69257429 | 9.309590424 | 0.2854202 | 8.73E−08 | 0.0009125 |
| PH-4 | 8.44860875 | 8.040043116 | 0.28529648 | 1.02E−07 | 0.00106063 |
| UROD | 8.72715765 | 8.461461841 | 0.28526946 | 5.45E−09 | 5.88E−05 |
| PFN1 | 10.5034038 | 10.15094857 | 0.28499722 | 5.00E−08 | 0.00052711 |
| COLEC12 | 7.25168312 | 6.457771626 | 0.28497347 | 4.55E−09 | 4.93E−05 |
| WDR41 | 8.46686242 | 8.098015619 | 0.28493701 | 6.06E−09 | 6.54E−05 |
| NLRX1 | 6.1797909 | 5.895644103 | 0.28478977 | 1.74E−09 | 1.90E−05 |
| KIAA1033 | 8.12588141 | 7.801810791 | 0.28473598 | 8.18E−09 | 8.81E−05 |
| LEFTY2 | 6.44829266 | 5.833301256 | 0.28437762 | 1.48E−08 | 0.00015817 |
| IL1B | 6.41030322 | 5.74940746 | 0.28419268 | 3.53E−11 | 3.96E−07 |
| CENTD3 | 7.46128562 | 6.80797739 | 0.28397802 | 7.32E−08 | 0.00076784 |
| CHCHD7 | 7.39460916 | 7.050720016 | 0.28386841 | 9.42E−09 | 0.00010124 |
| TEGT | 10.8849705 | 10.62611125 | 0.28385734 | 2.23E−10 | 2.48E−06 |
| TRIM38 | 6.04068874 | 5.602589882 | 0.28371989 | 1.19E−10 | 1.32E−06 |
| CSTA | 7.27523213 | 6.475889655 | 0.28357304 | 2.75E−15 | 3.20E−11 |
| RIPK1 | 5.55791406 | 5.248955974 | 0.28353328 | 7.91E−09 | 8.52E−05 |
| PLCG2 | 6.32157107 | 5.851287608 | 0.28348712 | 1.92E−10 | 2.13E−06 |
| SYK | 6.54569855 | 6.060456692 | 0.28347686 | 5.35E−10 | 5.89E−06 |
| FOLR2 | 6.76824738 | 6.17789461 | 0.28306477 | 2.21E−07 | 0.00228128 |
| ADCK2 | 6.59700867 | 6.294537471 | 0.28300083 | 2.96E−09 | 3.22E−05 |
| IRF2 | 6.84552036 | 6.444100256 | 0.28283711 | 1.58E−08 | 0.00016937 |
| ENTPD1 | 6.39901524 | 6.049488287 | 0.28281131 | 5.30E−09 | 5.72E−05 |
| BIRC3 | 5.54666508 | 4.939811494 | 0.28249358 | 2.40E−14 | 2.78E−10 |
| CPSF4 | 8.05480054 | 7.790584294 | 0.28236024 | 9.57E−08 | 0.00099821 |
| PDE4B | 8.75807954 | 8.107389254 | 0.28205393 | 5.42E−08 | 0.00057148 |
| EGF | 4.66294507 | 4.355165595 | 0.28203977 | 1.08E−12 | 1.23E−08 |
| BDKRB2 | 5.3032422 | 4.946965239 | 0.28180406 | 1.90E−10 | 2.10E−06 |
| ORAI3 | 6.84225654 | 6.521780554 | 0.28175921 | 5.36E−08 | 0.0005653 |
| BRP44L | 9.7206281 | 9.346821132 | 0.28169057 | 2.68E−07 | 0.00275515 |
| TLR3 | 5.48387896 | 5.076972993 | 0.28164228 | 3.15E−09 | 3.42E−05 |
| RCN1 | 8.62702406 | 8.275417975 | 0.28162687 | 1.37E−07 | 0.00142214 |
| FMOD | 6.82382559 | 6.098446831 | 0.28151896 | 2.30E−10 | 2.55E−06 |
| HSPA2 | 8.3987665 | 7.639217008 | 0.28148925 | 5.51E−08 | 0.00057987 |
| RAB8B | 6.09483503 | 5.735117897 | 0.28138746 | 8.68E−09 | 9.34E−05 |
| OGDH | 7.22272204 | 6.834145844 | 0.28138591 | 2.90E−08 | 0.00030741 |
| DDB2 | 6.37245376 | 5.9238495 | 0.28112398 | 1.66E−09 | 1.81E−05 |
| POSTN | 8.97918653 | 7.36991361 | 0.28043182 | 1.24E−08 | 0.00013266 |
| MKNK1 | 7.45074967 | 7.077184559 | 0.2804266 | 3.70E−08 | 0.0003919 |
| TMED5 | 8.48959003 | 8.182305727 | 0.28030457 | 2.66E−08 | 0.00028199 |
| BTK | 5.70994475 | 5.374069406 | 0.28027424 | 8.94E−12 | 1.01E−07 |
| GPSN2 | 9.17389112 | 8.84207947 | 0.27956994 | 7.40E−08 | 0.00077621 |
| C12orf5 | 7.94001353 | 7.49148674 | 0.27950476 | 1.21E−09 | 1.32E−05 |
| SYPL1 | 9.15643208 | 8.664288395 | 0.27930254 | 1.81E−07 | 0.00186806 |
| APOC1 | 10.7310116 | 10.03808244 | 0.27916017 | 1.40E−07 | 0.0014476 |
| GRAMD3 | 8.9806462 | 8.307473488 | 0.27887323 | 8.00E−07 | 0.0080988 |
| RDH5 | 5.79870764 | 5.524188725 | 0.27871216 | 8.91E−10 | 9.78E−06 |
| PMFBP1 | 4.97129525 | 4.804056965 | 0.27870503 | 2.22E−08 | 0.00023634 |
| FNBP1 | 7.89484069 | 7.534333641 | 0.27843034 | 5.42E−09 | 5.86E−05 |
| DUSP6 | 8.67345928 | 8.100313605 | 0.27827442 | 3.57E−07 | 0.00365725 |
| CSNK1A1 | 8.8757578 | 8.633289141 | 0.27822436 | 2.53E−08 | 0.00026858 |
| KIAA1539 | 5.90503406 | 5.687354705 | 0.27796218 | 3.62E−07 | 0.00370798 |
| ATP6V1B2 | 9.45112264 | 9.073532096 | 0.2779576 | 3.35E−08 | 0.00035458 |
| PRKCSH | 8.60113386 | 8.211587693 | 0.27789021 | 2.56E−07 | 0.00263332 |
| VAT1 | 8.84625681 | 8.490293269 | 0.27775556 | 2.98E−07 | 0.00305746 |
| RNF24 | 7.40284835 | 7.038865107 | 0.2776655 | 6.63E−08 | 0.00069606 |
| OR3A1 | 4.11300797 | 4.015912085 | 0.27751133 | 1.13E−11 | 1.28E−07 |
| ITM2B | 11.001338 | 10.73120974 | 0.27749688 | 9.71E−08 | 0.0010123 |
| LRPAP1 | 8.33535855 | 8.050675445 | 0.27740314 | 8.31E−09 | 8.94E−05 |
| C6orf62 | 9.04796755 | 8.713978531 | 0.27737271 | 1.84E−08 | 0.00019647 |
| TRIM14 | 6.12166536 | 5.882893386 | 0.27735568 | 2.02E−08 | 0.00021512 |
| TRAF6 | 5.93021618 | 5.666760728 | 0.27734093 | 6.77E−09 | 7.30E−05 |
| STAC | 6.37808975 | 5.667141221 | 0.2772698 | 1.89E−10 | 2.10E−06 |
| ABCB9 | 4.93031094 | 4.735488383 | 0.27725704 | 6.25E−10 | 6.87E−06 |
| FTH1 | 12.0349648 | 11.71346226 | 0.27713405 | 1.28E−08 | 0.00013731 |
| RUNX1 | 4.57222349 | 4.478793971 | 0.2770261 | 7.82E−11 | 8.72E−07 |
| PVRL2 | 6.12865306 | 5.796019917 | 0.2766143 | 1.30E−08 | 0.0001389 |
| PRF1 | 5.39140011 | 4.964109386 | 0.27658634 | 1.41E−11 | 1.60E−07 |
| PDGFD | 7.05465781 | 6.285876065 | 0.27652399 | 1.91E−08 | 0.00020402 |
| EMR1 | 4.5822057 | 4.217542273 | 0.27648399 | 1.76E−11 | 1.98E−07 |
| SLC27A3 | 6.95057532 | 6.450818014 | 0.27636674 | 8.48E−08 | 0.00088691 |
| ATP6V1D | 9.52268565 | 9.23303962 | 0.27617603 | 6.11E−09 | 6.59E−05 |
| DOK1 | 5.19026608 | 5.02433499 | 0.27606623 | 1.73E−09 | 1.89E−05 |
| TMEM47 | 8.79807099 | 8.10978019 | 0.27605617 | 8.03E−07 | 0.00812554 |
| FVT1 | 7.62666747 | 7.346396107 | 0.27592251 | 2.12E−08 | 0.00022599 |
| APOBEC3C | 6.76953023 | 6.465191881 | 0.2758191 | 5.28E−09 | 5.70E−05 |
| GMPR | 6.0559466 | 5.534505035 | 0.27579232 | 5.04E−10 | 5.55E−06 |
| SELPLG | 6.04551588 | 5.736628897 | 0.27565278 | 9.25E−10 | 1.01E−05 |
| SCN1B | 5.8824321 | 5.536697986 | 0.27564223 | 2.61E−08 | 0.00027679 |
| TMEM43 | 8.1208781 | 7.786866653 | 0.27562673 | 1.24E−08 | 0.00013266 |
| CAPZB | 9.23223817 | 8.98586143 | 0.27526718 | 2.05E−07 | 0.00211104 |
| PRKACA | 5.70663615 | 5.560839299 | 0.27517367 | 1.32E−08 | 0.00014157 |
| YKT6 | 7.15735403 | 6.803374883 | 0.27505439 | 2.86E−08 | 0.00030284 |
| MYH9 | 7.03153315 | 6.622355334 | 0.27493022 | 6.91E−11 | 7.72E−07 |
| MOCS1 | 5.4585523 | 5.234335798 | 0.27491194 | 2.45E−08 | 0.00026075 |
| ATP6AP1 | 9.59643392 | 9.286518339 | 0.27441592 | 3.32E−09 | 3.60E−05 |
| SNX10 | 9.21901585 | 8.454638512 | 0.27426758 | 9.46E−09 | 0.00010161 |
| NCSTN | 7.8356783 | 7.491821807 | 0.27424733 | 1.98E−07 | 0.00203833 |
| FAM63B | 5.19899677 | 4.954438366 | 0.27418924 | 5.17E−08 | 0.00054497 |
| CNR1 | 5.42826591 | 5.114033224 | 0.27413401 | 7.00E−10 | 7.69E−06 |
| F8 | 7.48001806 | 7.010615057 | 0.27373188 | 3.62E−07 | 0.00370798 |
| PPT1 | 11.5853808 | 11.36965435 | 0.27335852 | 4.39E−08 | 0.00046338 |
| FXYD5 | 7.47438362 | 6.927690048 | 0.27317402 | 5.99E−10 | 6.59E−06 |
| PIGB | 6.07793239 | 5.65694712 | 0.27313708 | 4.25E−09 | 4.61E−05 |
| MGP | 9.80628074 | 8.938379044 | 0.27310377 | 1.83E−07 | 0.00188766 |
| PARVB | 5.25926333 | 4.914470983 | 0.27303768 | 9.10E−12 | 1.03E−07 |
| TEX2 | 7.58897325 | 7.293494593 | 0.27266301 | 5.93E−08 | 0.00062371 |
| WDR1 | 8.17608913 | 7.84491416 | 0.27231274 | 2.21E−08 | 0.00023547 |
| COL6A2 | 6.16219821 | 5.545179163 | 0.27228044 | 4.07E−11 | 4.56E−07 |
| ELOVL1 | 7.50806781 | 7.193461057 | 0.27222304 | 4.71E−08 | 0.0004971 |
| MYO7A | 4.78699975 | 4.633209387 | 0.27217469 | 4.79E−09 | 5.19E−05 |
| KCNQ1 | 4.76279793 | 4.659999628 | 0.27196908 | 2.94E−09 | 3.19E−05 |
| KDELR1 | 7.32142335 | 6.938425838 | 0.27195519 | 2.96E−09 | 3.22E−05 |
| TPCN1 | 7.33217626 | 6.967808287 | 0.27185501 | 2.28E−07 | 0.00234654 |
| C9orf167 | 4.99331452 | 4.756999713 | 0.27113733 | 1.59E−12 | 1.81E−08 |
| HOMER3 | 6.14640539 | 5.882745519 | 0.2709145 | 4.93E−09 | 5.33E−05 |
| KLF6 | 7.44984572 | 7.002071506 | 0.27088969 | 5.02E−10 | 5.53E−06 |
| CD58 | 7.12194058 | 6.69497581 | 0.27057333 | 3.31E−07 | 0.00339162 |
| ALDOA | 12.0153185 | 11.74584158 | 0.27047757 | 3.39E−07 | 0.00347375 |
| PPP1R15A | 5.92406817 | 5.590402371 | 0.27043141 | 1.96E−11 | 2.21E−07 |
| ALPK3 | 5.29228798 | 4.983422644 | 0.27035773 | 4.18E−11 | 4.68E−07 |
| RRBP1 | 5.96595711 | 5.735393661 | 0.27032901 | 1.11E−09 | 1.21E−05 |
| TPK1 | 5.89773496 | 5.521707752 | 0.26994895 | 2.95E−08 | 0.00031319 |
| GM2A | 6.4706004 | 6.187006256 | 0.26987274 | 3.80E−09 | 4.12E−05 |
| NINJ1 | 7.82118179 | 7.4717602 | 0.26965136 | 3.92E−08 | 0.00041438 |
| TGOLN2 | 7.89809069 | 7.613921313 | 0.26947834 | 9.49E−09 | 0.00010199 |
| ACSL3 | 8.9722267 | 8.516063928 | 0.26944063 | 2.71E−08 | 0.00028734 |
| SNX24 | 6.62718002 | 6.268025396 | 0.26940107 | 2.80E−07 | 0.00287256 |
| APOBEC3F | 4.26232515 | 4.111728049 | 0.26914192 | 1.97E−09 | 2.15E−05 |
| TMED7 | 8.20889638 | 7.932836074 | 0.2690227 | 8.35E−08 | 0.00087425 |
| CASP4 | 6.83665239 | 6.314635531 | 0.26878344 | 9.53E−10 | 1.04E−05 |
| TRADD | 5.67574779 | 5.498538273 | 0.2687536 | 2.38E−10 | 2.64E−06 |
| TSPO | 8.01297667 | 7.611769516 | 0.26846892 | 1.89E−08 | 0.00020171 |
| HK1 | 8.97803519 | 8.639749977 | 0.26845493 | 1.35E−08 | 0.00014486 |
| CLIP1 | 6.58332788 | 6.245353353 | 0.26842073 | 1.74E−07 | 0.00180345 |
| ACSBG1 | 7.641933 | 6.818109401 | 0.26841825 | 4.22E−07 | 0.00431232 |
| ZNF395 | 7.86877017 | 7.439603953 | 0.26830056 | 7.79E−08 | 0.0008166 |
| EHBP1 | 8.36070666 | 8.01442999 | 0.2681461 | 2.38E−07 | 0.0024546 |
| RAB11FIP5 | 6.31513987 | 6.017594706 | 0.26801077 | 1.30E−07 | 0.00134805 |
| PLCD1 | 7.31644933 | 6.912285488 | 0.26770479 | 1.14E−07 | 0.00118981 |
| GPX3 | 8.93252636 | 8.12364933 | 0.26750265 | 8.63E−08 | 0.00090287 |
| HBEGF | 5.86905714 | 5.576630847 | 0.2674025 | 1.74E−10 | 1.93E−06 |
| ITGB4 | 5.04410134 | 4.804484852 | 0.26733194 | 9.42E−09 | 0.00010124 |
| C17orf62 | 8.04917073 | 7.792893774 | 0.26727792 | 1.27E−07 | 0.00131486 |
| TMSB10 | 12.8561889 | 12.63572376 | 0.26691723 | 6.85E−08 | 0.00071923 |
| PTK2B | 4.82926908 | 4.643562268 | 0.26675758 | 3.63E−11 | 4.06E−07 |
| DHX58 | 5.35959971 | 5.089737505 | 0.2666875 | 1.65E−08 | 0.00017662 |
| MPV17 | 8.9808367 | 8.655227623 | 0.26668359 | 1.41E−08 | 0.00015109 |
| YTHDF1 | 9.58855796 | 9.266934342 | 0.26666046 | 4.39E−07 | 0.00447927 |
| BACH1 | 5.57653584 | 5.394733905 | 0.26663432 | 1.54E−08 | 0.00016491 |
| GNPDA1 | 8.56222441 | 8.273249744 | 0.26636372 | 2.15E−08 | 0.00022853 |
| ARSF | 5.05952571 | 4.606023963 | 0.26633465 | 7.92E−13 | 9.05E−09 |
| P2RY1 | 5.24306979 | 4.838654575 | 0.2661691 | 1.19E−10 | 1.33E−06 |
| C7orf42 | 9.16899858 | 8.845738888 | 0.26560612 | 3.10E−07 | 0.00317577 |
| DAG1 | 7.31139444 | 7.016990269 | 0.26535493 | 2.88E−07 | 0.0029532 |
| BCL2L1 | 5.92823247 | 5.647049706 | 0.26514137 | 2.73E−09 | 2.97E−05 |
| KIAA0409 | 6.33732866 | 6.077064692 | 0.26510957 | 4.82E−08 | 0.00050811 |
| CANX | 10.1030424 | 9.750981029 | 0.26500397 | 1.33E−09 | 1.46E−05 |
| NMI | 8.09196507 | 7.652869088 | 0.26469802 | 2.84E−07 | 0.00291275 |
| FABP5 | 10.0545391 | 9.168545459 | 0.26462832 | 1.13E−07 | 0.00117277 |
| WBP2 | 7.36206659 | 6.918689789 | 0.26425903 | 7.20E−07 | 0.00729921 |
| HLA-G | 8.03748727 | 7.769789305 | 0.26424643 | 2.20E−08 | 0.00023461 |
| TMEM149 | 6.10262925 | 5.761595319 | 0.26408839 | 1.72E−07 | 0.00178487 |
| CHRNE | 6.61525093 | 6.368477683 | 0.26379928 | 7.88E−08 | 0.00082542 |
| VWF | 8.75035854 | 8.141341378 | 0.2637705 | 6.52E−07 | 0.00662231 |
| MGST2 | 8.0690967 | 7.65307757 | 0.26366138 | 2.30E−08 | 0.00024446 |
| FILIP1L | 7.74562695 | 7.180476716 | 0.26344216 | 2.58E−08 | 0.00027368 |
| RDX | 8.55865659 | 8.165004412 | 0.2634307 | 1.68E−07 | 0.0017351 |
| GLT25D1 | 6.55893948 | 6.284991437 | 0.26335331 | 1.32E−08 | 0.00014103 |
| XAF1 | 6.82162598 | 6.144076991 | 0.26316972 | 4.55E−09 | 4.93E−05 |
| SLC24A6 | 5.59430662 | 5.365552055 | 0.26316056 | 5.02E−07 | 0.00511642 |
| ECOP | 10.2500136 | 9.6589335 | 0.2628628 | 8.41E−10 | 9.23E−06 |
| MAP3K8 | 5.28058109 | 4.802376153 | 0.26252641 | 2.71E−09 | 2.95E−05 |
| IRAK1 | 9.34243397 | 9.025018943 | 0.26235814 | 9.06E−09 | 9.74E−05 |
| PLXNB2 | 6.6396946 | 6.42613882 | 0.26223418 | 4.44E−08 | 0.00046847 |
| FZD1 | 5.9572925 | 5.607226788 | 0.26220937 | 1.05E−09 | 1.15E−05 |
| GALNT11 | 8.61720422 | 8.294374278 | 0.26213251 | 1.81E−08 | 0.00019352 |
| PARP3 | 5.41038397 | 5.163629247 | 0.26187198 | 3.01E−09 | 3.27E−05 |
| GPR4 | 4.66692213 | 4.499173809 | 0.26183903 | 3.12E−08 | 0.00033013 |
| TNIP1 | 7.63872605 | 7.298117352 | 0.26161473 | 8.57E−08 | 0.00089637 |
| SOCS6 | 5.70146128 | 5.403272718 | 0.26124811 | 2.83E−08 | 0.00030058 |
| C1orf166 | 6.32317334 | 6.072672149 | 0.26104116 | 1.02E−07 | 0.00106063 |
| ZNF217 | 7.01798485 | 6.475420164 | 0.26089833 | 3.85E−08 | 0.00040672 |
| GLT8D1 | 8.60941832 | 8.324867649 | 0.26087408 | 7.84E−07 | 0.00793939 |
| BACE2 | 7.1582386 | 6.590108105 | 0.26079212 | 5.65E−10 | 6.22E−06 |
| PTAFR | 5.25711335 | 5.004881201 | 0.26061629 | 1.56E−09 | 1.70E−05 |
| SLC12A7 | 7.38602206 | 7.02371355 | 0.2601743 | 1.66E−08 | 0.00017729 |
| LOC93349 | 5.99532275 | 5.563432826 | 0.26008547 | 1.88E−08 | 0.00020096 |
| ZC3HAV1 | 7.13532452 | 6.896290235 | 0.25983754 | 1.51E−08 | 0.00016122 |
| ASAHL | 5.31812159 | 5.105562954 | 0.25969665 | 1.96E−07 | 0.00202404 |
| PSAP | 10.7882683 | 10.5090557 | 0.25960567 | 4.70E−07 | 0.00479641 |
| TAX1BP3 | 6.34262387 | 6.126332736 | 0.25948323 | 2.52E−08 | 0.00026758 |
| ARSA | 4.99517152 | 4.764235175 | 0.25927761 | 2.96E−07 | 0.00303662 |
| APOL6 | 5.64785464 | 5.276832224 | 0.25886583 | 1.93E−09 | 2.11E−05 |
| PSMB9 | 8.66225847 | 8.151624446 | 0.25874373 | 8.35E−08 | 0.00087425 |
| KIAA0323 | 6.3479184 | 5.97380534 | 0.25868154 | 2.41E−08 | 0.00025588 |
| INSIG2 | 7.52022081 | 7.176137945 | 0.25854005 | 3.56E−07 | 0.00364485 |
| ITGB5 | 6.76443522 | 6.410670607 | 0.25849352 | 2.15E−08 | 0.00022936 |
| MTTP | 5.1346873 | 4.643056666 | 0.25837654 | 9.86E−14 | 1.14E−09 |
| LMBRD1 | 10.1665618 | 9.84233637 | 0.25819198 | 3.34E−07 | 0.00342644 |
| FAM82C | 8.34251549 | 8.043092309 | 0.257934 | 8.48E−07 | 0.00857428 |
| AP2A2 | 7.10866737 | 6.850439163 | 0.25751357 | 1.68E−07 | 0.0017351 |
| CRIM1 | 7.18518083 | 6.738536201 | 0.2573357 | 3.42E−07 | 0.00349678 |
| LAMC3 | 5.46764907 | 5.136218265 | 0.25726803 | 4.92E−08 | 0.00051946 |
| GIMAP5 | 6.46329409 | 6.164347065 | 0.25718746 | 9.60E−08 | 0.00100157 |
| ECM1 | 6.31279067 | 5.863641682 | 0.25709731 | 2.21E−10 | 2.46E−06 |
| DPP8 | 9.04803996 | 8.742652546 | 0.2570684 | 2.56E−07 | 0.00263332 |
| ATP6AP2 | 8.24693874 | 8.09387425 | 0.25681165 | 3.75E−08 | 0.00039627 |
| ETF1 | 8.51036664 | 8.259758271 | 0.25642081 | 8.71E−09 | 9.37E−05 |
| SOAT1 | 6.21367365 | 5.801081982 | 0.25637261 | 9.38E−09 | 0.00010086 |
| TUBA1C | 12.0568657 | 11.82951215 | 0.256358 | 4.74E−07 | 0.00482877 |
| NRP2 | 4.49022901 | 4.291091184 | 0.25618367 | 5.65E−10 | 6.22E−06 |
| LAMC1 | 8.24217367 | 7.827294031 | 0.25616833 | 1.53E−07 | 0.001588 |
| NLRP3 | 4.11199999 | 4.026681231 | 0.25611547 | 3.61E−08 | 0.0003819 |
| TMED9 | 8.80705994 | 8.550132942 | 0.25609833 | 4.18E−10 | 4.62E−06 |
| LEF1 | 5.55423593 | 5.295966727 | 0.25602707 | 7.65E−08 | 0.00080181 |
| B2M | 12.9124339 | 12.70122705 | 0.25591105 | 2.52E−07 | 0.00258794 |
| LRRC16 | 6.13766744 | 5.69657184 | 0.25584265 | 9.06E−08 | 0.0009456 |
| ZMYM6 | 6.44443767 | 6.18520342 | 0.25579289 | 8.14E−08 | 0.00085233 |
| ZNF226 | 7.48751893 | 7.012439556 | 0.25492256 | 1.48E−07 | 0.001538 |
| TWF2 | 5.91609115 | 5.719442126 | 0.25482441 | 5.00E−08 | 0.00052711 |
| TADA3L | 6.07868426 | 5.915186789 | 0.25477044 | 5.46E−07 | 0.00555279 |
| CSTB | 10.9232018 | 10.64503129 | 0.25473309 | 1.15E−11 | 1.29E−07 |
| S100A4 | 8.24580858 | 7.547710543 | 0.25472559 | 3.72E−10 | 4.10E−06 |
| PCDH8 | 6.11660496 | 5.388609235 | 0.25451548 | 2.71E−08 | 0.00028734 |
| CFLAR | 6.64126872 | 6.334515124 | 0.25431686 | 1.23E−08 | 0.00013217 |
| LYZ | 8.42891796 | 7.592593483 | 0.25426173 | 1.22E−08 | 0.00013065 |
| HPS5 | 7.1233975 | 6.849315391 | 0.25415897 | 1.87E−07 | 0.00192744 |
| NOD2 | 5.04560812 | 4.756139231 | 0.25402135 | 1.34E−09 | 1.47E−05 |
| HLA-F | 8.89788603 | 8.567519869 | 0.25395733 | 7.85E−08 | 0.00082247 |
| GRIK1 | 5.37658316 | 4.872269211 | 0.25374665 | 7.17E−10 | 7.88E−06 |
| BCL7B | 7.13431233 | 6.853597861 | 0.2536546 | 2.04E−07 | 0.00210374 |
| FAM49A | 6.37198923 | 6.007084309 | 0.25315998 | 8.73E−08 | 0.0009125 |
| PRRX1 | 6.00206334 | 5.472151738 | 0.25297214 | 3.64E−08 | 0.00038464 |
| PDLIM2 | 6.9532225 | 6.625865842 | 0.25284485 | 2.15E−08 | 0.00022853 |
| GALNAC4S-6ST | 7.78479868 | 7.263450406 | 0.25266469 | 5.57E−07 | 0.00566527 |
| FLII | 6.76288461 | 6.494345247 | 0.25263571 | 1.17E−08 | 0.00012574 |
| IDUA | 4.48964129 | 4.397078707 | 0.25259566 | 2.66E−07 | 0.00273631 |
| PTPN18 | 6.65989702 | 6.366748002 | 0.25218345 | 6.94E−07 | 0.00703605 |
| CXCL6 | 4.49646818 | 4.08976771 | 0.25215279 | 4.54E−07 | 0.00463546 |
| FYCO1 | 6.75028952 | 6.407095832 | 0.25173807 | 1.30E−07 | 0.00134805 |
| CHST1 | 6.56993633 | 6.035618517 | 0.25132899 | 1.44E−08 | 0.00015399 |
| RGS10 | 7.09578218 | 6.706873082 | 0.25109898 | 1.65E−07 | 0.00170477 |
| CAPRIN2 | 7.84411382 | 7.433007954 | 0.25101917 | 5.55E−07 | 0.00564748 |
| ALDH3B1 | 5.38950926 | 5.127070986 | 0.2505393 | 1.69E−14 | 1.95E−10 |
| SLC39A1 | 7.48839101 | 7.236993755 | 0.25047119 | 2.14E−08 | 0.00022768 |
| WARS | 8.18248477 | 7.755818563 | 0.25035527 | 3.25E−09 | 3.53E−05 |
| EYA2 | 6.15562601 | 5.652183801 | 0.25028471 | 9.29E−10 | 1.02E−05 |
| TLN2 | 6.09724086 | 5.786865532 | 0.25020029 | 1.20E−07 | 0.00125083 |
| LASS2 | 9.02431286 | 8.742668152 | 0.24968908 | 2.30E−09 | 2.51E−05 |
| PPBP | 5.07208506 | 4.479493854 | 0.24930133 | 1.37E−08 | 0.00014652 |
| MAP3K5 | 6.87593884 | 6.446228356 | 0.24926097 | 6.84E−07 | 0.00694201 |
| CSNK1D | 7.20877254 | 6.934600609 | 0.24925634 | 1.03E−07 | 0.00107578 |
| RETSAT | 7.40438025 | 7.107148532 | 0.24916899 | 3.17E−07 | 0.00325317 |
| PARP4 | 8.0414263 | 7.736784283 | 0.24912011 | 3.49E−08 | 0.00036928 |
| TMED1 | 7.33754152 | 7.054300669 | 0.24896252 | 4.21E−07 | 0.00429821 |
| MTHFS | 6.7838363 | 6.536354685 | 0.248672 | 6.01E−09 | 6.49E−05 |
| NOL3 | 6.07372662 | 5.731776319 | 0.24849617 | 1.94E−07 | 0.00200289 |
| TNS1 | 6.24956853 | 5.994560883 | 0.24811004 | 1.85E−07 | 0.00190763 |
| CLPB | 6.17740856 | 5.896700791 | 0.24771584 | 5.55E−07 | 0.00564748 |
| RTN4 | 11.8689277 | 11.65156494 | 0.24755078 | 7.38E−07 | 0.00747165 |
| C19orf28 | 7.60608203 | 7.243138532 | 0.2474223 | 3.96E−08 | 0.000419 |
| TMEM127 | 7.30242221 | 7.067547927 | 0.24727744 | 4.54E−07 | 0.00463546 |
| KIAA0152 | 8.85105533 | 8.520052488 | 0.24714365 | 8.48E−08 | 0.00088691 |
| TRPM3 | 4.62351943 | 4.398813409 | 0.24713301 | 7.59E−08 | 0.00079609 |
| TBXA2R | 4.84581128 | 4.717627294 | 0.24707734 | 6.46E−08 | 0.00067855 |
| EFHC1 | 7.56203465 | 7.126251948 | 0.2468971 | 4.70E−07 | 0.00479641 |
| NOX4 | 5.97933343 | 5.533150533 | 0.24686699 | 2.81E−07 | 0.00288241 |
| GMIP | 5.71886062 | 5.485173834 | 0.24661058 | 5.55E−07 | 0.00564748 |
| ABCD1 | 4.9757251 | 4.791698208 | 0.24654781 | 1.74E−07 | 0.00179718 |
| FGFR1 | 5.98778452 | 5.760517657 | 0.24652939 | 3.88E−09 | 4.20E−05 |
| CALU | 8.35484736 | 7.938487866 | 0.24652154 | 3.32E−07 | 0.00340319 |
| STAT5A | 5.23815255 | 5.021817149 | 0.24641816 | 1.20E−08 | 0.00012867 |
| HLA-DQB1 | 7.82439393 | 7.122238581 | 0.24628739 | 5.24E−07 | 0.00533057 |
| SSFA2 | 8.90489571 | 8.501204899 | 0.24621968 | 8.42E−07 | 0.00851722 |
| TNFSF4 | 5.39383357 | 5.039150929 | 0.2458152 | 2.76E−10 | 3.05E−06 |
| P4HA2 | 6.92846886 | 6.37755501 | 0.24569754 | 1.21E−08 | 0.00012965 |
| PLD3 | 7.10727701 | 6.845737825 | 0.24514804 | 1.20E−07 | 0.00125083 |
| AP2S1 | 10.0722305 | 9.844685129 | 0.24507897 | 7.18E−07 | 0.00727582 |
| ACTG1 | 13.0077038 | 12.84028523 | 0.2450041 | 7.28E−07 | 0.00737129 |
| NQO2 | 8.24687881 | 7.907705087 | 0.24485479 | 2.39E−07 | 0.00246307 |
| CDH5 | 6.97767855 | 6.489590512 | 0.24484681 | 9.74E−08 | 0.00101581 |
| VASP | 6.61690122 | 6.397137577 | 0.24482181 | 3.62E−08 | 0.00038323 |
| IRAK3 | 4.76447655 | 4.541256074 | 0.24460161 | 2.24E−10 | 2.49E−06 |
| SEC61G | 11.7612916 | 11.16235763 | 0.24432616 | 3.41E−08 | 0.00036116 |
| IRF1 | 6.55594519 | 6.14280272 | 0.24425421 | 2.41E−09 | 2.62E−05 |
| RNF14 | 7.72468183 | 7.372639189 | 0.2441513 | 5.01E−07 | 0.00509929 |
| OR2F2 | 4.06850787 | 3.991083201 | 0.24381571 | 3.70E−07 | 0.00378461 |
| AMD1 | 9.01536001 | 8.677581682 | 0.24364264 | 7.29E−08 | 0.00076509 |
| CHST12 | 7.43652484 | 7.154048588 | 0.24354952 | 9.81E−10 | 1.07E−05 |
| SERINC3 | 8.97686377 | 8.720532667 | 0.24315266 | 1.04E−07 | 0.00108739 |
| IL1R1 | 5.26355877 | 4.962474776 | 0.24304655 | 1.62E−10 | 1.80E−06 |
| ACSL1 | 7.63751783 | 7.158439628 | 0.24290357 | 3.85E−07 | 0.00393068 |
| COL3A1 | 9.27078333 | 8.326587174 | 0.24288062 | 1.81E−07 | 0.00186806 |
| IL1R2 | 5.40342449 | 4.863102148 | 0.24264345 | 3.29E−09 | 3.57E−05 |
| PTPN14 | 4.49234842 | 4.338481869 | 0.24259289 | 1.07E−09 | 1.17E−05 |
| PHF15 | 5.40689709 | 5.180914072 | 0.24237949 | 1.77E−07 | 0.0018353 |
| SNTA1 | 6.38783667 | 5.973881684 | 0.24231237 | 3.35E−08 | 0.00035458 |
| THBS4 | 7.61576932 | 6.897218326 | 0.24224785 | 7.91E−08 | 0.00082822 |
| PIM1 | 5.78504718 | 5.503677607 | 0.24195795 | 3.44E−08 | 0.00036383 |
| ARL8B | 10.5053594 | 10.28529635 | 0.24185137 | 2.61E−07 | 0.00267947 |
| DNALI1 | 6.08928235 | 5.741484991 | 0.2418403 | 2.34E−07 | 0.00241266 |
| LOXL2 | 5.39713397 | 5.208874709 | 0.24176493 | 6.97E−11 | 7.78E−07 |
| FLT3LG | 4.30811696 | 4.137858182 | 0.24158595 | 2.27E−08 | 0.00024177 |
| G0S2 | 6.41146209 | 5.898124377 | 0.24145338 | 6.01E−07 | 0.00610454 |
| KIAA0828 | 6.74520253 | 6.389448443 | 0.24143881 | 4.79E−07 | 0.00487865 |
| SERPINB1 | 6.99377922 | 6.448096041 | 0.24134914 | 1.80E−08 | 0.00019205 |
| UBE1L | 6.02632107 | 5.739572055 | 0.24115392 | 4.18E−08 | 0.00044154 |
| IL18 | 5.5921916 | 5.154793837 | 0.24105619 | 6.80E−08 | 0.00071401 |
| PCDH12 | 6.2837303 | 5.948798304 | 0.24083221 | 4.80E−07 | 0.00489459 |
| SEC61A1 | 9.19481907 | 8.920577084 | 0.24081055 | 8.51E−08 | 0.00088999 |
| IFNAR1 | 4.88662145 | 4.703785627 | 0.24068516 | 9.35E−07 | 0.00944076 |
| PDIA3 | 10.2573595 | 9.948563597 | 0.24064205 | 1.20E−08 | 0.00012867 |
| CA3 | 6.08938065 | 5.262527394 | 0.24040879 | 8.05E−09 | 8.67E−05 |
| IFT122 | 6.96941079 | 6.663320927 | 0.24038464 | 2.06E−07 | 0.00212592 |
| TRIP10 | 6.11391595 | 5.834801374 | 0.240254 | 5.84E−08 | 0.00061476 |
| PXN | 5.41523428 | 5.257699231 | 0.24013286 | 3.82E−09 | 4.14E−05 |
| CA9 | 6.07800723 | 5.553179458 | 0.23996642 | 3.99E−09 | 4.32E−05 |
| CAMK2B | 5.99664684 | 5.395938623 | 0.23986089 | 9.64E−08 | 0.00100514 |
| WHDC1L1 | 4.83770884 | 4.592064057 | 0.23974274 | 4.78E−08 | 0.00050443 |
| ZCCHC6 | 7.35430715 | 7.052193223 | 0.23962985 | 9.64E−07 | 0.00972307 |
| ARHGAP15 | 6.17499922 | 5.773255586 | 0.23916711 | 9.12E−08 | 0.00095245 |
| HGSNAT | 7.41807616 | 7.044163953 | 0.2389064 | 1.41E−07 | 0.00145766 |
| LITAF | 9.21642738 | 8.892650195 | 0.23867288 | 8.86E−07 | 0.00895375 |
| DENNDIC | 4.84727233 | 4.66599622 | 0.23803999 | 2.50E−09 | 2.72E−05 |
| GSTM4 | 6.02133028 | 5.726720355 | 0.23775989 | 9.10E−07 | 0.00919279 |
| SERGEF | 5.19740502 | 5.061122975 | 0.23723982 | 2.84E−07 | 0.00291275 |
| SGCB | 7.94543741 | 7.545504984 | 0.23707772 | 3.04E−07 | 0.003122 |
| RBPMS | 5.59847755 | 5.278763441 | 0.23693768 | 2.46E−08 | 0.00026169 |
| OLFML2B | 6.56773955 | 6.060774282 | 0.23692995 | 9.80E−07 | 0.00988339 |
| COL4A3BP | 7.4497961 | 7.160880732 | 0.23674128 | 1.84E−07 | 0.00190101 |
| ISG20 | 6.31237427 | 5.873243371 | 0.23630744 | 2.71E−10 | 3.00E−06 |
| 9-Sep | 8.20064007 | 7.942027606 | 0.23607217 | 1.22E−07 | 0.00127296 |
| FLNC | 7.18972621 | 6.637294098 | 0.23602065 | 6.11E−08 | 0.00064223 |
| MAP2K3 | 5.98555587 | 5.769919187 | 0.23593701 | 6.98E−08 | 0.00073248 |
| TBC1D2B | 6.51848801 | 6.186048921 | 0.23578281 | 7.58E−07 | 0.00767633 |
| GATAD1 | 7.89079103 | 7.571812484 | 0.23521862 | 6.80E−07 | 0.00689545 |
| FLJ20254 | 7.34151478 | 7.047680753 | 0.23505849 | 1.05E−07 | 0.00109125 |
| PDK3 | 5.16342285 | 4.935202198 | 0.23459182 | 1.22E−09 | 1.34E−05 |
| AOAH | 5.47353787 | 5.160177552 | 0.23446643 | 3.32E−07 | 0.00340319 |
| VCL | 6.76154237 | 6.563017266 | 0.234096 | 1.18E−08 | 0.00012671 |
| LEPREL1 | 6.21583724 | 5.770324114 | 0.23408115 | 8.80E−10 | 9.66E−06 |
| FZD5 | 5.50806056 | 5.226257876 | 0.23399712 | 1.35E−07 | 0.00140189 |
| SAP30L | 6.54047832 | 6.31453555 | 0.23395176 | 1.71E−07 | 0.0017723 |
| LENG4 | 6.48060079 | 6.30842603 | 0.23349257 | 9.36E−08 | 0.00097685 |
| NR1D1 | 6.39859464 | 6.122587313 | 0.2333343 | 5.59E−08 | 0.00058833 |
| CDH4 | 5.75281962 | 5.235869301 | 0.23287208 | 3.85E−09 | 4.17E−05 |
| LAMB1 | 7.55068879 | 6.95586805 | 0.23271937 | 1.16E−07 | 0.00121137 |
| CYP1B1 | 6.136804 | 5.531286614 | 0.23259924 | 7.59E−08 | 0.00079609 |
| TNFRSF14 | 5.33767059 | 5.086480832 | 0.23232555 | 1.37E−09 | 1.49E−05 |
| MCC | 5.49518458 | 5.135207122 | 0.23197084 | 7.00E−08 | 0.00073505 |
| GPI | 9.73442817 | 9.374550235 | 0.23190687 | 3.12E−07 | 0.00319752 |
| RNH1 | 8.61028882 | 8.343909484 | 0.23179508 | 2.33E−07 | 0.00240436 |
| SELL | 6.08473279 | 5.549666985 | 0.23172581 | 5.64E−09 | 6.10E−05 |
| FUCA1 | 8.58531502 | 8.177632193 | 0.23153445 | 3.24E−07 | 0.00332202 |
| PCOLCE | 7.29595943 | 6.682380979 | 0.23128299 | 1.38E−07 | 0.00142714 |
| CRELD2 | 8.13739488 | 7.878101479 | 0.23107381 | 1.94E−07 | 0.00200289 |
| EDNRA | 6.82891467 | 6.343596416 | 0.2307039 | 8.68E−07 | 0.00877644 |
| VNN2 | 5.23838235 | 4.830396147 | 0.2302543 | 6.96E−09 | 7.50E−05 |
| DNAJC3 | 6.2549009 | 6.041910259 | 0.23013284 | 4.89E−08 | 0.00051565 |
| NFE2L2 | 10.2967907 | 10.04897667 | 0.23012335 | 5.49E−08 | 0.00057783 |
| SLC16A6 | 5.32082894 | 5.017437211 | 0.22918528 | 1.80E−09 | 1.96E−05 |
| LRCH4 | 5.62909759 | 5.508144053 | 0.22890469 | 1.63E−07 | 0.00169291 |
| COL5A1 | 6.37861417 | 5.745074102 | 0.22866622 | 1.39E−09 | 1.52E−05 |
| DPP4 | 4.86721296 | 4.494208474 | 0.22803815 | 1.47E−10 | 1.64E−06 |
| MCL1 | 7.22008949 | 6.976230769 | 0.22788575 | 5.93E−08 | 0.00062371 |
| OAZ2 | 8.16107571 | 7.92242378 | 0.22770711 | 9.61E−07 | 0.00969227 |
| CLPTM1 | 6.96632744 | 6.678572329 | 0.2272014 | 9.38E−07 | 0.0094708 |
| IL32 | 6.08146531 | 5.635410607 | 0.22682952 | 4.57E−10 | 5.04E−06 |
| PALMD | 6.14581705 | 5.749413686 | 0.22650545 | 9.96E−08 | 0.00103799 |
| BAX | 6.09340955 | 5.802055392 | 0.22631352 | 3.90E−07 | 0.00398467 |
| DNASE2 | 6.9146205 | 6.674145696 | 0.22622914 | 5.18E−07 | 0.00527623 |
| DNAH9 | 4.8634463 | 4.57986864 | 0.22479546 | 8.44E−09 | 9.08E−05 |
| IRF7 | 6.4777779 | 6.170338278 | 0.22438723 | 3.54E−08 | 0.00037481 |
| ZNF576 | 6.46018078 | 6.220946783 | 0.22429833 | 9.32E−07 | 0.00940988 |
| GLG1 | 9.66839584 | 9.336173076 | 0.22407495 | 1.87E−07 | 0.00192744 |
| CIITA | 4.37171942 | 4.289034308 | 0.2237424 | 2.03E−07 | 0.00208901 |
| CNOT2 | 8.54521398 | 8.25594173 | 0.22357389 | 4.48E−07 | 0.00457254 |
| VRK3 | 6.91839736 | 6.707448659 | 0.22353974 | 3.42E−07 | 0.00349678 |
| HERPUD1 | 9.75789678 | 9.492019531 | 0.22254623 | 4.47E−07 | 0.00455717 |
| CCL20 | 5.30413656 | 4.69535749 | 0.22201264 | 1.71E−12 | 1.94E−08 |
| P2RY6 | 4.95219415 | 4.728683648 | 0.22095688 | 4.80E−13 | 5.50E−09 |
| NEO1 | 6.57300595 | 6.253817228 | 0.22094878 | 9.74E−07 | 0.00981994 |
| RAB21 | 8.40054364 | 8.100212545 | 0.22039 | 1.59E−07 | 0.00165138 |
| FURIN | 5.87940649 | 5.703244496 | 0.22006865 | 4.21E−07 | 0.00429821 |
| SLC16A3 | 5.6428811 | 5.34001456 | 0.21991975 | 3.23E−10 | 3.57E−06 |
| SAA4 | 4.53651357 | 4.370692991 | 0.21981924 | 5.73E−07 | 0.00582094 |
| SLC39A8 | 5.60657776 | 5.322158618 | 0.21940333 | 1.19E−11 | 1.35E−07 |
| IL21R | 4.50672821 | 4.321140304 | 0.21892931 | 5.71E−09 | 6.17E−05 |
| SFRP4 | 6.22366956 | 5.68496153 | 0.21879843 | 5.36E−08 | 0.0005653 |
| DKFZp434K191 | 4.5202936 | 4.32881098 | 0.21808378 | 4.18E−07 | 0.00426928 |
| TNXB | 5.15009514 | 5.056560246 | 0.21713313 | 8.08E−08 | 0.00084634 |
| CRYBB1 | 4.62294164 | 4.469718263 | 0.2165404 | 1.73E−07 | 0.00179093 |
| SLC6A9 | 4.93132926 | 4.672047235 | 0.21596296 | 9.71E−08 | 0.0010123 |
| PPP1R3D | 5.3399953 | 5.125570282 | 0.21530666 | 6.22E−07 | 0.00631556 |
| SGEF | 5.62767269 | 5.247816625 | 0.21463935 | 1.55E−07 | 0.00161068 |
| GNG11 | 8.82157586 | 8.412240196 | 0.21415837 | 9.48E−07 | 0.00956525 |
| CKMT2 | 5.28171379 | 5.030111499 | 0.21302488 | 2.00E−08 | 0.0002135 |
| FLJ22662 | 6.67841967 | 6.228802865 | 0.21279418 | 2.35E−09 | 2.56E−05 |
| EMX2 | 5.82671531 | 5.154465736 | 0.21261661 | 8.99E−08 | 0.0009388 |
| IER3 | 7.76079262 | 7.172496236 | 0.21217048 | 6.00E−08 | 0.0006305 |
| FLOT2 | 6.25743926 | 6.04600731 | 0.21035388 | 3.62E−07 | 0.00370798 |
| NAGPA | 5.87984785 | 5.684490133 | 0.21001189 | 5.65E−07 | 0.00574316 |
| HPD | 4.24771116 | 4.045613193 | 0.20972522 | 3.03E−08 | 0.00032154 |
| LY75 | 5.77410466 | 5.317958927 | 0.20941954 | 4.79E−07 | 0.00487865 |
| NPEPL1 | 5.7042506 | 5.55062027 | 0.2080573 | 1.03E−07 | 0.00107578 |
| ERBB2 | 5.30640469 | 5.116096032 | 0.20715344 | 6.96E−07 | 0.0070594 |
| TNFRSF10B | 5.76428156 | 5.514007823 | 0.20686884 | 7.88E−08 | 0.00082542 |
| GDF15 | 5.10804143 | 4.873577672 | 0.20630586 | 1.51E−08 | 0.00016122 |
| UBD | 6.26374467 | 5.610704515 | 0.20557985 | 2.15E−09 | 2.34E−05 |
| FPRL1 | 4.17974928 | 4.08494769 | 0.20548385 | 1.14E−08 | 0.0001224 |
| ANXA4 | 8.15489665 | 7.729063559 | 0.20529205 | 1.52E−07 | 0.00157111 |
| CDH6 | 5.04390197 | 4.840001069 | 0.20469397 | 3.27E−11 | 3.67E−07 |
| ITGAX | 5.39606553 | 5.155985847 | 0.20349445 | 2.78E−07 | 0.00285266 |
| GPR37L1 | 5.25080216 | 5.016515749 | 0.20191233 | 6.32E−07 | 0.00642338 |
| EBI3 | 4.96913266 | 4.729601861 | 0.20175465 | 1.72E−08 | 0.00018348 |
| PML | 4.90144388 | 4.786998291 | 0.20135051 | 6.25E−08 | 0.00065652 |
| RAB27A | 6.31686424 | 5.939320509 | 0.20076276 | 7.68E−11 | 8.57E−07 |
| C9orf116 | 5.79515057 | 5.535695535 | 0.19993087 | 8.73E−08 | 0.0009125 |
| TMEM109 | 7.15472745 | 6.903725067 | 0.19983546 | 7.25E−07 | 0.00734695 |
| CUL7 | 5.60127647 | 5.437394795 | 0.19920125 | 1.02E−07 | 0.0010643 |
| PTGS2 | 5.45455805 | 4.945962382 | 0.1987489 | 5.92E−09 | 6.39E−05 |
| CSF2RB | 5.55348742 | 5.215008918 | 0.19871553 | 7.92E−07 | 0.00801911 |
| AFF1 | 5.49954899 | 5.405585031 | 0.1986133 | 9.41E−07 | 0.00950187 |
| SPAG4 | 5.58780787 | 5.19491659 | 0.19781546 | 4.57E−08 | 0.00048256 |
| LTBP2 | 6.81871531 | 6.380627943 | 0.19676842 | 9.20E−09 | 9.89E−05 |
| CLEC4A | 5.36690653 | 5.099370861 | 0.19661562 | 6.96E−07 | 0.0070594 |
| GAS6 | 6.75609783 | 6.479313292 | 0.19631751 | 4.15E−07 | 0.00424013 |
| ANPEP | 4.69158412 | 4.340862198 | 0.19429943 | 5.67E−08 | 0.00059701 |
| IKZF1 | 4.89190658 | 4.760171629 | 0.1941751 | 4.82E−08 | 0.00050811 |
| CXCL5 | 4.55256666 | 4.167150108 | 0.19378325 | 2.52E−09 | 2.74E−05 |
| LCP1 | 6.76556461 | 6.422477725 | 0.19376154 | 5.55E−08 | 0.00058414 |
| HFE | 4.77001334 | 4.67045998 | 0.19358871 | 2.24E−08 | 0.00023813 |
| COL1A1 | 6.50828645 | 6.017162051 | 0.1935398 | 2.29E−08 | 0.00024356 |
| CD300C | 4.71538249 | 4.592402768 | 0.19292898 | 3.45E−08 | 0.00036516 |
| AIM1 | 6.18134351 | 5.67309044 | 0.19218243 | 2.00E−07 | 0.00206004 |
| SLPI | 7.36518168 | 6.588383304 | 0.19067981 | 3.68E−07 | 0.00375865 |
| SLFN12 | 4.96512436 | 4.701571409 | 0.18944529 | 9.57E−08 | 0.00099821 |
| PROCR | 5.98300312 | 5.722281719 | 0.18893275 | 8.51E−08 | 0.00088999 |
| CCDC46 | 5.44978997 | 5.181949385 | 0.18838762 | 1.60E−08 | 0.00017131 |
| PIK3CD | 5.14486508 | 5.023336179 | 0.18780695 | 1.35E−08 | 0.00014486 |
| TRPV2 | 5.02250077 | 4.828043415 | 0.18770239 | 1.19E−07 | 0.00123754 |
| EAF2 | 5.87089661 | 5.591052479 | 0.18702499 | 7.50E−07 | 0.00759914 |
| CASP8 | 5.56420001 | 5.341519294 | 0.18553284 | 1.23E−08 | 0.00013217 |
| TSPAN4 | 6.14182507 | 5.904312009 | 0.18453478 | 6.66E−09 | 7.19E−05 |
| FSTL3 | 5.14687989 | 4.953914168 | 0.18359318 | 1.39E−09 | 1.52E−05 |
| DDIT3 | 7.6092382 | 7.217317537 | 0.18346721 | 9.35E−07 | 0.00944076 |
| LMNA | 6.79002296 | 6.581233451 | 0.18153316 | 2.75E−08 | 0.00029166 |
| BNC2 | 5.24404082 | 4.937991252 | 0.17984303 | 5.65E−07 | 0.00574316 |
| OXA1L | 8.6622018 | 8.438485739 | 0.17797108 | 9.04E−07 | 0.00913269 |
| IGFBP1 | 4.96835818 | 4.652890255 | 0.1776759 | 1.17E−09 | 1.28E−05 |
| SERPINA5 | 5.49529518 | 5.176871526 | 0.1755949 | 3.80E−10 | 4.19E−06 |
| PRKCD | 5.35158936 | 5.122089694 | 0.1724013 | 3.81E−07 | 0.00389094 |
| CFB | 4.82479943 | 4.624080941 | 0.17162153 | 9.87E−09 | 0.00010603 |
| PF4V1 | 3.67780109 | 3.610110768 | 0.16713296 | 3.39E−07 | 0.00347375 |
| STXBP2 | 5.23504429 | 5.057281088 | 0.16609296 | 9.61E−07 | 0.00969227 |
| C4orf18 | 4.80205393 | 4.556029169 | 0.16115845 | 9.36E−08 | 0.00097685 |
| GALNT4 | 4.10392448 | 3.969996874 | 0.15881577 | 4.09E−09 | 4.43E−05 |
| TEAD3 | 4.86364034 | 4.719912513 | 0.15630205 | 9.04E−07 | 0.00913269 |
| KCTD14 | 4.94332352 | 4.688348302 | 0.15412004 | 8.59E−07 | 0.00868949 |
| MARCO | 4.82646429 | 4.547430221 | 0.15042033 | 1.15E−08 | 0.00012382 |
| PTPN7 | 4.86345137 | 4.752396645 | 0.14754025 | 3.87E−07 | 0.00395778 |
| MMP14 | 5.61610825 | 5.474220762 | 0.14696308 | 1.93E−07 | 0.00198884 |
| IL6 | 5.61938466 | 5.220046461 | 0.14110198 | 6.05E−07 | 0.00614535 |
| IL7R | 5.55587046 | 5.293172947 | 0.13666448 | 3.25E−08 | 0.00034411 |
| PHKG1 | 5.40976392 | 5.180225209 | 0.12917074 | 5.27E−07 | 0.00536691 |
| RAC2 | 5.95878988 | 5.761999295 | 0.12859824 | 1.23E−10 | 1.37E−06 |
| FBP1 | 5.59065016 | 5.381704569 | 0.12576577 | 5.95E−08 | 0.00062591 |
| PTHR1 | 4.73785018 | 4.609425331 | 0.12196801 | 9.13E−07 | 0.00922298 |
| CDCP1 | 5.22629628 | 5.006850635 | 0.12114743 | 2.28E−08 | 0.00024267 |
| MAN1A1 | 4.64153407 | 4.497713952 | 0.11987707 | 2.00E−07 | 0.00206004 |
| IL1A | 4.40515513 | 4.260150972 | 0.11545085 | 1.07E−07 | 0.00111513 |
| KDELR3 | 5.78970995 | 5.58726221 | 0.11156176 | 7.08E−07 | 0.00717799 |
| TRAF3IP3 | 4.35404888 | 4.26981806 | 0.08925621 | 5.42E−07 | 0.00551577 |
| IL15RA | 4.99726025 | 4.907893182 | 0.07199878 | 3.94E−07 | 0.00402613 |
| VDR | 4.5893918 | 4.553478514 | 0.05482279 | 2.94E−07 | 0.00301563 |
| GPRC5A | 5.63125938 | 5.558846162 | 0.02693881 | 5.75E−07 | 0.0058404 |
| MUC1 | 5.24737679 | 5.260185835 | −0.0067934 | 6.38E−10 | 7.02E−06 |
| KYNU | 4.87352108 | 4.918700982 | −0.0296076 | 8.17E−08 | 0.00085538 |
| MARCKSL1 | 10.2935904 | 10.57307833 | −0.1341241 | 7.97E−07 | 0.00807215 |
| GHRH | 3.90443985 | 4.049732035 | −0.1636316 | 2.20E−07 | 0.00226557 |
| CBX1 | 8.57033841 | 8.799542274 | −0.1695763 | 8.39E−08 | 0.00087729 |
| MAGEH1 | 7.76134959 | 8.126288917 | −0.1723627 | 1.63E−07 | 0.00168701 |
| HMGB2 | 9.94166185 | 10.23539561 | −0.1803122 | 3.83E−07 | 0.00391777 |
| GP2 | 4.0926731 | 4.21189151 | −0.1818661 | 1.40E−08 | 0.00014936 |
| CLC | 4.01719067 | 4.191514086 | −0.1840334 | 6.70E−07 | 0.00680456 |
| AHSG | 4.16386073 | 4.352582329 | −0.1910634 | 1.96E−09 | 2.13E−05 |
| RHAG | 3.91827151 | 4.017467497 | −0.1913377 | 6.70E−07 | 0.00680456 |
| KLRC4 | 3.61852358 | 3.860005098 | −0.1965145 | 1.95E−11 | 2.20E−07 |
| GSTA4 | 9.6701478 | 10.00015628 | −0.196894 | 1.71E−12 | 1.94E−08 |
| PAIP2B | 5.20386519 | 5.521036337 | −0.1990691 | 1.28E−07 | 0.00132908 |
| ATP1A3 | 4.65353462 | 4.963582931 | −0.2032923 | 2.20E−07 | 0.00226557 |
| SATB1 | 8.21161059 | 8.676668153 | −0.205353 | 3.17E−09 | 3.44E−05 |
| PTPRS | 4.53713402 | 4.689227005 | 0.2059282 | 6.68E−07 | 0.00678202 |
| MUC13 | 4.08058477 | 4.266670981 | −0.2060094 | 2.34E−07 | 0.00241266 |
| POU4F2 | 3.75308039 | 3.901408469 | −0.2070968 | 5.21E−09 | 5.64E−05 |
| GNAL | 4.00037052 | 4.131072428 | −0.2090726 | 7.84E−07 | 0.00793939 |
| TOP2B | 9.58984589 | 9.875121816 | −0.2092588 | 2.79E−08 | 0.00029609 |
| POU4F1 | 4.2624976 | 4.630284042 | −0.2118528 | 3.48E−09 | 3.78E−05 |
| MLF1IP | 8.36929946 | 8.903038348 | −0.2121168 | 2.49E−08 | 0.00026465 |
| SIX6 | 4.29781278 | 4.63255082 | −0.212141 | 4.82E−07 | 0.00491106 |
| BCL2L14 | 3.93687287 | 4.069091113 | 0.2135512 | 4.82E−07 | 0.00491106 |
| FAAH | 4.43038013 | 4.606622622 | −0.2146484 | 1.84E−08 | 0.00019647 |
| TPX2 | 7.34972406 | 7.851020622 | 0.2160638 | 1.20E−07 | 0.00125083 |
| LAPTM4B | 9.98048233 | 10.32996041 | −0.2199328 | 1.17E−08 | 0.00012526 |
| CCNB2 | 7.43277712 | 7.965276937 | −0.220167 | 4.24E−07 | 0.0043269 |
| COLQ | 4.29488629 | 4.425654807 | −0.2213986 | 3.89E−09 | 4.22E−05 |
| PTCH1 | 4.9903461 | 5.220715782 | −0.2214792 | 1.55E−07 | 0.00160506 |
| MCM2 | 7.31198621 | 7.752686592 | −0.2215802 | 6.46E−07 | 0.00655535 |
| FGB | 4.18660404 | 4.468201887 | −0.2216122 | 3.51E−07 | 0.0035946 |
| RIMS1 | 4.70022639 | 4.829127035 | −0.2217448 | 8.44E−10 | 9.27E−06 |
| ASPSCR1 | 5.50673472 | 5.733346498 | −0.2228586 | 1.66E−07 | 0.00171671 |
| SMARCE1 | 9.09452624 | 9.291339295 | −0.2233141 | 5.49E−08 | 0.00057783 |
| ACACA | 7.74893333 | 8.033450141 | −0.2243451 | 5.67E−07 | 0.00576181 |
| CLIC5 | 4.16917597 | 4.430906775 | −0.2260797 | 1.26E−09 | 1.37E−05 |
| KCNK7 | 4.04711715 | 4.127027814 | −0.2270886 | 3.38E−07 | 0.00346194 |
| MAGEC1 | 3.87948231 | 4.036533377 | −0.2274282 | 1.86E−07 | 0.00192076 |
| HOXC8 | 4.53827181 | 4.672480717 | −0.227489 | 4.56E−07 | 0.00465017 |
| CCT5 | 10.1313942 | 10.39832632 | −0.227623 | 5.75E−07 | 0.0058404 |
| KIAA0907 | 8.75646697 | 9.04215897 | −0.2281126 | 1.29E−07 | 0.00134331 |
| XRCC5 | 9.54849077 | 9.798517095 | −0.228177 | 4.69E−08 | 0.00049529 |
| TCP1 | 9.0739942 | 9.326570053 | −0.2287729 | 8.81E−09 | 9.48E−05 |
| HLTF | 8.60537199 | 8.904223585 | −0.2295456 | 2.02E−08 | 0.00021512 |
| BRCA2 | 4.28574133 | 4.477807258 | −0.2302112 | 1.25E−07 | 0.00130079 |
| WASF1 | 7.68487418 | 8.214850584 | −0.2303785 | 1.97E−08 | 0.00021032 |
| TMPRSS2 | 4.11521174 | 4.272096695 | −0.2311836 | 8.80E−07 | 0.00889515 |
| MEOX1 | 3.9414416 | 4.148289796 | −0.2315931 | 1.34E−08 | 0.00014375 |
| UBE2S | 7.68154046 | 8.129076866 | −0.2319868 | 1.91E−07 | 0.00196823 |
| CDC20 | 7.03452927 | 7.577673455 | −0.2322998 | 3.93E−08 | 0.0004159 |
| MAGEB4 | 4.07608466 | 4.132940872 | −0.2324068 | 3.40E−07 | 0.0034849 |
| PIK3R3 | 5.88384437 | 6.149732093 | −0.2324804 | 6.77E−07 | 0.00687262 |
| NOL11 | 8.28572502 | 8.533861333 | −0.2336381 | 1.48E−07 | 0.001538 |
| EZH2 | 6.98743005 | 7.513423128 | −0.2347982 | 1.64E−07 | 0.00169883 |
| GINS2 | 7.25612882 | 7.738097233 | −0.2348284 | 9.54E−07 | 0.00962903 |
| PAX1 | 3.85462642 | 4.010234528 | −0.2349656 | 8.05E−08 | 0.00084332 |
| HSD17B2 | 3.66376484 | 3.806743145 | 0.2350734 | 7.03E−07 | 0.00713061 |
| SLC22A3 | 3.64645671 | 3.858791879 | 0.2352362 | 1.09E−07 | 0.00113548 |
| TAF2 | 7.83486871 | 8.076094335 | −0.2358957 | 7.32E−08 | 0.00076784 |
| INTS8 | 8.17834127 | 8.416690555 | 0.2358996 | 9.54E−07 | 0.00962903 |
| TSHR | 4.03682706 | 4.220655569 | −0.2363002 | 6.98E−08 | 0.00073248 |
| CTBP2 | 8.29585832 | 8.546240988 | −0.2376175 | 8.79E−08 | 0.00091885 |
| CDC123 | 7.97387656 | 8.286473842 | −0.2378329 | 4.48E−07 | 0.00457254 |
| SFRS3 | 9.66024988 | 9.880941958 | −0.2380471 | 9.39E−08 | 0.00098024 |
| SMC3 | 7.48121595 | 7.850272232 | −0.2384852 | 2.30E−07 | 0.00237096 |
| DTL | 6.62164472 | 7.198558782 | −0.2385348 | 6.29E−08 | 0.00066134 |
| JAR1D1B | 6.98216783 | 7.325403789 | −0.2395912 | 3.11E−07 | 0.00318663 |
| EYA1 | 4.9223036 | 5.518914411 | −0.2399323 | 1.22E−07 | 0.00126401 |
| SRC | 4.72055416 | 4.810521253 | 0.2399831 | 4.58E−07 | 0.00466585 |
| RFC4 | 8.26511479 | 8.702870245 | −0.2413171 | 1.74E−07 | 0.00180345 |
| SGCD | 4.29242772 | 4.407443031 | −0.2414952 | 1.46E−08 | 0.00015576 |
| ARMC1 | 8.06517582 | 8.388878016 | 0.2415175 | 6.90E−08 | 0.00072449 |
| PQBP1 | 6.51440203 | 6.724224789 | −0.2420019 | 2.28E−07 | 0.00234654 |
| RACGAP1 | 8.07372846 | 8.508456047 | −0.2421085 | 1.79E−08 | 0.0001906 |
| SRPK3 | 4.71133773 | 4.881171523 | −0.2421534 | 3.37E−08 | 0.0003572 |
| UBE2C | 8.35277633 | 8.930107205 | −0.2431169 | 1.59E−09 | 1.74E−05 |
| VDAC3 | 8.06203923 | 8.252087007 | −0.2431581 | 7.23E−07 | 0.0073234 |
| ELAVL2 | 4.24473295 | 4.526012902 | −0.2431634 | 2.35E−07 | 0.00242053 |
| TAS2R14 | 3.81488686 | 3.90713267 | −0.2434109 | 1.90E−07 | 0.00196179 |
| TOPBP1 | 8.27647388 | 8.620676484 | −0.243811 | 3.58E−09 | 3.88E−05 |
| ZSCAN16 | 5.59556578 | 5.873597792 | −0.243949 | 3.44E−07 | 0.00352065 |
| NVL | 6.77253308 | 7.058994591 | −0.2440416 | 7.03E−07 | 0.00713061 |
| SS18L1 | 6.86725142 | 7.258392939 | −0.244122 | 9.64E−07 | 0.00972307 |
| HNRNPL | 8.52592588 | 8.808703584 | −0.2446771 | 7.27E−08 | 0.00076235 |
| C8orf51 | 4.42265505 | 4.532163748 | −0.2447545 | 8.95E−07 | 0.00904235 |
| B4GALT6 | 4.23249658 | 4.499357445 | −0.2454977 | 1.90E−07 | 0.00196179 |
| C11orf30 | 4.72161965 | 4.892602852 | −0.245614 | 5.16E−07 | 0.00525859 |
| HIST1H4L | 4.18491266 | 4.266161775 | −0.245627 | 3.37E−08 | 0.0003572 |
| BZW2 | 9.47773937 | 9.849832306 | −0.2462106 | 1.90E−07 | 0.00196179 |
| CKS2 | 9.29694661 | 9.817628757 | −0.2462404 | 5.97E−09 | 6.44E−05 |
| UBAP2 | 7.59140183 | 7.931374417 | −0.2470252 | 1.49E−07 | 0.00154325 |
| GPSM2 | 7.29012448 | 7.702231098 | −0.2472555 | 5.61E−08 | 0.00059046 |
| MSH5 | 4.8937923 | 5.20604013 | −0.2474571 | 1.40E−07 | 0.0014476 |
| NARS2 | 7.03958132 | 7.392474197 | −0.2474649 | 3.39E−07 | 0.00347375 |
| PAX9 | 3.94109369 | 4.038487827 | −0.2478323 | 9.29E−07 | 0.0093791 |
| KCNIP2 | 3.83038981 | 3.925658329 | −0.248363 | 1.62E−07 | 0.0016751 |
| NXN | 7.23939444 | 7.711038623 | −0.2490804 | 3.40E−08 | 0.00035984 |
| CKAP2 | 7.53708472 | 7.94900826 | −0.2490978 | 9.42E−09 | 0.00010124 |
| CRHR1 | 3.98975701 | 4.051759985 | −0.2498956 | 3.09E−07 | 0.00316495 |
| RAD51AP1 | 6.2049129 | 6.673162942 | −0.2499979 | 7.58E−07 | 0.00767633 |
| DNASE1 | 3.96444589 | 4.091295168 | −0.2500924 | 8.99E−10 | 9.86E−06 |
| CCNB1IP1 | 8.23011931 | 8.5965359 | −0.2510141 | 1.56E−07 | 0.00161633 |
| ZNF32 | 7.47222624 | 7.786649935 | −0.2515589 | 4.00E−07 | 0.00408218 |
| MMP17 | 4.10122078 | 4.201299179 | −0.2519237 | 2.19E−07 | 0.00225776 |
| C16orf80 | 9.39777199 | 9.72394053 | −0.2526514 | 1.09E−09 | 1.19E−05 |
| MTX2 | 7.63772309 | 7.931541447 | −0.2530173 | 3.34E−07 | 0.00342644 |
| AKAP1 | 6.19430025 | 6.419179344 | −0.253247 | 8.28E−07 | 0.00837577 |
| TMEM97 | 7.88477286 | 8.278972689 | −0.2532545 | 1.74E−08 | 0.00018559 |
| RCL1 | 6.40601425 | 6.671645188 | −0.2533042 | 7.23E−07 | 0.0073234 |
| IL26 | 4.0618069 | 4.148190416 | −0.2534555 | 1.42E−08 | 0.00015166 |
| GLRA3 | 3.79314759 | 3.869401154 | −0.2536178 | 1.79E−07 | 0.00185161 |
| PAQR5 | 3.93128935 | 4.032499493 | −0.2540352 | 7.18E−07 | 0.00727582 |
| KCNK1 | 5.5353769 | 6.188277721 | −0.2541111 | 6.87E−07 | 0.00696506 |
| LOC400506 | 6.30465502 | 6.567467526 | −0.254826 | 6.43E−07 | 0.0065336 |
| BNC1 | 3.63988912 | 3.891199993 | −0.2549905 | 6.50E−08 | 0.00068352 |
| HOXD1 | 4.7355617 | 5.012704998 | −0.2554362 | 1.31E−08 | 0.00013996 |
| HSPA14 | 7.48981045 | 7.855727629 | −0.2554888 | 2.28E−07 | 0.00234654 |
| TTC13 | 6.79077394 | 7.12914525 | −0.2555691 | 1.10E−07 | 0.00114771 |
| GTSE1 | 5.64256388 | 5.996606532 | −0.2559433 | 8.89E−07 | 0.00898319 |
| VASH2 | 4.57943196 | 4.957772922 | −0.2559843 | 1.16E−07 | 0.00120272 |
| MYH14 | 4.35053119 | 4.439842188 | −0.2560812 | 7.97E−08 | 0.00083416 |
| NCAPD2 | 6.54361622 | 6.968081075 | −0.2561375 | 2.66E−07 | 0.00273631 |
| CDSN | 4.12218177 | 4.199852948 | −0.2566954 | 6.22E−07 | 0.00631556 |
| CSE1L | 7.81858153 | 8.052477749 | −0.2568342 | 2.63E−08 | 0.00027885 |
| LEFTY1 | 4.62804674 | 4.829196807 | −0.2568824 | 1.00E−10 | 1.12E−06 |
| TIMELESS | 6.3769994 | 6.782199451 | −0.2568955 | 1.31E−07 | 0.00135744 |
| SLC38A1 | 7.76229568 | 8.430022836 | −0.2575534 | 1.14E−08 | 0.00012193 |
| SIX1 | 4.9156578 | 5.223935109 | −0.2575857 | 6.63E−08 | 0.00069606 |
| PTTG3 | 5.17455503 | 5.419770293 | −0.2583838 | 8.95E−07 | 0.00904235 |
| PCGF2 | 6.1679761 | 6.443403187 | −0.2584691 | 2.73E−07 | 0.00280336 |
| LOC541469 | 4.18521374 | 4.281789646 | −0.2586101 | 9.81E−08 | 0.00102315 |
| HNRPH3 | 6.96903479 | 7.278440839 | −0.2586143 | 6.75E−08 | 0.0007089 |
| NR4A1 | 5.30979853 | 5.486240869 | −0.2586505 | 6.73E−07 | 0.0068265 |
| E2F1 | 5.06497193 | 5.225587535 | −0.2587552 | 8.89E−07 | 0.00898319 |
| MKRN3 | 4.63241827 | 4.936244056 | −0.2588283 | 5.40E−07 | 0.00549736 |
| CENPM | 5.75767865 | 6.109481062 | −0.2590805 | 3.44E−07 | 0.00352065 |
| CCNJ | 4.7058224 | 4.998614132 | −0.2595081 | 3.03E−07 | 0.00311136 |
| ANKZF1 | 5.92368693 | 6.135195621 | −0.2595772 | 6.03E−07 | 0.00612491 |
| RPL35A | 11.3767515 | 11.61273746 | −0.2602275 | 1.19E−07 | 0.00124191 |
| PKP4 | 6.69778009 | 7.162850163 | −0.2602864 | 2.90E−08 | 0.00030741 |
| NASP | 6.96699585 | 7.351467155 | −0.2603605 | 2.30E−07 | 0.00237096 |
| FANCI | 6.49003885 | 7.05669194 | −0.260424 | 1.11E−07 | 0.00115597 |
| LOC196993 | 4.03333613 | 4.128831198 | −0.2608762 | 3.65E−07 | 0.00373287 |
| EDC4 | 6.13395885 | 6.357908698 | −0.2611709 | 2.99E−08 | 0.00031674 |
| STYK1 | 4.05131997 | 4.252490527 | −0.2613851 | 4.84E−07 | 0.00492662 |
| DLL3 | 5.84364609 | 6.657463864 | −0.2616999 | 4.54E−07 | 0.00463546 |
| SFRS6 | 7.85866644 | 8.163113644 | −0.2618425 | 2.20E−08 | 0.00023461 |
| TACC2 | 6.32277363 | 6.737619492 | −0.2619788 | 3.04E−07 | 0.003122 |
| COMMD3 | 8.81771845 | 9.150925415 | −0.26213 | 1.69E−07 | 0.00174725 |
| OSR2 | 4.39243236 | 4.836258953 | −0.26245 | 8.11E−08 | 0.00084929 |
| TOP2A | 7.97444481 | 8.696041943 | −0.2627593 | 8.53E−12 | 9.66E−08 |
| MCF2 | 3.87087256 | 3.978654752 | −0.26296 | 2.73E−07 | 0.00280336 |
| CKS1B | 8.6125857 | 9.150660208 | −0.2629729 | 1.02E−07 | 0.0010643 |
| MAGI1 | 4.70839157 | 5.151692119 | −0.2633869 | 9.92E−08 | 0.00103431 |
| ACTR5 | 5.2220885 | 5.40079579 | −0.2649785 | 2.72E−07 | 0.00279376 |
| NOLC1 | 6.5498394 | 6.805874522 | −0.2656666 | 7.95E−07 | 0.00804559 |
| DCC | 4.03090891 | 4.187831439 | 0.2658894 | 1.85E−07 | 0.00190763 |
| COQ3 | 6.16108884 | 6.512699081 | −0.2660698 | 3.83E−07 | 0.00391777 |
| PCDH21 | 4.89912259 | 5.127303905 | −0.2669027 | 7.24E−08 | 0.00075962 |
| RAB26 | 4.73987424 | 5.07863066 | −0.2669108 | 8.63E−08 | 0.00090287 |
| HSF2 | 5.4984985 | 5.790334863 | −0.2670472 | 1.18E−07 | 0.0012245 |
| HNRNPU | 6.31531649 | 6.426251621 | −0.2674108 | 2.49E−08 | 0.00026465 |
| RAD52 | 4.46852706 | 4.588500354 | −0.2676226 | 1.06E−07 | 0.00110715 |
| HIST3H2A | 6.41398352 | 7.093593625 | −0.267789 | 1.81E−07 | 0.00187439 |
| HOXA11 | 4.25198127 | 4.478031503 | −0.2680306 | 3.93E−09 | 4.25E−05 |
| RBMX | 8.97887462 | 9.280711187 | −0.2688365 | 1.62E−08 | 0.00017262 |
| LMNB1 | 6.1187111 | 6.637222017 | −0.2692445 | 7.88E−08 | 0.00082542 |
| STXBP6 | 4.52473897 | 5.007139284 | −0.269319 | 3.04E−07 | 0.003122 |
| NDST4 | 3.97424026 | 4.189731337 | −0.2693231 | 8.13E−10 | 8.93E−06 |
| UBE2I | 6.77032087 | 6.913531375 | 0.2694712 | 2.25E−07 | 0.00231369 |
| GPR85 | 4.44894211 | 4.617195866 | −0.2694913 | 7.54E−08 | 0.00079033 |
| ZNF323 | 5.67961046 | 6.035826017 | −0.269673 | 5.48E−07 | 0.00557138 |
| BIRC5 | 6.49667945 | 7.070036536 | 0.2697858 | 7.77E−09 | 8.37E−05 |
| KIT | 5.98549202 | 6.640778977 | −0.2698037 | 8.59E−07 | 0.00868949 |
| BAAT | 4.24790258 | 4.366650905 | −0.2702167 | 5.42E−07 | 0.00551577 |
| FXR1 | 8.20417391 | 8.506416193 | −0.2702407 | 8.86E−08 | 0.00092534 |
| PSMD4 | 7.17803584 | 7.328057547 | −0.2704575 | 1.34E−07 | 0.00139194 |
| EPHB3 | 5.12894775 | 5.554758177 | −0.2704661 | 4.18E−07 | 0.00426928 |
| MLLT10 | 4.7302404 | 4.915233273 | −0.2706549 | 6.18E−08 | 0.00064934 |
| RFC5 | 6.047329 | 6.461802971 | −0.2709404 | 4.04E−07 | 0.00412461 |
| CENPE | 5.5377087 | 6.036649871 | −0.2714848 | 8.32E−08 | 0.00087113 |
| ZSCAN2 | 3.96334751 | 4.054593049 | −0.2716837 | 2.37E−07 | 0.00243751 |
| CHD7 | 6.62887841 | 7.166700726 | −0.2717362 | 2.48E−09 | 2.70E−05 |
| RAD21 | 9.38328214 | 9.66335648 | −0.2718759 | 2.36E−10 | 2.62E−06 |
| DLG3 | 4.86106622 | 5.039873273 | −0.2719145 | 4.76E−08 | 0.0005026 |
| ITFG2 | 4.52539058 | 4.684767242 | −0.2721823 | 9.19E−08 | 0.00095935 |
| EIF2B5 | 6.92734834 | 7.183802984 | −0.2723738 | 6.88E−09 | 7.42E−05 |
| KLK1 | 4.39714982 | 4.530572588 | −0.2724213 | 1.18E−07 | 0.0012245 |
| LHX1 | 4.00854808 | 4.183029244 | −0.2726227 | 9.22E−08 | 0.00096277 |
| ISL1 | 4.23067623 | 4.411096902 | −0.2732584 | 6.21E−09 | 6.70E−05 |
| SIM2 | 4.35126008 | 4.610396889 | −0.2741933 | 9.64E−08 | 0.00100514 |
| MKI67 | 5.2410282 | 5.697843469 | −0.2742904 | 1.76E−07 | 0.00182257 |
| MXD3 | 5.41913433 | 5.726576733 | −0.2746356 | 7.00E−08 | 0.00073505 |
| ZWINT | 7.5686437 | 8.116005663 | −0.2747811 | 2.51E−09 | 2.73E−05 |
| TLX1 | 4.08739517 | 4.252216884 | −0.2748359 | 4.40E−08 | 0.00046503 |
| DKFZp762E1312 | 6.11000363 | 6.625391192 | −0.2755365 | 3.51E−08 | 0.00037204 |
| KIF15 | 5.61640679 | 6.191852203 | −0.275945 | 4.22E−08 | 0.00044648 |
| ILF2 | 9.32660746 | 9.707958141 | −0.2762422 | 8.61E−12 | 9.74E−08 |
| FCHO1 | 4.26584374 | 4.414496235 | 0.2765188 | 7.48E−08 | 0.00078461 |
| TRIT1 | 5.46590003 | 5.650106401 | −0.2765695 | 5.95E−07 | 0.00604263 |
| CDCA3 | 5.34892616 | 5.837142314 | −0.2770658 | 5.51E−07 | 0.00560931 |
| MEGF6 | 4.53297629 | 4.726623684 | −0.2772827 | 3.70E−09 | 4.01E−05 |
| DCT | 4.50059085 | 4.906044176 | −0.2775696 | 1.19E−10 | 1.32E−06 |
| AIM1L | 4.56206694 | 4.679667943 | −0.2785754 | 2.58E−07 | 0.00265148 |
| GDF9 | 4.15669423 | 4.31155494 | −0.2788584 | 1.26E−08 | 0.00013521 |
| KCNB2 | 3.75918092 | 3.854857533 | −0.2789074 | 6.80E−09 | 7.33E−05 |
| DLX4 | 4.06992268 | 4.151938234 | −0.2790253 | 1.77E−07 | 0.00182565 |
| C1orf35 | 5.51989679 | 5.699910702 | −0.2793526 | 1.72E−07 | 0.00178487 |
| HSPB3 | 4.46163857 | 4.838654969 | −0.2796097 | 4.26E−08 | 0.0004498 |
| PLCB1 | 5.6371092 | 5.963040549 | −0.2796191 | 6.74E−09 | 7.27E−05 |
| AZI1 | 5.21714304 | 5.407366391 | −0.2799095 | 2.62E−08 | 0.00027782 |
| DCX | 6.26846599 | 7.492678622 | −0.2801915 | 2.08E−07 | 0.00214069 |
| SFPQ | 8.23745549 | 8.500960328 | −0.2801985 | 4.29E−09 | 4.64E−05 |
| ATAD2 | 5.95224735 | 6.461116785 | −0.2805694 | 8.63E−08 | 0.00090287 |
| PFDN2 | 8.70707483 | 9.035888779 | −0.2808754 | 1.64E−08 | 0.00017528 |
| KIF14 | 5.39583921 | 5.857697012 | −0.2812359 | 8.08E−08 | 0.00084634 |
| TRIP13 | 6.38676028 | 6.969292325 | −0.2813578 | 3.27E−07 | 0.00335648 |
| ZNF750 | 3.6321787 | 3.93096377 | −0.2817624 | 1.43E−07 | 0.00148403 |
| GRID2 | 3.99069965 | 4.187308001 | −0.2817716 | 4.44E−08 | 0.00046847 |
| HMGCS1 | 6.04728527 | 6.475472755 | −0.2819943 | 4.93E−09 | 5.33E−05 |
| ZNF639 | 4.89737838 | 5.062256462 | −0.2820002 | 1.42E−08 | 0.00015166 |
| FANCG | 6.58021912 | 7.038424516 | −0.2821502 | 5.78E−08 | 0.00060802 |
| TFAP2A | 4.78079924 | 5.138877818 | −0.2825553 | 1.85E−08 | 0.00019719 |
| TDRKH | 4.06055024 | 4.208193816 | −0.2826921 | 7.40E−08 | 0.00077621 |
| COIL | 7.03441538 | 7.315896175 | −0.2827244 | 9.57E−09 | 0.00010279 |
| FGFR4 | 4.15008204 | 4.319518451 | −0.2832754 | 1.31E−09 | 1.43E−05 |
| GUCY1B2 | 4.07663575 | 4.211720733 | −0.2833609 | 3.89E−07 | 0.0039712 |
| CENPF | 6.68017236 | 7.312772307 | −0.2833742 | 2.58E−09 | 2.81E−05 |
| PAFAH1B3 | 7.65838591 | 8.125376049 | −0.2837097 | 5.92E−09 | 6.39E−05 |
| KIF11 | 5.78323012 | 6.400747659 | −0.2843204 | 4.39E−08 | 0.00046338 |
| SCML2 | 5.3872609 | 5.586262755 | −0.2843242 | 1.58E−08 | 0.00016937 |
| ECT2 | 6.67776808 | 7.291173934 | −0.2847391 | 1.49E−08 | 0.00015938 |
| RELN | 4.74504186 | 5.272870662 | −0.2848276 | 9.57E−08 | 0.00099821 |
| TEX10 | 7.00987895 | 7.382541392 | −0.2851686 | 3.75E−08 | 0.00039627 |
| ATP6V0A4 | 3.81763811 | 4.091180721 | −0.2853479 | 2.56E−09 | 2.78E−05 |
| C17orf75 | 5.71851947 | 6.125007943 | −0.2854577 | 1.40E−07 | 0.00145255 |
| KRTAP2-4 | 4.12786408 | 4.230834331 | −0.285709 | 4.47E−08 | 0.0004719 |
| PTGER3 | 4.02321735 | 4.080738588 | −0.2857965 | 5.55E−08 | 0.00058414 |
| NCAPG | 5.62237523 | 6.182000817 | −0.285857 | 9.13E−09 | 9.82E−05 |
| HIC2 | 4.76173236 | 4.945617657 | −0.2860591 | 6.50E−08 | 0.00068352 |
| POLR2B | 9.05126018 | 9.356510175 | −0.2860911 | 1.63E−10 | 1.81E−06 |
| ATP2C2 | 4.20351552 | 4.434157904 | −0.2863157 | 1.88E−09 | 2.05E−05 |
| GPR87 | 3.66778837 | 4.125683529 | −0.2864831 | 1.30E−07 | 0.00135267 |
| NEIL3 | 5.02587263 | 5.309478974 | −0.286525 | 1.71E−08 | 0.00018209 |
| PLEKHG3 | 4.81562869 | 4.932976986 | −0.2876877 | 1.75E−09 | 1.91E−05 |
| CTPS | 6.56843529 | 7.035700618 | −0.2883052 | 1.53E−08 | 0.00016366 |
| RBM4 | 7.14251627 | 7.383572538 | −0.2886416 | 2.02E−09 | 2.20E−05 |
| FANCE | 5.06393972 | 5.31724529 | −0.2889138 | 5.67E−08 | 0.00059701 |
| DFFB | 4.56258118 | 4.794287163 | −0.2889206 | 2.51E−08 | 0.00026659 |
| C13orf27 | 6.43124286 | 6.892480206 | −0.2892649 | 8.24E−09 | 8.87E−05 |
| C19orf40 | 4.59320985 | 4.758762902 | −0.289558 | 2.05E−08 | 0.0002184 |
| ICA1 | 4.93370885 | 5.160433094 | −0.2902755 | 2.91E−07 | 0.00298426 |
| HOXD9 | 4.32673264 | 4.460147554 | −0.2905399 | 1.93E−08 | 0.00020556 |
| RP4-691N24.1 | 5.4161712 | 5.822927339 | −0.2907941 | 8.79E−08 | 0.00091885 |
| AMOTL2 | 7.49560408 | 8.095385993 | −0.2908406 | 2.19E−09 | 2.38E−05 |
| BUB3 | 7.87952195 | 8.254228395 | −0.2915365 | 3.58E−09 | 3.88E−05 |
| OTUB2 | 4.17202762 | 4.353564352 | −0.2915504 | 2.69E−09 | 2.92E−05 |
| FUBP1 | 5.68176733 | 5.96116595 | −0.2916868 | 7.38E−09 | 7.96E−05 |
| C10orf95 | 4.00551666 | 4.10599219 | −0.2922399 | 2.47E−08 | 0.00026267 |
| NMU | 5.68793882 | 6.491283039 | −0.2923176 | 8.05E−09 | 8.67E−05 |
| ITGB6 | 3.71006185 | 3.966539859 | −0.292388 | 3.41E−09 | 3.70E−05 |
| KIAA1324 | 4.19470895 | 4.458068444 | −0.2924252 | 6.04E−09 | 6.52E−05 |
| B3GNT3 | 4.30769226 | 4.487148814 | −0.29244 | 9.80E−07 | 0.00988339 |
| NKX2-1 | 3.96701018 | 4.210123888 | −0.2924401 | 4.98E−11 | 5.57E−07 |
| ASPM | 6.3346036 | 7.068204817 | −0.2924703 | 7.96E−10 | 8.75E−06 |
| SLC6A14 | 3.59296788 | 3.928011437 | −0.2929545 | 8.33E−07 | 0.00843191 |
| ACVR2B | 5.16587207 | 5.464798317 | −0.2930543 | 4.05E−09 | 4.39E−05 |
| TSC22D2 | 5.06183179 | 5.170884158 | −0.2932168 | 7.07E−09 | 7.62E−05 |
| EDAR | 4.33002086 | 4.525766945 | −0.2934962 | 4.47E−11 | 5.01E−07 |
| PATZ1 | 4.9097621 | 5.092618705 | −0.2938704 | 1.09E−09 | 1.20E−05 |
| INTS7 | 6.21871496 | 6.623907646 | −0.293968 | 1.86E−08 | 0.00019869 |
| TNR | 4.16031557 | 4.271181642 | −0.2940388 | 2.74E−09 | 2.98E−05 |
| RALGPS1 | 4.96789536 | 5.286720464 | −0.2943149 | 4.79E−09 | 5.19E−05 |
| CDC2 | 6.54093013 | 7.224990561 | −0.2946629 | 1.53E−08 | 0.00016304 |
| CENPA | 5.78836575 | 6.307776502 | −0.2950219 | 1.75E−08 | 0.00018629 |
| TIAL1 | 6.21002007 | 6.390414428 | −0.2951962 | 1.32E−07 | 0.0013721 |
| ZSCAN21 | 4.02901794 | 4.140784287 | −0.2955116 | 1.30E−08 | 0.00013943 |
| ACRV1 | 3.92805101 | 4.072021958 | −0.2960558 | 8.86E−08 | 0.00092534 |
| PAPD1 | 6.31813396 | 6.717903612 | −0.2970263 | 3.61E−08 | 0.0003819 |
| LDB1 | 5.52605662 | 5.785578474 | −0.2970781 | 2.19E−09 | 2.38E−05 |
| CDC6 | 4.54470413 | 5.015019549 | −0.297859 | 1.18E−07 | 0.0012245 |
| DUSP26 | 5.43970995 | 5.919388883 | −0.2978833 | 2.49E−08 | 0.00026465 |
| TEX14 | 4.00572958 | 4.27169926 | −0.2984685 | 4.92E−08 | 0.00051946 |
| CEP152 | 4.43252695 | 4.739901496 | −0.2985852 | 7.11E−08 | 0.00074582 |
| TCF20 | 5.25192404 | 5.394157691 | −0.2987379 | 1.83E−08 | 0.00019574 |
| ARVCF | 5.30114531 | 5.466220693 | −0.2989865 | 9.13E−09 | 9.82E−05 |
| GPRIN2 | 4.60315306 | 4.796624012 | −0.2991799 | 3.12E−07 | 0.00319752 |
| CARD10 | 4.38091913 | 4.496124041 | −0.299432 | 3.57E−07 | 0.00365725 |
| TMEM28 | 4.29066249 | 4.581733437 | −0.2996032 | 8.57E−12 | 9.70E−08 |
| MCM7 | 7.49338386 | 8.028067249 | −0.2996244 | 7.74E−10 | 8.50E−06 |
| TTK | 6.19991085 | 6.92693319 | −0.2997736 | 1.30E−09 | 1.43E−05 |
| RFC3 | 6.48155974 | 6.963762552 | −0.3001869 | 3.13E−09 | 3.40E−05 |
| HPGD | 4.22065783 | 4.565434045 | −0.3003409 | 2.54E−07 | 0.00261503 |
| DLK2 | 4.73422617 | 4.921981287 | −0.3007456 | 5.91E−08 | 0.00062152 |
| CACYBP | 7.89111906 | 8.1738833 | −0.3007853 | 3.28E−10 | 3.63E−06 |
| C1orf159 | 4.83282639 | 4.978536019 | −0.301082 | 2.48E−09 | 2.70E−05 |
| SCD5 | 5.02515361 | 5.3853242 | −0.3017129 | 1.01E−07 | 0.00105698 |
| C17orf53 | 4.48574919 | 4.624464831 | −0.3017467 | 1.17E−09 | 1.28E−05 |
| CEP72 | 4.90312114 | 5.230737581 | −0.3023087 | 7.82E−08 | 0.00081953 |
| KIAA1166 | 5.66991513 | 6.125926938 | −0.3023993 | 1.75E−09 | 1.90E−05 |
| HYAL1 | 4.3349903 | 4.697976476 | −0.3025633 | 9.77E−07 | 0.00985113 |
| MAGEA9 | 4.05535563 | 4.354124206 | −0.3031307 | 1.19E−10 | 1.33E−06 |
| KRT5 | 4.18880404 | 4.700896291 | −0.3037796 | 2.61E−09 | 2.84E−05 |
| KCNK2 | 4.08298383 | 4.419361093 | −0.303886 | 1.23E−11 | 1.39E−07 |
| ANKRD26 | 4.21447749 | 4.374334061 | −0.304025 | 3.77E−09 | 4.09E−05 |
| DACH1 | 4.45806973 | 4.937991228 | −0.3042156 | 3.25E−07 | 0.00333336 |
| CELSR3 | 5.5677544 | 6.084609031 | −0.3043864 | 6.54E−10 | 7.19E−06 |
| SPATS2 | 6.219552 | 6.615715859 | −0.3044327 | 9.06E−10 | 9.94E−06 |
| WDR62 | 4.83993099 | 5.025755577 | −0.3046244 | 1.62E−09 | 1.76E−05 |
| ALCAM | 8.68001745 | 9.283798814 | −0.3046658 | 1.19E−11 | 1.35E−07 |
| CDT1 | 3.85037902 | 4.140828433 | −0.3047322 | 2.07E−08 | 0.00022004 |
| NTF3 | 4.44841652 | 4.61577322 | −0.3048678 | 2.34E−07 | 0.00241266 |
| POLG2 | 5.95771956 | 6.298241781 | −0.3048804 | 1.01E−08 | 0.0001081 |
| TBPL1 | 7.68062456 | 8.163014418 | −0.305583 | 1.44E−10 | 1.60E−06 |
| SUPV3L1 | 6.26247434 | 6.637012209 | −0.3059631 | 2.35E−09 | 2.56E−05 |
| CYP2E1 | 3.96614254 | 4.077580607 | −0.3063975 | 2.00E−08 | 0.00021273 |
| KIF21B | 5.79747683 | 6.528094755 | −0.3065028 | 4.87E−09 | 5.27E−05 |
| SQLE | 6.51969482 | 6.939888981 | −0.3066743 | 4.96E−08 | 0.00052325 |
| LRRC1 | 6.04561722 | 6.567011047 | −0.3067274 | 9.72E−09 | 0.0001044 |
| MAD2L1 | 7.41781847 | 8.13098634 | −0.3071449 | 8.62E−11 | 9.61E−07 |
| CDC7 | 6.57345823 | 7.228584693 | −0.3073464 | 1.67E−09 | 1.82E−05 |
| RPS6KA5 | 5.4953849 | 5.934861771 | −0.3077396 | 1.86E−10 | 2.06E−06 |
| CSDC2 | 4.90521193 | 5.306529071 | −0.3078094 | 7.27E−09 | 7.84E−05 |
| SPAG5 | 6.12505853 | 6.648811202 | −0.3081752 | 4.55E−09 | 4.93E−05 |
| MMP15 | 4.81070638 | 5.141617295 | −0.3081883 | 5.35E−10 | 5.89E−06 |
| C2orf27 | 4.95155962 | 5.164400612 | −0.3084313 | 1.28E−07 | 0.00133376 |
| C10orf18 | 7.44129532 | 7.879104405 | −0.3086466 | 9.33E−10 | 1.02E−05 |
| TOX3 | 5.80903102 | 6.836237856 | −0.3089275 | 4.10E−10 | 4.52E−06 |
| COL11A2 | 4.3201312 | 4.494817009 | −0.3092466 | 3.45E−11 | 3.87E−07 |
| CDCA8 | 5.99753895 | 6.504795222 | −0.3103547 | 3.08E−09 | 3.35E−05 |
| SETDB1 | 5.69665527 | 5.93714768 | −0.3112541 | 7.75E−11 | 8.65E−07 |
| MYBL2 | 5.27445159 | 5.681791157 | −0.3120067 | 9.02E−09 | 9.70E−05 |
| POLE | 5.27668834 | 5.584311825 | −0.312302 | 5.51E−10 | 6.06E−06 |
| POF1B | 3.97476641 | 4.153455042 | −0.312884 | 5.19E−10 | 5.72E−06 |
| ELMO3 | 3.89447889 | 4.171390332 | −0.3129564 | 2.70E−07 | 0.00277439 |
| CCT3 | 9.66036873 | 9.968333337 | −0.3130012 | 4.91E−11 | 5.50E−07 |
| PRIM1 | 6.890224 | 7.456068802 | −0.3135941 | 2.83E−10 | 3.13E−06 |
| HCRP1 | 4.43612823 | 4.576911681 | −0.3142088 | 2.65E−08 | 0.00028095 |
| RECQL4 | 4.82488854 | 5.049388873 | −0.3154986 | 7.36E−09 | 7.93E−05 |
| ALDH3B2 | 3.75672024 | 3.873228371 | −0.3155876 | 1.09E−07 | 0.0011395 |
| TAS2R1 | 4.5724485 | 4.700868665 | −0.3156005 | 1.71E−09 | 1.87E−05 |
| PODXL2 | 4.94074314 | 5.334138225 | −0.3156862 | 7.75E−11 | 8.65E−07 |
| GADD45G | 5.68441794 | 6.209917978 | −0.3157128 | 2.86E−07 | 0.00293277 |
| MUC4 | 3.89754374 | 4.185785561 | −0.3158114 | 2.74E−11 | 3.08E−07 |
| ODC1 | 10.1994069 | 10.68540768 | −0.3160566 | 6.25E−13 | 7.15E−09 |
| MAST1 | 4.24707252 | 4.433565149 | −0.3163263 | 1.33E−12 | 1.52E−08 |
| TRAF4 | 5.58250005 | 6.004394569 | −0.3168759 | 8.84E−10 | 9.70E−06 |
| DUSP9 | 3.8198459 | 4.00564462 | −0.3175938 | 1.60E−11 | 1.80E−07 |
| TMPRSS4 | 4.01068269 | 4.459783099 | −0.3176593 | 2.28E−08 | 0.00024267 |
| RPRM | 5.49897246 | 6.145234525 | −0.3181966 | 8.55E−10 | 9.38E−06 |
| PCDHA9 | 4.29264098 | 4.653455695 | −0.3185092 | 8.34E−12 | 9.44E−08 |
| CTSE | 4.24200698 | 4.599754671 | −0.3190321 | 8.77E−11 | 9.78E−07 |
| KLRK1 | 4.50181591 | 5.073109494 | −0.3190876 | 4.15E−10 | 4.58E−06 |
| CYP2C9 | 4.054285 | 4.133260251 | −0.3193467 | 1.82E−10 | 2.03E−06 |
| CCNA2 | 5.61424036 | 6.306153678 | −0.3195739 | 5.19E−10 | 5.72E−06 |
| TARBP1 | 6.09088726 | 6.536891136 | −0.3204999 | 3.38E−10 | 3.74E−06 |
| NBLA00301 | 3.80592623 | 3.974548995 | −0.3213186 | 9.58E−12 | 1.08E−07 |
| IDI1 | 7.75051656 | 8.212979916 | −0.3218012 | 3.65E−10 | 4.03E−06 |
| ZBTB5 | 7.29883865 | 7.670005938 | −0.3233937 | 1.33E−10 | 1.48E−06 |
| ATP7B | 4.32458122 | 4.621268821 | −0.3237983 | 2.88E−10 | 3.18E−06 |
| PCDH7 | 4.04281751 | 4.212426799 | −0.3243586 | 5.65E−12 | 6.40E−08 |
| DLX5 | 4.48588568 | 5.386595711 | −0.3250369 | 1.91E−08 | 0.00020402 |
| RAB3B | 4.11269793 | 4.366197114 | −0.3252117 | 4.13E−12 | 4.69E−08 |
| HN1 | 8.62572652 | 9.185577491 | −0.3253813 | 1.64E−11 | 1.85E−07 |
| DDX11 | 5.08889979 | 5.409979403 | −0.3261753 | 3.34E−09 | 3.63E−05 |
| BUB1 | 4.57796877 | 4.921844029 | −0.3268259 | 1.69E−09 | 1.84E−05 |
| CACNA2D2 | 4.68780952 | 5.203302954 | −0.3268312 | 1.72E−10 | 1.92E−06 |
| TNRC4 | 4.68684099 | 4.810202119 | −0.3283205 | 6.94E−10 | 7.62E−06 |
| KIF2C | 5.72792991 | 6.311041099 | −0.3284403 | 2.90E−10 | 3.21E−06 |
| TAF4B | 3.87380909 | 4.037946339 | −0.3284462 | 2.97E−07 | 0.00304702 |
| SKP2 | 5.62386123 | 5.948506366 | −0.3286306 | 5.40E−09 | 5.84E−05 |
| SMPD3 | 3.97746889 | 4.210418829 | 0.3287151 | 3.51E−12 | 3.98E−08 |
| SEPHS1 | 7.16086179 | 7.57749709 | −0.3287861 | 1.76E−11 | 1.98E−07 |
| SUSD5 | 4.40225269 | 5.043484054 | −0.3294778 | 1.65E−07 | 0.00170477 |
| VRK1 | 6.51852766 | 7.076057364 | −0.3297241 | 6.35E−10 | 6.99E−06 |
| RAD51 | 4.95486963 | 5.229666948 | −0.3298757 | 8.70E−08 | 0.00090925 |
| TFAM | 5.96923592 | 6.31875664 | −0.3299647 | 7.86E−09 | 8.47E−05 |
| HR | 4.41048703 | 4.532155474 | −0.3305666 | 1.25E−10 | 1.39E−06 |
| HMG4L | 5.39149723 | 5.732413091 | −0.3312051 | 1.67E−10 | 1.86E−06 |
| TROAP | 5.31508105 | 5.767170817 | −0.3314587 | 3.64E−10 | 4.02E−06 |
| KIF22 | 4.79379197 | 5.111852565 | −0.3322137 | 5.00E−11 | 5.59E−07 |
| PLK4 | 4.88372215 | 5.194094142 | −0.3328563 | 2.03E−10 | 2.25E−06 |
| CDH1 | 4.47965859 | 4.981896895 | −0.3334414 | 5.91E−08 | 0.00062152 |
| MTSS1 | 5.43435669 | 5.616844511 | −0.3351052 | 1.58E−11 | 1.78E−07 |
| C12orf48 | 5.14896453 | 5.5658318 | −0.3351426 | 8.03E−07 | 0.00812554 |
| RLN2 | 3.87506897 | 4.149512854 | −0.3352434 | 7.60E−07 | 0.00770096 |
| AOF2 | 7.42455294 | 7.895433076 | 0.3352731 | 1.44E−13 | 1.65E−09 |
| LIG3 | 4.83083355 | 5.103243712 | −0.3355104 | 2.73E−10 | 3.03E−06 |
| PKP2 | 3.88437433 | 4.071625088 | −0.3360116 | 7.68E−09 | 8.28E−05 |
| RFXAP | 4.37989638 | 4.566832781 | −0.3361698 | 2.20E−10 | 2.44E−06 |
| FDFT1 | 9.20014675 | 9.635928638 | −0.3367695 | 2.62E−11 | 2.95E−07 |
| BCL11B | 4.19426553 | 4.629210807 | −0.3374702 | 6.87E−12 | 7.78E−08 |
| GABRA3 | 3.94439323 | 4.334496142 | −0.3378931 | 7.61E−15 | 8.83E−11 |
| SUV39H2 | 3.74477916 | 3.919708021 | −0.3379363 | 5.54E−11 | 6.19E−07 |
| CABYR | 4.18423827 | 4.568252698 | −0.3379913 | 1.46E−07 | 0.00151616 |
| BCCIP | 6.24525383 | 6.645897406 | −0.3380699 | 7.20E−10 | 7.91E−06 |
| KLRG1 | 4.48613458 | 4.684736949 | −0.338339 | 7.96E−13 | 9.10E−09 |
| HIST1H4C | 9.96443281 | 10.44189717 | −0.3387262 | 1.28E−10 | 1.42E−06 |
| NPR3 | 3.9916951 | 4.296259157 | −0.3388502 | 5.21E−09 | 5.64E−05 |
| MAPK8 | 4.22111219 | 4.371799973 | −0.3391241 | 2.84E−10 | 3.14E−06 |
| SRPK1 | 6.61453785 | 6.941942037 | −0.3394347 | 2.67E−10 | 2.96E−06 |
| ZNF643 | 4.10857461 | 4.390037717 | −0.340097 | 3.73E−12 | 4.23E−08 |
| CENPJ | 4.3391811 | 4.666024256 | −0.3404192 | 3.28E−11 | 3.68E−07 |
| GNG4 | 6.73075094 | 7.474926529 | −0.3406666 | 1.42E−10 | 1.58E−06 |
| BRIP1 | 4.08590962 | 4.377397484 | −0.3408822 | 1.01E−10 | 1.13E−06 |
| CLGN | 4.49080774 | 5.260188445 | −0.3410847 | 1.11E−11 | 1.26E−07 |
| KLRC3 | 4.86449442 | 5.710584237 | −0.3413556 | 5.39E−10 | 5.94E−06 |
| CXXC4 | 5.41801099 | 6.050581791 | −0.3416618 | 3.17E−11 | 3.56E−07 |
| KCNS3 | 5.11032772 | 5.74667505 | −0.3421298 | 8.73E−08 | 0.0009125 |
| DLEU2 | 4.68587535 | 4.92355529 | −0.3430334 | 1.87E−10 | 2.08E−06 |
| REPS2 | 5.03714109 | 5.421542138 | −0.3454852 | 3.98E−10 | 4.39E−06 |
| POLB | 8.07566815 | 8.550668007 | −0.3459186 | 1.16E−11 | 1.31E−07 |
| PTH2R | 3.89686142 | 4.37163476 | −0.3465821 | 3.53E−16 | 4.13E−12 |
| WDR67 | 5.45398288 | 5.839264101 | −0.3466597 | 1.02E−10 | 1.13E−06 |
| C14orf93 | 4.44778109 | 4.653843937 | −0.3473239 | 3.51E−11 | 3.94E−07 |
| NCAPH | 5.44599443 | 5.940709382 | −0.3474032 | 9.41E−11 | 1.05E−06 |
| PYCR1 | 5.99386295 | 6.333746123 | −0.348309 | 5.00E−11 | 5.59E−07 |
| C16orf59 | 4.91402778 | 5.160156015 | −0.3498075 | 2.44E−11 | 2.74E−07 |
| TAF5 | 5.58231589 | 6.03132178 | −0.3500242 | 9.19E−12 | 1.04E−07 |
| FUT1 | 4.39091263 | 4.637428143 | −0.3502888 | 1.16E−09 | 1.27E−05 |
| VIPR2 | 4.63945777 | 5.066920162 | −0.3504226 | 4.84E−14 | 5.58E−10 |
| MYT1 | 4.7187164 | 5.17801262 | −0.3506207 | 2.90E−11 | 3.25E−07 |
| MYCN | 4.74318299 | 4.909473565 | −0.351213 | 3.42E−11 | 3.84E−07 |
| C16orf67 | 4.75028176 | 5.016178753 | −0.3513714 | 2.18E−11 | 2.45E−07 |
| RMND5A | 5.12760139 | 5.396718034 | −0.351757 | 4.59E−10 | 5.06E−06 |
| FABP6 | 4.08675909 | 4.636545017 | −0.3525991 | 2.46E−12 | 2.80E−08 |
| C10orf137 | 5.50504964 | 5.848238967 | −0.3536365 | 4.20E−10 | 4.64E−06 |
| ESPL1 | 5.78315693 | 6.29218383 | −0.35405 | 1.15E−10 | 1.28E−06 |
| HMGB3 | 6.94753053 | 7.491883302 | −0.3549998 | 5.72E−13 | 6.55E−09 |
| NSMCE4A | 6.8345674 | 7.275584912 | −0.3550761 | 2.70E−10 | 2.99E−06 |
| LAD1 | 4.58459493 | 4.827009508 | −0.3553388 | 1.91E−07 | 0.00197489 |
| MNX1 | 5.09819779 | 5.554479092 | −0.355408 | 9.29E−13 | 1.06E−08 |
| CORO2A | 3.95346502 | 4.369588514 | −0.355535 | 1.34E−08 | 0.00014321 |
| ZNF804A | 4.57419836 | 5.252259451 | −0.3562643 | 2.40E−09 | 2.61E−05 |
| HDAC2 | 8.40634781 | 8.896032446 | −0.3566327 | 7.95E−14 | 9.15E−10 |
| CCNF | 4.78395959 | 5.047940272 | −0.3566693 | 7.82E−12 | 8.85E−08 |
| BCOR | 5.05152659 | 5.562571367 | −0.3570126 | 1.79E−12 | 2.04E−08 |
| KLF12 | 4.55833246 | 4.740557214 | 0.3570587 | 1.94E−13 | 2.23E−09 |
| FAM60A | 7.89485757 | 8.650698601 | −0.3579235 | 6.97E−11 | 7.78E−07 |
| SHANK2 | 4.38842561 | 4.585315271 | −0.3581288 | 9.32E−12 | 1.05E−07 |
| ARHGEF16 | 4.4302362 | 4.646541781 | −0.3592009 | 6.60E−10 | 7.25E−06 |
| DAB1 | 4.1470362 | 4.321864684 | −0.3595353 | 7.74E−12 | 8.77E−08 |
| AURKB | 5.44293208 | 5.99378848 | −0.360171 | 6.19E−11 | 6.91E−07 |
| MAP2K6 | 4.90310089 | 5.123787208 | −0.3611141 | 1.11E−12 | 1.27E−08 |
| RALGPS2 | 4.05887596 | 4.220240013 | −0.3624962 | 2.07E−12 | 2.35E−08 |
| KLHL23 | 6.72671695 | 7.296452249 | −0.3626974 | 4.62E−13 | 5.29E−09 |
| NET1 | 5.65811873 | 6.403039275 | −0.3646364 | 3.77E−11 | 4.23E−07 |
| SOX4 | 7.29263107 | 8.086938243 | −0.365573 | 1.57E−13 | 1.80E−09 |
| HOOK1 | 4.52739227 | 4.91084651 | −0.365989 | 6.43E−10 | 7.07E−06 |
| CDC14A | 4.04976316 | 4.134872695 | −0.3667701 | 1.51E−13 | 1.73E−09 |
| CDC45L | 5.43693677 | 6.046907194 | −0.3669751 | 8.43E−11 | 9.40E−07 |
| DCUNID2 | 5.34846682 | 5.748745176 | −0.3670178 | 1.57E−13 | 1.80E−09 |
| PRSS16 | 3.84151846 | 4.126460205 | −0.3671894 | 9.31E−09 | 0.00010008 |
| GSDML | 4.59095671 | 4.912503508 | −0.3688196 | 8.65E−12 | 9.79E−08 |
| PRAME | 4.17487926 | 4.914034866 | −0.3691331 | 6.46E−12 | 7.32E−08 |
| POLQ | 4.60701622 | 4.949032769 | −0.3699411 | 4.54E−12 | 5.14E−08 |
| NEK2 | 4.89403224 | 5.438922687 | −0.3708646 | 3.55E−11 | 3.97E−07 |
| RASL11B | 4.94286859 | 5.833316104 | −0.3713591 | 3.42E−17 | 4.02E−13 |
| FXYD3 | 4.55849702 | 5.246114394 | −0.3724032 | 1.03E−09 | 1.12E−05 |
| C1orf135 | 5.18350814 | 5.585735476 | −0.3724703 | 1.44E−12 | 1.65E−08 |
| SPINK5 | 4.40267198 | 4.807387214 | −0.3730214 | 5.47E−11 | 6.11E−07 |
| DNMT3B | 4.9134054 | 5.354978541 | −0.3739166 | 3.17E−14 | 3.66E−10 |
| ORC6L | 6.82907995 | 7.383543379 | −0.374065 | 3.26E−13 | 3.74E−09 |
| FAM77C | 4.79175204 | 5.267065931 | −0.3761467 | 4.67E−14 | 5.39E−10 |
| EXO1 | 5.23417159 | 5.848234546 | −0.3770214 | 4.23E−12 | 4.80E−08 |
| NR0B1 | 4.18115804 | 5.157453104 | −0.3777606 | 2.70E−08 | 0.00028628 |
| ZNF10 | 4.54795915 | 4.822702894 | −0.3781679 | 3.01E−15 | 3.50E−11 |
| PKMYT1 | 4.31845644 | 4.598016653 | −0.3783144 | 1.24E−13 | 1.42E−09 |
| FUT3 | 3.96774266 | 4.196156629 | −0.3785747 | 1.35E−13 | 1.55E−09 |
| BCL7A | 5.46469299 | 5.999539121 | −0.3790048 | 1.05E−13 | 1.21E−09 |
| H2AFY2 | 5.19387611 | 5.792509783 | −0.3792362 | 2.45E−11 | 2.76E−07 |
| ERBB3 | 5.54018308 | 6.860574452 | −0.3797215 | 6.37E−13 | 7.28E−09 |
| PRSS8 | 3.96817115 | 4.426299041 | −0.3806425 | 8.56E−07 | 0.00866098 |
| CDC25A | 4.97451979 | 5.266584549 | −0.3817818 | 2.39E−13 | 2.75E−09 |
| GPR63 | 4.09328235 | 4.244433749 | −0.3826796 | 1.01E−14 | 1.18E−10 |
| LRRC20 | 5.31632388 | 5.618361559 | −0.3830915 | 2.91E−14 | 3.36E−10 |
| PEO1 | 5.37760605 | 5.785011897 | −0.3843784 | 8.60E−13 | 9.82E−09 |
| ATAD5 | 5.09559636 | 5.384195664 | −0.3847176 | 2.73E−13 | 3.14E−09 |
| C20orf42 | 5.43200084 | 6.491510726 | −0.3891414 | 1.49E−11 | 1.69E−07 |
| RANBP1 | 6.26290641 | 6.570794865 | −0.3891892 | 3.69E−12 | 4.19E−08 |
| RBM35B | 4.17474358 | 4.721773953 | −0.3921572 | 3.05E−07 | 0.00313208 |
| EDG4 | 4.53844632 | 4.791819697 | −0.3923041 | 2.29E−12 | 2.61E−08 |
| LOC81691 | 4.75966624 | 5.325843771 | −0.392889 | 2.43E−15 | 2.83E−11 |
| TRAIP | 4.48856526 | 4.756591415 | −0.3952135 | 1.46E−13 | 1.68E−09 |
| DOK4 | 4.28095214 | 4.473791171 | −0.3971606 | 4.01E−13 | 4.59E−09 |
| RAC3 | 4.29322012 | 4.666326785 | −0.3992155 | 2.60E−12 | 2.96E−08 |
| FGF9 | 4.77434617 | 5.546758078 | −0.4004968 | 2.09E−15 | 2.44E−11 |
| ORC1L | 4.37440922 | 4.753477851 | −0.4022945 | 1.77E−12 | 2.02E−08 |
| MCM10 | 4.23869877 | 4.932611279 | −0.4026766 | 8.06E−15 | 9.35E−11 |
| PASK | 4.74904691 | 5.032778258 | −0.4030846 | 9.47E−18 | 1.12E−13 |
| BCL11A | 5.20878969 | 5.900075041 | −0.4036537 | 2.32E−14 | 2.68E−10 |
| MCM3APAS | 4.78244999 | 5.365650857 | −0.4042739 | 9.42E−17 | 1.11E−12 |
| RBM35A | 3.90030897 | 4.631312748 | −0.4050661 | 6.58E−08 | 0.00069101 |
| ZNF492 | 4.79229123 | 5.055012465 | −0.4065086 | 3.85E−14 | 4.44E−10 |
| CDC25C | 4.36009993 | 4.598585769 | −0.4066299 | 9.27E−12 | 1.05E−07 |
| FANCA | 4.09267496 | 4.295984787 | −0.4119721 | 3.56E−15 | 4.15E−11 |
| RAD54L | 4.95503565 | 5.39871261 | −0.4138446 | 1.59E−13 | 1.83E−09 |
| HOXD3 | 4.16967484 | 4.485322323 | −0.4168655 | 1.94E−13 | 2.23E−09 |
| STMN1 | 7.78423224 | 8.396763879 | −0.4173836 | 1.86E−15 | 2.17E−11 |
| PAK7 | 4.14649546 | 4.842780226 | −0.4183573 | 1.52E−16 | 1.78E−12 |
| TACSTD1 | 3.92961977 | 5.24712316 | −0.4195415 | 5.30E−10 | 5.84E−06 |
| ZNF248 | 5.40816318 | 5.921705089 | −0.4219302 | 3.60E−15 | 4.19E−11 |
| RANBP17 | 4.64960385 | 4.886017322 | −0.4260892 | 2.10E−12 | 2.39E−08 |
| PHF16 | 5.51140384 | 6.135036868 | −0.426982 | 9.58E−17 | 1.12E−12 |
| MYCL1 | 4.87400728 | 5.167656214 | −0.4280878 | 6.40E−15 | 7.43E−11 |
| C1orf106 | 5.55818591 | 6.362082251 | −0.4287467 | 3.76E−15 | 4.37E−11 |
| KIFC1 | 5.64245941 | 6.185653514 | −0.4295965 | 3.62E−15 | 4.21E−11 |
| ZNF74 | 4.4411421 | 4.800743113 | −0.4361066 | 9.98E−18 | 1.18E−13 |
| MYB | 4.12255984 | 4.726463065 | −0.4398156 | 8.46E−19 | 1.00E−14 |
| PLS1 | 4.65049632 | 5.640713117 | −0.455088 | 5.30E−15 | 6.15E−11 |
| PLCB4 | 4.47109356 | 5.187874186 | −0.466277 | 2.79E−24 | 3.34E−20 |
| DNA2L | 4.32554838 | 4.77481105 | −0.472247 | 4.44E−18 | 5.24E−14 |
| CNKSR1 | 4.29908303 | 4.570677232 | −0.4875836 | 1.37E−17 | 1.61E−13 |

TABLE 3
Candidates that interact with CHI3L1 by liquid chromatography-mass spectrometry (LC-MS) assay

| | | |
|---|---|---|
| E9PL22_HUMAN Hypoxia up-regulated protein 1 OS = Homo sapiens | E9PL22 | 105 kDa |
| GN = HYOU1 PE = 2 SV = 1 | (+1) | |
| ENOA_HUMAN Alpha-enolase OS = Homo sapiens GN = ENO1 PE = 1 SV = 2 | P06733 | 47 kDa |
| 1433Z_HUMAN 14-3-3 protein zeta/delta OS = Homo sapiens GN = YWHAZ PE = 1 | P63104 | 28 kDa |
| SV = 1 | | |
| 1433G_HUMAN 14-3-3 protein gamma OS = Homo sapiens GN = YWHAG PE = 1 | P61981 | 28 kDa |
| SV = 2 | | |
| 1433E_HUMAN 14-3-3 protein epsilon OS = Homo sapiens GN = YWHAE PE = 1 | P62258 | 29 kDa |
| SV = 1 | | |
| LG3BP_HUMAN Galectin-3-binding protein OS = Homo sapiens GN = LGALS3BP | Q08380 | 65 kDa |
| PE = 1 SV = 1 | | |
| TFR1_HUMAN Transferrin receptor protein 1 OS = Homo sapiens GN = TFRC | P02786 | 85 kDa |
| PE = 1 SV = 2 | | |
| BAP31_HUMAN B-cell receptor-associated protein 31 OS = Homo sapiens | P51572 | 28 kDa |
| GN = BCAP31 PE = 1 SV = 3 | (+1) | |
| J3QRD1_HUMAN Fatty aldehyde dehydrogenase OS = Homo sapiens | J3QRD1 | 45 kDa |
| GN = ALDH3A2 PE = 2 SV = 1 | (+2) | |
| ANXA1_HUMAN Annexin A1 OS = Homo sapiens GN = ANXA1 PE = 1 SV = 2 | P04083 | 39 kDa |
| G3P_HUMAN Glyceraldehyde-3-phosphate dehydrogenase OS = Homo sapiens | P04406 | 36 kDa |
| GN = GAPDH PE = 1 SV = 3 | (+1) | |
| PRS10_HUMAN 26S protease regulatory subunit 10B OS = Homo sapiens | P62333 | 44 kDa |
| GN = PSMC6 PE = 1 SV = 1 | | |
| C1QBP_HUMAN Complement component 1 Q subcomponent-binding protein, | Q07021 | 31 kDa |
| mitochondrial OS = Homo sapiens GN = C1QBP PE = 1 SV = 1 | | |
| PRAF3_HUMAN PRA1 family protein 3 OS = Homo sapiens GN = ARL6IP5 PE = 1 | O75915 | 22 kDa |
| SV = 1 | | |
| SC61B_HUMAN Protein transport protein Sec61 subunit beta OS = Homo sapiens | P60468 | 10 kDa |
| GN = SEC61B PE = 1 SV = 2 | | |
| STML2_HUMAN Stomatin-like protein 2, mitochondrial OS = Homo sapiens | Q9UJZ1 | 39 kDa |
| GN = STOML2 PE = 1 SV = 1 | | |
| M2OM_HUMAN Mitochondrial 2-oxoglutarate/malate carrier protein OS = Homo | Q02978 | 34 kDa |
| sapiens GN = SLC25A11 PE = 1 SV = 3 | | |
| PGAM5_HUMAN Isoform 2 of Serine/threonine-protein phosphatase PGAM5, | Q96HS1-2 | 28 kDa |
| mitochondrial OS = Homo sapiens GN = PGAM5 | | |
| 1433F_HUMAN 14-3-3 protein eta OS = Homo sapiens GN = YWHAH PE = 1 SV = 4 | Q04917 | 28 kDa |
| SRPRB_HUMAN Signal recognition particle receptor subunit beta OS = Homo | Q9Y5M8 | 30 kDa |
| sapiens GN = SRPRB PE = 1 SV = 3 | | |
| TSN_HUMAN Translin OS = Homo sapiens GN = TSN PE = 1 SV = 1 | Q15631 | 26 kDa |
| MARCS_HUMAN Myristoylated alanine-rich C-kinase substrate OS = Homo | P29966 | 32 kDa |
| sapiens GN = MARCKS PE = 1 SV = 4 | | |

TABLE 4
Oligonucleotides

| Gene | Forward Seq | Reverse Seq | Source |
|---|---|---|---|
| Human | | | |
| RPL39 | CAGCTTCCCTC | GCCAGGAATCG | Sigma- |
| | CTCTTCCTT | CTTAATCC | Aldrich |
| | (SEQ ID | (SEQ ID | |
| | NO: 10) | NO: 11) | |
| CHI3L1 | GTGAAGGCGTC | CTTCCCGGTA | Sigma- |
| | TCAAACAGG | CTGGGACCA | Aldrich |
| | (SEQ ID | (SEQ ID | |
| | NO: 12) | NO: 13) | |
| Mouse | | | |
| Chi3l1 | GTACAAGCTGG | ATGTGCTAAG | Sigma- |
| | TCTGCTACTTC | CATGTTGTCGC | Aldrich |
| | (SEQ ID | (SEQ ID | |
| | NO: 14) | NO: 15) | |
| Lgals3bp | TGCTGGTTCCAG | CCACCGGCCT | Sigma- |
| | GGACTCAA | CTGTAGAAGA | Aldrich |
| | (SEQ ID | (SEQ ID | |
| | NO: 16) | NO: 17) | |
| Lgals3 | AGACAGCTTTT | GGGTAGGCACT | Sigma- |
| | CGCTTAACGA | AGGAGGAGC | Aldrich |
| | (SEQ ID | (SEQ ID | |
| | NO: 18) | NO: 19) | |
| Commercial | | | GeneGlobe |
| Primer | Source | Cat# | ID |
| Mouse | | | |
| 18S rRNA | QIAGEN | 249900 | QT02448075 |
| Ccl2 | QIAGEN | 249900 | QT00167832 |
| Arg1 | QIAGEN | 249900 | QT00134288 |
| Chil3 (Ym1) | QIAGEN | 249900 | QT00108829 |
| Il-10 | QIAGEN | 249900 | QT00106169 |
| Nos2 | QIAGEN | 249900 | QT00068740 |
| IL-1β | QIAGEN | 249900 | QT01048355 |

### REFERENCES

Wen P Y, and Kesari S. Malignant gliomas in adults. N Engl J Med. 2008; 359(5):492-507.
Desjardins A. Neuro-oncology: What is the optimal use of bevacizumab in glioblastoma? Nat Rev Neurol. 2015.
Fumnari F B, Fenton T, Bachoo R M V, Mukasa A, Stommel J M, Stegh A, et al. Genes Dev. 2007:2683-710.
Buerki R A, Chheda Z S, and Okada H. Immunotherapy of Primary Brain Tumors: Facts and Hopes. Clin Cancer Res. 2018; 24(21):5198-205.
Lim M, Xia Y, Bettegowda C, and Weller M. Current state of immunotherapy for glioblastoma. Nat Rev Clin Oncol. 2018; 15(7):422-42.
Sampson J H, Gunn M D, Fecci P E, and Ashley D M. Brain immunology and immunotherapy in brain tumours. Nat Rev Cancer. 2020; 20(1):12-25.
Hambardzumyan D, Gutmann D H, and Kettenmann H. The role of microglia and macrophages in glioma maintenance and progression. Nat Neurosci. 2016; 19(1):20-7.
Ginhoux F, Greter M, Leboeuf M, Nandi S, See P, Gokhan S, et al. Fate mapping analysis reveals that adult microglia derive from primitive macrophages. Science. 2010; 330(6005):841-5.
Chen Z, Feng X, Herting C J, Garcia V A, Nie K, Pong W W, et al. Cellular and Molecular Identity of Tumor-Associated Macrophages in Glioblastoma. Cancer Res. 2017; 77(9):2266-78.
Pinton L, Masetto E, Vettore M, Solito S, Magri S, D'Andolfi M, et al. The immune suppressive microenvironment of human gliomas depends on the accumulation of bone marrow-derived macrophages in the center of the lesion. J Immunother Cancer. 2019; 7(1):58.
Komohara Y, Ohnishi K, Kuratsu J, and Takeya M. Possible involvement of the M2 anti-inflammatory macrophage phenotype in growth of human gliomas. J Pathol. 2008; 216(1):15-24.
Wang Q, Hu B, Hu X, Kim H, Squatrito M, Scarpace L, et al. Tumor Evolution of Glioma-Intrinsic Gene Expression Subtypes Associates with Immunological Changes in the Microenvironment. Cancer Cell. 2017; 32(1):42-56 e6.
Quail D F, and Joyce J A. The Microenvironmental Landscape of Brain Tumors. Cancer Cell. 2017; 31(3):326-41.
Hu B, Wang Q, Wang Y A, Hua S, Sauve C G, Ong D, et al. Epigenetic Activation of WNT5A Drives Glioblastoma Stem Cell Differentiation and Invasive Growth. Cell. 2016; 167(5):1281-95 e18.
Fusetti F, Pijning T, Kalk K H, Bos E, and Dijkstra B W. Crystal structure and carbohydrate-binding properties of the human cartilage glycoprotein-39. J Biol Chem. 2003; 278(39):37753-60.
Lee C G, Da Silva C A, Dela Cruz C S, Ahangari F, Ma B, Kang M J, et al. Role of chitin and chitinase/chitinase-like proteins in inflammation, tissue remodeling, and injury. Annu Rev Physiol. 2011; 73:479-501.
Iwamoto F M, Hottinger A F, Karimi S, Riedel E, Dantis J, Jahdi M, et al. Serum YKL-40 is a marker of prognosis and disease status in high-grade gliomas. Neuro Oncol. 2011; 13(11):1244-51.
Pelloski C E, Mahajan A, Maor M, Chang E L, Woo S, Gilbert M, et al. YKL-40 expression is associated with poorer response to radiation and shorter overall survival in glioblastoma. Clin Cancer Res. 2005; 11(9):3326-34.
Darmanis S, Sloan S A, Croote D, Mignardi M, Chernikova S, Samghababi P, et al. Single-Cell RNA-Seq Analysis of Infiltrating Neoplastic Cells at the Migrating Front of Human Glioblastoma. Cell Rep. 2017; 21(5):1399-410.
Shingu T, Ho A L, Yuan L, Zhou X, Dai C, Zheng S, et al. Qki deficiency maintains stemness of glioma stem cells in suboptimal environment by downregulating endolysosomal degradation. Nat Genet. 2016.
Genoud V, Marinari E, Nikolaev S I, Castle J C, Bukur V, Dietrich P Y, et al. Responsiveness to anti-PD-1 and anti-CTLA-4 immune checkpoint blockade in SB28 and GL261 mouse glioma models. Oncoimmunology. 2018; 7(12):e1501137.
Kawada M, Seno H, Kanda K, Nakanishi Y, Akitake R, Komekado H, et al. Chitinase 3-like 1 promotes macrophage recruitment and angiogenesis in colorectal cancer. Oncogene. 2012; 31(26):3111-23.
Xu N, Bo Q, Shao R, Liang J, Zhai Y, Yang S, et al. Chitinase-3-Like-1 Promotes M2 Macrophage Differentiation and Induces Choroidal Neovascularization in Neovascular Age-Related Macular Degeneration. Invest Ophthalmol Vis Sci. 2019; 60(14):4596-605.
Haynes S E, Hollopeter G, Yang G, Kurpius D, Dailey M E, Gan W B, et al. The P2Y12 receptor regulates microglial activation by extracellular nucleotides. Nat Neurosci. 2006; 9(12):1512-9.
Butovsky O, Jedrychowski M P, Moore C S, Cialic R, Lanser A J, Gabriely G, et al. Identification of a unique TGF-beta-dependent molecular and functional signature in microglia. Nat Neurosci. 2014; 17(1):131-43.
Ying W, Cheruku P S, Bazer F W, Safe S H, and Zhou B. Investigation of macrophage polarization using bone marrow derived macrophages. J Vis Exp. 2013(76).
Chen B, Khodadoust M S, Liu C L, Newman A M, and Alizadeh A A. Profiling Tumor Infiltrating Immune Cells with CIBERSORT. Methods Mol Biol. 2018; 1711:243-59.
Muller S, Kohanbash G, Liu S J, Alvarado B, Carrera D, Bhaduri A, et al. Single-cell profiling of human gliomas reveals macrophage ontogeny as a basis for regional differences in macrophage activation in the tumor microenvironment. Genome Biol. 2017; 18(1):234.
Loimaranta V, Hepojoki J, Laaksoaho O, and Pulliainen A T. Galectin-3-binding protein: A multitask glycoprotein with innate immunity functions in viral and bacterial infections. J Leukoc Biol. 2018; 104(4):777-86.
MacKinnon A C, Famworth S L, Hodkinson P S, Henderson N C, Atkinson K M, Leffler H, et al. Regulation of alternative macrophage activation by galectin-3. J Immunol. 2008; 180(4):2650-8.
Sano H, Hsu D K, Yu L, Apgar J R, Kuwabara I, Yamanaka T, et al. Human galectin-3 is a novel chemoattractant for monocytes and macrophages. J Immunol. 2000; 165(4):2156-64.
Inohara H, Akahani S, Koths K, and Raz A. Interactions between galectin-3 and Mac-2-binding protein mediate cell-cell adhesion. Cancer Res. 1996; 56(19):4530-4.
Champ P C, and Camacho C J. FastContact: a free energy scoring tool for protein-protein complex structures. Nucleic Acids Res. 2007; 35(Web Server issue):W556-60.
Pyonteck S M, Akkari L, Schuhmacher A J, Bowman R L, Sevenich L, Quail D F, et al. CSF-1R inhibition alters macrophage polarization and blocks glioma progression. Nat Med. 2013; 19(10):1264-72.
Kaneda M M, Messer K S, Ralainirina N, Li H, Leem C J, Gorjestani S, et al. PI3Kgamma is a molecular switch that controls immune suppression. Nature. 2016; 539(7629):437-42.
van Rooijen N, Kors N, ter Hart H, and Claassen E. In vitro and in vivo elimination of macrophage tumor cells using liposome-encapsulated dichloromethylene diphosphonate. Virchows Arch B Cell Pathol Incl Mol Pathol. 1988; 54(4):241-5.
Fulci G, Dmitrieva N, Gianni D, Fontana E J, Pan X, Lu Y, et al. Depletion of peripheral macrophages and brain microglia increases brain tumor titers of oncolytic viruses. Cancer Res. 2007; 67(19):9398-406.
Woroniecka K, Chongsathidkiet P, Rhodin K, Kemeny H, Dechant C, Farber S H, et al. T-Cell Exhaustion Signatures Vary with Tumor Type and Are Severe in Glioblastoma. Clin Cancer Res. 2018; 24(17):4175-86.
Medikonda R, Dunn G, Rahman M, Fecci P, and Lim M. A review of glioblastoma immunotherapy. J Neurooncol. 2020.
Topalian S L, Drake C G, and Pardoll D M. Immune checkpoint blockade: a common denominator approach to cancer therapy. Cancer Cell. 2015; 27(4):450-61.
Zhao J, Chen A X, Gartrell R D, Silverman A M, Aparicio L, Chu T, et al. Immune and genomic correlates of response to anti-PD-1 immunotherapy in glioblastoma. Nat Med. 2019; 25(3):462-9.
Wellenstein M D, and de Visser K E. Cancer-Cell-Intrinsic Mechanisms Shaping the Tumor Immune Landscape. Immunity. 2018; 48(3):399-416.
Novak R, Dabelic S, and Dumic J. Galectin-1 and galectin-3 expression profiles in classically and alternatively activated human macrophages. Biochim Biophys Acta. 2012; 1820(9):1383-90.
Gleissner C A, Erbel C, Linden F, Domschke G, Akhavanpoor M, Helmes C M, et al. Galectin-3 binding protein, coronary artery disease and cardiovascular mortality: Insights from the LURIC study. Atherosclerosis. 2017; 260:121-9.
Shaked I, Hanna D B, Gleissner C, Marsh B, Plants J, Tracy D, et al. Macrophage inflammatory markers are associated with subclinical carotid artery disease in women with human immunodeficiency virus or hepatitis C virus infection. Arterioscler Thromb Vasc Biol. 2014; 34(5):1085-92.
Tomaszewski W, Sanchez-Perez L, Gajewski T F, and Sampson J H. Brain Tumor Microenvironment and Host State: Implications for Immunotherapy. Clin Cancer Res. 2019; 25(14):4202-10.
Jackson C M, Choi J, and Lim M. Mechanisms of immunotherapy resistance: lessons from glioblastoma. Nat Immunol. 2019; 20(9):1100-9.
Jallal B, Powell J, Zachwieja J, Brakebusch C, Germain L, Jacobs J, et al. Suppression of tumor growth in vivo by local and systemic 90K level increase. Cancer Res. 1995; 55(15):3223-7.
Ullrich A, Sures I, D'Egidio M, Jallal B, Powell T J, Herbst R, et al. The secreted tumor-associated antigen 90K is a potent immune stimulator. J Biol Chem. 1994; 269(28):18401-7.
Kalayci O, Birben E, Tinari N, Oguma T, Iacobelli S, and Lilly C M. Role of 90K protein in asthma and TH2-type cytokine expression. Ann Allergy Asthma Immunol. 2004; 93(5):485-92.
Mitsialis V, Wall S, Liu P, Ordovas-Montanes J, Parmet T, Vukovic M, et al. Single-Cell Analyses of Colon and Blood Reveal Distinct Immune Cell Signatures of Ulcerative Colitis and Crohn's Disease. Gastroenterology. 2020; 159(2):591-608 e10.
Comeau S R, Gatchell D W, Vajda S, and Camacho C J. ClusPro: a fully automated algorithm for protein-protein docking. Nucleic Acids Res. 2004; 32(Web Server issue):W96-9.
Salomon-Ferrer R, Gotz A W, Poole D, Le Grand S, and Walker R C. Routine Microsecond Molecular Dynamics Simulations with AMBER on GPUs. 2. Explicit Solvent Particle Mesh Ewald. J Chem Theory Comput. 2013; 9(9):3878-88.
Maier J A, Martinez C, Kasavajhala K, Wickstrom L, Hauser K E, and Simmerling C. ff14SB: Improving the Accuracy of Protein Side Chain and Backbone Parameters from ff99SB. J Chem Theory Comput. 2015; 11(8):3696-713.
Buerki, R. A., Chheda, Z. S., and Okada, H. (2018). Immunotherapy of Primary Brain Tumors: Facts and Hopes. Clin Cancer Res 24, 5198-5205.
Chen, H., Lau, M. C., Wong, M. T., Newell, E. W., Poidinger, M., and Chen, J. (2016). Cytofkit: A Bioconductor Package for an Integrated Mass Cytometry Data Analysis Pipeline. PLoS Comput Biol 12, e1005112.
Fusetti, F., Pijning, T., Kalk, K. H., Bos, E., and Dijkstra, B. W. (2003). Crystal structure and carbohydrate-binding properties of the human cartilage glycoprotein-39. J Biol Chem 278, 37753-37760.
Gotz, A. W., Williamson, M. J., Xu, D., Poole, D., Le Grand, S., and Walker, R. C. (2012). Routine Microsecond Molecular Dynamics Simulations with AMBER on GPUs. 1. Generalized Born. J Chem Theory Comput 8, 1542-1555
Kamba, A., Lee, I. A., and Mizoguchi, E. (2013). Potential association between TLR4 and chitinase 3-like 1 (CHI3L1/YKL-40) signaling on colonic epithelial cells in inflammatory bowel disease and colitis-associated cancer. Curr Mol Med 13, 1110-1121.
Kozakov, D., Hall, D. R., Xia, B., Porter, K. A., Padhomy, D., Yueh, C., Beglov, D., and Vajda, S. (2017). The ClusPro web server for protein-protein docking. Nat Protoc 12, 255-278.
Lal, S., Lacroix, M., Tofilon, P., Fuller, G. N., Sawaya, R., and Lang, F. F. (2000). An implantable guide-screw system for brain tumor studies in small animals. J Neurosurg 92, 326-333
Lewis, G. D., and Metcalf, T. G. (1988). Polyethylene glycol precipitation for recovery of pathogenic viruses, including hepatitis A virus and human rotavirus, from oyster, water, and sediment samples. Appl Environ Microbiol 54, 1983-1988.
Luttrell, L. M., Ferguson, S. S., Daaka, Y., Miller, W. E., Maudsley, S., Della Rocca, G. J., Lin, F., Kawakatsu, H., Owada, K., Luttrell, D. K., et al. (1999). Beta-arrestin-dependent formation of beta2 adrenergic receptor-Src protein kinase complexes. Science 283, 655-661.
Thakur, D., Rejtar, T., Wang, D., Bones, J., Cha, S., Clodfelder-Miller, B., Richardson, E., Binns, S., Dahiya, S., Sgroi, D., et al. (2011). Microproteomic analysis of 10,000 laser captured microdissected breast tumor cells using short-range sodium dodecyl sulfate-polyacrylamide gel electrophoresis and porous layer open tubular liquid chromatography tandem mass spectrometry. J Chromatogr A 1218, 8168-8174
Van Gassen, S., Callebaut, B., Van Helden, M. J., Lambrecht, B. N., Demeester, P., Dhaene, T., and Saeys, Y. (2015). FlowSOM: Using self-organizing maps for visualization and interpretation of cytometry data. Cytometry A 87, 636-645
Yushkevich, P. A., Yang, G., and Gerig, G. (2016). ITK-SNAP: An interactive tool for semi-automatic segmentation of multi-modality biomedical images. Annu Int Conf IEEE Eng Med Biol 655 Soc 2016, 3342-3345

1. A composition comprising a Galectin-3 (Gal3)-binding protein (Gal3BP) polypeptide, wherein the Gal3BP polypeptide sequence is at least 80% identical to SEQ ID NO: 4 or SEQ ID NO: 5.

2. The composition of claim 1, wherein the Gal3BP polypeptide sequence is at least 95% identical to SEQ ID NO: 4.

3. The composition of claim 1, wherein the Gal3BP polypeptide has the sequence of SEQ ID NO: 4.

4. (canceled)

5. (canceled)

6. (canceled)

7. A method for treating a cancer in a subject comprising administering to the subject a therapeutically effective amount of a Galectin-3 (Gal3)-binding protein (Gal3BP) polypeptide, wherein the Gal3BP polypeptide comprises a sequence at least 80% identical to SEQ ID NO: 4 or SEQ ID NO: 5.

8. The method of claim 7, wherein the Gal3BP polypeptide comprises a sequence at least 95% identical to SEQ ID NO: 4.

9. The method of claim 7, wherein the Gal3BP polypeptide comprises the sequence of SEQ ID NO: 4.

10. The method of claim 7, wherein the subject is determined to have
a) a higher level of a CHI3L1 polypeptide and/or a Gal3 polypeptide compared to a first and/or second reference control, and/or
b) a lower level of a Gal3BP polypeptide compared to a third reference control.

11. The method of claim 7, wherein administration of the Gal3BP polypeptide decreases a level of an immune checkpoint polypeptide on an immune cell or a tumor cell.

12. The method of claim 11, wherein the immune checkpoint polypeptide is selected from the group consisting of PD-1, PD-L1, PD-L2, and CTLA-4.

13. The method of claim 7, further comprising administering to the subject a therapeutically effective amount of an immune checkpoint inhibitor.

14. The method of claim 13, wherein the immune checkpoint inhibitor is selected from a PD-1 inhibitor, a PD-L1 inhibitor, and a CLTA-4 inhibitor.

15. (canceled)

16. The method of claim 14, wherein the immune checkpoint inhibitor is selected from nivolumab, pembrolizumab, cemiplimab, atezolizumab, avelumab, durvalumab, and ipilimumab.

17. The method of claim 7, wherein the cancer is a glioblastoma.

18. A method for identifying a subject's responsiveness to an immune checkpoint inhibitor, said method comprising
a) obtaining a biological sample from the subject;
b) quantifying a level of a biomarker relative to a reference control, wherein the biomarker is selected from a CHI3L1 polypeptide, a Galectin-3 (Gal3) polypeptide, and a Galectin-3(Gal3)-binding protein (Gal3BP) polypeptide; and
c) determining the subject as responsive to the immune checkpoint inhibitor when the level of one or more of the CHI3L1 polypeptide or the Gal3 polypeptide is lower in the biological sample than its reference control, or the level of the Gal3BP polypeptide is higher in the biological sample than its reference control, or a combination thereof; or
d) determining the subject as non-responsive to the immune checkpoint therapy when the level of one or more of the CHI3L1 polypeptide or the Gal3 polypeptide is higher in the biological sample than its reference control, or the level of the Gal3BP polypeptide is lower in the biological sample than its reference control, or a combination thereof.

19. The method of claim 18, further comprising administering to the subject responsive to the immune checkpoint inhibitor a therapeutically effective amount of the immune checkpoint inhibitor.

20. The method of claim 18, further comprising administering to the subject non-responsive to the immune checkpoint inhibitor a therapeutically effective amount of a Gal3PB polypeptide, wherein the Gal3BP polypeptide comprises a sequence at least 80% identical to SEQ ID NO: 4 or SEQ ID NO: 5.

21. The method of claim 20, further comprising subsequently administering to the subject a therapeutically effective amount of the immune checkpoint inhibitor.

22. The method of claim 18, wherein the immune checkpoint inhibitor is selected from a PD-1 inhibitor, a PD-L1 inhibitor, and a CLTA-4 inhibitor.

23. (canceled)

24. The method of claim 18, wherein the immune checkpoint inhibitor is selected from nivolumab, pembrolizumab, cemiplimab, atezolizumab, avelumab, durvalumab, and ipilimumab.

25. The method of claim 18, wherein the biological sample is selected from serum, plasma, whole blood, cerebrospinal fluid (CSF), and tumor tissue.

26. The method of claim 18, wherein the subject has a brain cancer.

27. (canceled)


SEQUENCE LISTING (20 sequences)

SEQ ID NO: 1 - protein, 250 aa, Homo sapiens [sequence omitted]

SEQ ID NO: 2 - protein, 585 aa, Homo sapiens
MTPPRLFWVWLLVAGTQGVNDGDMRLADGGATNQGRVEIFYRGQWGTVCDNLWDLTDASVVCRALGFENATQALGRAAFGQGSGPIMLDEVQCTGTEASLADCKSLGWLKSNCRHERDAGVVCTNETRSTHTLDLSRELSEALGQIFDSQRGCDLSISVNVQGEDALGFCGHTVILTANLEAQALWKEPGSNVTMSVDAECVPMVRDLLRYFYSRRIDITLSSVKCFHKLASAYGARQLQGYCASLFAILLPQDPSFQMPLDLYAYAVATGDALLEKLCLQFLAWNFEALTQAEAWPSVPTDLLQLLLPRSDLAVPSELALLKAVDTWSWGERASHEEVEGLVEKIRFPMMLPEELFELQFNLSLYWSHEALFQKKTLQALEFHTVPFQLLARYKGLNLTEDTYKPRIYTSPTWSAFVTDSSWSARKSQLVYQSRRGPLVKYSSDYFQAPSDYRYYPYQSFQTPQHPSFLFQDKRVSWSLVYLPTIQSCWNYGFSCSSDELPVLGLTKSGGSDRTIAYENKALMLCEGLFVADVTDFEGWKAAIPSALDTNSSKSTSSFPCPAGHFNGFRTVIRPFYLTNSSGVD

SEQ ID NO: 3 - protein, 383 aa, Homo sapiens
MGVKASQTGFVVLVLLQCCSAYKLVCYYTSWSQYREGDGSCFPDALDRFLCTHIIYSFANISNDHIDTWEWNDVTLYGMLNTLKNRNPNLKTLLSVGGWNFGSQRFSKIASNTQSRRTFIKSVPPFLRTHGFDGLDLAWLYPGRRDKQHFTTLIKEMKAEFIKEAQPGKKQLLLSAALSAGKVTIDSSYDIAKISQHLDFISIMTYDFHGAWRGTTGHHSPLFRGQEDASPDRFSNTDYAVGYMLRLGAPASKLVMGIPTFGRSFTLASSETGVGAPISGPGIPGRFTKEAGTLAYYEICDFLRGATVHRILGQQVPYATKGNQWVGYDDQESVKSKVQYLKDRQLAGAMVWALDLDDFQGSFCGQDLRFPLTNAIKDALAAT

SEQ ID NO: 4 - protein, 15 aa, Artificial Sequence
  Synthetic construct
TLDLSRELSEALGQI

SEQ ID NO: 5 - protein, 15 aa, Artificial Sequence
  Synthetic construct
TRSTHTLDLSRELSE

SEQ ID NO: 6 - protein, 223 aa, Homo sapiens
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSKMLKKRSPLTTGVYVKMPPTEPECEKQFQPYFIPIN

SEQ ID NO: 7 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 8 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 9 - protein, 273 aa, Homo sapiens
MIFLLLMLSLELQLHQIAALFTVTVPKELYIIEHGSNVTLECNFDTGSHVNLGAITASLQKVENDTSPHRERATLLEEQLPLGKASFHIPQVQVRDEGQYQCIIIYGVAWDYKYLTLKVKASYRKINTHILKVPETDEVELTCQATGYPLAEVSWPNVSVPANTSHSRTPEGLYQVTSVLRLKPPPGRNFSCVFWNTHVRELTLASIDLQSQMEPRTHPTWLLHIFIPFCIIAFIFIATVIALRKQLCQKLYSSKDTTKRPVTTTKREVNSAI

SEQ ID NO: 10 - DNA, 20 bp, Artificial Sequence
  Synthetic construct
cagcttccctcctcttcctt

SEQ ID NO: 11 - DNA, 19 bp, Artificial Sequence
  Synthetic construct
gccaggaatcgcttaatcc

SEQ ID NO: 12 - DNA, 20 bp, Artificial Sequence
  Synthetic construct
gtgaaggcgtctcaaacagg

SEQ ID NO: 13 - DNA, 19 bp, Artificial Sequence
  Synthetic construct
cttcccggtactgggacca

SEQ ID NO: 14 - DNA, 22 bp, Artificial Sequence
  Synthetic construct
gtacaagctggtctgctacttc

SEQ ID NO: 15 - DNA, 21 bp, Artificial Sequence
  Synthetic construct
atgtgctaagcatgttgtcgc

SEQ ID NO: 16 - DNA, 20 bp, Artificial Sequence
  Synthetic construct
tgctggttccagggactcaa

SEQ ID NO: 17 - DNA, 20 bp, Artificial Sequence
  Synthetic construct
ccaccggcctctgtagaaga

SEQ ID NO: 18 - DNA, 21 bp, Artificial Sequence
  Synthetic construct
agacagcttttcgcttaacga

SEQ ID NO: 19 - DNA, 20 bp, Artificial Sequence
  Synthetic construct
gggtaggcactaggaggagc

SEQ ID NO: 20 - protein, 15 aa, Artificial Sequence
  Synthetic construct
LRTRLEETLSSDTSH